(19) United States
(12) Patent Application Publication
(10) Pub. No.: US 2014/0256011 A1
(43) Pub. Date: Sep. 11, 2014

(54) Method for Acetate Consumption During Ethanolic Fermentaion of Cellulosic Feedstocks

(71) Applicant: Mascoma Corporation, Lebanon, NH (US)

(72) Inventors: Rintze Meindert ZELLE, Hanover, NH (US); Arthur J. Shaw, IV, Grantham, NH (US); Johannes Pieter Van Dijken, Schiedam (NL)

(21) Appl. No.: 14/075,846

(22) Filed: Nov. 8, 2013

Related U.S. Application Data
(60) Provisional application No. 61/793,716, filed on Mar. 15, 2013.
(60) Provisional application No. 61/724,831, filed on Nov. 9, 2012.

(51) Int. Cl. C12N 15/81; C12P 7/06

(52) U.S. Cl. CPC C12N 15/81; C12P 7/06
USPC 435/161; 435/254.2

(57) ABSTRACT

The present invention provides for novel metabolic pathways to detoxify biomass-derived acetate via metabolic conversion to ethanol, acetone, or isopropanol. More specifically, the invention provides for a recombinant microorganism comprising one or more native and/or heterologous enzymes that function in one or more first engineered metabolic pathways to achieve: (1) conversion of acetate to ethanol; (2) conversion of acetate to acetone; or (3) conversion of acetate to isopropanol; and one or more native and/or heterologous enzymes that function in one or more second engineered metabolic pathways to produce an electron donor used in the conversion of acetate to less inhibitory compounds; wherein the one or more native and/or heterologous enzymes is activated, upregulated, or downregulated.

### REFERENCE TO RELATED APPLICATIONS

Related applications U.S. 61/724,831, filed on Nov. 9, 2012, and 61/793,716, filed on Mar. 15, 2013, are herein incorporated by reference in their entireties.

### REFERENCE TO SEQUENCE LISTING SUBMITTED ELECTRONICALLY VIA EFS-WEB

The content of the electronically submitted sequence listing (Name: 2608_{—}0670002_US_SequenceListing_ascii.txt; Size: 189,173 bytes; and Date of Creation: Nov. 8, 2013) is incorporated by reference in its entirety.

### BACKGROUND OF THE INVENTION

Energy conversion, utilization and access underlie many of the great challenges of our time, including those associated with sustainability, environmental quality, security, and poverty. New applications of emerging technologies are required to respond to these challenges. Biotechnology, one of the most powerful of the emerging technologies, can give rise to important new energy conversion processes. Plant biomass and derivatives thereof are a resource for the biological conversion of energy to forms useful to humanity.

Among forms of plant biomass, lignocellulosic biomass (“biomass”) is particularly well-suited for energy applications because of its large-scale availability, low cost, and environmentally benign production. In particular, many energy production and utilization cycles based on cellulosic biomass have near-zero greenhouse gas emissions on a life-cycle basis. The primary obstacle impeding the more widespread production of energy from biomass feedstocks is the general absence of low-cost technology for overcoming the recalcitrance of these materials to conversion into useful products. Lignocellulosic biomass contains carbohydrate fractions (e.g., cellulose and hemicellulose) that can be converted into ethanol or other products such as lactic acid and acetic acid. In order to convert these fractions, the cellulose and hemicellulose must ultimately be converted or hydrolyzed into monosaccharides; it is the hydrolysis that has historically proven to be problematic.

Biologically mediated processes are promising for energy conversion. Biomass processing schemes involving enzymatic or microbial hydrolysis commonly involve four biologically mediated transformations: (1) the production of saccharolytic enzymes (cellulases and hemicellulases); (2) the hydrolysis of carbohydrate components present in pretreated biomass to sugars; (3) the fermentation of hexose sugars (e.g., glucose, mannose, and galactose); and (4) the fermentation of pentose sugars (e.g., xylose and arabinose). These four transformations occur in a single step in a process configuration called consolidated bioprocessing (CBP), which is distinguished from other less highly integrated configurations in that it does not involve a dedicated process step for cellulase and/or hemicellulase production.

CBP offers the potential for lower cost and higher efficiency than processes featuring dedicated cellulase production. The benefits result in part from avoided capital costs, substrate and other raw materials, and utilities associated with cellulase production. In addition, several factors support the realization of higher rates of hydrolysis, and hence reduced reactor volume and capital investment using CBP, including enzyme-microbe synergy and the use of thermophilic organisms and/or complexed cellulase systems. Moreover, cellulose-adherent cellulolytic microorganisms are likely to compete successfully for products of cellulose hydrolysis with non-adhered microbes, e.g., contaminants, which could increase the stability of industrial processes based on microbial cellulose utilization. Progress in developing CBP-enabling microorganisms is being made through two strategies: engineering naturally occurring cellulolytic microorganisms to improve product-related properties, such as yield and titer; and engineering non-cellulolytic organisms that exhibit high product yields and titers to express a heterologous cellulase and hemicellulase system enabling cellulose and hemicellulose utilization.

Biological conversion of lignocellulosic biomass to ethanol or other chemicals requires a microbial catalyst to be metabolically active during the extent of the conversion. For CBP, a further requirement is placed on the microbial catalyst—it must also grow and produce sufficient cellulolytic and other hydrolytic enzymes in addition to metabolic products. A significant challenge for a CBP process occurs when the lignocellulosic biomass contains compounds inhibitory to microbial growth, which is common in natural lignocellulosic feedstocks. Arguably the most important inhibitory compound is acetic acid (acetate), which is released during deacetylation of polymeric substrates. Acetate is particularly inhibitory for CBP processes, as cells must constantly expend energy to export acetate anions, which then freely diffuse back into the cell as acetic acid. This phenomena, combined with the typically low sugar release and energy availability during the fermentation, limits the cellular energy that can be directed towards cell mass generation and enzyme production, which further lowers sugar release.

Removal of acetate prior to fermentation would significantly improve CBP dynamics; however, chemical and physical removal systems are typically too expensive or impractical for industrial application. Thus, there is a need for an alternate acetate removal system for CBP that does not suffer from the same problems associated with these chemical and physical removal systems. As a novel alternative, this invention describes the metabolic conversion of acetate to a less inhibitory compound, such as a non-charged solvent, including but not limited to, acetone, isopropanol, or ethanol. The metabolic conversion of acetate requires the input of electrons. Under anaerobic conditions, the surplus of NADH that is generated during biomass formation is reoxidized via glycerol formation. While the electrons from the surplus NADH can be used for acetate conversion when glycerol production is reduced, the amount of NADH available is limited and is insufficient to completely consume acetate in high concentrations. The present invention combines the metabolic conversion of acetate with processes that produce surplus electron donors, including, but not limited to, processes involved in xylose fermentation and the oxidative branch of the phosphate pentose pathway, to free up more electrons for efficient acetate consumption. In addition, the improved conversion of acetate also results in several process benefits described below.

### BRIEF SUMMARY OF THE INVENTION

The invention is generally directed to the improved reduction or removal of acetate from biomass processing such as the CBP processing of lignocellulosic biomass. The invention is also generally directed to the adaptation of CBP organisms to growth in the presence of inhibitory compounds, including, but not limited to, acetate.

One aspect of the invention relates to a recombinant microorganism comprising one or more native and/or heterologous enzymes that function in one or more first engineered metabolic pathways to convert acetate to an alcohol, wherein said one or more native and/or heterologous enzymes is activated, upregulated or downregulated; and one or more native and/or heterologous enzymes that function in one or more second engineered metabolic pathways to produce an electron donor used in the conversion of acetate to an alcohol, wherein said one or more native and/or heterologous enzymes is activated, upregulated or downregulated. In certain embodiments, the acetate is produced as a by-product of biomass processing. In certain embodiments, the recombinant microorganism produces an alcohol selected from the group consisting of ethanol, isopropanol, or a combination thereof. In some embodiments, the electron donor is selected from the group consisting of NADH, NADPH, or a combination thereof.

In particular aspects, the one or more second engineered metabolic pathways to produce an electron donor is a xylose fermentation pathway. In certain embodiments, the engineered xylose fermentation pathway comprises upregulation of the native and/or heterologous enzymes xylose reductase (XR) and xylitol dehydrogenase (XDH). In some embodiments, the XR reaction has a preference for NADPH or is NADPH-specific, and/or the XDH reaction has a preference for NADH or is NADH-specific. In certain embodiments, the native and/or heterologous XDH enzyme is from Scheffersomyces stipitis. In further embodiments, the XDH enzyme is encoded by a xyl2 polynucleotide. In some embodiments, the native and/or heterologous XR enzyme is from Scheffersomyces stipitis, Neurospora crassa, or Candida boidinii. In certain embodiments, the XR enzyme is encoded by a xyl1 polynucleotide or an aldolase reductase.

In some embodiments, the first and second engineered metabolic pathways in the recombinant microorganism result in ATP production. In further embodiments, the first and second engineered metabolic pathways in the recombinant microorganism result in net ATP production. In certain embodiments, the one or more first engineered metabolic pathways comprises activating or upregulating one or more heterologous enzymes selected from the group consisting of acetyl-CoA acetyltransferase (thiolase), acetoacetyl-CoA transferase, acetoacetate decarboxylase, a secondary alcohol dehydrogenase, or combinations thereof. In some embodiments, the one or more first engineered metabolic pathways comprises activating or upregulating a heterologous ADP-producing acetyl-CoA synthase enzyme. In some embodiments, the one or more first engineered metabolic pathways comprises activating or upregulating the acetate kinase/phosphotransacetylase (AK/PTA) couple. In particular aspects, the first and second engineered metabolic pathways result in ATP production.

In certain embodiments, the one or more second engineered metabolic pathways to produce an electron donor is the oxidative branch of the pentose phosphate pathway (PPP). In some embodiments, the engineered PPP comprises activation or upregulation of the native enzyme glucose-6-P dehydrogenase. In certain embodiments, the native glucose-6-P dehydrogenase enzyme is from Saccharomyces cerevisiae. In further embodiments the glucose-6-P dehydrogenase is encoded by a zwf1 polynucleotide.

In some embodiments, the recombinant microorganism that converts acetate to an alcohol further comprises altering the expression of transcription factors that regulate expression of enzymes of the PPP pathway. In certain embodiments, the transcription factor is Stb5p. In further embodiments, the Stb5p is from Saccharomyces cerevisiae.

In certain embodiments, the one or more second engineered metabolic pathways of the recombinant microorganism that converts acetate to an alcohol to produce an electron donor is a pathway that competes with the oxidative branch of the PPP. In some embodiments, the engineered pathway that competes with the oxidative branch of the PPP comprises downregulation of the native enzyme glucose-6-P isomerase. In further embodiments, the native glucose-6-P isomerase enzyme is from Saccharomyces cerevisiae. In some embodiments, the glucose-6-P isomerase is encoded by a pgi1 polynucleotide.

In certain embodiments, the one or more second engineered metabolic pathways of the recombinant microorganism that converts acetate to an alcohol to produce an electron donor comprises the ribulose-monophosphate pathway (RuMP). In some embodiments, the engineered RuMP pathway converts fructose-6-P to ribulose-5-P and formaldehyde. In further embodiments, the engineered RuMP pathway comprises upregulating a heterologous enzyme selected from the group consisting of 6-phospho-3-hexuloisomerase, 3-hexulose-6-phosphate synthase, and the combination thereof.

In certain embodiments, the one or more second engineered metabolic pathways of the recombinant microorganism that converts acetate to an alcohol to produce an electron donor comprises upregulating native enzymes that degrade formaldehyde or formate. In some embodiments, the formaldehyde degrading enzymes convert formaldehyde to formate. In further embodiments, the formaldehyde degrading enzymes are formaldehyde dehydrogenase and S-formylglutathione hydrolase. In some embodiments, the formate degrading enzyme converts formate to CO₂. In further embodiments, the formate degrading enzyme is formate dehydrogenase. In some embodiments, the formaldehyde is oxidized to form CO₂.

In some embodiments, the formate dehydrogenase is from a yeast microorganism. In some embodiments, the yeast microorganism is S. cerevisiae or Candida boidinii. In further embodiments, the formate dehydrogenase from S. cerevisiae is FDH1. In some embodiments, the formate dehydrogenase from Candida boidinii is FDH3. In some embodiments, the microorganism consumes or uses more acetate than a microorganism not comprising the enzyme that degrades formate. In further embodiments, the recombinant microorganism has an acetate uptake (g/L) under anaerobic conditions from: (a) at least about 1.0 fold more acetate uptake than that taken up by a recombinant microorganism not comprising said enzyme that degrades formate; (b) at least about 1.1 fold more acetate uptake than that taken up by a recombinant microorganism not comprising said enzyme that degrades formate; (c) at least about 1.2 fold more acetate uptake than that taken up by a recombinant microorganism not comprising said enzyme that degrades formate; (d) at least about 1.3 fold more acetate uptake than that taken up by a recombinant microorganism not comprising said enzyme that degrades formate; (e) at least about 1.4 fold more acetate uptake than that taken up by a recombinant microorganism not comprising said enzyme that degrades formate; (f) at least about 1.5 fold more acetate uptake than that taken up by a recombinant microorganism not comprising said enzyme that degrades formate; (g) at least about 2.0 fold more acetate uptake than that taken up by a recombinant microorganism not comprising said enzyme that degrades formate; (h) at least about 2.5 fold more acetate uptake than that taken up by a recombinant microorganism not comprising said enzyme that degrades formate; (i) at least about 3.0 fold more acetate uptake than that taken up by a recombinant microorganism not comprising said enzyme that degrades formate; (j) at least about 4.0 fold more acetate uptake than that taken up by a recombinant microorganism not comprising said enzyme that degrades formate; (k) at least about 5.0 fold more acetate uptake than that taken up by a recombinant microorganism not comprising said enzyme that degrades formate; or (l) at least about 10 fold more acetate uptake than that taken up by a recombinant microorganism not comprising said enzyme that degrades formate. In some embodiments, the recombinant microorganism has an acetate uptake under anaerobic conditions at least about 0.32 g/L, at least about 0.37 g/L, at least about 0.46 g/L, or at least about 0.48 g/L.

In certain embodiments, the recombinant microorganism comprises a) one or more native and/or heterologous enzymes that function in one or more first engineered metabolic pathways to convert acetate to an alcohol, wherein said one or more native and/or heterologous enzymes is activated, upregulated or down-regulated; and b) one or more native and/or heterologous zwf1 polynucleotides; wherein one or more native and/or heterologous enzymes is an NADPH-specific alcohol dehydrogenase. In other embodiments, the NADPH-specific alcohol dehydrogenase is from a microorganism selected from the group consisting of T. pseudethanolicus, C. beijerinckii, Entamoeba histolytica, Cucumis melo, and S. cerevisiae. In further embodiments, the NADPH-specific alcohol dehydrogenase is T. pseudethanolicus adhB. In other embodiments, the NADPH-specific alcohol dehydrogenase is C. beijerinckii 2° Adh. In other embodiments, the NADPH-specific alcohol dehydrogenase is S. cerevisiae ARI1. In some embodiments, the NADPH-specific alcohol dehydrogenase is Entamoeba histolytica ADH1. In other embodiments, the NADPH-specific alcohol dehydrogenase is Cucumis melo ADH1.

In certain embodiments, the one or more native enzymes that function in one or more first engineered metabolic pathways to convert acetate to an alcohol is an NADH-specific alcohol dehydrogenase. In other embodiments, the alcohol dehydrogenase is downregulated. In further embodiments, the downregulated alcohol dehydrogenase is an NADH-ADH selected from ADH1, ADH2, ADH3, ADH4, ADH5, or SFA1 from Saccharomyces. In some embodiments, the recombinant microorganism consumes or uses more acetate than a microorganism not comprising said NADPH-specific alcohol dehydrogenase.

In other embodiments, the recombinant microorganism has an acetate uptake (g/L) under anaerobic conditions: (a) at least about 1.1 fold more acetate uptake than that taken up by a recombinant microorganism not comprising said NADPH-specific alcohol dehydrogenase; (b) at least about 1.2 fold more acetate uptake than that taken up by a recombinant microorganism not comprising said NADPH-specific alcohol dehydrogenase; (c) at least about 1.3 fold more acetate uptake than that taken up by a recombinant microorganism not comprising said NADPH-specific alcohol dehydrogenase; (d) at least about 1.4 fold more acetate uptake than that taken up by a recombinant microorganism not comprising said NADPH-specific alcohol dehydrogenase; (e) at least about 1.5 fold more acetate uptake than that taken up by a recombinant microorganism not comprising said NADPH-specific alcohol dehydrogenase; (f) at least about 1.6 fold more acetate uptake than that taken up by a recombinant microorganism not comprising said NADPH-specific alcohol dehydrogenase; (g) at least about 1.9 fold more acetate uptake than that taken up by a recombinant microorganism not comprising said NADPH-specific alcohol dehydrogenase; (h) at least about 2.0 fold more acetate uptake than that taken up by a recombinant microorganism not comprising said NADPH-specific alcohol dehydrogenase; (i) at least about 2.1 fold more acetate uptake than that taken up by a recombinant microorganism not comprising said NADPH-specific alcohol dehydrogenase; (j) at least about 2.3 fold more acetate uptake than that taken up by a recombinant microorganism not comprising said NADPH-specific alcohol dehydrogenase; (k) at least about 2.4 fold more acetate uptake than that taken up by a recombinant microorganism not comprising said NADPH-specific alcohol dehydrogenase; (l) at least about 2.5 fold more acetate uptake than that taken up by a recombinant microorganism not comprising said NADPH-specific alcohol dehydrogenase; (m) at least about 2.7 fold more acetate uptake than that taken up by a recombinant microorganism not comprising said NADPH-specific alcohol dehydrogenase; (n) at least about 2.8 fold more acetate uptake than that taken up by a recombinant microorganism not comprising said NADPH-specific alcohol dehydrogenase; (o) at least about 2.9 fold more acetate uptake than that taken up by a recombinant microorganism not comprising said NADPH-specific alcohol dehydrogenase; or (p) at least about 3.1 fold more acetate uptake than that taken up by a recombinant microorganism not comprising said NADPH-specific alcohol dehydrogenase.

In further embodiments, the recombinant microorganism has an acetate uptake under anaerobic conditions at least about 0.35 g/L, at least about 0.36 g/L, at least about 0.38 g/L, at least about 0.40 g/L, at least about 0.44 g/L, at least about 0.45 g/L, at least about 0.47 g/L, at least about 0.48 g/L, at least about 0.51 g/L, at least about 0.53 g/L, at least about 0.59 g/L, at least about 0.61 g/L, at least about 0.63 g/L, at least about 0.65 g/L, at least about 0.66 g/L, at least about 0.70 g/L, at least about 0.79 g/L, at least about 0.8 g/L, at least about 0.83 g/l, at least about 0.84 g/L, at least about 0.87 g/L, at least about 0.9 g/L, at least about 0.91 g/L, at least about 0.96 g/L, at least about 0.99 g/L, at least about 1.00 g/L, at least about 1.01 g/L at least about 1.02 g/L, at least about 1.18 g/L, at least about 1.20 g/L, at least about 1.23 g/L, at least about 3.2 g/L, or at least about 3.3 g/L. In other embodiments, the recombinant microorganism has an acetate uptake under anaerobic conditions from about 0.35 g/L to about 3.3 g/L.

In certain embodiments, the recombinant microorganism further comprises one or more native and/or heterologous acetyl-CoA synthetases, and wherein said one or more native and/or heterologous acetyl-CoA synthetases is activated or upregulated. In other embodiments, the acetyl-CoA synthetase is encoded by a polynucleotide selected from the group consisting of an ACS1 polynucleotide and an ACS2 polynucleotide. In further embodiments, the ACS1 polynucleotide or the ACS2 polynucleotide is from a yeast microorganism. In other embodiments, the ACS1 polynucleotide is from Saccharomyces cerevisiae or Saccharomyces kluyveri. In further embodiments, the ACS2 polynucleotide is from Saccharomyces cerevisiae or Saccharomyces kluyveri.

In certain embodiments, the one or more native and/or heterologous enzymes of the recombinant microorganism that converts acetate to an alcohol is from Mycobacterium gastri.

In certain embodiments, the one or more second engineered metabolic pathways of the recombinant microorganism that converts acetate to an alcohol to produce an electron donor comprises the dihydroxyacetone (DHA) pathway. In some embodiments, the engineered DHA pathway interconverts dihydroxyacetone and glyceraldehyde-3-P into xylose-5-P and formaldehyde. In further embodiments, the engineered DHA pathway comprises upregulating the heterologous enzyme formaldehyde transketolase (EC 2.2.1.3).

In certain embodiments, the one or more second engineered metabolic pathways of the recombinant microorganism that converts acetate to an alcohol to produce an electron donor comprises upregulating native and/or heterologous enzymes that produce dihydroxyacetone. In some embodiments, the native and/or heterologous enzymes that produce dihydroxyacetone are selected from the group consisting of glycerol dehydrogenase, dihydroxyacetone phosphatase, and a combination thereof. In further embodiments, the native and/or heterologous glycerol dehydrogenase is from a microorganism selected from the group consisting of Hansenula polymorpha, E. coli, Pichia angusta, and Saccharomyces cerevisiae. In some embodiments, the glycerol dehydrogenase is encoded by a polynucleotide selected from the group consisting of gdh, gldA, and gcy1.

In certain embodiments, the one or more second engineered metabolic pathways of the recombinant microorganism that converts acetate to an alcohol to produce an electron donor comprises downregulating a native dihydroxyacetone kinase enzyme. In some embodiments, the dihydroxyacetone kinase is encoded by a polynucleotide selected from the group consisting of dak1, dak2, and a combination thereof.

In certain embodiments, the recombinant microorganism that converts acetate to an alcohol further comprises overexpressing a glycerol/proton-symporter. In some embodiments, the glycerol/proton-symporter is encoded by a stl1 polynucleotide.

In certain embodiments, the recombinant microorganism that converts acetate to an alcohol farther comprises overexpression of a native and/or heterologous transhydrogenase enzyme. In some embodiments, the transhydrogenase catalyzes the interconversion of NADPH and NAD to NADP and NADH. In further embodiments, the transhydrogenase is from a microorganism selected from the group consisting of Escherichia coli and Azotobacter vinelandii.

In certain embodiments, the recombinant microorganism that converts acetate to an alcohol further comprises overexpression of a native and/or heterologous glutamate dehydrogenase enzyme. In some embodiments, the glutamate dehydrogenase is encoded by a gdh2 polynucleotide.

In certain embodiments of the invention, in the recombinant microorganism that converts acetate to an alcohol, one of the engineered metabolic pathways comprises the conversion of acetate to acetyl-CoA and conversion of acetyl-CoA to ethanol.

In certain embodiments, the one or more downregulated native enzymes of the microorganism that converts acetate to an alcohol is encoded by a gpd1 polynucleotide, a gpd2 polynucleotide, or both a gpd1 polynucleotide and a gpd2 polynucleotide.

In certain embodiments, the microorganism that converts acetate to an alcohol produces ethanol.

In certain embodiments, the microorganism that converts acetate to an alcohol is selected from the group consisting of Saccharomyces cerevisiae, Kluyveromyces lactis, Kluyveromyces marxianus, Pichia pastoris, Yarrowia lipolylica, Hansenula polymorpha, Phaffia rhodozyma, Candida utilisutilis, Arxula adeninivorans, Pichia stipitis, Debaryomyces hansenii, Debaryomyces polymorphus, Schizosaccharomyces pombe, Candida albicans, and Schwanniomycesoccidentalis. In some embodiments, the microorganism is Saccharomyces cerevisiae.

In certain embodiments, in the microorganism that converts acetate to an alcohol, acetate is converted to acetyl-CoA by an acetyl-CoA transferase (ACS). In some embodiments, the acetate is converted to acetyl-P by an acetate kinase and the acetyl-P is converted to acetyl-CoA by a phosphotransacetylase. In some embodiments, the acetyl-CoA transferase (ACS) is encoded by an ACS 1 polynucleotide. In further embodiments, the acetate kinase and the phosphotransacetylase are from one or more of an Escherichia, a Thermoanaerobacter, a Clostridia, or a Bacillus species. In some embodiments, acetyl-CoA is converted to acetaldehyde by an acetaldehyde dehydrogenase and the acetaldehyde is converted to ethanol by an alcohol dehydrogenase. In some embodiments, the acetaldehyde dehydrogenase is from C. phytofermentans. In further embodiments, the acetaldehyde dehydrogenase is an NADPH-specific acetaldehyde dehydrogenase. In some embodiments, the NADPH-specific acetaldehyde dehydrogenase is from T. pseudethanolicus. In further embodiments, the NADPH-specific acetaldehyde dehydrogenase is T. pseudethanolicus adhB. In some embodiments, the alcohol dehydrogenase is an NADPH-specific alcohol dehydrogenase. In further embodiments, the NADPH-specific alcohol dehydrogenase is from a microorganism selected from the group consisting of T. pseudethanolicus, C. beijerinckii, Entamoeba histolytica, Cucumis melo, and S. cerevisiae. In some embodiments, the NADPH-specific alcohol dehydrogenase is T. pseudethanolicus adhB. In some embodiments, the NADPH-specific alcohol dehydrogenase is C. beijerinckii 2° Adh. In certain embodiments, the NADPH-specific alcohol dehydrogenase is S. cerevisiae ARI1. In some embodiments, the NADPH-specific alcohol dehydrogenase is Entamoeba histolytica ADH1. In certain embodiments, the NADPH-specific alcohol dehydrogenase is Cucumis melo ADH1.

In certain embodiments, the microorganism consumes or uses more acetate than a microorganism not comprising said NADPH-specific alcohol dehydrogenase. In some embodiments, the recombinant microorganism has an acetate uptake (g/L) under anaerobic conditions: (a) at least about 1.1 fold more acetate uptake than that taken up by a recombinant microorganism not comprising said NADPH-specific alcohol dehydrogenase; (b) at least about 1.2 fold more acetate uptake than that taken up by a recombinant microorganism not comprising said NADPH-specific alcohol dehydrogenase; (c) at least about 1.3 fold more acetate uptake than that taken up by a recombinant microorganism not comprising said NADPH-specific alcohol dehydrogenase; (d) at least about 1.4 fold more acetate uptake than that taken up by a recombinant microorganism not comprising said NADPH-specific alcohol dehydrogenase; (e) at least about 1.5 fold more acetate uptake than that taken up by a recombinant microorganism not comprising said NADPH-specific alcohol dehydrogenase; (f) at least about 1.6 fold more acetate uptake than that taken up by a recombinant microorganism not comprising said NADPH-specific alcohol dehydrogenase; (g) at least about 1.9 fold more acetate uptake than that taken up by a recombinant microorganism not comprising said NADPH-specific alcohol dehydrogenase; (h) at least about 2.0 fold more acetate uptake than that taken up by a recombinant microorganism not comprising said NADPH-specific alcohol dehydrogenase; (i) at least about 2.1 fold more acetate uptake than that taken up by a recombinant microorganism not comprising said NADPH-specific alcohol dehydrogenase; (j) at least about 2.3 fold more acetate uptake than that taken up by a recombinant microorganism not comprising said NADPH-specific alcohol dehydrogenase; (k) at least about 2.4 fold more acetate uptake than that taken up by a recombinant microorganism not comprising said NADPH-specific alcohol dehydrogenase; (l) at least about 2.5 fold more acetate uptake than that taken up by a recombinant microorganism not comprising said NADPH-specific alcohol dehydrogenase; (m) at least about 2.7 fold more acetate uptake than that taken up by a recombinant microorganism not comprising said NADPH-specific alcohol dehydrogenase; (n) at least about 2.8 fold more acetate uptake than that taken up by a recombinant microorganism not comprising said NADPH-specific alcohol dehydrogenase; (o) at least about 2.9 fold more acetate uptake than that taken up by a recombinant microorganism not comprising said NADPH-specific alcohol dehydrogenase; or (p) at least about 3.1 fold more acetate uptake than that taken up by a recombinant microorganism not comprising said NADPH-specific alcohol dehydrogenase.

In further embodiments, the recombinant microorganism has an acetate uptake under anaerobic conditions at least about 0.35 g/L, at least about 0.36 g/L, at least about 0.38 g/L, at least about 0.40 g/L, at least about 0.44 g/L, at least about 0.45 g/L, at least about 0.47 g/L, at least about 0.48 g/L, at least about 0.51 g/L, at least about 0.53 g/L, at least about 0.59 g/L, at least about 0.61 g/L, at least about 0.63 g/L, at least about 0.65 g/L, at least about 0.66 g/L, at least about 0.70 g/L, at least about 0.79 g/L, at least about 0.8 g/L, at least about 0.83 g/L, at least about 0.84 g/L, at least about 0.87 g/L, at least about 0.9 g/L, at least about 0.91 g/L, at least about 0.96 g/L, at least about 0.99 g/L, at least about 1.00 g/L, at least about 1.01 g/L at least about 1.02 g/L, at least about 1.18 g/L, at least about 1.20 g/L, at least about 1.23 g/L, at least about 3.2 g/L, or at least about 3.3 g/L. In other embodiments, the recombinant microorganism has an acetate uptake under anaerobic conditions from about 0.35 g/L to about 3.3 g/L.

In certain embodiments, in the recombinant microorganism that converts acetate to an alcohol, acetyl-CoA is converted to ethanol by a bifunctional acetaldehyde/alcohol dehydrogenase. In some embodiments, the bifunctional acetaldehyde/alcohol dehydrogenase is from E. coli, C. acetobutylicum, T. saccharolyticum, C. thermocellum, or C. phytofermentans.

Another aspect of the invention relates to a recombinant microorganism comprising one or more native and/or heterologous enzymes that function in one or more engineered metabolic pathways to convert acetate to acetone, wherein the one or more native and/or heterologous enzymes is activated, upregulated or down-regulated; and one or more native and/or heterologous enzymes that function in one or more second engineered metabolic pathways to produce an electron donor used in the conversion of acetate to acetone, wherein the one or more native and/or heterologous enzymes is activated, upregulated or downregulated. In some embodiments, the acetate is produced as a by-product of biomass processing. In certain embodiments, one of the engineered metabolic pathways comprises the conversion of acetate to acetyl-CoA; conversion of acetyl-CoA to acetoacetyl-CoA; conversion of acetoacetyl-CoA to acetoacetate; and conversion of acetoacetate to acetone.

In certain embodiments, the recombinant microorganism that converts acetate to acetone produces acetone. In some embodiments, the recombinant microorganism is Escherichia coli. In certain embodiments, the recombinant microorganism is a thermophilic or mesophilic bacterium. In further embodiments, the recombinant microorganism is a species of the genera Thermoanaerobacterium, Thermoanaerobacter, Clostridium, Geobacillus, Saccharococcus, Paenibacillus, Bacillus, Caldicellulosiruptor, Anaerocellum, or Anoxybacillus. In some embodiments, the recombinant microorganism is a bacterium selected from the group consisting of Thermoanaerobacteriumthermosulfurigenes, Thermoanaerobacteriumaotearoense, Thermoanaerobacteriumpolysaccharolyticum, Thermoanaerobacteriumzeae, Thermoanaerobacteriumxylanolyticum, Thermoanaerobacterium saccharolyticum, Thermoanaerobiumbrockii, Thermoanaerobacteriumthermosaccharolyticum, Thermoanaerobacter thermohydrosulfuricus, Thermoanaerobacterethanolicus, Thermoanaerobacterbrocki, Clostridium thermocellum, Clostridium cellulolyticum, Clostridium phytofermentans, Clostridium straminosolvens, Geobacillus thermoglucosidasius, Geobacillus stearothermophilus, Saccharococcus caldoxylosilyticus, Saccharoccus thermophilus, Paenibacillus campinasensis, Bacillus flavothermus, Anoxybacillus kamchatkensis, Anoxybacillus gonensis, Caldicellulosiruptor acetigenus, Caldicellulosiruptor saccharolyticus, Caldicellulosiruptor kristjanssonii, Caldicellulosiruptor owensensis, Caldicellulosiruptor lactoaceticus, and Anaerocellumthermophilum.

In certain embodiments, the recombinant microorganism that converts acetate to acetone is selected from the group consisting of Clostridium thermocellum and Thermoanaerobacterium saccharolyticum. In some embodiments, the recombinant microorganism is selected from the group consisting of Saccharomyces cerevisiae, Kluyveromyces lactis, Kluyveromyces marxianus, Pichia pastoris, Yarrowia lipolytica, Hansenula polymorpha, Phaffia rhodozyma, Candida utilis, Arxula adeninivorans, Pichia stipitis, Debaryomyces hansenii, Debaryomyces polymorphus, Schizosaccharomyces pombe, Candida albicans, and Schwanniomycesoccidentalis. In further embodiments, the recombinant microorganism is Saccharomyces cerevisiae.

In certain embodiments, in the recombinant microorganism that converts acetate to acetone, the acetate is converted to acetyl-CoA by an acetyl-CoA synthetase. In some embodiments, the acetate is converted to acetyl-P by an acetate kinase and the acetyl-P is converted to acetyl-CoA by a phosphotransacetylase. In further embodiments, the acetyl-CoA is converted to acetoacetyl-CoA by a thiolase. In some embodiments, the acetoacetyl-CoA is converted to acetoacetate by a CoA transferase. In certain embodiments, the acetoacetate is converted to acetone by an acetoacetate decarboxylase. In some embodiments, the acetyl-CoA synthetase is encoded by a polynucleotide selected from the group consisting of a yeast ACS1 polynucleotide and a yeast ACS2 polynucleotide. In further embodiments, the yeast ACS1 polynucleotide is from Saccharomyces cerevisiae or Saccharomyces kluyveri. In certain embodiments, the yeast ACS2 polynucleotide is from Saccharomyces cerevisiae or Saccharomyces kluyveri. In some embodiments, the acetate kinase and phosphotransacetylase are from T. saccharolyticum. In some embodiments, the thiolase, CoA transferase, and acetoacetate decarboxylase are from C. acetobutylicum. In further embodiments, the thiolase is from C. acetobutylicum or T. thermosaccharolyticum. In some embodiments, the CoA transferase is from a bacterial source. In further embodiments, the bacterial source is selected from the group consisting of Thermoanaerobacter tengcongensis, Thermoanaerbacterium thermosaccharolyticum, Thermosipho africanus, and Paenibacillus macerans. In some embodiments, the acetoacetate decarboxylase is from a bacterial source. In further embodiments, the bacterial source is selected from the group consisting of C. acetobutylicum, Paenibacillus macerans, Acidothermus cellulolyticus, Bacillus amyloliquefaciens, and Rubrobacter xylanophilus.

In certain embodiments, in the recombinant microorganism that converts acetate to acetone, one of said engineered metabolic pathways comprises the conversion of acetate to acetyl-CoA; conversion of acetyl-CoA to acetoacetyl-CoA; conversion of acetoacetyl-CoA to acetoacetate; conversion of acetoacetate to acetone; and conversion of acetone to isopropanol. In further embodiments, the recombinant microorganism is selected from the group consisting of Saccharomyces cerevisiae, Kluyveromyces lactis, Kluyveromyces marxianus, Pichia pastoris, Yarrowia lipolytica, Hansenula polymorpha, Phaffia rhodozyma, Candida utilis, Arxula adeninivorans, Pichia stipitis, Debaryomyces hansenii, Debaryomyces polymorphus, Schizosaccharomyces pombe, Candida albicans, and Schwanniomycesoccidentalis. In some embodiments, the recombinant microorganism is Saccharomyces cerevisiae.

In certain embodiments, in the recombinant microorganism, acetate is converted to acetyl-CoA by an acetyl-CoA synthetase. In some embodiments, the acetyl-CoA is converted to acetoacetyl-CoA by a thiolase. In some embodiments, the acetoacetyl-CoA is converted to acetoacetate by a CoA transferase. In certain embodiments, the acetoacetate is converted to acetone by an acetoacetate decarboxylase. In some embodiments, the acetone is converted to isopropanol by an alcohol dehydrogenase. In further embodiments, the acetyl-CoA synthetase is encoded by a polynucleotide selected from the group consisting of a yeast ACS1 polynucleotide and a yeast ACS2 polynucleotide. In some embodiments, the CoA transferase is from a bacterial source. In certain embodiments, the acetoacetate decarboxylase is from a bacterial source.

In certain embodiments, the invention relates to a recombinant microorganism comprising one or more native and/or heterologous enzymes that function in one or more engineered metabolic pathways to convert acetate to an alcohol, wherein one of said native and/or heterologous enzymes is an NADPH-specific alcohol dehydrogenase. In some embodiments, the NADPH-specific alcohol dehydrogenase is from a microorganism selected from the group consisting of T. pseudethanolicus, C. beijerinckii, Entamoeba histolytica, Cucumis melo, and S. cerevisiae. In some embodiments, the NADPH-specific alcohol dehydrogenase is encoded by any one of SEQ ID NOs:30, 32, 33, 35, or 36 or a fragment, variant, or derivative thereof that retains the function of an alcohol dehydrogenase.

In certain embodiments, the invention relates to a recombinant microorganism comprising one or more native and/or heterologous enzymes that function in one or more engineered metabolic pathways to convert acetate to an alcohol, wherein a first native and/or heterologous enzyme is an NADPH-specific alcohol dehydrogenase and wherein a second native and/or heterologous enzyme is an acetyl-CoA synthetase. In some embodiments, the NADPH-specific alcohol dehydrogenase is from Entamoeba histolytica. In some embodiments, the NADPH-specific alcohol dehydrogenase is encoded by SEQ ID NO:35 or a fragment, variant, or derivative thereof that retains the function of an alcohol dehydrogenase. In some embodiments, the acetyl-CoA synthetase is from a yeast microorganism or from a bacterial microorganism. In some embodiments, the acetyl-CoA synthetase is from Saccharomyces cerevisiae, Saccharomyces kluyveri, Zygosaccharomyces bailii, or Acetobacter aceti. In other embodiments, the acetyl-CoA synthetase is encoded by any one of SEQ ID NOs:37-40, 57, 58 or a fragment, variant, or derivative thereof that retains the function of an acetyl-CoA synthetase.

In certain embodiments, the invention relates to a recombinant microorganism comprising one or more native and/or heterologous enzymes that function in one or more engineered metabolic pathways to convert acetate to an alcohol, wherein a first native and/or heterologous enzyme is an NADPH-specific alcohol dehydrogenase and wherein a second native and/or heterologous enzyme is an NADH-specific alcohol dehydrogenase. In some embodiments, the NADPH-specific alcohol dehydrogenase is from Entamoeba histolytica. In some embodiments, the NADPH-specific alcohol dehydrogenase is encoded by SEQ ID NO:35 or a fragment, variant, or derivative thereof that retains the function of an alcohol dehydrogenase. In some embodiments, the NADH-specific alcohol dehydrogenase is downregulated. In some embodiments, the downregulated NADH-specific alcohol dehydrogenase is selected from ADH1, ADH2, ADH3, ADH4, ADH5, or SFA1 from Saccharomyces.

In certain embodiments, the invention relates to a recombinant microorganism comprising a) one or more native and/or heterologous enzymes that function in one or more first engineered metabolic pathways to convert acetate to an alcohol, wherein said one or more native and/or heterologous enzymes is activated, upregulated or downregulated; and b) one or more native and/or heterologous enzymes that function in one or more second engineered metabolic pathways to produce an electron donor used in the conversion of acetate to an alcohol, wherein one of said native and/or heterologous enzymes is a formate dehydrogenase. In some embodiments, the formate dehydrogenase is from a yeast microorganism. In some embodiments, the yeast microorganism is S. cerevisiae or Candida boidinii. In other embodiments, the formate dehydrogenase from S. cerevisiae is FDH1 or from Candida boidinii is FDH3. In some embodiments, the formate dehydrogenase from is encoded by SEQ ID NO:46, 47, or a fragment, variant, or derivative thereof that retains the function of a formate dehydrogenase.

Another aspect of the invention relates to a method for increasing acetate uptake from a biomass comprising contacting said biomass with a recombinant microorganism of the invention. In further embodiments, the method further comprises increasing the amount of sugars of the biomass. In other embodiments, the sugars are increased by the addition of an exogenous sugar source to the biomass. In further embodiments, the sugars are increased by the addition of one or more enzymes to the biomass or the recombinant microorganisms of the invention that use or break-down cellulose, hemicellulose and/or other biomass components. In other embodiments, the sugars are increased by the addition of a CBP microorganism that uses or breaks-down cellulose, hemicellulose and/or other biomass components.

Another aspect of the invention relates to a process for converting biomass to ethanol, acetone, or isopropanol comprising contacting biomass with a recombinant microorganism of the invention. In some embodiments, the biomass comprises lignocellulosic biomass. In further embodiments, the lignocellulosic biomass is selected from the group consisting of grass, switch grass, cord grass, rye grass, reed canary grass, mixed prairie grass, miscanthus, sugar-processing residues, sugarcane bagasse, sugarcane straw, agricultural wastes, rice straw, rice hulls, barley straw, corn cobs, cereal straw, wheat straw, canola straw, oat straw, oat hulls, corn fiber, stover, soybean stover, corn stover, forestry wastes, recycled wood pulp fiber, paper sludge, sawdust, hardwood, softwood, agave, and combinations thereof.

In certain embodiments, the process reduces or removes acetate from the consolidated bioprocessing (CBP) media. In some embodiments, the reduction or removal of acetate occurs during fermentation.

The invention further relates to an engineered metabolic pathway for reducing or removing acetate from consolidated bioprocessing (CBP) media.

### BRIEF DESCRIPTION OF THE DRAWINGS/FIGURES

FIG. 1 shows a schematic for a pathway for converting acetate to ethanol using the endogenous acetyl-CoA synthetase (ACS).

FIG. 2 shows a schematic for a pathway for converting acetate to ethanol using an ADP-ACS or the acetate kinase/phospho-transacetylase (AK/PTA) couple.

FIG. 3 shows a schematic for a pathway for converting acetate to isopropanol using ACS, acetyl-CoA acetyltransferase (ACoAAT), acetoacetyl-CoA transferase (ACoAT), acetoacetate decarboxylase (ADC), and secondary alcohol dehydrogenase (SADH).

FIG. 4 shows a schematic for a pathway for converting xylose to ethanol using either xylose isomerase, for which the conversion is redox neutral, or an NADP+-dependent xylose reductase and NADH-dependent xylitol dehydrogenase, in which case an NADPH shortage and NADH surplus is created. This NADPH shortage can be relieved by directing part of the carbon flux through the oxidative pentose phosphate pathway, which generates 2 NADPH for every CO₂ formed.

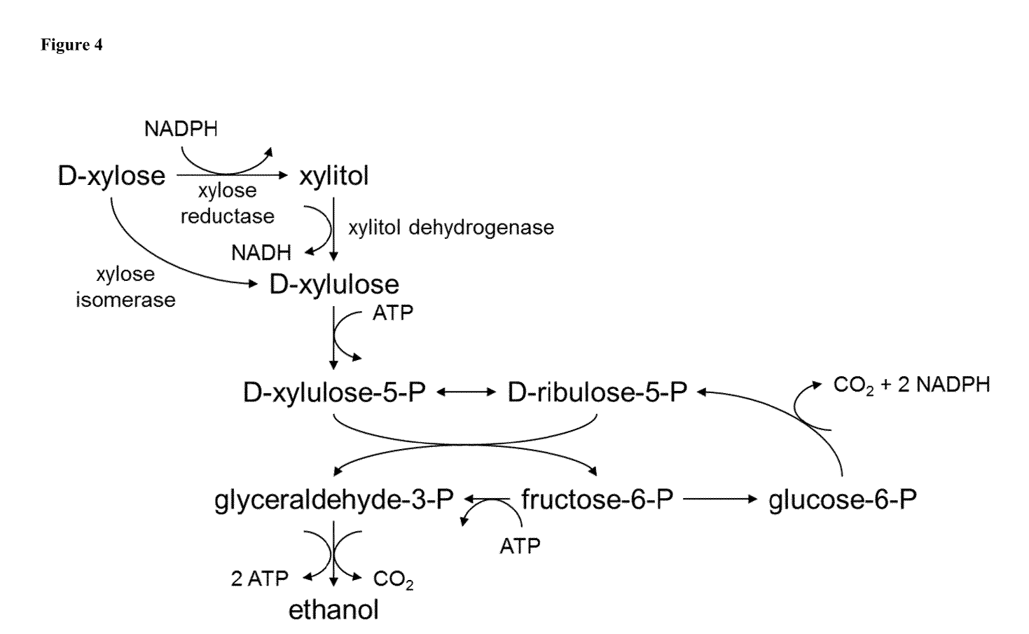

FIG. 5 shows a schematic for a ribulose-monophosphate (RuMP) pathway for converting fructose 6-P to ribulose 5-phosphate and CO₂ to generate 2 NADH.

FIG. 6 shows a schematic for a dihydroxyacetone (DHA) pathway for converting glycerol or dihydroxyacetone phosphate to DHA and its subsequent conversion to CO₂ to generate 2 NADH.

FIG. 7 shows a schematic for integration of B. adolescentis AdhE in the GPD1 locus.

FIG. 8 depicts a vector used for integration of B. adolescentis AdhE in the GPD1 locus.

FIG. 9 shows a schematic for integration of B. adolescentis AdhE in the GPD2 locus.

FIG. 10 depicts a vector used for integration of B. adolescentis AdhE in the GPD2 locus.

FIG. 11 shows a schematic for integration of GDH2 in the FCY1 locus.

FIG. 12 depicts a vector used for integration of GDH2 in the FCY1 locus.

FIG. 13 shows a schematic for integration of endogenous pentose phosphate genes TAL1, XKS1, TKL1, RPE1, and RKI1 in the GRE3 locus.

FIG. 14 depicts a vector used for integration of endogenous pentose phosphate genes TAL1, XKS1, TKL1, RPE1, and RKI1 in the GRE3 locus.

FIG. 15 shows a schematic for integration of Scheffersomyces stipites XYL1 and XYL2 genes and Piromyces sp. E2 adhE gene in the GPD1 locus.

FIG. 16 depicts a vector used for integration of Scheffersomyces stipites XYL1 and XYL2 genes and Piromyces sp. E2 adhE gene in the GPD1 locus.

FIG. 17 shows a schematic for integration of STB5 and GDH2 in the FCY1 locus.

FIG. 18 depicts a vector used for integration of STB5 and GDH2 in the FCY1 locus.

FIG. 19 shows a schematic for integration of Mycobacterium gastri rmpA, O. polymorpha formaldehyde dehydrogenase, O. polymorpha formate dehydrogenase, and Mycobacterium gastri rmpB in the FCY1 locus.

FIG. 20 depicts a vector used for integration of Mycobacterium gastri rmpA, O. polymorpha formaldehyde dehydrogenase, O. polymorpha formate dehydrogenase, and Mycobacterium gastri rmpB in the FCY1 locus.

FIG. 21 shows schematics for deletion of the DAK1 and DAK2 genes.

FIG. 22 shows a schematic for deletion of the DAK1 gene.

FIG. 23 shows a schematic for deletion of the DAK2 gene.

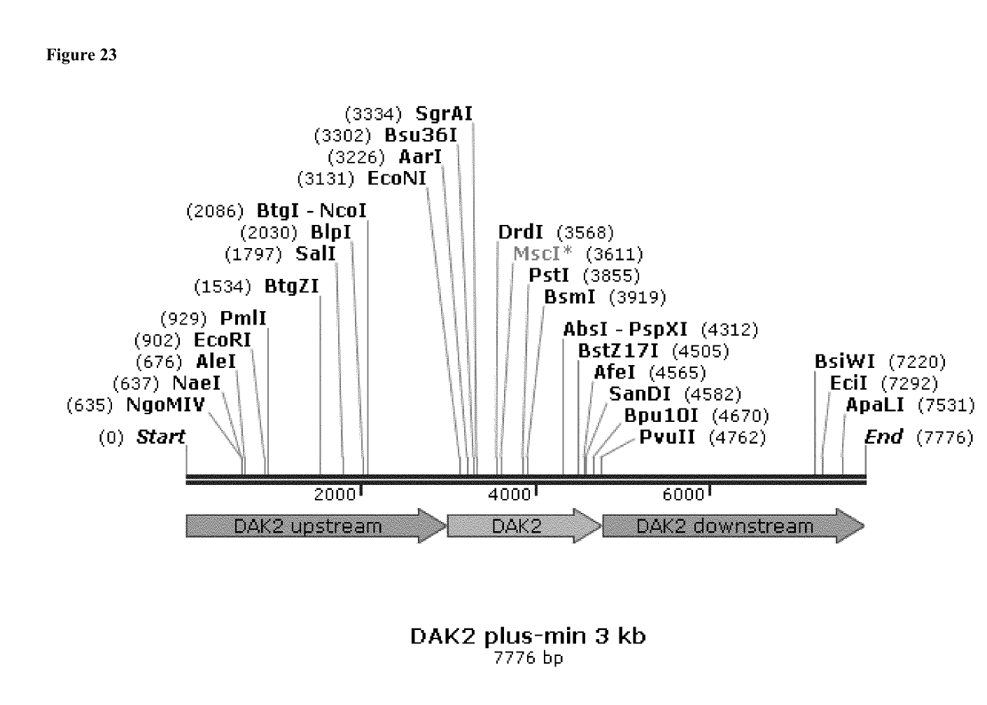

FIG. 24 shows a schematic for integration of O. polymorpha glycerol dehydrogenase, O. polymorpha formaldehyde dehydrogenase, O. polymorpha formate dehydrogenase, transketolase (TKL1), and Piromyces sp. E2 adhE in the FCY1 locus.

FIG. 25 depicts a vector used for integration of O. polymorpha glycerol dehydrogenase, O. polymorpha formaldehyde dehydrogenase, O. polymorpha formate dehydrogenase, transketolase (TKL1), and Piromyces sp. E2 adhE in the FCY1 locus.

FIG. 26 shows a schematic for replacing both chromosomal copies of GRE3 with an expression cassette containing genes from the pentose phosphate pathway.

FIG. 27 depicts a vector for replacing both chromosomal copies of GRE3 with an expression cassette containing genes from the pentose phosphate pathway.

FIG. 28 shows a schematic for integration of T. pseudethanolicus adhB with the Eno1 promoter in the FCY1 locus.

FIG. 29 shows a schematic for integration of T. pseudethanolicus adhB with the TPI1p promoter in the FCY1 locus.

FIG. 30 shows a schematic for integration of C. beijerinckii 2° Adh (Cbe adhB) with the Eno1p promoter in the FCY1 locus.

FIG. 31 shows a schematic for integration of C. beijerinckii 2° Adh with the TPI1p promoter in the FCY1 locus.

FIG. 32 shows a schematic for a construct used to express C. beijerinckii 2° Adh. Zeo depicts the Zeo cassette.

FIG. 33 shows a schematic for a construct used to express ARI1 using the Eno1 promoter. Zeo depicts the Zeo cassette.

FIG. 34 shows a schematic for a construct used to express ARI1 using the TPI1p promoter. Zeo depicts the Zeo cassette.

FIG. 35 shows a schematic for a construct used to express Entamoeba histolytica ADH1 from the Eno1 promoter. Zeo depicts the Zeo cassette.

FIG. 36 shows a schematic for a construct used to express Entamoeba histolytica ADH1 from the TPI1p promoter. Zeo depicts the Zeo cassette.

FIG. 37 shows a schematic for a construct used to express Cucumis melo ADH1 from the Eno1 promoter. Zeo depicts the Zeo cassette.

FIG. 38 shows a schematic for a construct used to express Cucumis melo ADH1 from the TPI1p promoter. Zeo depicts the Zeo cassette.

FIG. 39 shows a schematic of a construct to delete ADH1.

FIG. 40 shows a schematic of a construct to delete ADH1.

FIG. 41 shows acetate consumption for C. beijerinckii 2° Adh and Entamoeba histolytica ADH expressed in an ADH1 wild-type, single copy deletion, or double copy deletion yeast mutants.

FIG. 42 shows a schematic of an ADH1 deletion.

FIG. 43 shows a schematic for a construct (MA741) used to express two copies of Entamoeba histolytica ADH1 (EhADH1) from the TPI1p promoter for integration at YLR296W.

FIG. 44 shows a schematic for a construct (MA743) used to express two copies of Entamoeba histolytica ADH1 (EhADH1) from the TPI1p promoter and a copy of ZWF1 (glucose-6-P dehydrogenase) from the Eno1 promoter for integration at YLR296W.

FIG. 45 shows a schematic for a construct (MA742) used to express two copies of Entamoeba histolytica ADH1 (EhADH1) from the TPI1p promoter and a copy of STB5 from the Eno1 promoter for integration at YLR296W.

FIG. 46 shows a schematic for ethanol production and NAD(P)H balance without ADH engineering.

FIG. 47 shows a schematic for ethanol production and NAD(P)H balance with ADH engineering.

FIG. 48 shows a schematic for a construct (MA421) used to express a copy of S. cerevisiae FDH1 from the ADH1 promoter.

FIG. 49 shows a schematic for a construct (MA422) used to express two copies of C. boidinii FDH3 from the TPI1 and PFK1 promoters.

FIG. 50 shows a schematic for a construct used to express two copies of Entamoeba histolytica ADH1 (EhADH1) from the TPI1p promoter, S. cerevisiae STB5 from the Eno1 promoter, and S. cerevisiae ACS2 from the PYK1 promoter.

FIG. 51 shows a schematic for a construct used to express two copies of Entamoeba histolytica ADH1 (EhADH1) from the TPI1p promoter, S. cerevisiae ZWF1 from the Eno1 promoter, and S. cerevisiae ACS2 from the PYK1 promoter.

FIG. 52 shows a schematic for a construct used to express two copies of Entamoeba histolytica ADH1 (EhADH1) from the TPI1p promoter and S. cerevisiae ACS2 from the PYK1 promoter.

FIG. 53 shows a schematic for a construct used to express the NADPH-ADH from E. histolytica.

FIG. 54 shows a schematic for assembly MA1181 used to replace the endogenous FCY1 ORF with a two-copy expression cassette of E. histolytica ADH1.

FIG. 55 shows a schematic for assembly MA905 used to introduce two copies of E. coli udhA into the apt2 locus.

FIG. 56 shows a schematic for assembly MA483 used to introduce two copies of E. coli udhA into the YLR296W locus.

FIG. 57A shows ethanol production from pressure bottle fermentations on pre-treated agricultural waste by control strains and strains expressing E. coli udhA.

FIG. 57B shows acetate consumption from pressure bottle fermentations on pre-treated agricultural waste by control strains and strains expressing E. coli udhA.

FIG. 57C shows glycerol production from pressure bottle fermentations on pre-treated agricultural waste by control strains and strains expressing E. coli udhA.

FIG. 58A shows ethanol production from pressure bottle fermentations on pre-treated corn stover by control strains and strains expressing E. coli udhA.

FIG. 58B shows acetate consumption from pressure bottle fermentations on pre-treated corn stover by control strains and strains expressing E. coli udhA.

FIG. 58C shows glycerol production from pressure bottle fermentations on pre-treated corn stover by control strains and strains expressing E. coli udhA.

FIG. 59 shows a schematic for a contstruct that can used to express Azotobacter vinelandii sthA.

### DETAILED DESCRIPTION OF THE INVENTION

Aspects of the present invention relate to the engineering of a microorganism to detoxify biomass-derived acetate via metabolic conversion to ethanol, acetone, or isopropanol by improving the availability of redox cofactors NADH or NADPH. To overcome the inhibitory effects of acetate, the acetate can be converted to a less inhibitory compound that is a product of bacterial or yeast fermentation, as described herein. Less inhibitory compounds such as ethanol, acetone, or isopropanol, can be readily recovered from the fermentation media. In addition, the present invention relates to the engineering of a microorganism to provide additional electron donors, thereby producing additional electrons, which facilitate more efficient conversion of acetate to the less inhibitory compounds. Additional advantages of the present invention over existing means for reducing acetate include:

Reduced cost compared to chemical or physical acetate removal systems;

Reduced loss of sugar yield (washing) compared to chemical or physical acetate removal systems;

Reduced demand for base addition during fermentation;

Reduced overall fermentation cost;

Improved pH control;

Reduced costs, including capital, operating, and environmental, for wastewater treatment and water recycling; and

Improved metabolic conversion of acetate by optimization of pathways that produce or balance electron donors.

### DEFINITIONS

Unless defined otherwise, all technical and scientific terms used herein have the same meaning as commonly understood by one of ordinary skill in the art to which this invention belongs. Also, unless otherwise required by context, singular terms shall include pluralities and plural terms shall include the singular. All publications, patents and other references mentioned herein are incorporated by reference in their entireties for all purposes.

The term “heterologous” when used in reference to a polynucleotide, a gene, a polypeptide, or an enzyme refers to a polynucleotide, gene, polypeptide, or an enzyme not normally found in the host organism. “Heterologous” also includes a native coding region, or portion thereof, that is reintroduced into the source organism in a form that is different from the corresponding native gene, e.g., not in its natural location in the organism's genome. The heterologous polynucleotide or gene may be introduced into the host organism by, e.g., gene transfer. A heterologous gene may include a native coding region that is a portion of a chimeric gene including non-native regulatory regions that is reintroduced into the native host. Foreign genes can comprise native genes inserted into a non-native organism, or chimeric genes.

The term “heterologous polynucleotide” is intended to include a polynucleotide that encodes one or more polypeptides or portions or fragments of polypeptides. A heterologous polynucleotide may be derived from any source, e.g., eukaryotes, prokaryotes, viruses, or synthetic polynucleotide fragments.

The terms “promoter” or “surrogate promoter” is intended to include a polynucleotide that can transcriptionally control a gene-of-interest that it does not transcriptionally control in nature. In certain embodiments, the transcriptional control of a surrogate promoter results in an increase in expression of the gene-of-interest. In certain embodiments, a surrogate promoter is placed 5′ to the gene-of-interest. A surrogate promoter may be used to replace the natural promoter, or may be used in addition to the natural promoter. A surrogate promoter may be endogenous with regard to the host cell in which it is used, or it may be a heterologous polynucleotide sequence introduced into the host cell, e.g., exogenous with regard to the host cell in which it is used.

The terms “gene(s)” or “polynucleotide” or “polynucleotide sequence(s)” are intended to include nucleic acid molecules, e.g., polynucleotides which include an open reading frame encoding a polypeptide, and can further include non-coding regulatory sequences, and introns. In addition, the terms are intended to include one or more genes that map to a functional locus. In addition, the terms are intended to include a specific gene for a selected purpose. The gene may be endogenous to the host cell or may be recombinantly introduced into the host cell, e.g., as a plasmid maintained episomally or a plasmid (or fragment thereof) that is stably integrated into the genome. In addition to the plasmid form, a gene may, for example, be in the form of linear DNA. In certain embodiments, the gene or polynucleotide is involved in at least one step in the bioconversion of acetate to a non-charged solvent, including but not limited to, acetone, isopropanol, or ethanol. Accordingly, the term is intended to include any gene encoding a polypeptide, such as the enzymes acetate kinase (ACK), phosphotransacetylase (PTA), lactate dehydrogenase (LDH), pyruvate formate lyase (PFL), aldehyde dehydrogenase (ADH) and/or alcohol dehydrogenase (ADH), acetyl-CoA transferase (ACS), acetaldehyde dehydrogenase, acetaldehyde/alcohol dehydrogenase (e.g., a bifunctional acetaldehyde/alcohol dehydrogenase), glycerol-3-phosphate dehydrogenase (GPD), acetyl-CoA synthetase, thiolase, CoA transferase, acetoacetate decarboxylase, alcohol acetyltransferase enzymes in the D-xylose pathway, such as xylose isomerase and xylulokinase, enzymes in the L-arabinose pathway, such as L-arabinose isomerase and L-ribulose-5-phosphate 4-epimerase. The term gene is also intended to cover all copies of a particular gene, e.g., all of the DNA sequences in a cell encoding a particular gene product.

The term “transcriptional control” is intended to include the ability to modulate gene expression at the level of transcription. In certain embodiments, transcription, and thus gene expression, is modulated by replacing or adding a surrogate promoter near the 5′ end of the coding region of a gene-of-interest, thereby resulting in altered gene expression. In certain embodiments, the transcriptional control of one or more genes is engineered to result in the optimal expression of such genes, e.g., in a desired ratio. The term also includes inducible transcriptional control as recognized in the art.

The term “expression” is intended to include the expression of a gene at least at the level of mRNA production.

The term “expression product” is intended to include the resultant product, e.g., a polypeptide, of an expressed gene.

The term “increased expression” is intended to include an alteration in gene expression at least at the level of increased mRNA production and, preferably, at the level of polypeptide expression. The term “increased production” is intended to include an increase in the amount of a polypeptide expressed, in the level of the enzymatic activity of the polypeptide, or a combination thereof, as compared to the native production of, or the enzymatic activity, of the polypeptide.

The terms “activity,” “activities,” “enzymatic activity,” and “enzymatic activities” are used interchangeably and are intended to include any functional activity normally attributed to a selected polypeptide when produced under favorable conditions. Typically, the activity of a selected polypeptide encompasses the total enzymatic activity associated with the produced polypeptide. The polypeptide produced by a host cell and having enzymatic activity may be located in the intracellular space of the cell, cell-associated, secreted into the extracellular milieu, or a combination thereof. Techniques for determining total activity as compared to secreted activity are described herein and are known in the art.

The term “xylanolytic activity” is intended to include the ability to hydrolyze glycosidic linkages in oligopentoses and polypentoses.

The term “cellulolytic activity” is intended to include the ability to hydrolyze glycosidic linkages in oligohexoses and polyhexoses. Cellulolytic activity may also include the ability to depolymerize or debranch cellulose and hemicellulose.

As used herein, the term “lactate dehydrogenase” or “LDH” is intended to include the enzymes capable of converting pyruvate into lactate. It is understood that LDH can also catalyze the oxidation of hydroxybutyrate. LDH includes those enzymes that correspond to Enzyme Commission Number 1.1.1.27.

As used herein the term “alcohol dehydrogenase” or “ADH” is intended to include the enzymes capable of converting acetaldehyde into an alcohol, such as ethanol. ADH also includes the enzymes capable of converting acetone to isopropanol. ADH includes those enzymes that correspond to Enzyme Commission Number 1.1.1.1.

As used herein, the term “phosphotransacetylase” or “PTA” is intended to include the enzymes capable of converting acetyl-phosphate into acetyl-CoA. PTA includes those enzymes that correspond to Enzyme Commission Number 2.3.1.8.

As used herein, the term “acetate kinase” or “ACK” is intended to include the enzymes capable of converting acetate into acetyl-phosphate. ACK includes those enzymes that correspond to Enzyme Commission Number 2.7.2.1.

As used herein, the term “pyruvate formate lyase” or “PFL” is intended to include the enzymes capable of converting pyruvate into acetyl-CoA and formate. PFL includes those enzymes that correspond to Enzyme Commission Number 2.3.1.54.

As used herein, the term “acetaldehyde dehydrogenase” or “ACDH” is intended to include the enzymes capable of converting acetyl-CoA to acetaldehyde. ACDH includes those enzymes that correspond to Enzyme Commission Number 1.2.1.3.

As used herein, the term “acetaldehyde/alcohol dehydrogenase” is intended to include the enzymes capable of converting acetyl-CoA to ethanol. Acetaldehyde/alcohol dehydrogenase includes those enzymes that correspond to Enzyme Commission Numbers 1.2.1.10 and 1.1.1.1.

As used herein, the term “glycerol-3-phosphate dehydrogenase” or “GPD” is intended to include the enzymes capable of converting dihydroxyacetone phosphate to glycerol-3-phosphate. GPD includes those enzymes that correspond to Enzyme Commission Number 1.1.1.8.

As used herein, the term “acetyl-CoA synthetase” or “ACS” is intended to include the enzymes capable of converting acetate to acetyl-CoA. Acetyl-CoA synthetase includes those enzymes that correspond to Enzyme Commission Number 6.2.1.1.

As used herein, the term “thiolase” is intended to include the enzymes capable of converting acetyl-CoA to acetoacetyl-CoA. Thiolase includes those enzymes that correspond to Enzyme Commission Number 2.3.1.9.

As used herein, the term “CoA transferase” is intended to include the enzymes capable of converting acetate and acetoacetyl-CoA to acetoacetate and acetyl-CoA. CoA transferase includes those enzymes that correspond to Enzyme Commission Number 2.8.3.8.

As used herein, the term “acetoacetate decarboxylase” is intended to include the enzymes capable of converting acetoacetate to acetone and carbon dioxide. Acetoacetate decarboxylase includes those enzymes that correspond to Enzyme Commission Number 4.1.1.4.

As used herein, the term “alcohol acetyltransferase” is intended to include the enzymes capable of converting acetyl-CoA and ethanol to ethyl acetate. Alcohol acetyltransferase includes those enzymes that correspond to Enzyme Commission Number 2.3.1.84.

The term “pyruvate decarboxylase activity” is intended to include the ability of a polypeptide to enzymatically convert pyruvate into acetaldehyde and carbon dioxide (e.g., “pyruvate decarboxylase” or “PDC”). Typically, the activity of a selected polypeptide encompasses the total enzymatic activity associated with the produced polypeptide, comprising, e.g., the superior substrate affinity of the enzyme, thermostability, stability at different pHs, or a combination of these attributes. PDC includes those enzymes that correspond to Enzyme Commission Number 4.1.1.1.

A “xylose metabolizing enzyme” can be any enzyme involved in xylose digestion, metabolism and/or hydrolysis, including a xylose isomerase, xylulokinase, xylose reductase, xylose dehydrogenase, xylitol dehydrogenase, xylonate dehydratase, a transketolase, and a transaldolase protein.

A “xylulokinase” (XK) as used herein, is meant for refer to an enzyme that catalyzes the chemical reaction: ATP+D-xylulose⇄ADP+D-xylulose 5-phosphate. Thus, the two substrates of this enzyme are ATP and D-xylulose, whereas its two products are ADP and D-xylulose 5-phosphate. This enzyme belongs to the family of transferases, specifically those transferring phosphorus-containing groups (phosphotransferases) with an alcohol group as acceptor. The systematic name of this enzyme class is ATP:D-xylulose 5-phosphotransferase. Other names in common use include xylulokinase (phosphorylating), and D-xylulokinase. This enzyme participates in pentose and glucuronate interconversions. XK includes those enzymes that correspond to Enzyme Commission Number 2.7.1.17.

A “xylose isomerase” (XI) as used herein, is meant to refer to an enzyme that catalyzes the chemical reaction: D-xylose D-xylulose. This enzyme belongs to the family of isomerases, specifically those intramolecular oxidoreductases interconverting aldoses and ketoses. The systematic name of this enzyme class is D-xylose aldose-ketose-isomerase. Other names in common use include D-xylose isomerase, D-xylose ketoisomerase, and D-xylose ketol-isomerase. This enzyme participates in pentose and glucuronate interconversions and fructose and mannose metabolism. The enzyme is used industrially to convert glucose to fructose in the manufacture of high-fructose corn syrup. It is sometimes referred to as “glucose isomerase”. XI includes those enzymes that correspond to Enzyme Commission Number 5.3.1.5.

As used herein, the term “glucose-6-phosphate isomerase” is intended to include the enzymes capable of converting glucose-6-phosphate into fructose-6-phosphate. Glucose-6-phosphate isomerases include those enzymes that correspond to Enzyme Commission Number 5.3.1.9.

As used herein, the term “transhydrogenase” is intended to include the enzymes capable of converting NADPH and NAD⁺ to NADP⁺ and NADH. Transhydrogenases include those enzymes that correspond to Enzyme Commission Number 1.6.1.1.

As used herein, the term “xylose reductase” is intended to include the enzymes capable of converting xylose and NADP⁺ to NADPH and xylitol. Xylose reductases include those enzymes that correspond to Enzyme Commission Number 1.1.1.307.

As used herein, the term “xylitol dehydrogenase” is intended to include the enzymes capable of converting xylitol and NAD⁺ to NADH and xylulose. Xylitol dehydrogenases include those enzymes that correspond to Enzyme Commission Numbers 1.1.1.9, 1.1.1.10, and 1.1.1. B19.

As used herein, the term “glucose-6-phosphate dehydrogenase” or “glucose-6-P dehydrogenase” is intended to include the enzymes capable of converting glucose-6-phosphate and NADP⁺ to NADPH and 6-phosphoglucono-δ-lactone. Glucose-6-phosphate dehydrogenases include those enzymes that correspond to Enzyme Commission Number 1.1.1.49.

As used herein, the term “6-phospho-3-hexuloisomerase” or “PHI” is intended to include the enzymes capable of converting fructose-6-P to D-arabino-3-hexulose-6-P. 6-phospho-3-hexuloisomerases include those enzymes that correspond to Enzyme Commission Number 5.3.1.27.

As used herein, the term “3-hexulose-6-phosphate synthase” or “HPS” is intended to include the enzymes capable of converting D-arabino-3-hexulose-6-P to ribulose-5-phosphate and formaldehyde. 3-hexulose-6-phosphate synthases include those enzymes that correspond to Enzyme Commission Number 4.1.2.43.

As used herein, the term “formaldehyde dehydrogenase” is intended to include the enzymes capable of converting formaldehyde and NAD⁺ to NADH and formate. Formaldehyde dehydrogenases include those enzymes that correspond to Enzyme Commission Number 1.2.1.46.

As used herein, the term “S-formylglutathione hydrolase” is intended to include the enzymes capable of converting s-formylglutathione to glutathione and formate. S-formylglutathione hydrolases include those enzymes that correspond to Enzyme Commission Number 3.1.2.12.

As used herein, the term “formate dehydrogenase” is intended to include the enzymes capable of converting formate and NAD⁺ to NADH and CO₂. Formate dehydrogenases include those enzymes that correspond to Enzyme Commission Number 1.2.1.2.

As used herein, the term “formaldehyde transketolase” is intended to include the enzymes capable of converting dihydroxyacetone and glyceraldehyde-3-P to xylulose-5-P and formaldehyde. Formaldehyde transketolases include those enzymes that correspond to Enzyme Commission Number 2.2.1.3.

As used herein, the term “dihydroxyacetone phosphatase” is intended to include the enzymes capable of converting dihydroxyacetone-phosphate to dihydroxyacetone. Dihydroxyacetone phosphatases include those enzymes that correspond to Enzyme Commission Number 3.1.3.1. See also Filburn, C. R., “Acid Phosphatase Isozymes of Xenoupus laevis Tadpole Tails: I. Spearation and Partial Characterization,” Archives of Biochem. And Biophysics 159:683-93 (1973).

As used herein, the term “dihydroxyacetone kinase” is intended to include the enzymes capable of converting dihydroxyacetone to dihydroxyacetone phosphate. Dihydroxyacetone kinases include those enzymes that correspond to Enzyme Commission Number 2.7.1.29.

As used herein, the term “glutamate dehydrogenase” is intended to include the enzymes capable of converting L-glutamate and NAD(P)⁺ to 2-oxoglutarate and NAD(P)H. Glutamate dehydrogenases include those enzymes that correspond to Enzyme Commission Numbers 1.4.1.2, 1.4.1.3, and 1.4.1.4.

The term “ethanologenic” is intended to include the ability of a microorganism to produce ethanol from a carbohydrate as a fermentation product. The term is intended to include, but is not limited to, naturally occurring ethanologenic organisms, ethanologenic organisms with naturally occurring or induced mutations, and ethanologenic organisms which have been genetically modified.

The terms “fermenting” and “fermentation” are intended to include the enzymatic process (e.g., cellular or acellular, e.g., a lysate or purified polypeptide mixture) by which ethanol is produced from a carbohydrate, in particular, as a product of fermentation.

The term “secreted” is intended to include the movement of polypeptides to the periplasmic space or extracellular milieu. The term “increased secretion” is intended to include situations in which a given polypeptide is secreted at an increased level (i.e., in excess of the naturally-occurring amount of secretion). In certain embodiments, the term “increased secretion” refers to an increase in secretion of a given polypeptide that is at least about 10% or at least about 100%, 200%, 300%, 400%, 500%, 600%, 700%, 800%, 900%, 1000%, or more, as compared to the naturally-occurring level of secretion.

The term “secretory polypeptide” is intended to include any polypeptide(s), alone or in combination with other polypeptides, that facilitate the transport of another polypeptide from the intracellular space of a cell to the extracellular milieu. In certain embodiments, the secretory polypeptide(s) encompass all the necessary secretory polypeptides sufficient to impart secretory activity to a Gram-negative or Gram-positive host cell or to a yeast host cell. Typically, secretory proteins are encoded in a single region or locus that may be isolated from one host cell and transferred to another host cell using genetic engineering. In certain embodiments, the secretory polypeptide(s) are derived from any bacterial cell having secretory activity or any yeast cell having secretory activity. In certain embodiments, the secretory polypeptide(s) are derived from a host cell having Type II secretory activity. In certain embodiments, the host cell is a thermophilic bacterial cell. In certain embodiments, the host cell is a yeast cell.

The term “derived from” is intended to include the isolation (in whole or in part) of a polynucleotide segment from an indicated source or the purification of a polypeptide from an indicated source. The term is intended to include, for example, direct cloning, PCR amplification, or artificial synthesis from or based on a sequence associated with the indicated polynucleotide source.

By “thermophilic” is meant an organism that thrives at a temperature of about 45° C. or higher.

By “mesophilic” is meant an organism that thrives at a temperature of about 20-45° C.

The term “organic acid” is art-recognized. “Organic acid,” as used herein, also includes certain organic solvents such as ethanol. The term “lactic acid” refers to the organic acid 2-hydroxypropionic acid in either the free acid or salt form. The salt form of lactic acid is referred to as “lactate” regardless of the neutralizing agent, i.e., calcium carbonate or ammonium hydroxide. The term “acetic acid” refers to the organic acid methanecarboxylic acid, also known as ethanoic acid, in either free acid or salt form. The salt form of acetic acid is referred to as “acetate.”

Certain embodiments of the present invention provide for the “insertion,” (e.g., the addition, integration, incorporation, or introduction) of certain genes or particular polynucleotide sequences within thermophilic or mesophilic microorganisms, which insertion of genes or particular polynucleotide sequences may be understood to encompass “genetic modification(s)” or “transformation(s)” such that the resulting strains of said thermophilic or mesophilic microorganisms may be understood to be “genetically modified” or “transformed.” In certain embodiments, strains may be of bacterial, fungal, or yeast origin.

Certain embodiments of the present invention provide for the “inactivation” or “deletion” of certain genes or particular polynucleotide sequences within thermophilic or mesophilic microorganisms, which “inactivation” or “deletion” of genes or particular polynucleotide sequences may be understood to encompass “genetic modification(s)” or “transformation(s)” such that the resulting strains of said thermophilic or mesophilic microorganisms may be understood to be “genetically modified” or “transformed.” In certain embodiments, strains may be of bacterial, fungal, or yeast origin.

The term “CBP organism” is intended to include microorganisms of the invention, e.g., microorganisms that have properties suitable for CBP.

In one aspect of the invention, the genes or particular polynucleotide sequences are inserted to activate the activity for which they encode, such as the expression of an enzyme. In certain embodiments, genes encoding enzymes in the metabolic production of ethanol, e.g., enzymes that metabolize pentose and/or hexose sugars, may be added to a mesophilic or thermophilic organism. In certain embodiments of the invention, the enzyme may confer the ability to metabolize a pentose sugar and be involved, for example, in the D-xylose pathway and/or L-arabinose pathway. In certain embodiments of the invention, genes encoding enzymes in the conversion of acetate to a non-charged solvent, including but not limited to, acetone, isopropanol, or ethanol, may be added to a mesophilic or thermophilic organism.

In one aspect of the invention, the genes or particular polynucleotide sequences are partially, substantially, or completely deleted, silenced, inactivated, or down-regulated in order to inactivate the activity for which they encode, such as the expression of an enzyme. Deletions provide maximum stability because there is no opportunity for a reverse mutation to restore function. Alternatively, genes can be partially, substantially, or completely deleted, silenced, inactivated, or down-regulated by insertion of nucleic acid sequences that disrupt the function and/or expression of the gene (e.g., P1 transduction or other methods known in the art). The terms “eliminate,” “elimination,” and “knockout” are used interchangeably with the terms “deletion,” “partial deletion,” “substantial deletion,” or “complete deletion.” In certain embodiments, strains of thermophilic or mesophilic microorganisms of interest may be engineered by site directed homologous recombination to knockout the production of organic acids. In still other embodiments, RNAi or antisense DNA (asDNA) may be used to partially, substantially, or completely silence, inactivate, or down-regulate a particular gene of interest.

In certain embodiments, the genes targeted for deletion or inactivation as described herein may be endogenous to the native strain of the microorganism, and may thus be understood to be referred to as “native gene(s)” or “endogenous gene(s).” An organism is in “a native state” if it has not been genetically engineered or otherwise manipulated by the hand of man in a manner that intentionally alters the genetic and/or phenotypic constitution of the organism. For example, wild-type organisms may be considered to be in a native state. In other embodiments, the gene(s) targeted for deletion or inactivation may be non-native to the organism.

Similarly, the enzymes of the invention as described herein can be endogenous to the native strain of the microorganism, and can thus be understood to be referred to as “native” or “endogenous.”

The term “upregulated” means increased in activity, e.g., increase in enzymatic activity of the enzyme as compared to activity in a native host organism.

The term “downregulated” means decreased in activity, e.g., decrease in enzymatic activity of the enzyme as compared to activity in a native host organism.

The term “activated” means expressed or metabolically functional.

The term “adapted for growing” means selection of an organism for growth under conditions in which the organism does not otherwise grow or in which the organism grows slowly or minimally. Thus, an organism that is said to be adapted for growing under the selected condition, grows better than an organism that has not been adapted for growing under the selected conditions. Growth can be measured by any methods known in the art, including, but not limited to, measurement of optical density or specific growth rate.

The term “biomass inhibitors” means the inhibitors present in biomass that inhibit processing of the biomass by organisms, including but not limited to, CBP organisms. Biomass inhibitors include, but are not limited to, acids, including without limitation, acetic, lactic, 2-furoic, 3,4-dihydroxybenzoic, 3,5-dihydroxybenzoic, vanillic, homovanillic, syringic, gallic, and ferulic acids; aldehydes, including without limitation, 5-hydroxymethylfurfural, furfural, 3,4-hydroxybenzaldehyde, vanillin, and syringaldehyde. Biomass inhibitors include products removed from pretreated cellulosic material or produced as a result of treating or processing cellulosic material, including but not limited to, inhibitors removed from pretreated mixed hardwood or any other pretreated biomass.

### Biomass

Biomass can include any type of biomass known in the art or described herein. The terms “lignocellulosic material,” “lignocellulosic substrate,” and “cellulosic biomass” mean any type of biomass comprising cellulose, hemicellulose, lignin, or combinations thereof, such as but not limited to woody biomass, forage grasses, herbaceous energy crops, non-woody-plant biomass, agricultural wastes and/or agricultural residues, forestry residues and/or forestry wastes, paper-production sludge and/or waste paper sludge, waste-water-treatment sludge, municipal solid waste, corn fiber from wet and dry mill corn ethanol plants, and sugar-processing residues. The terms “hemicellulosics,” “hemicellulosic portions,” and “hemicellulosic fractions” mean the non-lignin, non-cellulose elements of lignocellulosic material, such as but not limited to hemicellulose (i.e., comprising xyloglucan, xylan, glucuronoxylan, arabinoxylan, mannan, glucomannan, and galactoglucomannan, among others), pectins (e.g., homogalacturonans, rhamnogalacturonan I and II, and xylogalacturonan), and proteoglycans (e.g., arabinogalactan-protein, extensin, and proline-rich proteins).

In a non-limiting example, the lignocellulosic material can include, but is not limited to, woody biomass, such as recycled wood pulp fiber, sawdust, hardwood, softwood, and combinations thereof; grasses, such as switch grass, cord grass, rye grass, reed canary grass, miscanthus, or a combination thereof; sugar-processing residues, such as but not limited to sugar cane bagasse; agricultural wastes, such as but not limited to rice straw, rice hulls, barley straw, corn cobs, cereal straw, wheat straw, canola straw, oat straw, oat hulls, and corn fiber; stover, such as but not limited to soybean stover, corn stover; succulents, such as but not limited to, Agave; and forestry wastes, such as but not limited to, recycled wood pulp fiber, sawdust, hardwood (e.g., poplar, oak, maple, birch, willow), softwood, or any combination thereof. Lignocellulosic material may comprise one species of fiber; alternatively, lignocellulosic material may comprise a mixture of fibers that originate from different lignocellulosic materials. Other lignocellulosic materials are agricultural wastes, such as cereal straws, including wheat straw, barley straw, canola straw and oat straw; corn fiber; stovers, such as corn stover and soybean stover; grasses, such as switch grass, reed canary grass, cord grass, and miscanthus; or combinations thereof.

Paper sludge is also a viable feedstock for lactate or acetate production. Paper sludge is solid residue arising from pulping and paper-making, and is typically removed from process wastewater in a primary clarifier. At a disposal cost of $30/wet ton, the cost of sludge disposal equates to $5/ton of paper that is produced for sale. The cost of disposing of wet sludge is a significant incentive to convert the material for other uses, such as conversion to ethanol. Processes provided by the present invention are widely applicable. Moreover, the saccharification and/or fermentation products may be used to produce ethanol or higher value added chemicals, such as organic acids, aromatics, esters, acetone and polymer intermediates.

### Acetate

Acetate is produced from acetyl-CoA in two reaction steps catalyzed by phosphotransacetylyase (PTA) and acetate kinase (ACK). The reactions mediated by these enzymes are shown below:

PTA reaction: acetyl-CoA+phosphate=CoA+acetyl phosphate (EC 2.3.1.8)

ACK reaction: ADP+acetyl phosphate=ATP+acetate (EC 2.7.2.1)

Both C. thermocellum and C. cellulolyticum make acetate under standard fermentation conditions and have well annotated genes encoding PTA and ACK (see Table 7 of Published U.S. Appl. No. 2012/0094343 A1, which is incorporated by reference herein in its entirety).

### Consolidated Bioprocessing

Consolidated bioprocessing (CBP) is a processing strategy for cellulosic biomass that involves consolidating into a single process step four biologically-mediated events: enzyme production, hydrolysis, hexose fermentation, and pentose fermentation. Implementing this strategy requires development of microorganisms that both utilize cellulose, hemicellulosics, and other biomass components while also producing a product of interest at sufficiently high yield and concentrations. The feasibility of CBP is supported by kinetic and bioenergetic analysis. See van Walsum and Lynd (1998) Biotech. Bioeng. 58:316.

### Xylose Metabolism

Xylose is a five-carbon monosaccharide that can be metabolized into useful products by a variety of organisms. There are two main pathways of xylose metabolism, each unique in the characteristic enzymes they utilize. One pathway is called the “Xylose Reductase-Xylitol Dehydrogenase” or XR-XDH pathway. Xylose reductase (XR) and xylitol dehydrogenase (XDH) are the two main enzymes used in this method of xylose degradation. XR, encoded by the XYL1 gene, is responsible for the reduction of xylose to xylitol and is aided by cofactors NADH or NADPH. Xylitol is then oxidized to xylulose by XDH, which is expressed through the XYL2 gene, and accomplished exclusively with the cofactor NAD+. Because of the varying cofactors needed in this pathway and the degree to which they are available for usage (e.g., XR consumes NADPH and XDH produces NADH), an imbalance can result in an overproduction of xylitol byproduct and an inefficient production of desirable ethanol. Varying expression of the XR and XDH enzyme levels have been tested in the laboratory in the attempt to optimize the efficiency of the xylose metabolism pathway.

The other pathway for xylose metabolism is called the “Xylose Isomerase” (XI) pathway. Enzyme XI is responsible for direct conversion of xylose into xylulose, and does not proceed via a xylitol intermediate. Both pathways create xylulose, although the enzymes utilized are different. After production of xylulose both the XR-XDH and XI pathways proceed through enzyme xylulokinase (XK), encoded on gene XKS1, to further modify xylulose into xylulose-5-P where it then enters the pentose phosphate pathway for further catabolism. XI includes those enzymes that correspond to Enzyme Commission Number 5.3.1.5. Suitable xylose isomerases of the present invention include xylose isomerases derived from Piromyces sp., and B. thetaiotamicron, although any xylose isomerase that functions when expressed in host cells of the invention can be used.

Studies on flux through the pentose phosphate pathway during xylose metabolism have revealed that limiting the speed of this step may be beneficial to the efficiency of fermentation to ethanol. Modifications to this flux that may improve ethanol production include a) lowering phosphoglucose isomerase activity, b) deleting the GND1 gene, and c) deleting the ZWF1 gene. Jeppsson, M., et al., “The level of glucose-6-phosphate dehydrogenase activity strongly influences xylose fermentation and inhibitor sensitivity in recombinant Saccharomyces cerevisiae strains,” Yeast 20:1263-1272 (2003). Since the pentose phosphate pathway produces additional NADPH during metabolism, limiting this step will help to correct the already evident imbalance between NAD(P)H and NAD+ cofactors and reduce xylitol byproduct. An alternative approach is to improve the kinetics of the oxidative branch of the PPP over those of competing pathways. This could be achieved by various approaches, e.g., by directly increasing the expression of the rate-limiting enzyme(s) of the oxidative branch of the PPP pathway, such as glucose-6-P dehydrogenase (encoded endogenously by ZWF1), changing the expression of regulating transcription factors like Stb5p (Cadière, A., et al., “The Saccharomyces cerevisiae zinc factor protein Stb5p is required as a basal regulator of the pentose phosphate pathway,” FEMS Yeast Research 10:819-827 (2010)), or directly down-regulating the expression of genes involved in competing pathways like glucose-6-β isomerase (encoded by PGI1). Producing more CO₂ in the oxidative branch of the PPP would increase the availability of NADPH and increase the NADPH/NADP ratio. This would stimulate the flux of acetate-consuming pathways that (at least partially) consume NADPH, as would for example be the case for ethanol-to-isopropanol conversion that relies on a NADPH-consuming secondary alcohol dehydrogenase to convert acetone to isopropanol, or an acetate-to-ethanol pathway that uses a NADPH-consuming acetaldehyde dehydrogenase and/or alcohol dehydrogenase. Another experiment comparing the two xylose metabolizing pathways revealed that the XI pathway was best able to metabolize xylose to produce the greatest ethanol yield, while the XR-XDH pathway reached a much faster rate of ethanol production (Karhumaa et al., Microb Cell Fact. 2007 Feb. 5; 6:5). See also U.S. Published Appl. No. 2008/0261287 A1, incorporated herein by reference in its entirety.

In one embodiment, the invention comprises combining the XR/XDH pathway for ethanolic xylose fermentation with acetate-to-ethanol conversion through the ACDH pathway. In the proposed pathway, the NADPH consumed in the XR/XDH pathway is regenerated through the pentose phosphate pathway (PPP), while the NADH produced in the XR/XDH pathway is consumed through the acetate-to-ethanol conversion. In contrast to NADH oxidation via glycerol formation, acetate consumption via ACDH results in an overall positive ATP yield. The overall pathway would allow for anaerobic growth on xylose and acetate, providing a selective pressure for improved xylose and acetate consumption and reduced glycerol and xylitol production. It would uncouple acetate uptake from biomass formation, instead providing a fixed stoichiometry between xylose and acetate uptake. This solution to the redox imbalance of the XR/XDH conversion might make the kinetically faster XR/XDH pathway a viable candidate for industrial ethanol production, while the acetate consumption can improve the ethanol yield on xylose by up to 20%. Acetate consumption would furthermore reduce the toxicity of the cellulosic feedstock hydrolysate.

### Ribulose-Monophosphate Pathway

In another embodiment, the invention comprises introducing the heterologous ribulose-monophosphate (RuMP) pathway found in various bacteria and archaea, which also produces CO₂ while conferring electrons to redox carriers. The RuMP pathway relies on the expression of two heterologous genes: 6-phospho-3-hexuloisomerase (PHI) and 3-hexulose-6-phosphate synthase (HPS). PHI converts fructose-6-P to D-arabino-3-hexulose-6-P, and HPS converts the latter to ribulose-5-P and formaldehyde. While this conversion is redox neutral, the produced formaldehyde can then be converted to CO₂ by the action of the endogenous enzymes formaldehyde dehydrogenase and S-formylglutathione hydrolase (which produce formate and NADH) and formate dehydrogenase (which convert the formate to CO₂, producing a second NADH).

The RuMP pathway has been characterized as a reversible pathway, and many of the characterized enzymes have been found in thermophiles. Candidate genes can be derived from the mesophilic Mycobacterium gastri, Bacillus subtilis, Methylococcus capsulatus, and Thermococcus kodakaraensis. See Mitsui, R., et al., “A Novel Operon Encoding Formaldehyde Fixation: the Ribulose Monophosphate Pathway in the Gram-Positive Facultative Methylotrophic Bacterium Mycobacterium gastri MB19,”Journal of Bacteriology 182:944-948 (2000); Yasueda, H., et al., “Bacillus subtilis yckG and yckF Encode Two Key Enzymes of the Ribulose Monophosphate Pathway Used by Methylotrophs, and yckH is Required for Their Expression,” J. of Bacteriol. 181:7154-60 (1999); Ferenci, T., et al., “Purification and properties of 3-hexulose phosphate synthase and phospho-3-hexuloisomerase from Methylococcus capsulatus,” Biochem J. 144:477-86 (1974); Orita, I., et al., “The Ribulose Monophosphate Pathway Substitutes for the Missing Pentose Phosphate Pathway in the Archaeon Thermococcus kodakaraensis,” J. Bacteriol. 188:4698-4704 (2006).

### Dihydroxyacetone Pathway

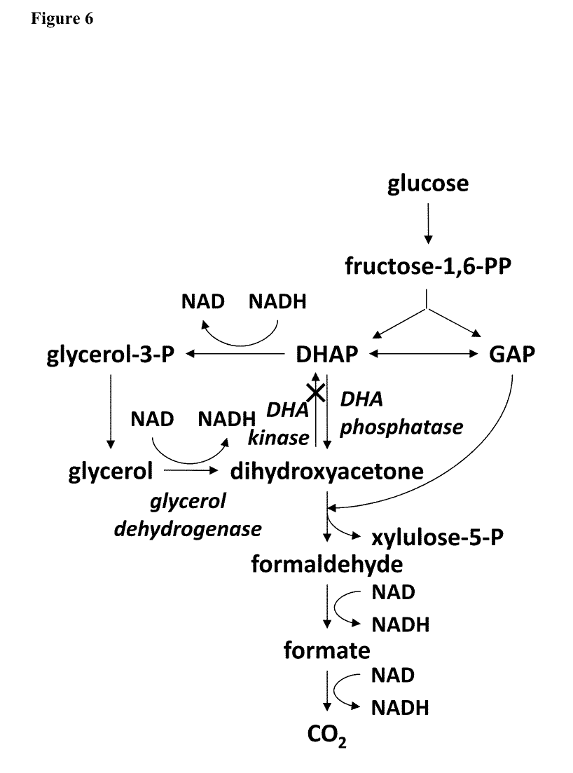

In another embodiment, the invention comprises using the dihydroxyacetone pathway (DHA), which also produces CO₂ while conferring electrons to redox carriers. In one embodiment, the invention comprises a DHA pathway that is endogenous to S. cerevisiae and comprises the genes glycerol dehydrogenase and formaldehyde transketolase and results in formaldehyde oxidation to CO₂. In another embodiment, the invention comprises a DHA pathway that comprises heterologous enzymes such as gdh from Ogataea polymorpha. See Nguyen, H. T. T. & Nevoigt, E., “Engineering of Saccharomyces cerevisiae for the production of dihydroxyacetone (DHA) from sugars: A proof of concept,” Metabolic Engineering 11:335-46 (2009). The DHA pathway is conceptually similar to the RuMP pathway as both rely on the formation of formaldehyde and the subsequent oxidation of the formaldehyde to CO₂, producing NADH. With the DHA pathway, formaldehyde is produced by the action of formaldehyde transketolase (EC 2.2.1.3), which interconverts dihydroxyacetone and glyceraldehyde-3-P into xylulose-5-P and formaldehyde. See FIG. 6. The required dihydroxyacetone can be produced by either glycerol dehydrogenase or dihydroxyacetone phosphatase:

glycerol+NAD(P)→dihydroxyacetone+NAD(P)H (glycerol dehydrogenase) or

dihydroxyacetone-P→dihydroxyacetone (dihydroxyacetone phosphatase)

dihydroxyacetone+glyceraldehyde-3-P→xylulose-5-P+formaldehyde (formaldehyde transketolase)

formaldehyde→CO₂+2 NADH (formaldehyde dehydrogenase, S-formylglutathione hydrolase, and formate dehydrogenase)

DHA degradation via formaldehyde transketolase has been described for S. cerevisiae, and baker's yeast has an endogenous glycerol dehydrogenase, encoded by GCY1. See Molin, M., and A. Blomberg, “Dihydroxyacetone detoxification in Saccharomyces cerevisiae involves formaldehyde dissimilation,” Mol. Microbiol. 60:925-938 (2006) and Yu, K. O., et al., “Engineering of glycerol utilization pathway for ethanol production by Saccharomyces cerevisiae,” Bioresource Technol. 101:4157-4161 (2010). Glycerol dehydrogenases from several organisms, including Hansenula polymorpha (gdh), E. coli (gldA) and Pichia angusta (gdh), have also been functionally expressed in S. cerevisiae. See Jung, J.-Y., et al., “Production of 1,2-propanediol from glycerol in Saccharomyces cerevisiae,” J. Microbiol. Biotechnol. 21:846-853 (2011) and Nguyen, H. T. T., and Nevoigt, E., “Engineering of Saccharomyces cerevisiae for the production of dihydroxyacetone (DHA) from sugars: A proof of concept, “Metabolic Engineering 11:335-346 (2009). Dihydroxyacetone-P-specific phosphatase-activity has been found in the bacterium Zymomonas mobilis. See Horbach, S., et al., “Enzymes involved in the formation of glycerol 3-phosphate and the by-products dihydroxyacetone and glycerol in Zymomonas mobilis,” FEMS Microbiology Letters 120:37-44 (1994).

### Transhydrogenase

In another embodiment, the invention comprises the introduction of a transhydrogenase for the production of electron donors to be used in the conversion of acetate to ethanol or isopropanol.

As the (cytosolic) NADPH/NADP ratio in S. cerevisiae is typically assumed to be higher than the NADH/NAD ratio, introduction of a transhydrogenase should create a flux towards NADH formation as transhydrogenases catalyze the following reaction: NADPH+NAD⁺ [symbol] NADP⁺+NADH. Transhydrogenases from Escherichia coli and Azotobacter vinelandii have been successfully expressed in S. cerevisiae, and observed changes in the metabolic profiles (increased glycerol, acetate and 2-oxoglutarate production, decreased xylitol production) indeed pointed to a net conversion of NADPH into NADH. See Anderlund, M., et al., “Expression of the Escherichia coli pntA and pntB Genes, Encoding Nicotinamide Nucleotide Transhydrogenase, in Saccharomyces cerevisiae and Its Effect on Product Formation during Anaerobic Glucose Fermentation,” Appl. Envirol. Microbiol. 65:2333-340 (1999); Heux, S., et al., “Glucose utilization of strains lacking PGI1 and expressing a transhydrogenase suggests differences in the pentose phosphate capacity among Saccharomyces cerevisiae strains,” FEMS Yeast Research 8:217-224 (2008); Jeppsson, M., et al., (2003); Jeun, Y.-S., et al., “Expression of Azotobacter vinelandii soluble transhydrogenase perturbs xylose reductase-mediated conversion of xylose to xylitol by recombinant Saccharomyces cerevisiae,” Journal of Molecular Catalysis B: Enzymatic 26:251-256 (2003); and Nissen, T. L., et al., “Expression of a cytoplasmic transhydrogenase in Saccharomyces cerevisiae results in formation of 2-oxoglutarate due to depletion of the NADPH pool,” Yeast 18:19-32 (2001).

With this approach, additional NADH becomes available for acetate-to-ethanol conversion, and the consumed NADPH could be replenished by increasing the flux through the pentose phosphate pathway.

### Glutamate Dehydrogenase

In another embodiment, the invention comprises the introduction of a NADPH/NADH-cycling reaction. One such cycle consists of the combination of cytosolic NAD-specific and NADP-specific glutamate dehydrogenases (GDH), which catalyze the reversible reaction:

L-glutamate+H₂O+NAD(P)+ [symbol] 2-oxoglutarate+NH₃+NAD(P)H+H⁺

Overexpressing the native NAD-GDH encoded by GDH2 (SEQ ID NO:1) has been shown to rescue growth in a phosphoglucose isomerase pgi1 S. cerevisiae deletion mutant, but only as long as glucose-6-phosphate dehydrogenase and the NADP-GDH encoded by GDHJ were left intact. See Boles, E., et al., “The role of the NAD-dependent glutamate dehydrogenase in restoring growth on glucose of a Saccharomyces cerevisiae phosphoglucose isomerase mutant,” European Journal of Biochemistry 217:469-477 (1993). This strongly suggests that the increased NADPH production, the result of redirection of glucose into the pentose phosphate pathway, which normally proves fatal, could be balanced by conversion of NADPH to NADH by this GDH-cycle, with the produced NADH being reoxidized via respiration.

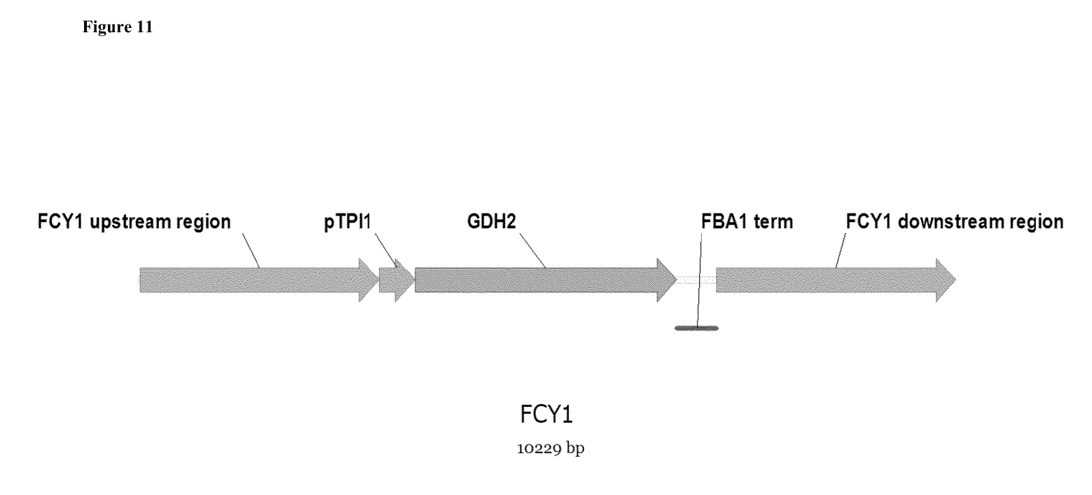

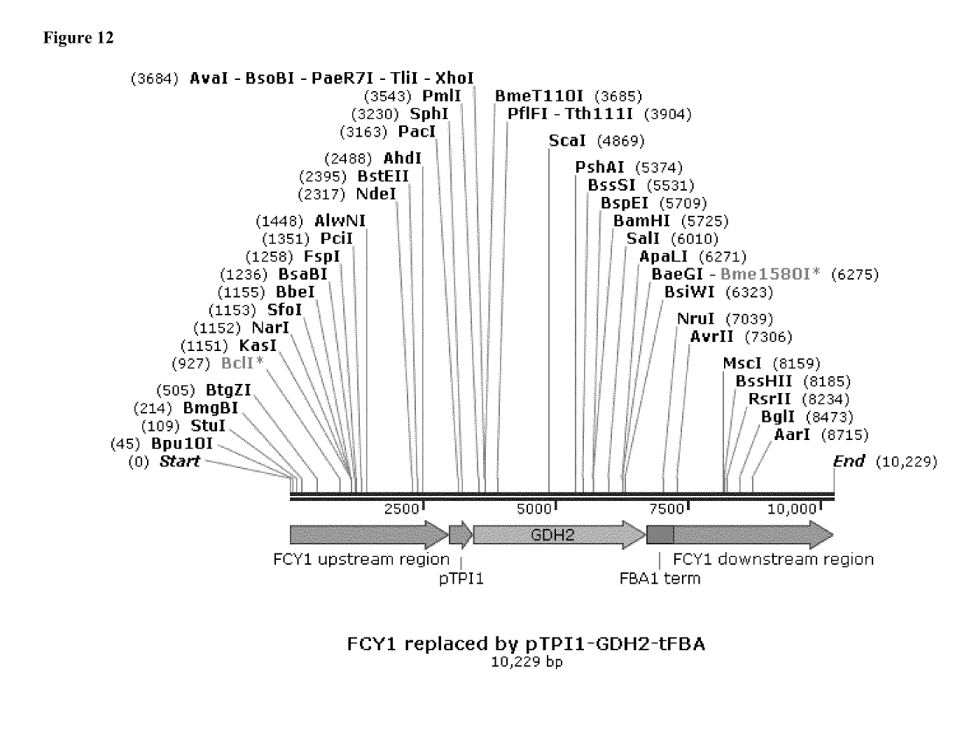

As with transhydrogenase, when the cytosolic NADPH/NADP ratio is higher than the NADH/NAD ratio, introducing a GDH-cycling reaction would generate additional NADH at the expense of NADPH. The latter can then again be replenished by an increased flux through the pentose phosphate pathway. In one embodiment, the invention comprises a copy of GDH2 under the control of a strong constitutive promoter (e.g., pTPI1) that is integrated in the genomic DNA of S. cerevisiae which also expresses a NADH-specific acetaldehyde dehydrogenase. See FIGS. 11 and 12.

The DNA and amino acid sequences for S. cerevisiae GDH2 are provided as SEQ ID NOs:1 and 2, respectively. The sequence for the strong constitutive promoter pTPI1 is provided as SEQ ID NO:3.

### Glycerol Reduction

Anaerobic growth conditions require the production of endogenous electron acceptors, such as the coenzyme nicotinamide adenine dinucleotide (NAD⁺). In cellular redox reactions, the NAD⁺/NADH couple plays a vital role as a reservoir and carrier of reducing equivalents. Ansell, R., et al., EMBO J. 16:2179-87 (1997). Cellular glycerol production, which generates an NAD⁺, serves as a redox valve to remove excess reducing power during anaerobic fermentation in yeast. Glycerol production is, however, an energetically wasteful process that expends ATP and results in the loss of a reduced three-carbon compound. Ansell, R., et al., EMBO J. 16:2179-87 (1997). To generate glycerol from a starting glucose molecule, glycerol 3-phosphate dehydrogenase (GPD) reduces dihydroxyacetone phosphate to glycerol 3-phosphate and glycerol 3-phosphatase (GPP) dephosphorylates glycerol 3-phosphate to glycerol. Despite being energetically wasteful, glycerol production is a necessary metabolic process for anaerobic growth as deleting GPD activity completely inhibits growth under anaerobic conditions. See Ansell, R., et al., EMBO J. 16:2179-87 (1997).

GPD is encoded by two isogenes, gpd1 and gpd2. GPD1 encodes the major isoform in anaerobically growing cells, while GPD2 is required for glycerol production in the absence of oxygen, which stimulates its expression. Pahlman, A-K., et al., J. Biol. Chem. 276:3555-63 (2001). The first step in the conversion of dihydroxyacetone phosphate to glycerol by GPD is rate controlling. Guo, Z. P., et al., Metab. Eng. 13:49-59 (2011). GPP is also encoded by two isogenes, gpp1 and gpp2. The deletion of GPP genes arrests growth when shifted to anaerobic conditions, demonstrating that GPP is important for cellular tolerance to osmotic and anaerobic stress. See Pahlman, A-K., et al., J. Biol. Chem. 276:3555-63 (2001).

Because glycerol is a major by-product of anaerobic production of ethanol, many efforts have been made to delete cellular production of glycerol. However, because of the reducing equivalents produced by glycerol synthesis, deletion of the glycerol synthesis pathway cannot be done without compensating for this valuable metabolic function. Attempts to delete glycerol production and engineer alternate electron acceptors have been made. Lidén, G., et al., Appl. Env. Microbial. 62:3894-96 (1996); Medina, V. G., et al., Appl. Env. Microbiol. 76:190-195 (2010). Lidén and Medina both deleted the gpd1 and gpd2 genes and attempted to bypass glycerol formation using additional carbon sources. Lidén engineered a xylose reductase from Pichia stipitis into an S. cerevisiae gpd1/2 deletion strain. The xylose reductase activity facilitated the anaerobic growth of the glycerol-deleted strain in the presence of xylose. See Lidén, G., et al., Appl. Env. Microbiol. 62:3894-96 (1996). Medina engineered an acetylaldehyde dehydrogenase, mhpF, from E. coli into an S. cerevisiae gpd1/2 deletion strain to convert acetyl-CoA to acetaldehyde. The acetylaldehyde dehydrogenase activity facilitated the anaerobic growth of the glycerol-deletion strain in the presence of acetic acid but not in the presence of glucose as the sole source of carbon. Medina, V. G., et al., Appl. Env. Microbiol. 76:190-195 (2010); see also EP 2277989. Medina noted several issues with the mhpF-containing strain that needed to be addressed before implementing industrially, including significantly reduced growth and product formation rates than yeast comprising GPD1 and GPD2.

Thus, in some embodiments of the invention, the recombinant host cells comprise a deletion or alteration of one or more glycerol producing enzymes. Additional deletions or alterations to modulate glycerol production include, but are not limited to, engineering a pyruvate formate lyase in a recombinant host cell, and are described in U.S. Appl. No. 61/472,085, incorporated by reference herein in its entirety.

### Microorganisms

The present invention includes multiple strategies for the development of microorganisms with the combination of substrate-utilization and product-formation properties required for CBP. The “native cellulolytic strategy” involves engineering naturally occurring cellulolytic microorganisms to improve product-related properties, such as yield and titer. The “recombinant cellulolytic strategy” involves engineering natively non-cellulolytic organisms that exhibit high product yields and titers to express a heterologous cellulase system that enables cellulose utilization or hemicellulose utilization or both.

Many bacteria have the ability to ferment simple hexose sugars into a mixture of acidic and pH-neutral products via the process of glycolysis. The glycolytic pathway is abundant and comprises a series of enzymatic steps whereby a six carbon glucose molecule is broken down, via multiple intermediates, into two molecules of the three-carbon compound pyruvate. This process results in the net generation of ATP (biological energy supply) and the reduced cofactor NADH.

Pyruvate is an important intermediary compound of metabolism. For example, under aerobic conditions pyruvate may be oxidized to acetyl coenzyme A (acetyl-CoA), which then enters the tricarboxylic acid cycle (TCA), which in turn generates synthetic precursors, CO₂, and reduced cofactors. The cofactors are then oxidized by donating hydrogen equivalents, via a series of enzymatic steps, to oxygen resulting in the formation of water and ATP. This process of energy formation is known as oxidative phosphorylation.

Under anaerobic conditions (no available oxygen), fermentation occurs in which the degradation products of organic compounds serve as hydrogen donors and acceptors. Excess NADH from glycolysis is oxidized in reactions involving the reduction of organic substrates to products, such as lactate and ethanol. In addition, ATP is regenerated from the production of organic acids, such as acetate, in a process known as substrate level phosphorylation. Therefore, the fermentation products of glycolysis and pyruvate metabolism include a variety of organic acids, alcohols and CO₂.

Most facultative anaerobes metabolize pyruvate aerobically via pyruvate dehydrogenase (PDH) and the tricarboxylic acid cycle (TCA). Under anaerobic conditions, the main energy pathway for the metabolism of pyruvate is via pyruvate-formate-lyase (PFL) pathway to give formate and acetyl-CoA. Acetyl-CoA is then converted to acetate, via phosphotransacetylase (PTA) and acetate kinase (ACK) with the co-production of ATP, or reduced to ethanol via acetaldehyde dehydrogenase (ACDH) and alcohol dehydrogenase (ADH). In order to maintain a balance of reducing equivalents, excess NADH produced from glycolysis is re-oxidized to NAD+ by lactate dehydrogenase (LDH) during the reduction of pyruvate to lactate. NADH can also be re-oxidized by ACDH and ADH during the reduction of acetyl-CoA to ethanol, but this is a minor reaction in cells with a functional LDH.

### Host Cells

Host cells useful in the present invention include any prokaryotic or eukaryotic cells; for example, microorganisms selected from bacterial, algal, and yeast cells. Among host cells thus suitable for the present invention are microorganisms, for example, of the genera Aeromonas, Aspergillus, Bacillus, Escherichia, Kluyveromyces, Pichia, Rhodococcus, Saccharomyces and Streptomyces.

In some embodiments, the host cells are microorganisms. In one embodiment the microorganism is a yeast. According to the present invention the yeast host cell can be, for example, from the genera Saccharomyces, Kluyveromyces, Candida, Pichia, Schizosaccharomyces, Hansenula, Kloeckera, Schwanniomyces, and Yarrowia. Yeast species as host cells may include, for example, S. cerevisiae, S. bulderi, S. barnetti, S. exiguus, S. uvarum, S. diastaticus, K. lactis, K marxianus, or K. fragilis. In some embodiments, the yeast is selected from the group consisting of Saccharomyces cerevisiae, Schizzosaccharomyces pombe, Candida albicans, Pichia pastoris, Pichia stipitis, Yarrowia lipolytica, Hansenula polymorpha, Phaffia rhodozyma, Candida utilis, Arxula adeninivorans, Debaryomyces hansenii, Debaryomyces polymorphus, Schizosaccharomyces pombe and Schwanniomyces occidentalis. In one particular embodiment, the yeast is Saccharomyces cerevisiae. In another embodiment, the yeast is a thermotolerant Saccharomyces cerevisiae. The selection of an appropriate host is deemed to be within the scope of those skilled in the art from the teachings herein.

In some embodiments, the host cell is an oleaginous cell. The oleaginous host cell can be an oleaginous yeast cell. For example, the oleaginous yeast host cell can be from the genera Blakeslea, Candida, Cryptococcus, Cunninghamella, Lipomyces, Mortierella, Mucor, Phycomyces, Pythium, Rhodosporidum, Rhodotorula, Trichosporon or Yarrowia. According to the present invention, the oleaginous host cell can be an oleaginous microalgae host cell. For example, the oleaginous microalgae host cell can be from the genera Thraustochytrium or Schizochytrium. Biodiesel could then be produced from the triglyceride produced by the oleaginous organisms using conventional lipid transesterification processes. In some particular embodiments, the oleaginous host cells can be induced to secrete synthesized lipids. Embodiments using oleaginous host cells are advantageous because they can produce biodiesel from lignocellulosic feedstocks which, relative to oilseed substrates, are cheaper, can be grown more densely, show lower life cycle carbon dioxide emissions, and can be cultivated on marginal lands.

In some embodiments, the host cell is a thermotolerant host cell. Thermotolerant host cells can be particularly useful in simultaneous saccharification and fermentation processes by allowing externally produced cellulases and ethanol-producing host cells to perform optimally in similar temperature ranges.

Thermotolerant host cells can include, for example, Issatchenkia orientalis, Pichia mississippiensis, Pichia mexicana, Pichia farinosa, Clavispora opuntiae, Clavispora lusitaniae, Candida mexicana, Hansenula polymorpha and Kluyveromyces host cells. In some embodiments, the thermotolerant cell is an S. cerevisiae strain, or other yeast strain, that has been adapted to grow in high temperatures, for example, by selection for growth at high temperatures in a cytostat.

In some particular embodiments, the host cell is a Kluyveromyces host cell. For example, the Kluyveromyces host cell can be a K. lactis, K. marxianus, K. blattae, K. phaffii, K. yarrowii, K. aestuarii, K. dobzhanskii, K. wickerhamii, K. thermotolerans, or K. waltii host cell. In one embodiment, the host cell is a K. lactis, or K. marxianus host cell. In another embodiment, the host cell is a K. marxianus host cell.

In some embodiments, the thermotolerant host cell can grow at temperatures above about 30° C., about 31° C., about 32° C., about 33° C., about 34° C., about 35° C., about 36° C., about 37° C., about 38° C., about 39° C., about 40° C., about 41° C. or about 42° C. In some embodiments of the present invention the thermotolerant host cell can produce ethanol from cellulose at temperatures above about 30° C., about 31° C., about 32° C., about 33° C., about 34° C., about 35° C., about 36° C., about 37° C., about 38° C., about 39° C., about 40° C., about 41° C., about 42° C., or about 43° C., or about 44° C., or about 45° C., or about 50° C.

In some embodiments of the present invention, the thermotolerant host cell can grow at temperatures from about 30° C. to 60° C., about 30° C. to 55° C., about 30° C. to 50° C., about 40° C. to 60° C., about 40° C. to 55° C. or about 40° C. to 50° C. In some embodiments of the present invention, the thermotolerant host cell can produce ethanol from cellulose at temperatures from about 30° C. to 60° C., about 30° C. to 55° C., about 30° C. to 50° C., about 40° C. to 60° C., about 40° C. to 55° C. or about 40° C. to 50° C.

In some embodiments, the host cell has the ability to metabolize xylose. Detailed information regarding the development of the xylose-utilizing technology can be found in the following publications: Kuyper M. et al., FEMS Yeast Res. 4: 655-64 (2004), Kuyper M. et al., FEMS Yeast Res. 5:399-409 (2005), and Kuyper M. et al., FEMS Yeast Res. 5:925-34 (2005), which are herein incorporated by reference in their entirety. For example, xylose-utilization can be accomplished in S. cerevisiae by heterologously expressing the xylose isomerase gene, XylA, e.g., from the anaerobic fungus Piromyces sp. E2, overexpressing five S. cerevisiae enzymes involved in the conversion of xylulose to glycolytic intermediates (xylulokinase, ribulose 5-phosphate isomerase, ribulose 5-phosphate epimerase, transketolase and transaldolase) and deleting the GRE3 gene encoding aldose reductase to minimize xylitol production.

The host cells can contain antibiotic markers or can contain no antibiotic markers.

In certain embodiments, the host cell is a microorganism that is a species of the genera Thermoanaerobacterium, Thermoanaerobacter, Clostridium, Geobacillus, Saccharococcus, Paenibacillus, Bacillus, Caldicellulosiruptor, Anaerocellum, or Anoxybacillus. In certain embodiments, the host cell is a bacterium selected from the group consisting of: Thermoanaerobacterium thermosulfurigenes, Thermoanaerobacterium aotearoense, Thermoanaerobacterium polysaccharolyticum, Thermoanaerobacterium zeae, Thermoanaerobacterium xylanolyticum, Thermoanaerobacterium saccharolyticum, Thermoanaerobium brockii, Thermoanaerobacterium thermosaccharolyticum, Thermoanaerobacter thermohydrosulfuricus, Thermoanaerobacter ethanolicus, Thermoanaerobacter brocki, Clostridium thermocellum, Clostridium cellulolyticum, Clostridium phytofermentans, Clostridium straminosolvens, Geobacillus thermoglucosidasius, Geobacillus stearothermophilus, Saccharococcus caldoxylosilyticus, Saccharoccus thermophilus, Paenibacillus campinasensis, Bacillus flavothermus, Anoxybacillus kamchatkensis, Anoxybacillus gonensis, Caldicellulosiruptor acetigenus, Caldicellulosiruptor saccharolyticus, Caldicellulosiruptor kristjanssonii, Caldicellulosiruptor owensensis, Caldicellulosiruptor lactoaceticus, and Anaerocellumthermophilum. In certain embodiments, the host cell is Clostridium thermocellum, Clostridium cellulolyticum, or Thermoanaerobacterium saccharolyticum.

### Codon Optimized Polynucleotides

The polynucleotides encoding heterologous enzymes described herein can be codon-optimized. As used herein the term “codon-optimized coding region” means a nucleic acid coding region that has been adapted for expression in the cells of a given organism by replacing at least one, or more than one, or a significant number, of codons with one or more codons that are more frequently used in the genes of that organism.

In general, highly expressed genes in an organism are biased towards codons that are recognized by the most abundant transfer RNA (tRNA) species in that organism. One measure of this bias is the “codon adaptation index” or “CAI,” which measures the extent to which the codons used to encode each amino acid in a particular gene are those which occur most frequently in a reference set of highly expressed genes from an organism.

The CAI of codon optimized sequences of the present invention corresponds to between about 0.8 and 1.0, between about 0.8 and 0.9, or about 1.0. A codon optimized sequence may be further modified for expression in a particular organism, depending on that organism's biological constraints. For example, large runs of “As” or “Ts” (e.g., runs greater than 3, 4, 5, 6, 7, 8, 9, or 10 consecutive bases) can be removed from the sequences if these are known to effect transcription negatively. Furthermore, specific restriction enzyme sites may be removed for molecular cloning purposes. Examples of such restriction enzyme sites include PacI, AscI, BamHI, BglII, EcoRI and XhoI. Additionally, the DNA sequence can be checked for direct repeats, inverted repeats and mirror repeats with lengths of ten bases or longer, which can be modified manually by replacing codons with “second best” codons, i.e., codons that occur at the second highest frequency within the particular organism for which the sequence is being optimized.

Deviations in the nucleotide sequence that comprise the codons encoding the amino acids of any polypeptide chain allow for variations in the sequence coding for the gene. Since each codon consists of three nucleotides, and the nucleotides comprising DNA are restricted to four specific bases, there are 64 possible combinations of nucleotides, 61 of which encode amino acids (the remaining three codons encode signals ending translation). The “genetic code” which shows which codons encode which amino acids is reproduced herein as Table 1. As a result, many amino acids are designated by more than one codon. For example, the amino acids alanine and proline are coded for by Ebur triplets, serine and arginine by six, whereas tryptophan and methionine are coded by just one triplet. This degeneracy allows for DNA base composition to vary over a wide range without altering the amino acid sequence of the proteins encoded by the DNA.

TABLE 1
The Standard Genetic Code

| | T | C | A | G |
|---|---|---|---|---|
| T | TTT Phe(F) | TCT Ser(S) | TAT Tyr(Y) | TGT Cys(C) |
| | TTC Phe(F) | TCC Ser(S) | TAC Tyr(Y) | TGC |
| | TTA Leu(L) | TCA Ser(S) | TAA Ter | TGA Ter |
| | TTG Leu(L) | TCG Ser(S) | TAG Ter | TGG Trp(W) |
| C | CTT Leu(L) | CCT Pro(P) | CAT His(H) | CGT Arg(R) |
| | CTC Leu(L) | CCC Pro(P) | CAC His(H) | CGC Arg(R) |
| | CTA Leu(L) | CCA Pro(P) | CAA Gln(Q) | CGA Arg(R) |
| | CTG Leu(L) | CCG Pro(P) | CAG Gln(Q) | CGG Arg(R) |
| A | ATT Ile(I) | ACT Thr(T) | AAT Asn(N) | AGT Ser(S) |
| | ATC Ile(I) | ACC Thr(T) | AAC Asn(N) | AGC Ser(S) |
| | ATA Ile(I) | ACA Thr(T) | AAA Lys(K) | AGA Arg(R) |
| | ATG Met(M) | ACG Thr(T) | AAG Lys(K) | AGG Arg(R) |
| G | GTT Val(V) | GCT Ala(A) | GAT Asp(D) | GGT Gly(G) |
| | GTC Val(V) | GCC Ala(A) | GAC Asp(D) | GGC Gly(G) |
| | GTA Val(V) | GCA Ala(A) | GAA Glu(E) | GGA Gly(G) |
| | GTG Val(V) | GCG Ala(A) | GAG Glu(E) | GGG Gly(G) |

Many organisms display a bias for use of particular codons to code for insertion of a particular amino acid in a growing peptide chain. Codon preference or codon bias, differences in codon usage between organisms, is afforded by degeneracy of the genetic code, and is well documented among many organisms. Codon bias often correlates with the efficiency of translation of messenger RNA (mRNA), which is in turn believed to be dependent on, inter alia, the properties of the codons being translated and the availability of particular tRNA molecules. The predominance of selected tRNAs in a cell is generally a reflection of the codons used most frequently in peptide synthesis. Accordingly, genes can be tailored for optimal gene expression in a given organism based on codon optimization.

Given the large number of gene sequences available for a wide variety of animal, plant and microbial species, it is possible to calculate the relative frequencies of codon usage. Codon usage tables are readily available, for example, at kazusa.or.jp/codon/ (visited Aug. 10, 2012), and these tables can be adapted in a number of ways. See Nakamura, Y., et al. “Codon usage tabulated from the international DNA sequence databases: status for the year 2000,” Nucl. Acids Res. 28:292 (2000). Codon usage tables for yeast, calculated from GenBank Release 128.0 [15 Feb. 2002], are reproduced below as Table 2. This table uses mRNA nomenclature, and so instead of thymine (T) which is found in DNA, the tables use uracil (U) which is found in RNA. The table has been adapted so that frequencies are calculated for each amino acid, rather than for all 64 codons.

TABLE 2
Codon Usage Table for Saccharomyces cerevisiae Genes

| | | | Frequency per |
|---|---|---|---|
| Amino Acid | Codon | Number | hundred |
| Phe | UUU | 170666 | 26.1 |
| Phe | UUC | 120510 | 18.4 |
| Leu | UUA | 170884 | 26.2 |
| Leu | UUG | 177573 | 27.2 |
| Leu | CUU | 80076 | 12.3 |
| Leu | CUC | 35545 | 5.4 |
| Leu | CUA | 87619 | 13.4 |
| Leu | CUG | 68494 | 10.5 |
| Ile | AUU | 196893 | 30.1 |
| Ile | AUC | 112176 | 17.2 |
| Ile | AUA | 116254 | 17.8 |
| Met | AUG | 136805 | 20.9 |
| Val | GUU | 144243 | 22.1 |
| Val | GUC | 76947 | 11.8 |
| Val | GUA | 76927 | 11.8 |
| Val | GUG | 70337 | 10.8 |
| Ser | UCU | 153557 | 23.5 |
| Ser | UCC | 92923 | 14.2 |
| Ser | UCA | 122028 | 18.7 |
| Ser | UCG | 55951 | 8.6 |
| Ser | AGU | 92466 | 14.2 |
| Ser | AGC | 63726 | 9.8 |
| Pro | CCU | 88263 | 13.5 |
| Pro | CCC | 44309 | 6.8 |
| Pro | CCA | 119641 | 18.3 |
| Pro | CCG | 34597 | 5.3 |
| Thr | ACU | 132522 | 20.3 |
| Thr | ACC | 83207 | 12.7 |
| Thr | ACA | 116084 | 17.8 |
| Thr | ACG | 52045 | 8.0 |
| Ala | GCU | 138358 | 21.2 |
| Ala | GCC | 82357 | 12.6 |
| Ala | GCA | 105910 | 16.2 |
| Ala | GCG | 40358 | 6.2 |
| Tyr | UAU | 122728 | 18.8 |
| Tyr | UAC | 96596 | 14.8 |
| His | CAU | 89007 | 13.6 |
| His | CAC | 50785 | 7.8 |
| Gln | CAA | 178251 | 27.3 |
| Gln | CAG | 79121 | 12.1 |
| Asn | AAU | 233124 | 35.7 |
| Asn | AAC | 162199 | 24.8 |
| Lys | AAA | 273618 | 41.9 |
| Lys | AAG | 201361 | 30.8 |
| Asp | GAU | 245641 | 37.6 |
| Asp | GAC | 132048 | 20.2 |
| Glu | GAA | 297944 | 45.6 |
| Glu | GAG | 125717 | 19.2 |
| Cys | UGU | 52903 | 8.1 |
| Cys | UGC | 31095 | 4.8 |
| Trp | UGG | 67789 | 10.4 |
| Arg | CGU | 41791 | 6.4 |
| Arg | CGC | 16993 | 2.6 |
| Arg | CGA | 19562 | 3.0 |
| Arg | CGG | 11351 | 1.7 |
| Arg | AGA | 139081 | 21.3 |
| Arg | AGG | 60289 | 9.2 |
| Gly | GGU | 156109 | 23.9 |
| Gly | GGC | 63903 | 9.8 |
| Gly | GGA | 71216 | 10.9 |
| Gly | GGG | 39359 | 6.0 |
| Stop | UAA | 6913 | 1.1 |
| Stop | UAG | 3312 | 0.5 |
| Stop | UGA | 4447 | 0.7 |

By utilizing this or similar tables, one of ordinary skill in the art can apply the frequencies to any given polypeptide sequence, and produce a nucleic acid fragment of a codon-optimized coding region which encodes the polypeptide, but which uses codons optimal for a given species. Codon-optimized coding regions can be designed by various different methods.

In one method, a codon usage table is used to find the single most frequent codon used for any given amino acid, and that codon is used each time that particular amino acid appears in the polypeptide sequence. For example, referring to Table 2 above, for leucine, the most frequent codon is UUG, which is used 27.2% of the time. Thus all the leucine residues in a given amino acid sequence would be assigned the codon UUG.

In another method, the actual frequencies of the codons are distributed randomly throughout the coding sequence. Thus, using this method for optimization, if a hypothetical polypeptide sequence had 100 leucine residues, referring to Table 2 for frequency of usage in the S. cerevisiae, about 5, or 5% of the leucine codons would be CUC, about 11, or 11% of the leucine codons would be CUG, about 12, or 12% of the leucine codons would be CUU, about 13, or 13% of the leucine codons would be CUA, about 26, or 26% of the leucine codons would be UUA, and about 27, or 27% of the leucine codons would be UUG.

These frequencies would be distributed randomly throughout the leucine codons in the coding region encoding the hypothetical polypeptide. As will be understood by those of ordinary skill in the art, the distribution of codons in the sequence can vary significantly using this method; however, the sequence always encodes the same polypeptide.

When using the methods above, the term “about” is used precisely to account for fractional percentages of codon frequencies for a given amino acid. As used herein, “about” is defined as one amino acid more or one amino acid less than the value given. The whole number value of amino acids is rounded up if the fractional frequency of usage is 0.50 or greater, and is rounded down if the fractional frequency of use is 0.49 or less. Using again the example of the frequency of usage of leucine in human genes for a hypothetical polypeptide having 62 leucine residues, the fractional frequency of codon usage would be calculated by multiplying 62 by the frequencies for the various codons. Thus, 7.28 percent of 62 equals 4.51 UUA codons, or “about 5,” i.e., 4, 5, or 6 UUA codons, 12.66 percent of 62 equals 7.85 UUG codons or “about 8,” i.e., 7, 8, or 9 UUG codons, 12.87 percent of 62 equals 7.98 CUU codons, or “about 8,” i.e., 7, 8, or 9 CUU codons, 19.56 percent of 62 equals 12.13 CUC codons or “about 12,” i.e., 11, 12, or 13 CUC codons, 7.00 percent of 62 equals 4.34 CUA codons or “about 4,” i.e., 3, 4, or 5 CUA codons, and 40.62 percent of 62 equals 25.19 CUG codons, or “about 25,” i.e., 24, 25, or 26 CUG codons.

Randomly assigning codons at an optimized frequency to encode a given polypeptide sequence, can be done manually by calculating codon frequencies for each amino acid, and then assigning the codons to the polypeptide sequence randomly. Additionally, various algorithms and computer software programs are readily available to those of ordinary skill in the art. For example, the “EditSeq” function in the Lasergene Package, available from DNAstar, Inc., Madison, Wis., the backtranslation function in the Vector NTI Suite, available from InforMax, Inc., Bethesda, Md., and the “backtranslate” function in the GCG—Wisconsin Package, available from Accelrys, Inc., San Diego, Calif. In addition, various resources are publicly available to codon-optimize coding region sequences, e.g., the “backtranslation” function at entelechon.com/bioinformatics/backtranslation.php?lang=eng (visited Aug. 10, 2012) and the “backtranseq” function available at emboss.bioinformatics.nl/cgi-bin/emboss/backtranseq (visited Dec. 18, 2009). Constructing a rudimentary algorithm to assign codons based on a given frequency can also easily be accomplished with basic mathematical functions by one of ordinary skill in the art.

A number of options are available for synthesizing codon optimized coding regions designed by any of the methods described above, using standard and routine molecular biological manipulations well known to those of ordinary skill in the art. In one approach, a series of complementary oligonucleotide pairs of 80-90 nucleotides each in length and spanning the length of the desired sequence is synthesized by standard methods. These oligonucleotide pairs are synthesized such that upon annealing, they form double stranded fragments of 80-90 base pairs, containing cohesive ends, e.g., each oligonucleotide in the pair is synthesized to extend 3, 4, 5, 6, 7, 8, 9, 10, or more bases beyond the region that is complementary to the other oligonucleotide in the pair. The single-stranded ends of each pair of oligonucleotides are designed to anneal with the single-stranded end of another pair of oligonucleotides. The oligonucleotide pairs are allowed to anneal, and approximately five to six of these double-stranded fragments are then allowed to anneal together via the cohesive single stranded ends, and then they ligated together and cloned into a standard bacterial cloning vector, for example, a TOPO® vector available from Invitrogen Corporation, Carlsbad, Calif. The construct is then sequenced by standard methods. Several of these constructs consisting of 5 to 6 fragments of 80 to 90 base pair fragments ligated together, i.e., fragments of about 500 base pairs, are prepared, such that the entire desired sequence is represented in a series of plasmid constructs. The inserts of these plasmids are then cut with appropriate restriction enzymes and ligated together to form the final construct. The final construct is then cloned into a standard bacterial cloning vector, and sequenced. Additional methods would be immediately apparent to the skilled artisan. In addition, gene synthesis is readily available commercially.

In additional embodiments, a full-length polypeptide sequence is codon-optimized for a given species resulting in a codon-optimized coding region encoding the entire polypeptide, and then nucleic acid fragments of the codon-optimized coding region, which encode fragments, variants, and derivatives of the polypeptide, are made from the original codon-optimized coding region. As would be well understood by those of ordinary skill in the art, if codons have been randomly assigned to the full-length coding region based on their frequency of use in a given species, nucleic acid fragments encoding fragments, variants, and derivatives would not necessarily be fully codon optimized for the given species. However, such sequences are still much closer to the codon usage of the desired species than the native codon usage. The advantage of this approach is that synthesizing codon-optimized nucleic acid fragments encoding each fragment, variant, and derivative of a given polypeptide, although routine, would be time consuming and would result in significant expense.

### Vectors and Methods of Using Vectors in Host Cells

In another aspect, the present invention relates to vectors which include polynucleotides of the present invention, host cells which are genetically engineered with vectors of the invention and the production of polypeptides of the invention by recombinant techniques.

Host cells are genetically engineered (transduced or transformed or transfected) with the vectors of this invention which may be, for example, a cloning vector or an expression vector. The vector may be, for example, in the form of a plasmid, a viral particle, a phage, etc. The engineered host cells can be cultured in conventional nutrient media modified as appropriate for activating promoters, selecting transformants or amplifying the genes of the present invention. The culture conditions, such as temperature, pH and the like, are those previously used with the host cell selected for expression, and will be apparent to the ordinarily skilled artisan.

The polynucleotides of the present invention can be employed for producing polypeptides by recombinant techniques. Thus, for example, the polynucleotide may be included in any one of a variety of expression vectors for expressing a polypeptide. Such vectors include chromosomal, nonchromosomal and synthetic DNA sequences, e.g., derivatives of SV40; bacterial plasmids; and yeast plasmids. However, any other vector may be used as long as it is replicable and viable in the host.

The appropriate DNA sequence can be inserted into the vector by a variety of procedures. In general, the DNA sequence is inserted into an appropriate restriction endonuclease site(s) by procedures known in the art. Such procedures and others are deemed to be within the scope of those skilled in the art.

The DNA sequence in the expression vector is operatively associated with an appropriate expression control sequence(s) (promoter) to direct mRNA synthesis. Any suitable promoter to drive gene expression in the host cells of the invention may be used.

In addition, the expression vectors may contain one or more selectable marker genes to provide a phenotypic trait for selection of transformed host cells such as URA3, HIS3, LEU2, TRP1, LYS2 or ADE2, dihydrofolate reductase, neomycin (G418) resistance or zeocin resistance for eukaryotic cell culture, or tetracycline or ampicillin resistance in prokaryotic cell culture, e.g., Clostridium thermocellum.

The expression vector may also contain a ribosome binding site for translation initiation and/or a transcription terminator. The vector may also include appropriate sequences for amplifying expression, or may include additional regulatory regions.

The vector containing the appropriate DNA sequence as herein, as well as an appropriate promoter or control sequence, may be employed to transform an appropriate host to permit the host to express the protein.

Thus, in certain aspects, the present invention relates to host cells containing the above-described constructs. The host cell can be a host cell as described elsewhere in the application. The host cell can be, for example, a lower eukaryotic cell, such as a yeast cell, e.g., Saccharomyces cerevisiae or Kluyveromyces, or the host cell can be a prokaryotic cell, such as a bacterial cell, e.g., Clostridium thermocellum.

The selection of an appropriate host is deemed to be within the scope of those skilled in the art from the teachings herein. In one embodiment, the vector is integrated into the genome of the host cell. In another embodiment, the vector is present in the host cell as an extrachromosomal plasmid.

### Transposons

To select for foreign DNA that has entered a host it is preferable that the DNA be stably maintained in the organism of interest. With regard to plasmids, there are two processes by which this can occur. One is through the use of replicative plasmids. These plasmids have origins of replication that are recognized by the host and allow the plasmids to replicate as stable, autonomous, extrachromosomal elements that are partitioned during cell division into daughter cells. The second process occurs through the integration of a plasmid onto the chromosome. This predominately happens by homologous recombination and results in the insertion of the entire plasmid, or parts of the plasmid, into the host chromosome. Thus, the plasmid and selectable marker(s) are replicated as an integral piece of the chromosome and segregated into daughter cells. Therefore, to ascertain if plasmid DNA is entering a cell during a transformation event through the use of selectable markers requires the use of a replicative plasmid or the ability to recombine the plasmid onto the chromosome. These qualifiers cannot always be met, especially when handling organisms that do not have a suite of genetic tools.

One way to avoid issues regarding plasmid-associated markers is through the use of transposons. A transposon is a mobile DNA element, defined by mosaic DNA sequences that are recognized by enzymatic machinery referred to as a transposase. The function of the transposase is to randomly insert the transposon DNA into host or target DNA. A selectable marker can be cloned onto a transposon by standard genetic engineering. The resulting DNA fragment can be coupled to the transposase machinery in an in vitro reaction and the complex can be introduced into target cells by electroporation. Stable insertion of the marker onto the chromosome requires only the function of the transposase machinery and alleviates the need for homologous recombination or replicative plasmids.

The random nature associated with the integration of transposons has the added advantage of acting as a form of mutagenesis. Libraries can be created that comprise amalgamations of transposon mutants. These libraries can be used in screens or selections to produce mutants with desired phenotypes. For instance, a transposon library of a CBP organism could be screened for the ability to produce more ethanol, or less lactic acid and/or more acetate.

### Native Cellulolytic Strategy

Naturally occurring cellulolytic microorganisms are starting points for CBP organism development via the native strategy. Anaerobes and facultative anaerobes are of particular interest. The primary objective is to improve the engineering of the detoxification of biomass derived acetate to a non-charged solvent, including but not limited to, acetone, isopropanol, or ethanol. Metabolic engineering of mixed-acid fermentations in relation to, for example, ethanol production, has been successful in the case of mesophilic, non-cellulolytic, enteric bacteria. Recent developments in suitable gene-transfer techniques allow for this type of work to be undertaken with cellulolytic bacteria.

### Recombinant Cellulolytic Strategy

Non-cellulolytic microorganisms with desired product-formation properties are starting points for CBP organism development by the recombinant cellulolytic strategy. The primary objective of such developments is to engineer a heterologous cellulase system that enables growth and fermentation on pretreated lignocellulose. The heterologous production of cellulases has been pursued primarily with bacterial hosts producing ethanol at high yield (engineered strains of E. coli, Klebsiella oxytoca, and Zymomonas mobilis) and the yeast Saccharomyces cerevisiae. Cellulase expression in strains of K. oxytoca resulted in increased hydrolysis yields—but not growth without added cellulase—for microcrystalline cellulose, and anaerobic growth on amorphous cellulose. Although dozens of saccharolytic enzymes have been functionally expressed in S. cerevisiae, anaerobic growth on cellulose as the result of such expression has not been definitively demonstrated.

Aspects of the present invention relate to the use of thermophilic or mesophilic microorganisms as hosts for modification via the native cellulolytic strategy. Their potential in process applications in biotechnology stems from their ability to grow at relatively high temperatures with attendant high metabolic rates, production of physically and chemically stable enzymes, and elevated yields of end products. Major groups of thermophilic bacteria include eubacteria and archaebacteria. Thermophilic eubacteria include: phototropic bacteria, such as cyanobacteria, purple bacteria, and green bacteria; Gram-positive bacteria, such as Bacillus, Clostridium, Lactic acid bacteria, and Actinomyces; and other eubacteria, such as Thiobacillus, Spirochete, Desulfotomaculum, Gram-negative aerobes, Gram-negative anaerobes, and Thermotoga. Within archaebacteria are considered Methanogens, extreme thermophiles (an art-recognized term), and Thermoplasma. In certain embodiments, the present invention relates to Gram-negative organotrophic thermophiles of the genera Thermus, Gram-positive eubacteria, such as genera Clostridium, and also which comprise both rods and cocci, genera in group of eubacteria, such as Thermosipho and Thermotoga, genera of Archaebacteria, such as Thermococcus, Thermoproteus (rod-shaped), Thermofilum (rod-shaped), Pyrodictium, Acidianus. Sulfolobus, Pyrobaculum, Pyrococcus, Thermodiscus, Staphylothermus, Desulfurococcus, Archaeoglobus, and Methanopyrus. Some examples of thermophilic or mesophilic (including bacteria, procaryotic microorganism, and fungi), which may be suitable for the present invention include, but are not limited to: Clostridium thermosulfurogenes, Clostridium cellulolyticum, Clostridium thermocellum, Clostridium thermohydrosulfuricum, Clostridium thermoaceticum, Clostridium thermosaccharolyticum, Clostridium tartarivorum, Clostridium thermocellulaseum, Clostridium phytofermentans, Clostridium straminosolvens, Thermoanaerobacterium thermosaccarolyticum, Thermoanaerobacterium saccharolyticum, Thermobacteroides acetoethylicus, Thermoanaerobium brockii, Methanobacterium thermoautotrophicum, Anaerocellum thermophilium, Pyrodictium occultum, Thermoproteus neutrophilus, Thermofilum librum, Tnermothrix thioparus, Desulfovibrio thermophilus, Thermoplasma acidophilum, Hydrogenomonas thermophilus, Thermomicrobium roseum, Thermus flavas, Thermus ruber, Pyrococcus furiosus, Thermus aquaticus, Thermus thermophilus, Chlorojlexus aurantiacus, Thermococcus litoralis, Pyrodictium abyssi, Bacillus stearothermophilus, Cyanidium caldarium, Mastigociadus laminosus, Chlamydothrix calidissima, Chlamydothrix penicillata, Thiothrix carnea, Phormidium tenuissimum, Phormidium geysericola, Phormidium subterraneum, Phormidium bijahensi, Oscillatoria filiformis, Synechococcus lividus, Chloroflexus aurantiacus, Pyrodictium brockii, Thiobacillus thiooxidans, Sulfolobus acidocaldarius, Thiobacillus thermophilica, Bacillus stearothermophilus, Cercosulcifer hamathensis, Vahlkampfia reichi, Cyclidium citrullus, Dactylaria gallopava, Synechococcus lividus, Synechococcus elongatus, Synechococcus minervae, Synechocystis aquatilus, Aphanocapsa thermalis, Oscillatoria terebriformis, Oscillatoria amphibia, Oscillatoria germinata, Oscillatoria okenii, Phormidium laminosum, Phormidium parparasiens, Symploca thermalis, Bacillus acidocaldarias, Bacillus coagulans, Bacillus thermocatenalatus, Bacillus licheniformis, Bacillus pamilas, Bacillus macerans, Bacillus circulans, Bacillus laterosporus, Bacillus brevis, Bacillus subtilis, Bacillus sphaericus, Desulfotomaculum nigrificans, Streptococcus thermophilus, Lactobacillus thermophilus, Lactobacillus bulgaricus, Bifidobacterium thermophilum, Streptomyces fragmentosporus, Streptomyces thermonitrificans, Streptomyces thermovulgaris, Pseudonocardia thermophila, Thermoactinomyces vulgaris, Thermoactinomyces sacchari, Thermoactinomyces candidal, Thermomonospora curvata, Thermomonospora viridis, Thermomonospora citrina, Microbispora thermodiastatica, Microbispora aerata, Microbispora bispora, Actinobifida dichotomica, Actinobifida chromogena, Micropolyspora caesia, Micropolyspora faeni, Micropolyspora cectivugida, Micropolyspora cabrobrunea, Micropolyspora thermovirida, Micropolyspora viridinigra, Methanobacterium thermoautothropicum, Caldicellulosiruptor acetigenus, Caldicellulosiruptor saccharolyticus, Caldicellulosiruptor kristjanssonii, Caldicellulosiruptor owensensis, Caldicellulosiruptor lactoaceticus, variants thereof, and/or progeny thereof.

In particular embodiments, the present invention relates to thermophilic bacteria selected from the group consisting of Clostridium cellulolyticum, Clostridium thermocellum, and Thermoanaerobacterium saccharolyticum.

In certain embodiments, the present invention relates to thermophilic bacteria selected from the group consisting of Fervidobacterium gondwanense, Clostridium thermolacticum, Moorella sp., and Rhodothermus marinus.

In certain embodiments, the present invention relates to thermophilic bacteria of the genera Thermoanaerobacterium or Thermoanaerobacter, including, but not limited to, species selected from the group consisting of: Thermoanaerobacteriumthermosulfurigenes, Thermoanaerobacteriumaotearoense, Thermoanaerobacteriumpolysaccharolyticum, Thermoanaerobacteriumzeae, Thermoanaerobacteriumxylanolyticum, Thermoanaerobacterium saccharolyticum, Thermoanaerobiumbrockii, Thermoanaerobacteriumthermosaccharolyticum, Thermoanaerobacter thermohydrosulfuricus, Thermoanaerobacterethanolicus, Thermoanaerobacterbrockii, variants thereof, and progeny thereof.

In certain embodiments, the present invention relates to microorganisms of the genera Geobacillus, Saccharococcus, Paenibacillus, Bacillus, and Anoxybacillus, including, but not limited to, species selected from the group consisting of: Geobacillus thermoglucosidasius, Geobacillus stearothermophilus, Saccharococcus caldoxylosilyticus, Saccharoccus thermophilus, Paenibacillus campinasensis, Bacillus flavothermus, Anoxybacillus kamchatkensis, Anoxybacillus gonensis, variants thereof, and progeny thereof.

In certain embodiments, the present invention relates to mesophilic bacteria selected from the group consisting of Saccharophagus degradans; Flavobacterium johnsoniae; Fibrobacter succinogenes; Clostridium hungatei; Clostridium phytofermentans; Clostridium cellulolyticum; Clostridium aldrichii; Clostridium termitididis; Acetivibrio cellulolyticus; Acetivibrio ethanolgignens; Acetivibrio multivorans; Bacteroides cellulosolvens; and Alkalibacter saccharofomentans, variants thereof and progeny thereof.

### Organism Development Via the Native Cellulolytic Strategy

One approach to organism development for CBP begins with organisms that naturally utilize cellulose, hemicellulose and/or other biomass components, which are then genetically engineering to enhance product yield and tolerance. For example, Clostridium thermocellum is a thermophilic bacterium that has among the highest rates of cellulose utilization reported. Other organisms of interest are xylose-utilizing thermophiles such as Thermoanaerobacterium saccharolyticum and Thermoanaerobacterium thermosaccharolyticum. Organic acid production may be responsible for the low concentrations of produced ethanol generally associated with these organisms. Thus, one objective is to eliminate production of acetic and lactic acid in these organisms via metabolic engineering. Substantial efforts have been devoted to developing gene transfer systems for the above-described target organisms and multiple C. thermocellum isolates from nature have been characterized. See McLaughlin et al. (2002) Environ. Sci. Technol. 36:2122. Metabolic engineering of thermophilic, saccharolytic bacteria is an active area of interest, and knockout of lactate dehydrogenase in T. saccharolyticum has recently been reported. See Desai et al. (2004) Appl. Microbiol. Biotechnol. 65:600. Knockout of acetate kinase and phosphotransacetylase in this organism is also possible.

### Organism Development Via the Recombinant Cellulolytic Strategy

An alternative approach to organism development for CBP involves conferring the ability to grow on lignocellulosic materials to microorganisms that naturally have high product yield and tolerance via expression of a heterologous cellulosic system and perhaps other features. For example, Saccharomyces cerevisiae has been engineered to express over two dozen different saccharolytic enzymes. See Lynd et al. (2002) Microbiol. Mol. Biol. Rev. 66:506.

Whereas cellulosic hydrolysis has been approached in the literature primarily in the context of an enzymatically-oriented intellectual paradigm, the CBP processing strategy requires that cellulosic hydrolysis be viewed in terms of a microbial paradigm. This microbial paradigm naturally leads to an emphasis on different fundamental issues, organisms, cellulosic systems, and applied milestones compared to those of the enzymatic paradigm. In this context, C. thermocellum has been a model organism because of its high growth rate on cellulose together with its potential utility for CBP.

In certain embodiments, organisms useful in the present invention may be applicable to the process known as simultaneous saccharification and fermentation (SSF), which is intended to include the use of said microorganisms and/or one or more recombinant hosts (or extracts thereof, including purified or unpurified extracts) for the contemporaneous degradation or depolymerization of a complex sugar (i.e., cellulosic biomass) and bioconversion of that sugar residue into ethanol by fermentation.

### Ethanol Production

According to the present invention, a recombinant microorganism can be used to produce ethanol from biomass, which is referred to herein as lignocellulosic material, lignocellulosic substrate, or cellulosic biomass. Methods of producing ethanol can be accomplished, for example, by contacting the biomass with a recombinant microorganism as described herein, and as described in commonly owned International Appl. No. PCT/US2009/002902, International Appl. No. PCT/US2009/003972, International Appl. No. PCT/US2009/003970, Published International Appl. No. WO 2010/060056, International Appl. No. PCT/US2009/069443, International Appl. No. PCT/US2009/064128, International Appl. No. PCT/IB2009/005881, International Appl. No. PCT/US2011/039192, U.S. Appl. No. 61/116,981, U.S. Published Appl. No. 2012/0129229 A1, U.S. Appl. No. 61/351,165, U.S. application Ser. No. 13/701,652, and U.S. Appl. No. 61/420,142, the contents of each are incorporated by reference herein in their entirety.

In addition, to produce ethanol, the recombinant microorganisms as described herein can be combined, either as recombinant host cells or as engineered metabolic pathways in recombinant host cells, with the recombinant microorganisms described in commonly owned International Appl. No. PCT/US2009/002902, International Appl. No. PCT/US2009/003972, International. Appl. No. PCT/US2009/003970, International Patent Application Publication No. WO 2010/060056, International Appl. No. PCT/US2009/069443, International Appl. No. PCT/US2009/064128, International Appl. No. PCT/IB2009/005881, International Appl. No. PCT/US2011/039192, U.S. Appl. No. 61/351,165, U.S. application Ser. No. 13/701,652, and U.S. Appl. No. 61/420,142, the contents of each are incorporated by reference herein in their entirety. The recombinant microorganism as described herein can also be engineered with the enzymes and/or metabolic pathways described in commonly owned International Appl. No. PCT/US2009/002902, International Appl. No. PCT/US2009/003972, International Appl. No. PCT/US2009/003970, International Patent Application Publication No. WO 2010/060056, International Appl. No. PCT/US2009/069443, International Appl. No. PCT/US2009/064128, International Appl. No. PCT/IB2009/005881, International Appl. No. PCT/US2011/039192, U.S. Appl. No. 61/351,165, U.S. application Ser. No. 13/701,652, and U.S. Appl. No. 61/420,142, the contents of each are incorporated by reference herein in their entirety.

Numerous cellulosic substrates can be used in accordance with the present invention. Substrates for cellulose activity assays can be divided into two categories, soluble and insoluble, based on their solubility in water. Soluble substrates include cellodextrins or derivatives, carboxymethyl cellulose (CMC), or hydroxyethyl cellulose (HEC). Insoluble substrates include crystalline cellulose, microcrystalline cellulose (Avicel), amorphous cellulose, such as phosphoric acid swollen cellulose (PASC), dyed or fluorescent cellulose, and pretreated lignocellulosic biomass. These substrates are generally highly ordered cellulosic material and thus only sparingly soluble.

It will be appreciated that suitable lignocellulosic material may be any feedstock that contains soluble and/or insoluble cellulose, where the insoluble cellulose may be in a crystalline or non-crystalline form. In various embodiments, the lignocellulosic biomass comprises, for example, wood, corn, corn stover, sawdust, bark, leaves, agricultural and forestry residues, grasses such as switchgrass, ruminant digestion products, municipal wastes, paper mill effluent, newspaper, cardboard or combinations thereof.

In some embodiments, the invention is directed to a method for hydrolyzing a cellulosic substrate, for example a cellulosic substrate as described above, by contacting the cellulosic substrate with a recombinant microorganism of the invention. In some embodiments, the invention is directed to a method for hydrolyzing a cellulosic substrate, for example a cellulosic substrate as described above, by contacting the cellulosic substrate with a co-culture comprising yeast cells expressing heterologous cellulases.

In some embodiments, the invention is directed to a method for fermenting cellulose. Such methods can be accomplished, for example, by culturing a host cell or co-culture in a medium that contains insoluble cellulose to allow saccharification and fermentation of the cellulose.

The production of ethanol can, according to the present invention, be performed at temperatures of at least about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., or about 50° C. In some embodiments of the present invention the thermotolerant host cell can produce ethanol from cellulose at temperatures above about 30° C., about 31° C., about 32° C., about 33° C., about 34° C., about 35° C., about 36° C., about 37° C., about 38° C., about 39° C., about 40° C., about 41° C., about 42° C., or about 43° C., or about 44° C., or about 45° C., or about 50° C. In some embodiments of the present invention, the thermotolerant host cell can produce ethanol from cellulose at temperatures from about 30° C. to 60° C., about 30° C. to 55° C., about 30° C. to 50° C., about 40° C. to 60° C., about 40° C. to 55° C. or about 40° C. to 50° C.

In some embodiments, methods of producing ethanol can comprise contacting a cellulosic substrate with a recombinant microorganism or co-culture of the invention and additionally contacting the cellulosic substrate with externally produced cellulase enzymes. Exemplary externally produced cellulase enzymes are commercially available and are known to those of skill in the art.

In some embodiments, the methods comprise producing ethanol at a particular rate. For example, in some embodiments, ethanol is produced at a rate of at least about 0.1 mg per hour per liter, at least about 0.25 mg per hour per liter, at least about 0.5 mg per hour per liter, at least about 0.75 mg per hour per liter, at least about 1.0 mg per hour per liter, at least about 2.0 mg per hour per liter, at least about 5.0 mg per hour per liter, at least about 10 mg per hour per liter, at least about 15 mg per hour per liter, at least about 20.0 mg per hour per liter, at least about 25 mg per hour per liter, at least about 30 mg per hour per liter, at least about 50 mg per hour per liter, at least about 100 mg per hour per liter, at least about 200 mg per hour per liter, at least about 300 mg per hour per liter, at least about 400 mg per hour per liter, or at least about 500 mg per hour per liter.

In some embodiments, the host cells of the present invention can produce ethanol at a rate of at least about 0.1 mg per hour per liter, at least about 0.25 mg per hour per liter, at least about 0.5 mg per hour per liter, at least about 0.75 mg per hour per liter, at least about 1.0 mg per hour per liter, at least about 2.0 mg per hour per liter, at least about 5.0 mg per hour per liter, at least about 10 mg per hour per liter, at least about 15 mg per hour per liter, at least about 20.0 mg per hour per liter, at least about 25 mg per hour per liter, at least about 30 mg per hour per liter, at least about 50 mg per hour per liter, at least about 100 mg per hour per liter, at least about 200 mg per hour per liter, at least about 300 mg per hour per liter, at least about 400 mg per hour per liter, or at least about 500 mg per hour per liter more than a control strain (lacking heterologous cellulases) and grown under the same conditions. In some embodiments, the ethanol can be produced in the absence of any externally added cellulases.

Ethanol production can be measured using any method known in the art. For example, the quantity of ethanol in fermentation samples can be assessed using HPLC analysis. Many ethanol assay kits are commercially available that use, for example, alcohol oxidase enzyme based assays. Methods of determining ethanol production are within the scope of those skilled in the art from the teachings herein. The U.S. Department of Energy (DOE) provides a method for calculating theoretical ethanol yield. Accordingly, if the weight percentages are known of C6 sugars (i.e., glucan, galactan, mannan), the theoretical yield of ethanol in gallons per dry ton of total C6 polymers can be determined by applying a conversion factor as follows:
(1.11 pounds of C6 sugar/pound of polymeric sugar)×(0.51 pounds of ethanol/pound of sugar)×(2000 pounds of ethanol/ton of C6 polymeric sugar)×(1 gallon of ethanol/6.55 pounds of ethanol)×( 1/100%), wherein the factor (1 gallon of ethanol/6.55 pounds of ethanol) is taken as the specific gravity of ethanol at 20° C.

And if the weight percentages are known of C5 sugars (i.e., xylan, arabinan), the theoretical yield of ethanol in gallons per dry ton of total C5 polymers can be determined by applying a conversion factor as follows:
(1.136 pounds of C5 sugar/pound of C5 polymeric sugar)×(0.51 pounds of ethanol/pound of sugar)×(2000 pounds of ethanol/ton of C5 polymeric sugar)×(1 gallon of ethanol/6.55 pounds of ethanol)×( 1/100%), wherein the factor (1 gallon of ethanol/6.55 pounds of ethanol) is taken as the specific gravity of ethanol at 20° C.

It follows that by adding the theoretical yield of ethanol in gallons per dry ton of the total C6 polymers to the theoretical yield of ethanol in gallons per dry ton of the total C5 polymers gives the total theoretical yield of ethanol in gallons per dry ton of feedstock.

Applying this analysis, the DOE provides the following examples of theoretical yield of ethanol in gallons per dry ton of feedstock: corn grain, 124.4; corn stover, 113.0; rice straw, 109.9; cotton gin trash, 56.8; forest thinnings, 81.5; hardwood sawdust, 100.8; bagasse, 111.5; and mixed paper, 116.2. It is important to note that these are theoretical yields. The DOE warns that depending on the nature of the feedstock and the process employed, actual yield could be anywhere from 60% to 90% of theoretical, and further states that “achieving high yield may be costly, however, so lower yield processes may often be more cost effective.” (Ibid.)

### TDK Counterselection

In the field of genetic engineering, cells containing an engineering event are often identified through use of positive selections. This is done by creating genetic linkage between the positive selection encoded by a dominant marker such as an antibiotic resistance gene, the desired genetic modification, and the target loci. Once the modifications are identified, it is often desirable to remove the dominant marker so that it can be reused during subsequent genetic engineering events.

However, if a dominant marker does not also have a counter selection, a gene expressing a protein that confers a counter-selection, must be genetically linked to the dominant marker, the desired genetic modification, and the target loci. To avoid such limitations, the methods of the invention include linking and/or designing a transformation associated with recombination between the thymidine kinase gene (TDK) from the Herpes Simplex Virus Type 1 (GenBank Accession No. AAA45811; SEQ ID NO:4) and one or more antibiotic resistance genes. See, e.g., Argyros, D. A., et al., “High Ethanol Titers from Cellulose by Using Metabolically Engineered Thermophilic, Anaerobic Microbes,” Appl. Environ. Microbiol. 77(23):8288-94 (2011). Examples of such antibiotic resistant genes, include but are not limited to aminoglycoside phosphotransferase (Kan; resistant to G418), nourseothricin acetyltransferase (Nat; resistant to nourseothricin), hygromycin B phosphotransferase (hph; resistant to hygromycin B), or a product of the Sh ble gene 1 (ble; resistant to Zeocin). Using, such counter-selection methods with linked positive/negative selectable markers, transformants comprising the desired genetic modification have been obtained as described further in the examples below.

### EXAMPLES

The invention now being generally described, it will be more readily understood by reference to the following examples, which are included merely for purposes of illustration of certain aspects and embodiments of the present invention, and are not intended to limit the invention.

The following examples describe S. cerevisiae genotypes for improved acetate-to-ethanol conversion by improving the availability of redox cofactors NADH or NADPH. Homologous recombination within the yeast cell can be used for genomic integrations and the construction of plasmids. With this approach, DNA fragments (containing promoters, terminators and open reading frames) are synthesized by PCR, with overlapping regions to adjoining fragments and/or the integration site. After cotransformation of the yeast with the synthesized fragments, the yeast are screened for those containing complete assemblies. Anybody skilled in the art can design the necessary primers and perform the required transformations, and only the final DNA sequences are included in the examples below. In many cases the genomic integration site is first pre-marked with one of two antibiotic markers (to target both alleles in diploid strains) and a marker for counter-selection (such as the Herpes simplex HSV-1 thymidine kinase tdk gene, which introduces a sensitivity to fluoro-deoxyuracil, to facilitate the isolation of correct transformants. See Argyros, D. A., et al., (2011).

Promoter and terminator pairs in the following examples are exemplary.

Possible promoters include, but are not limited to: ADH1, TPI1, ENO1, PFK1, ADH5, XKS1. Possible terminators include, but are not limited to: FBA1, PDC1, ENO1, HXT2, ALD6, SOL3.

### Example 1

The present prophetic example describes engineering of a recombinant microorganism to increase flux through the oxidative pentose phosphate pathway (PPP) by creating a redox imbalance in xylose consumption using xylose reductase (XR) and xylitol dehydrogenase (XDH) that is coupled with the conversion of acetate to ethanol or isopropanol.

Current methods rely on xylose isomerase to enable S. cerevisiae to consume xylose. An alternative pathway that uses XR and XDH has been studied in the scientific literature, but achieving efficient ethanol production using this method has been difficult because of the pathway's redox imbalance. See Watanabe, S. et al., “Ethanol production from xylose by recombinant Saccharomyces cerevisiae expressing protein engineered NADP+-dependent xylitol dehydrogenase,” J. Biotechnol. 130:316-19 (2007). XRs typically have a higher affinity for the cofactor NADPH, whereas most XDHs are NAD-specific. See Watanabe, S. et al., (2007).

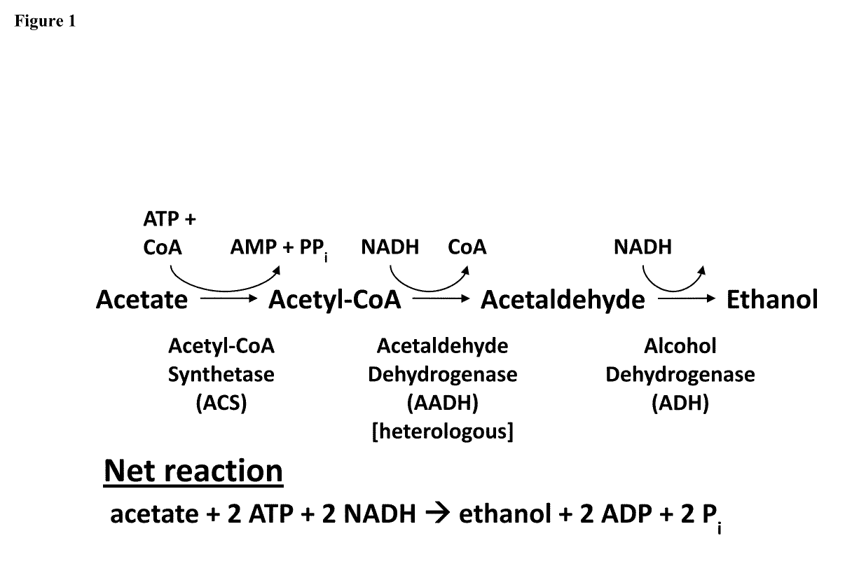

Recently an acetate-to-ethanol pathway has been described in U.S. patent application Ser. No. 13/696,207, which is incorporated by reference herein in its entirety. See also Medina, V. G., et al., “Elimination of Glycerol Production in Anaerobic Cultures of a Saccharomyces cerevisiae Strain Engineered To Use Acetic Acid as an Electron Acceptor,” Appl. Environ. Microbiol. 76:190-195 (2010). This pathway, which relies on the introduction of a heterologous acetaldehyde dehydrogenase (ACDH), consumes two NADH molecules per every molecule of acetate converted. See FIG. 1. As described herein, this NADH-consuming pathway can be used to balance the surplus NADH generated by XDH during xylose fermentation. The NADPH required by XR can be produced by redirecting part of the fructose-6-P produced by the PPP into the oxidative path of the PPP, which produces 2 NADPH per CO₂. Xylose fermentation via NADPH-specific XR and NAD-specific XDH together with acetate-to-ethanol conversion via ACDH generates a net amount of ATP (equation 1), whereas no ATP is generated when the surplus NADH is reoxidized via NADH-specific glycerol formation.

2 xylose+acetate→4 ethanol+4CO2+ATP (equation 1)

The pathway of the present invention stoichiometrically couples acetate consumption to xylose fermentation in a 1:2 molar ratio. The overall reaction results in the formation of sufficient ATP to allow for growth of the microorganisms. In the absence of other ATP-yielding reactions, it would also be possible to use natural selection to select for mutant microorganisms with faster anaerobic ethanolic fermentation on xylose/acetate mixtures and increased tolerance to industrial feedstocks.

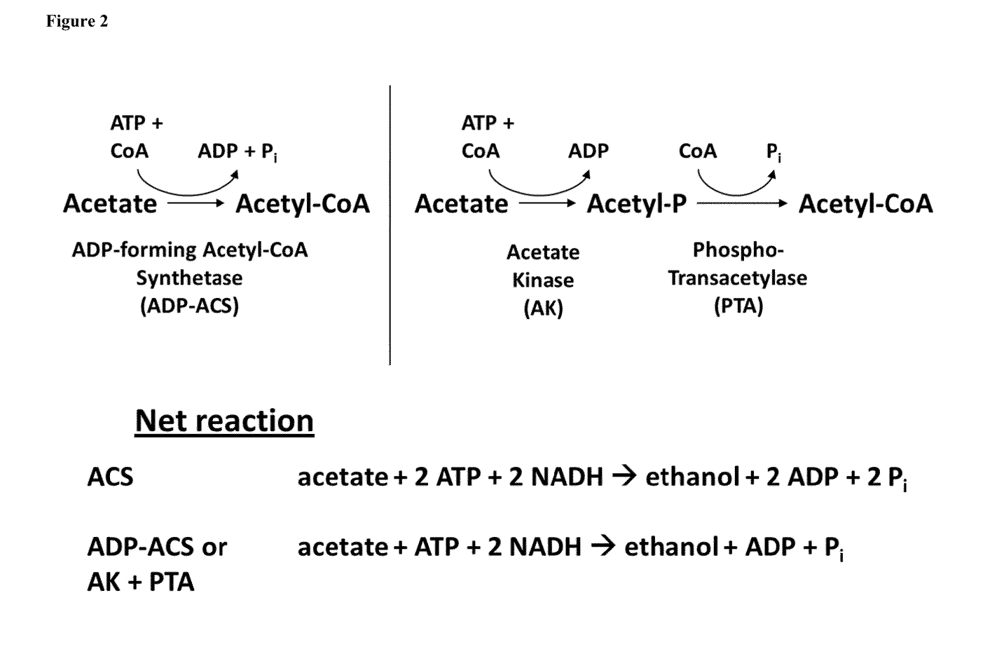

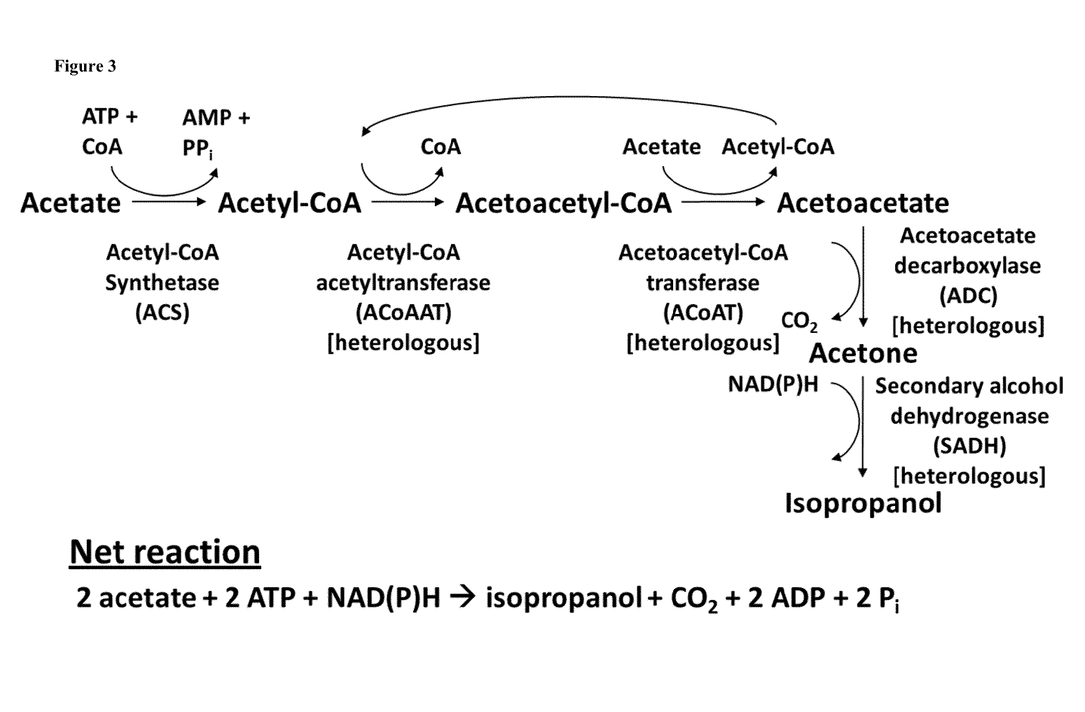

A similar strategy is employed for an acetate-to-isopropanol pathway based on the expression of the heterologous enzymes acetyl-CoA acetyltransferase (thiolase), acetoacetyl-CoA transferase, acetoacetate decarboxylase and a secondary alcohol dehydrogenase. See FIG. 3. However, to produce a positive ATP yield, additional engineering is done, e.g., by replacing or supplementing the endogenous AMP-producing acetyl-CoA synthetase (ACS) (also referred to as acetyl-CoA ligase) by an ADP-producing variant, or using the acetate kinase/phosphotransacetylase (AK/PTA) couple. The endogenous AMP-producing ACS consumes one ATP per acetate and produces AMP. The use of an ADP-producing ACS, or the enzymes acetate kinase and phosphotransacetylase, consumes one ATP molecule per acetate molecule, however ADP is produced instead of AMP. The energy released by the conversion of ATP to AMP is about twice that of the conversion of ATP to ADP, thus using an ATP-to-ADP conversion is more energy efficient (to stress this difference, ATP requirements in FIG. 2 have been normalized to ATP-to-ADP, so the ATP-to-AMP conversion of AMP-ACS counts as 2 ATP to 2 ADP). See FIG. 2. By replacing an AMP-forming acetyl-CoA synthetase with an ADP-forming variant or by AK/PTA, the resulting pathway increases the yield of ATP by four molecules (equation 2).

4 acetate+2 xylose+ATP→2 isopropanol+3 ethanol+6CO₂ (equation 2)

Testing this strategy involves engineering a yeast such as S. cerevisiae to use XR and XDH for xylose consumption and to convert acetate-to-ethanol by introducing an ACDH, and demonstrating anaerobic ethanol production with the combined consumption of xylose and acetate.

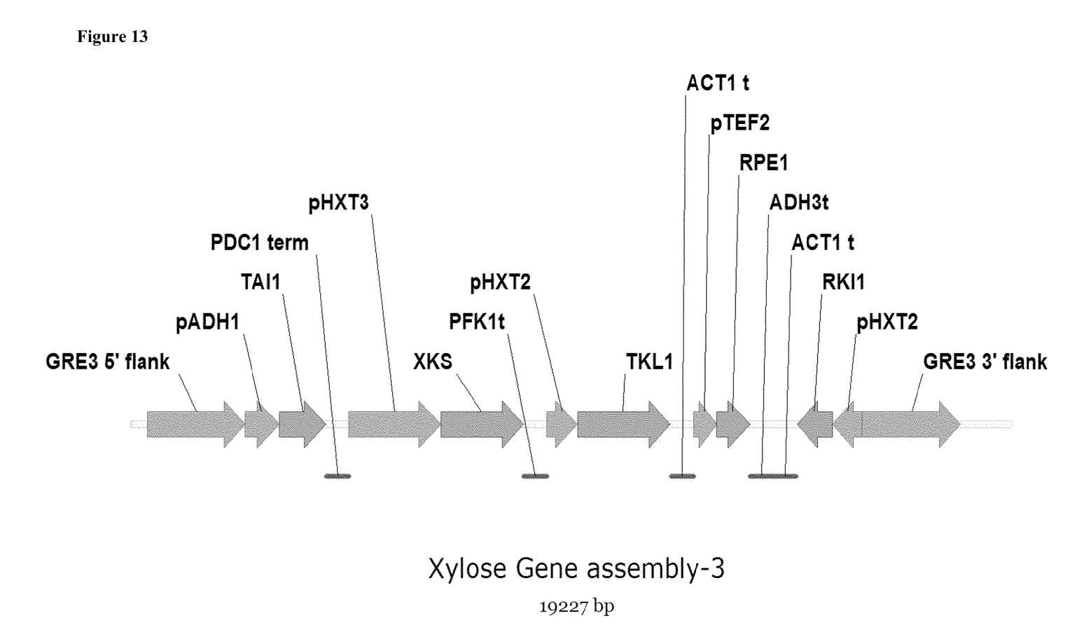

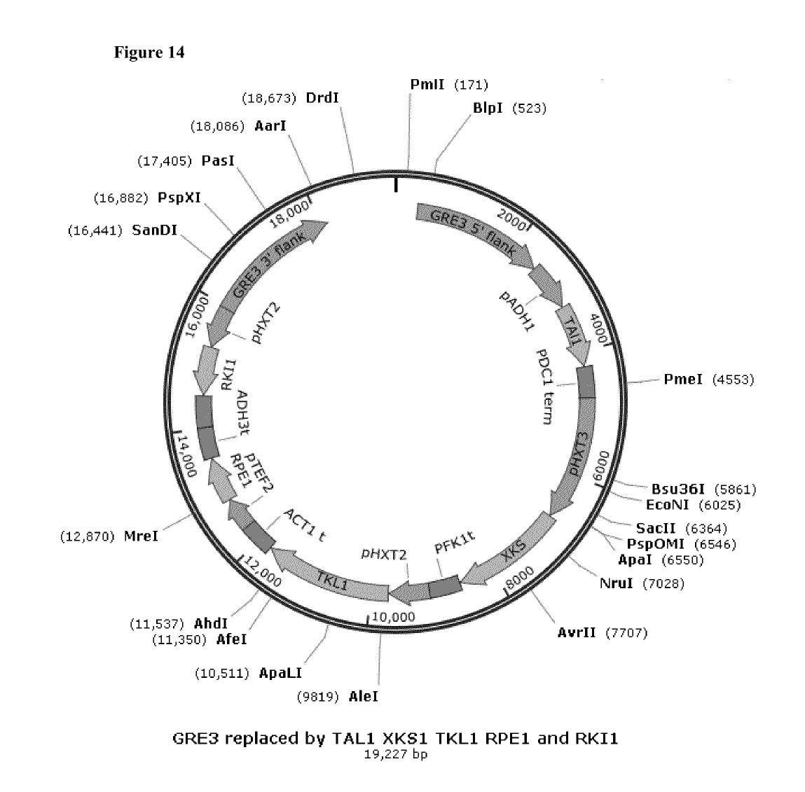

NADPH-specific XR and NADH-specific XDH are overexpressed in a strain overexpressing an NADH-dependent ACDH. To improve xylose consumption XKS1 may also be overexpressed. In one embodiment of the invention, one or more genes of the pentose phosphate pathway (either endogenous or heterologous genes) are also overexpressed, which can improve xylose metabolism. For example, the endogenous pentose phosphate genes transaldolase (TAL1), xylulokinase (XKS1), transketolase (TKL1), ribulose-phosphate 3-epimerase (RPE1) and ribulose 5-phosphate isomerase (RKI1) are overexpressed in the gre3 locus. See FIGS. 13 and 14.

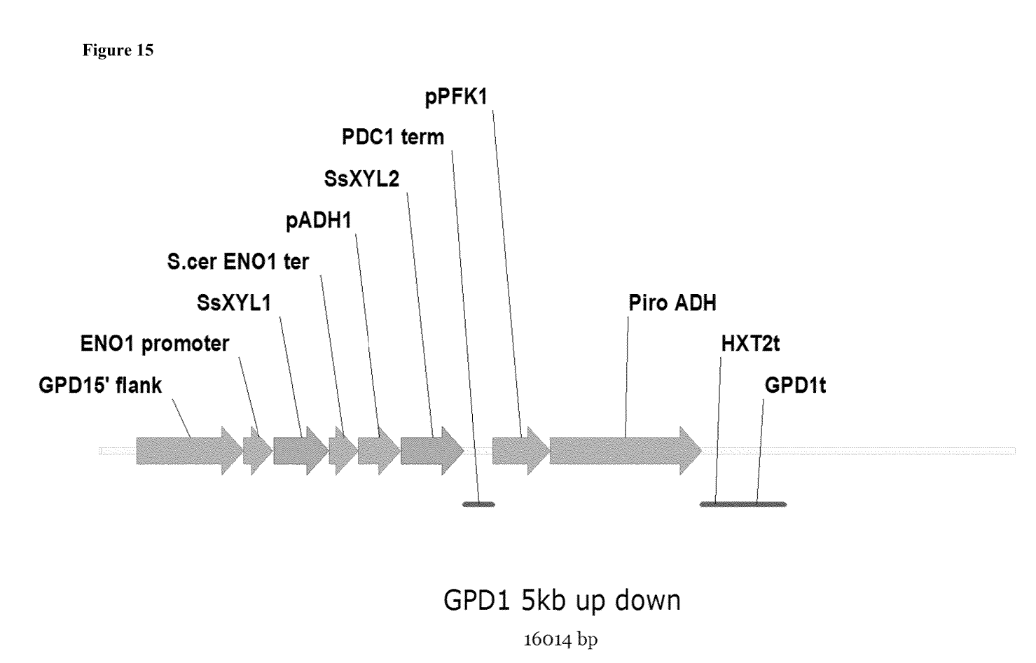

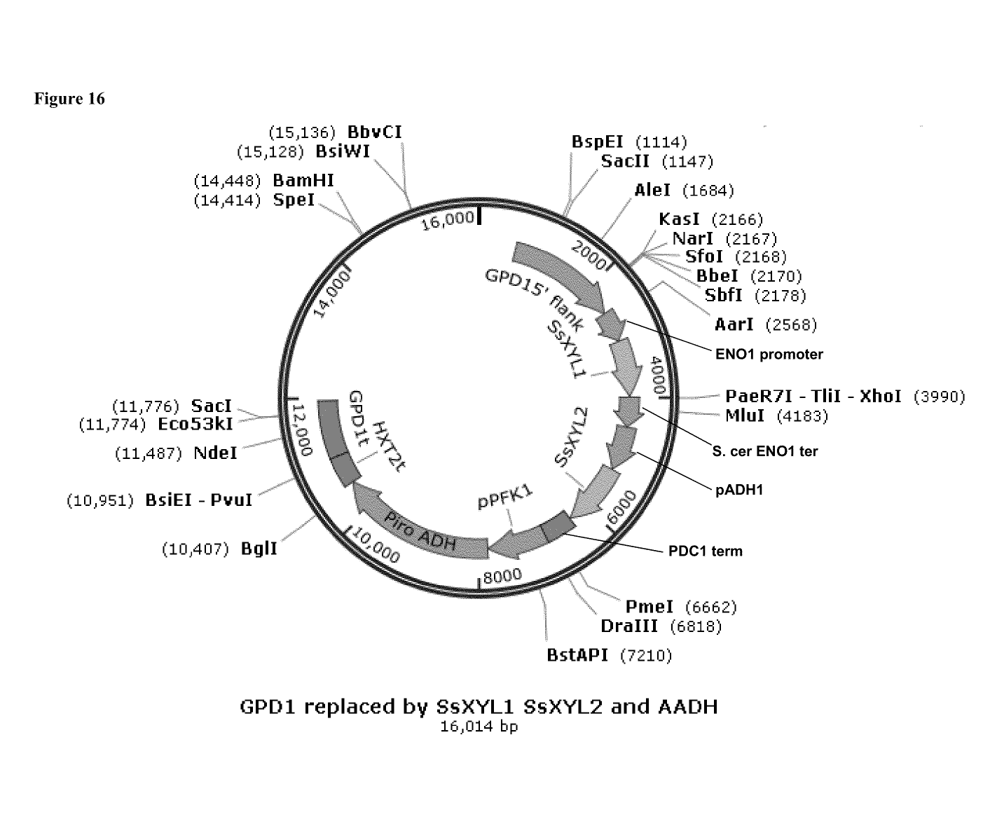

Glycerol production can also be reduced to enable growth, e.g., by deleting gpd1. See, e.g., U.S. patent application Ser. No. 13/696,207, which is incorporated by reference herein in its entirety. For example, the Scheffersomyces stipitis XYL1 and XYL2 genes and Piromyces adhE are overexpressed in the gpd1 locus. See FIGS. 15 and 16. XYL1 can be replaced by either the Candida boidinii AR or the Neurospora crassa XR gene.

This strain is grown under anaerobic conditions in media containing xylose as well as acetate. Because of the need to balance the use of redox cofactors and generate ATP, it is expected that the surplus NADH formed during the fermentation of xylose to ethanol is to a large extent used for the conversion of acetate to ethanol via the NADH-dependent ACDH.

Examples of XR sequences include: Scheffersomyces stipitis XYL1 (SEQ ID NO:5), Candida boidinii Aldolase Reductase (SEQ ID NO:6), and Neurospora crassa Xylose Reductase (codon-optimized for S. cerevisiae by DNA 2.0) (SEQ ID NO:7).

Examples of XDH sequences include: Scheffersomyces stipitis XYL2 (SEQ ID NO:8).

The nucleotide sequence for Piromyces adhE is provided as SEQ ID NO:9. Examples of ACS sequences include: Entamoeba histolytica ACS Q9NAT4 (ADP-forming) (SEQ ID NO: 10), Giardia intestinalis ACS (ADP-forming) (SEQ ID NO:11), Pyrococcus furiosus ACS Q9Y8L1 (ADP-forming) (SEQ ID NO:12), Pyrococcus furiosus ACS Q9Y8L0 (ADP-forming) (SEQ ID NO:13), Pyrococcus furiosus ACS E7FI45 (ADP-forming) (SEQ ID NO:14), and Pyrococcus furiosus ACS E7FHP1 (ADP-forming) (SEQ ID NO:15).

The amino acid sequence for S. cerevisiae TAL1 is provided in SEQ ID NO:16. The amino acid sequence for S. cerevisiae XKS1 is provided in SEQ ID NO:17. The amino acid sequence for S. cerevisiae TKL1 is provided in SEQ ID NO:18. The amino acid sequence for S. cerevisiae RPE1 is provided in SEQ ID NO:19. The amino acid sequence for S. cerevisiae RKI1 is provided in SEQ ID NO:20.

The upstream sequence used to delete S. cerevisiae GRE3 is provided in SEQ ID NO:21. The downstream sequence used to delete S. cerevisiae GRE3 is provided in SEQ ID NO:22.

2μ multi-copy vectors have been constructed expressing the XYL2 XDH from Scheffersomyces stipitis (formerly Pichia stipitis) and one of the following three XRs: XYL1 from S. stipitis (which has comparable affinity for NADH and NADPH), the more NADPH-specific XR from Neurospora crassa (codon-optimized), or aldolase reductase from Candida boidinii. See FIGS. 13 and 16.

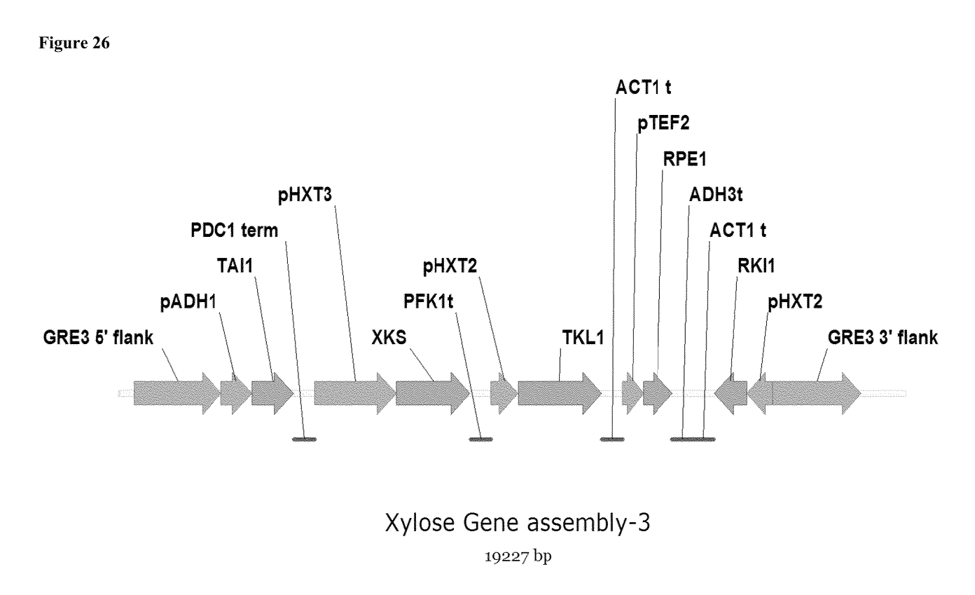

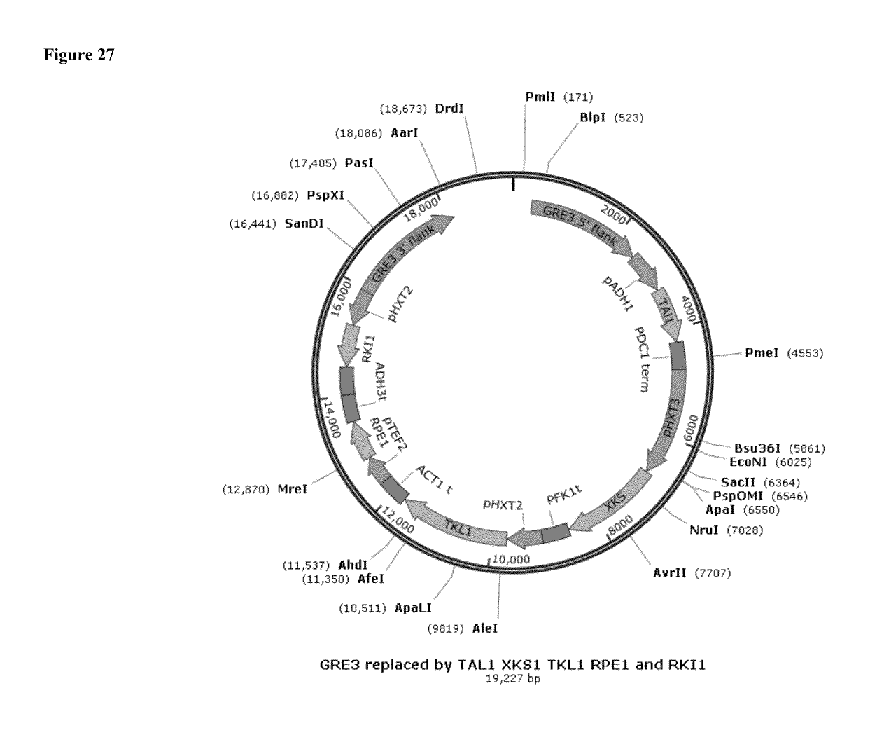

Transformation of strain M2566, in which GRE3 has been replaced by a cassette of PPP genes (including XKS1 under the HXT3 promoter), with the plasmid carrying S. stipitis XR and XDH and selection on aerobic YNX agar plates resulted in a low number of colonies. The M2566 strain was derived from strain M2390 (described in U.S. patent application Ser. No. 13/696,207 and U.S. patent application Ser. No. 13/701,652, both of which are incorporated by reference herein in their entirety). In M2566, both chromosomal copies of GRE3 (M2390 is a diploid strain) have been replaced with an expression cassette with genes from the pentose phosphate pathway, for example XKS and TKL1. Overexpressing these pentose phosphate pathway genes in S. cerevisiae generally improves xylose fermentation when either xylose isomerase or xylose reductase/xylitol dehydrogenase are expressed. A schematic and vector map of the cassette used to create the M2566 strain are depicted in FIGS. 26 and 27, respectively. To create this strain, YNX agar media containing 6.7 g/l yeast nitrogen base with amino acids (Sigma Y1250), 20 g/l bacta agar, and 20 g/l xylose was used. The YNX agar media was supplemented with nourseothricin to allow selection based on the presence of the plasmid and the agar plates were incubated at 35° C. for several days.

Further steps will encompass integrating XR, XDH and ACDH into the genome of M2566 using the techniques described above, for increased stability of expression, and selecting for growth under anaerobic conditions on xylose/acetate mixtures such as the synthetic medium described in Verduyn et al. “Effect of benzoic acid on metabolic fluxes in yeasts: a continuous-culture study on the regulation of respiration and alcoholic fermentation,” Yeast 8(7):501-17 (1992), supplemented with 420 mg/l Tween-80 and 10 mg/l ergosterol, to allow for anaerobic growth, and with xylose and acetate in an approximately 2:1 molar ratio. For example, endogenous GPD1 (encoding a glycerol-3-phosphate dehydrogenase) can be replaced with the XR/XDH/ACDH expression cassette (see FIG. 16) as glycerol formation competes with the acetate-to-ethanol conversion for NADH, and deleting GPD1 has previously been shown to reduce glycerol production in U.S. patent application Ser. No. 13/696,207, which is incorporated by reference herein.

### Example 2

The present example describes engineering of a recombinant microorganism to increase flux through the oxidative pentose phosphate pathway (PPP) by overexpressing pathway genes or reducing the expression of competing pathways that is coupled with the conversion of acetate to ethanol or isopropanol.

The strategy of Example 1 relies on two redox imbalanced pathways that counterbalance each other. An alternative approach is to improve the kinetics of the oxidative branch of the PPP over those of competing pathways. This is achieved by various approaches, including directly increasing the expression of the rate-limiting enzyme(s) of the oxidative branch of the PPP pathway, such as glucose-6-P dehydrogenase (encoded endogenously by ZWF1, SEQ ID NO:23), changing the expression of regulating transcription factors like Stb5p (also referred to as Stb5) (Cadière, A., et al., “The Saccharomyces cerevisiae zinc factor protein Stb5p is required as a basal regulator of the pentose phosphate pathway,” FEMS Yeast Research 10:819-827 (2010)). Which controls the flux distribution between glycolysis and the oxidative pentose phosphate pathway by modulating activities of enzymes involved in both pathways, or directly down-regulating the expression of genes involved in competing pathways (e.g., glycolysis), such as glucose-6-β isomerase (encoded by PGI1 in S. cerevisiae). A similar effect might be achieved by increasing the expression of the other genes of the oxidative pentose phosphate pathway, including the 6-phosphogluconolactonases SOL3 and S014, and the 6-phosphogluconate dehydrogenases GND1 and GND2.

The sequence for Saccharomyces cerevisiae stb5 is provided in SEQ ID NO:24.

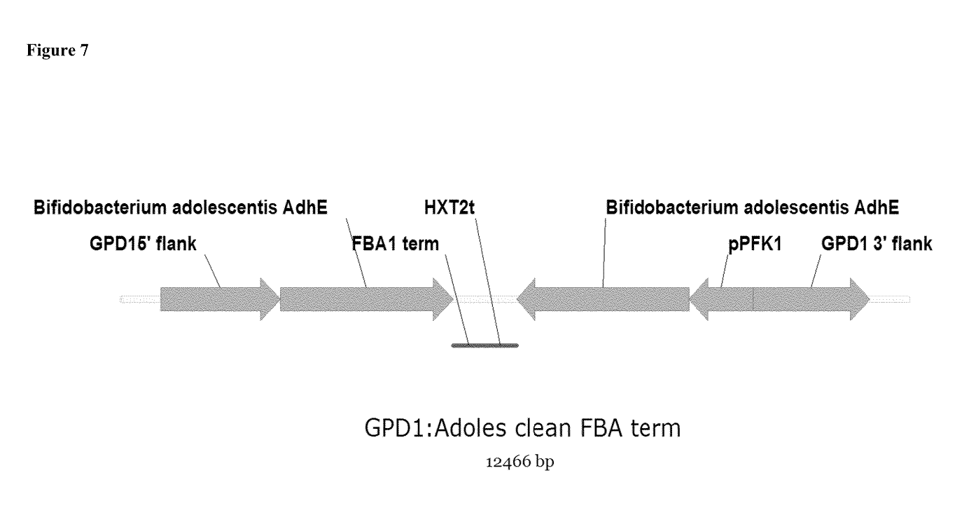

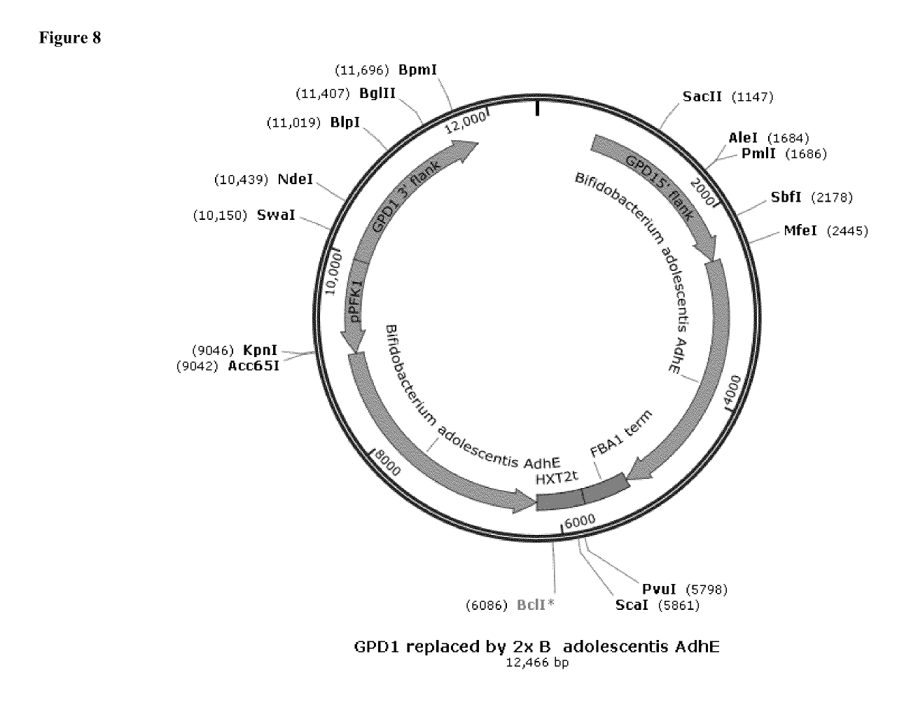

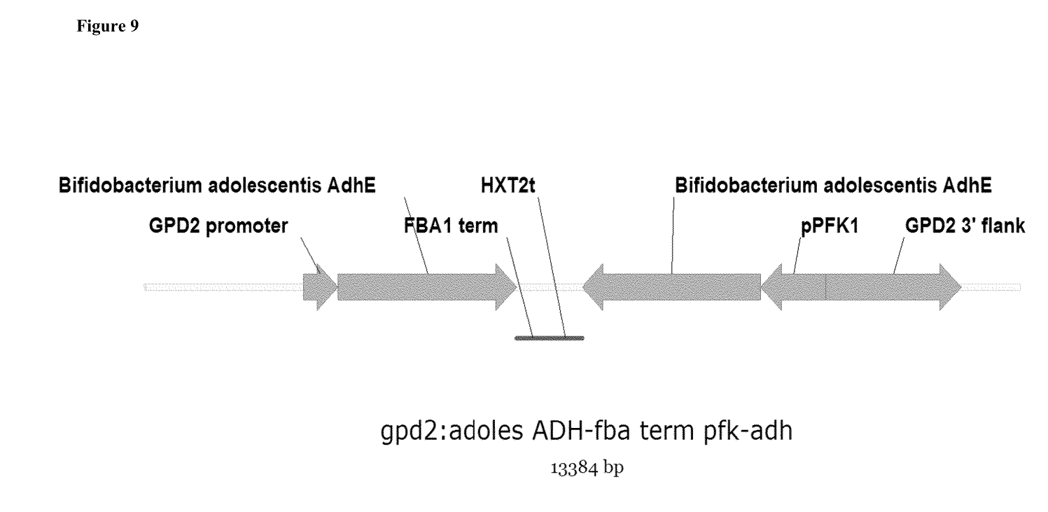

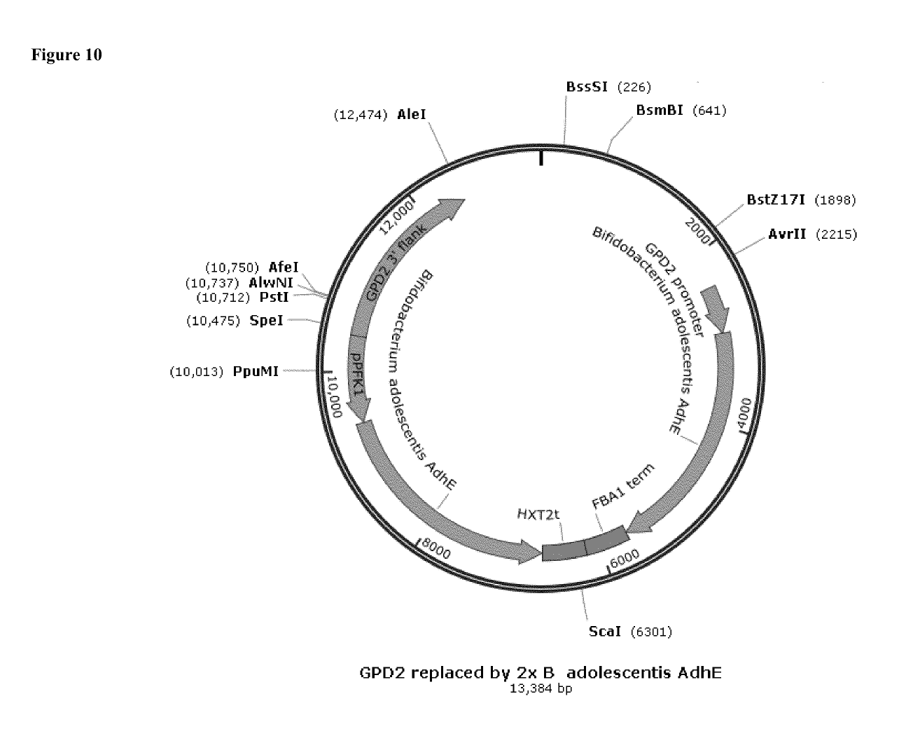

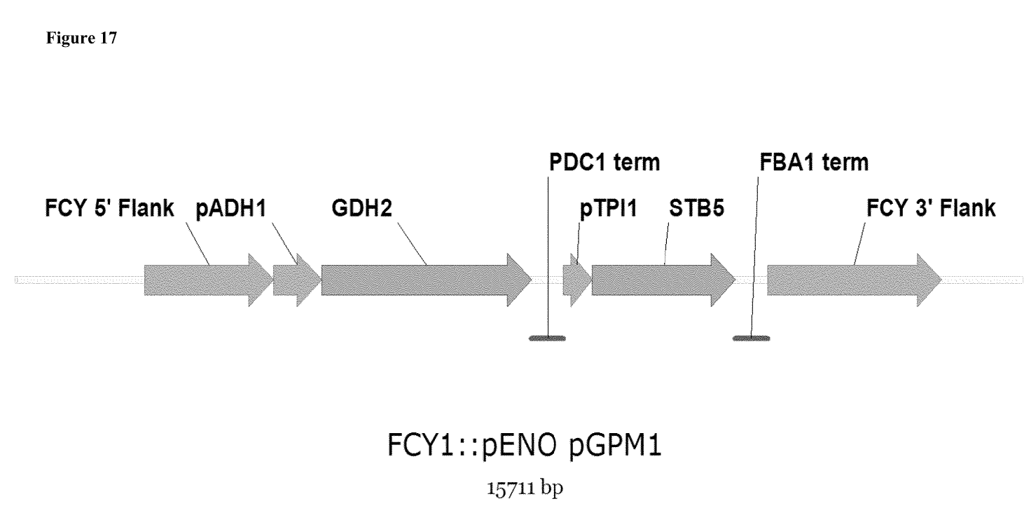

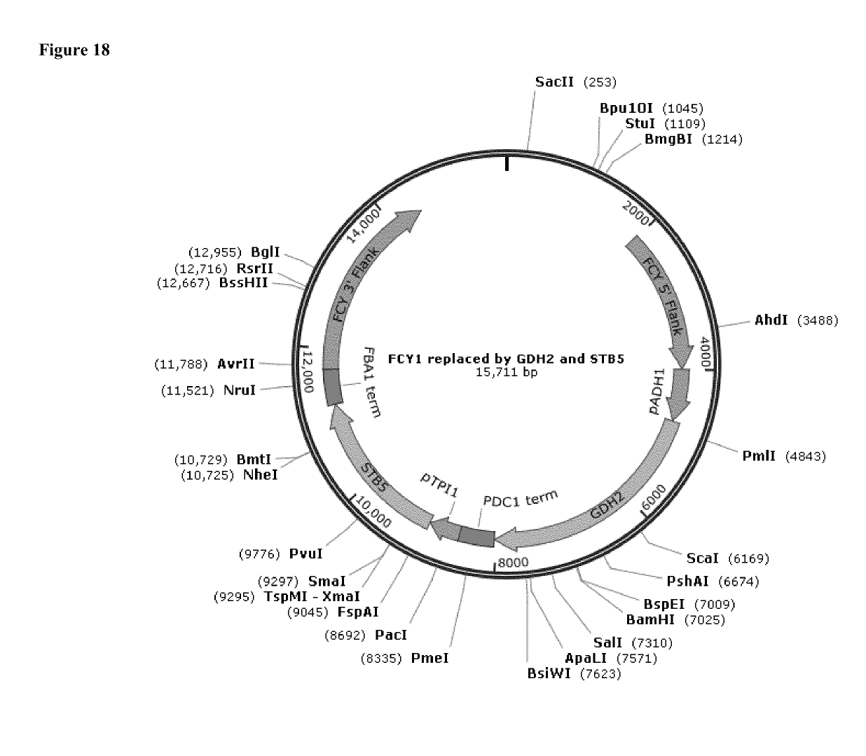

STB5 is overexpressed in a strain overexpressing either an NADPH-dependent acetaldehyde dehydrogenase, or an NADH-dependent acetaldehyde dehydrogenase, e.g., B. adolescentis adhE, in combination with genes that could affect the conversion of NADPH into NADH, such as gdh2 (SEQ ID NO:1) or a transhydrogenase (see Example 5). See FIGS. 17 and 18. In the latter case, competition with glycerol formation (another NADH-consuming reaction) can be prevented by deleting gpd1 and gpd2. See FIGS. 7-10.

The strain is grown under anaerobic conditions in media containing glucose as well as acetate. Overexpressing STB5 is expected to force more glucose through the oxidative pentose phosphate pathway, generating more NADPH, which will improve the conversion of acetate to ethanol via, e.g., an NADPH-dependent acetaldehyde dehydrogenase.

The amino acid sequence for B. adolescentis adhE is provided in SEQ ID NO:25. The upstream sequence used for deleting the Gpd1 gene is provided in SEQ ID NO:26. The downstream sequence used for deleting the Gpd1 gene is provided in SEQ ID NO:27. The sequence of the Gpd2 promoter region used for deleting the Gpd2 gene is provided in SEQ ID NO:28. The downstream sequence used for deleting the Gpd2 gene is provided in SEQ ID NO:29.

Producing more CO₂ in the oxidative branch of the PPP increases the availability of NADPH and increases the NADPH/NADP ratio. This stimulates the flux of acetate-consuming pathways, for example ethanol-to-isopropanol conversion that relies on a NADPH-consuming secondary alcohol dehydrogenase to convert acetone to isopropanol, or an acetate-to-ethanol pathway that uses a NADPH-consuming acetaldehyde dehydrogenase (ACDH) and/or alcohol dehydrogenase (ADH), that (at least partially) consume NADPH. Thus, while the supply of NADH is fairly limited, yeast have more flexibility to create NADPH via the oxidative pentose phosphate pathway where there is a demand for NADPH consumption. See Celton, M., et al., “A constraint-based model analysis of the metabolic consequences of increased NADPH oxidation in Saccharomyces cerevisiae,” Metabolic Eng 14(4):366-79 (2012).

For example, wild-type yeast do not possess endogenous ACDH activity and exogenously introduced ACDH enzymes are thought to only participate in the acetate-to-ethanol pathway. The adhB from T. pseudethanolicus is a gene that may have NADPH-specific ACDH activity and can be used in the above process. See Burdette D. and Zeikus, J. G., “Purification of acetaldehyde dehydrogenase and alcohol dehydrogenases from Thermoanaerobacter ethanolicus 39E and characterization of the secondary-alcohol dehydrogenase (2° Adh) as a bifunctional alcohol dehydrogenase-acetyl-CoA reductive thioesterase,” Biochem J. 302:163-70 (1994). The nucleotide sequence for T. pseudethanolicus adhB is provided in SEQ ID NO:30.

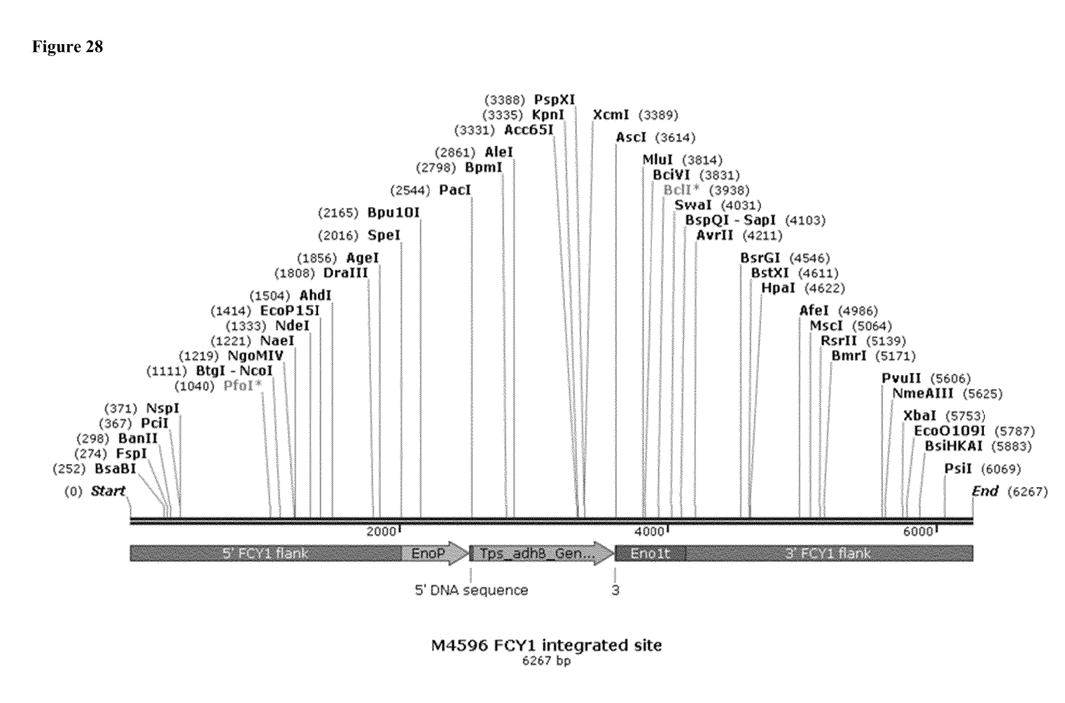

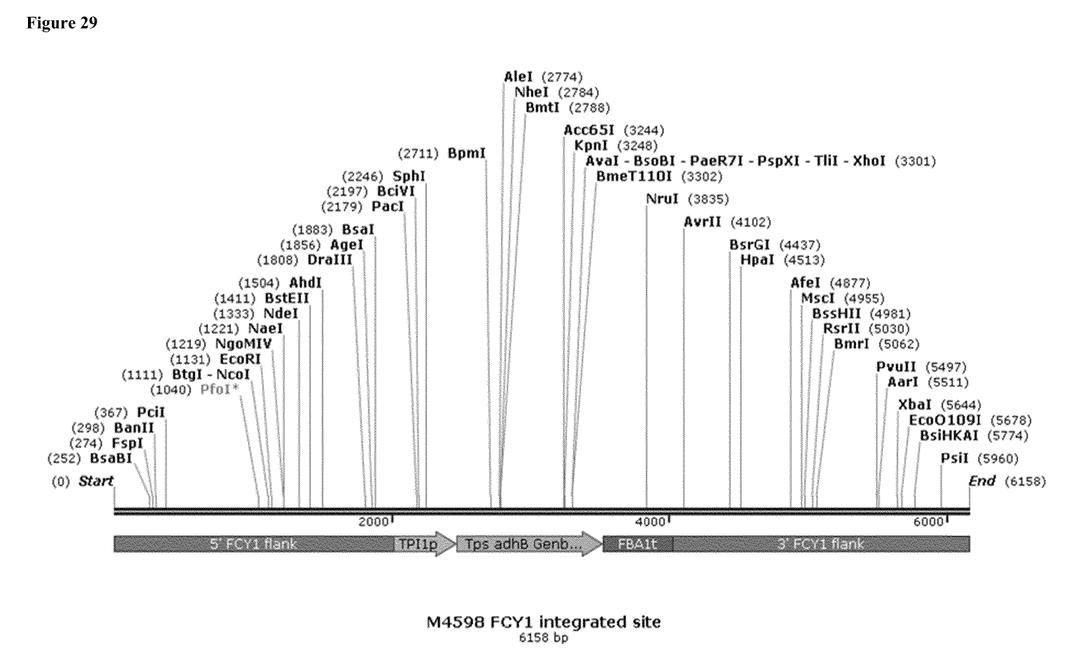

Preliminary screening of T. pseudethanolicus adhB in the M2390 strain, to create the M4596 and M4598 strains, did not result in an increase in acetate uptake compared to control strain M2390 (described in U.S. patent application Ser. No. 13/696,207 and U.S. patent application Ser. No. 13/701,652, both of which are incorporated by reference herein in their entirety). T. pseudethanolicus adhB was introduced in M2390 in the FCY1 locus (both chromosomal copies), using two different promoter/terminator pairs, as demonstrated by the schematics and vector maps depicted in FIGS. 28 and 29. The strains were grown anaerobically in YPD (40 g/l glucose, 4 g/l acetate, pH 5.5) media. Final acetate concentrations for M2390 and the M4596 and M4598 strains were very similar, suggesting that introduction of the T. pseudethanolicus adhB gene did not increase conversion of acetate to ethanol. Because the latter two strains showed improved conversion of acetone to IPA compared to M2390, this confirmed that the T. pseudethanolicus adhB gene was expressed. That the enzyme appears to be more active with acetone suggests that the intracellular metabolite levels and protein characteristics significantly favor conversion of acetone to IPA over conversion of acetyl-CoA to acetaldehyde and/or acetaldehyde to ethanol. See Burdette D. and Zeikus, J. G. However, additional NADPH-specific ACDH enzymes can be used and tested for increased acetate uptake.

Modifying ADH activity in yeast is different from modifying ACDH activity, which is not present endogenously. NADH-specific ADHs are present in very high levels in yeast (around 10 U/mg protein; see van den Brink, J, et al., “Dynamics of Glycolytic Regulation during Adaptation of Saccharomyces cerevisiae to Fermentative Metabolism,” Appl. Environ. Microbial. 74(18):5710-23 (2008)), and play an important role in standard ethanolic fermentation. As a result, high expression levels of NADPH-specific ADHs can be used, and may be needed, to compete with the activity of NADH-specific ADHs. As an alternative approach, the activity of NADH-specific ADHs can be reduced by deletion, modification, or downregulation of some of the endogenous enzymes with this activity. For example, ADH1 is an attractive target because it has been reported to be responsible for about 90% of all ADH activity. Other example ADHs depend on the host organism (including but not limited to ADH2-5 and SFA1 from Saccharomyces; see Ida, Y., et al., “Stable disruption of ethanol production by deletion of the genes encoding alcohol dehydrogenase isozymes in Saccharomyces cerevisiae,” J. Biosci. Bioeng. 113(2):192-95 (2012)), and can be identified through various genomic resources as available from the National Center for Biotechnology Information (ncbi.nlm.nih.gov) and the Saccharomyces Genome Database (yeastgenome.org). Full deletion of endogenous NADH-specific ADHs, however, would likely cripple the yeast. See Cordier, H., et al., “A metabolic and genomic study of engineered Saccharomyces cerevisiae strains for high glycerol production,” Metab. Engineer. 9(4):364-78. There is an advantage, however, to expressing NADPH-specific ADHs in the presence of native NADH-specific ADHs, because the total flux through ADH (sugar-to-ethanol+acetate-to-ethanol) is much larger than the acetate-to-ethanol flux. As a result, even if the NADPH-specific ADH flux is only 5% of the original NADH-specific ADH flux, that amount of NADPH-ADH flux would still allow for 0.8 g extra acetate uptake per 100 g sugar (any NADPH used in the sugar-to-ethanol conversion saves an equal amount of NADH that can be used in the acetate-to-ethanol route).

Most of the NADPH-specific ADHs described in the literature (EC 1.1.1.2; see, e.g., brenda-enzymes.org/php/result_flat.php4?ecno=1.1.1.2) are thought to be localized to the mitochondria or are from thermophiles, and most are thought to function best at high pH. While some may not function in the slightly acidic yeast cytosol, there are several candidate enzymes. First, there are the secondary alcohol dehydrogenases (2° Adh) from T. pseudethanolicus (adhB) and C. beijerinckii. The T. pseudethanolicus adhB is the same as that described above. The amino acid sequence for the C. beijerinckii 2° Adh is provided in SEQ ID NO:31.

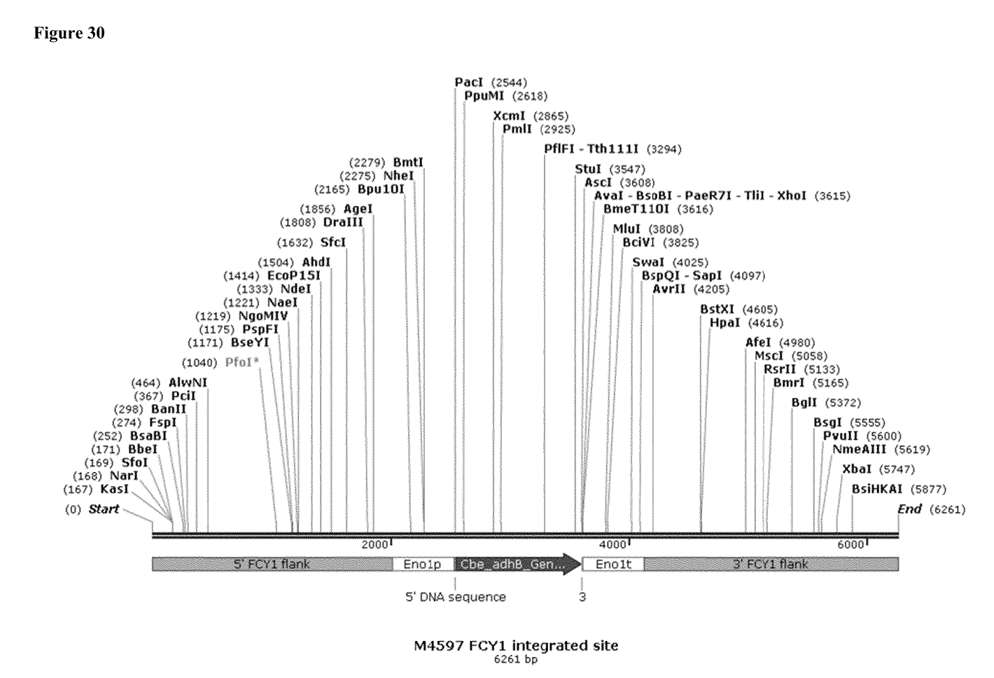

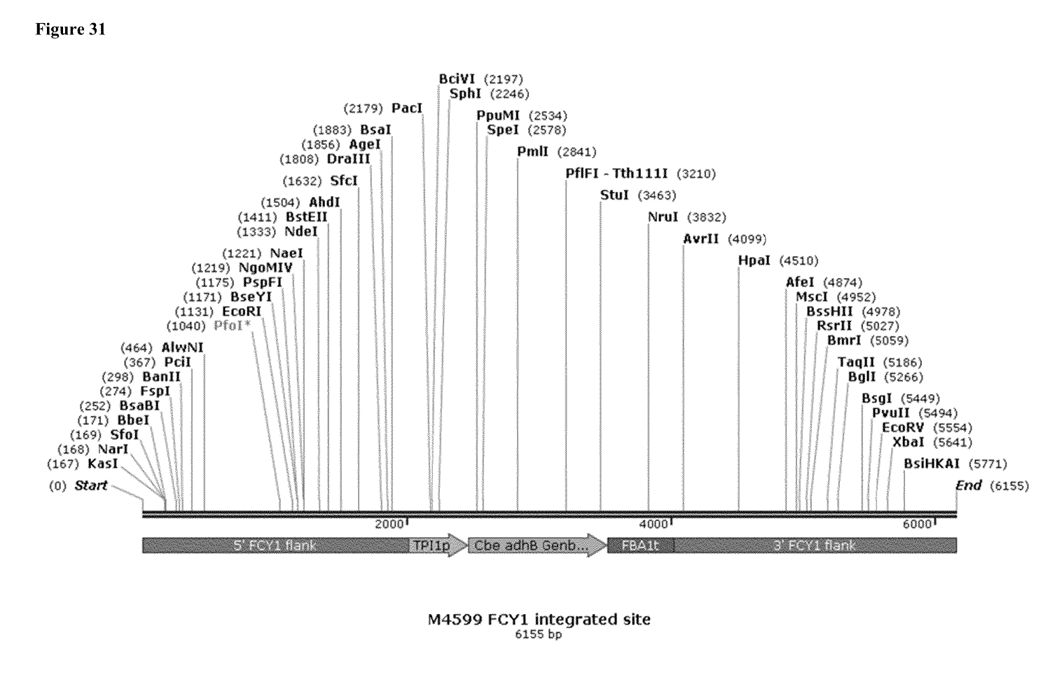

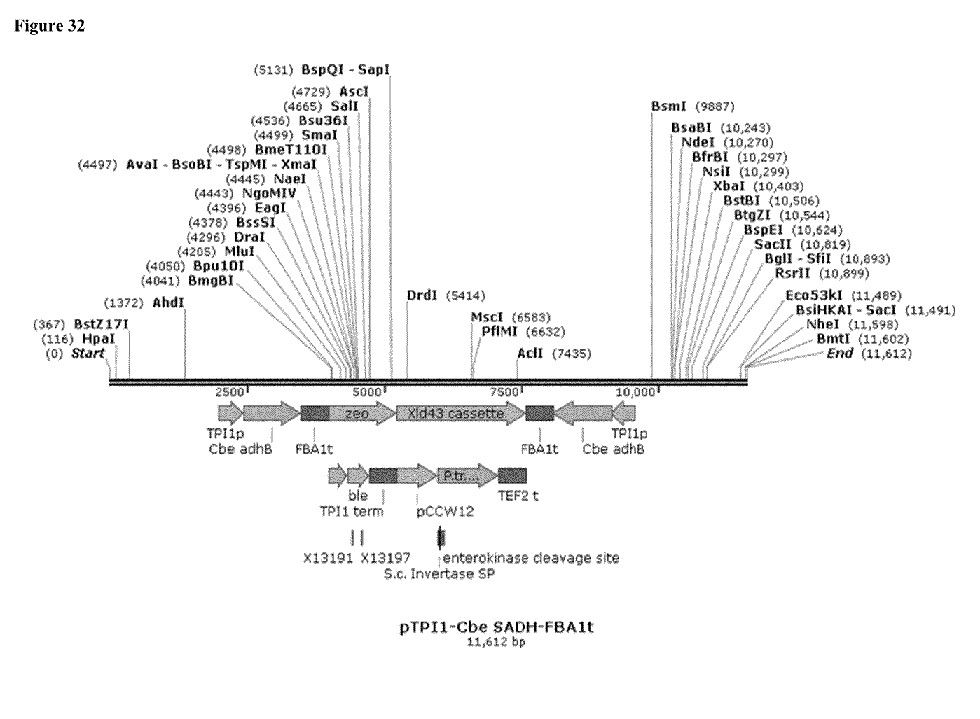

FIG. 32 depicts a schematic for the construct used to express C. beijerinckii 2° Adh (Cbe adhB). The constructs used to create strains M4597 and M4599, which contain C. beijerinckii 2° Adh expressed from the FCY1 locus, are depicted in FIGS. 30 and 31. It may be desirable to use a codon-optimized version of the C. beijerinckii 2° Adh. The nucleotide sequence for a codon-optimized C. beijerinckii 2° Adh is provided in SEQ ID NO:32.

While T. pseudethanolicus adhB and C. beijerinckii 2° Adh likely prefer acetone as a substrate, they can be tested for the desired NADPH specificity and function with acetaldehyde as a substrate. See Burdette D. and Zeikus, J. G. The secondary alcohol dehydrogenases from T. pseudethanolicus and C. beijerinckii in S. cerevisiae, were expressed and both improved the conversion of acetone to isopropanol. The strains were grown anaerobically in YPD media (40 g/l glucose, 10 g/l acetone, pH 5). After 5 days, 1.9 g/l IPA was detected in the M2390 (control) culture. With T. pseudethanolicus adhB, the IPA titers were 8.1 g/l (ENO1 promoter, ENO1 terminator) and 3.1 g/l (TPI1 promoter, FBA1 terminator). With the C. beijerinckii 2° Adh, the IPA titers were 4.1 g/l (ENO1 promoter, ENO1 terminator) and 5.1 g/l (TPI1 promoter, FBA1 terminator).

A third gene that may possess the desired NADPH-ADH activity is the S. cerevisiae gene ARI1. See GenBank Accession No, FJ851468. The nucleotide and amino acid sequences for ARI1 are provided in SEQ ID NOs:33 and 34, respectively.

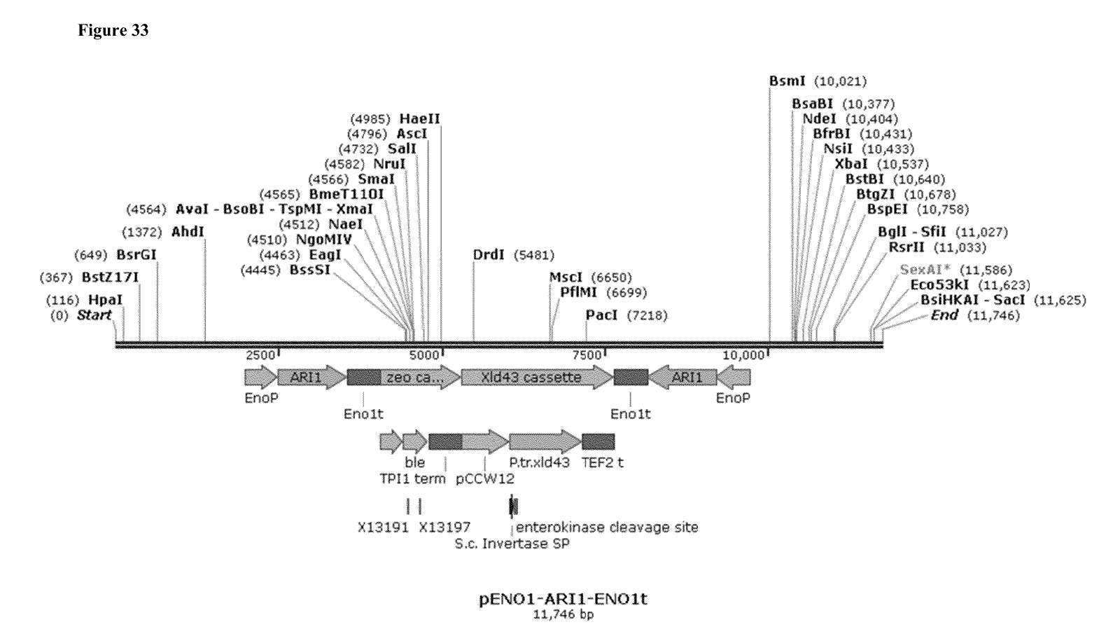

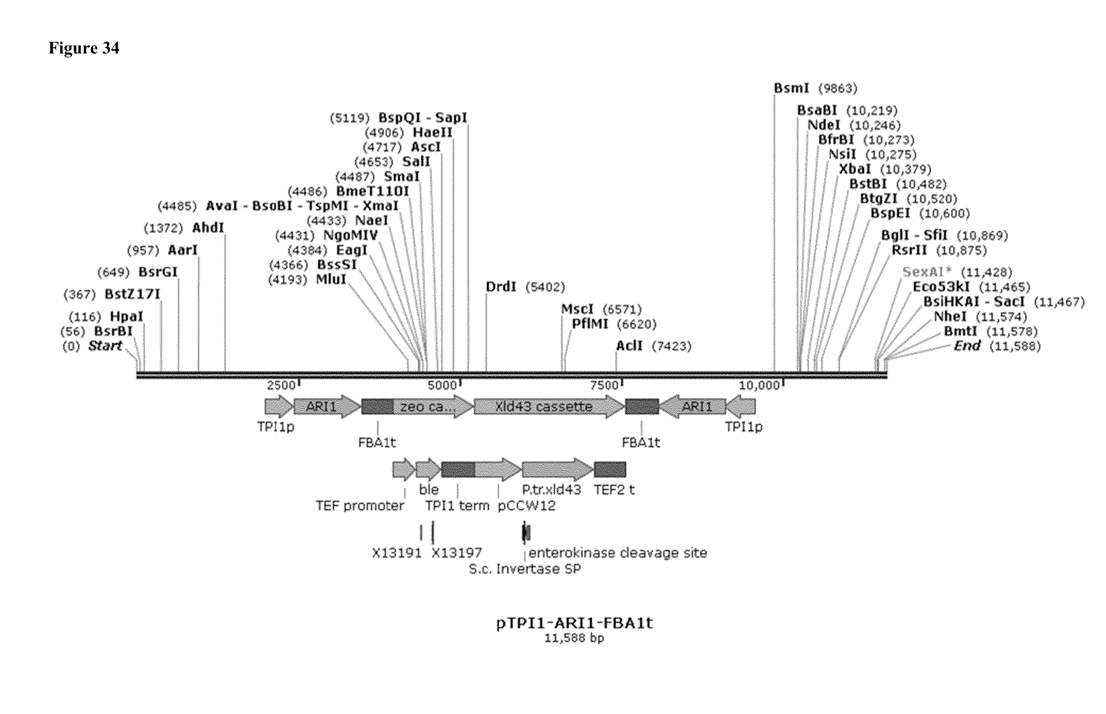

ARI1 has been shown to reduce a broad range of aldehydes. See Liu, Z. L., and Moon, J., “A novel NADPH-dependent aldehyde reductase gene from Saccharomyces cerevisiae NRRL Y-12632 involved in the detoxification of aldehyde inhibitors derived from lignocellulosic biomass conversion,” Gene 446(1):1-10 (2009). Overexpression of ARI1 improves tolerance to furfural and hydroxymethylfurfural and ARI1 has been demonstrated to act on acetaldehyde as a substrate. See Liu, Z. L., and Moon, J., (2009). Constructs used to create overexpression of ARI1 are depicted in FIGS. 33 and 34.

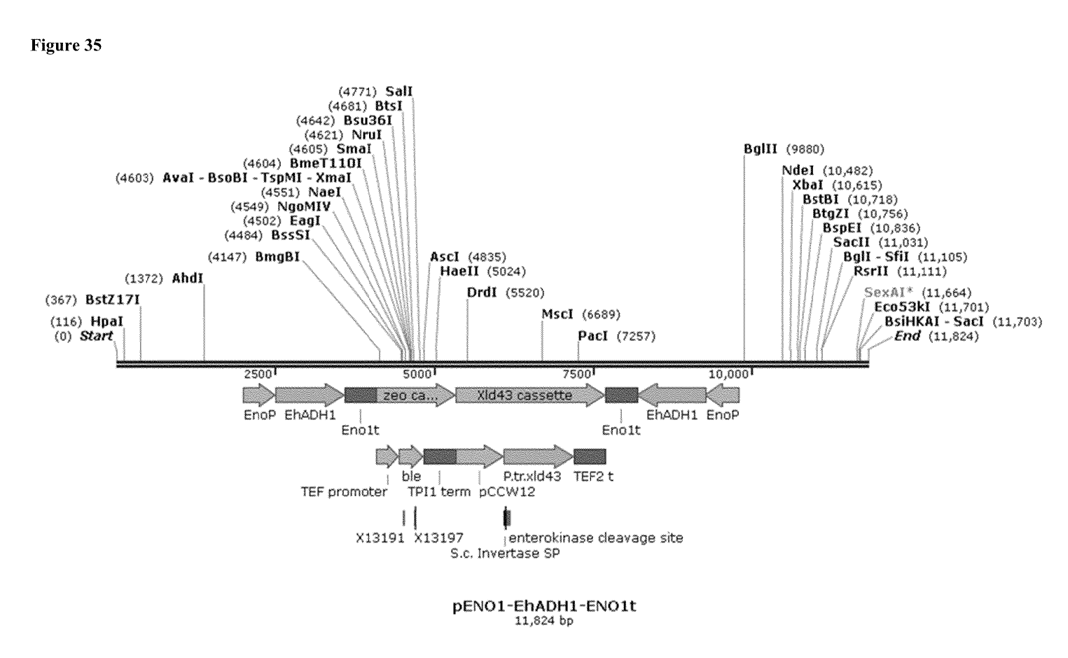

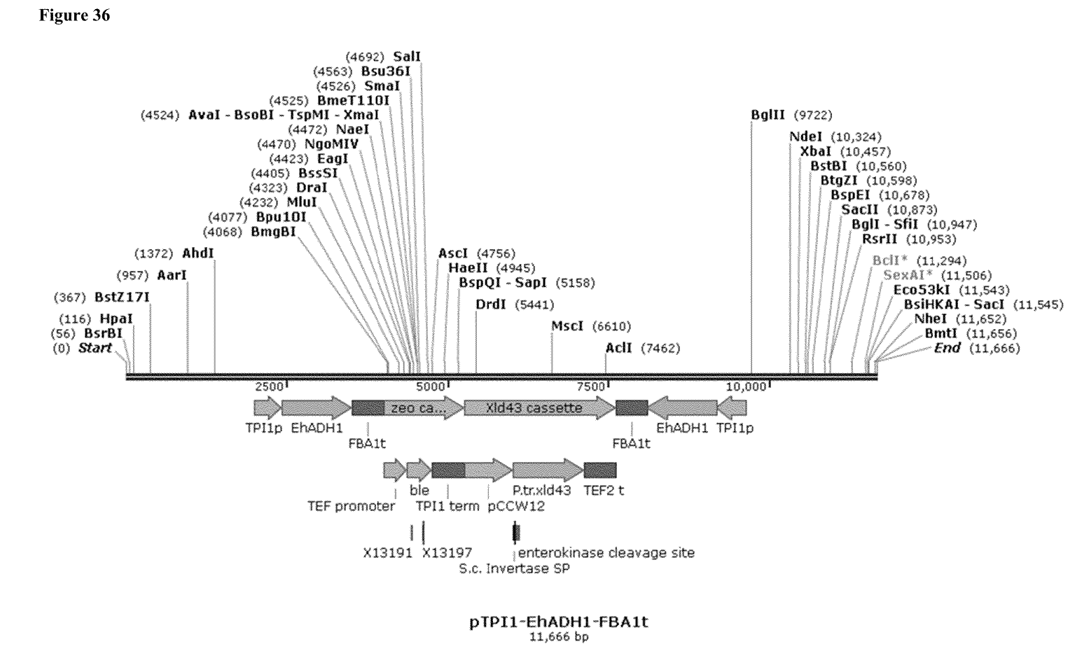

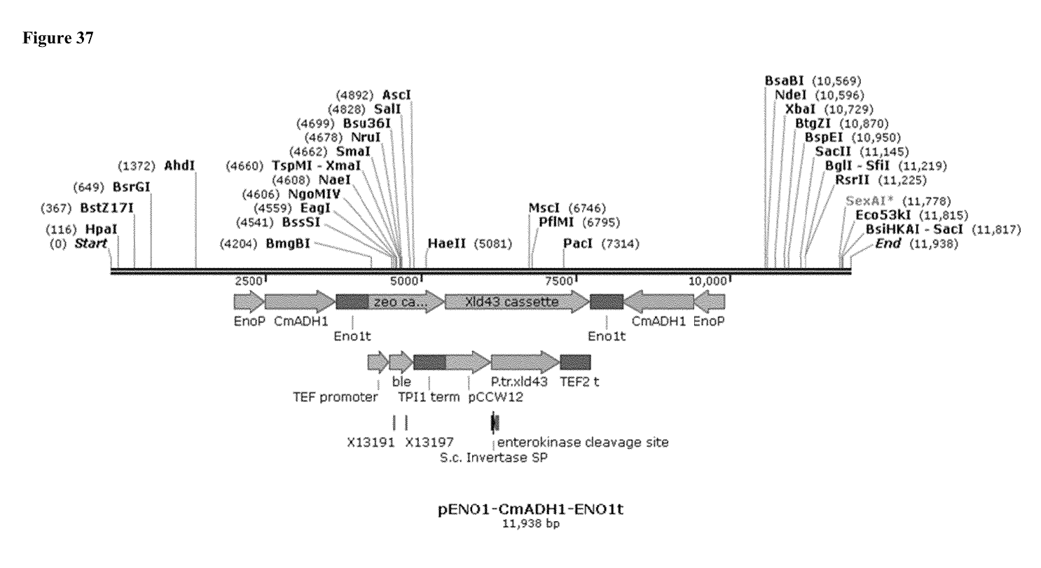

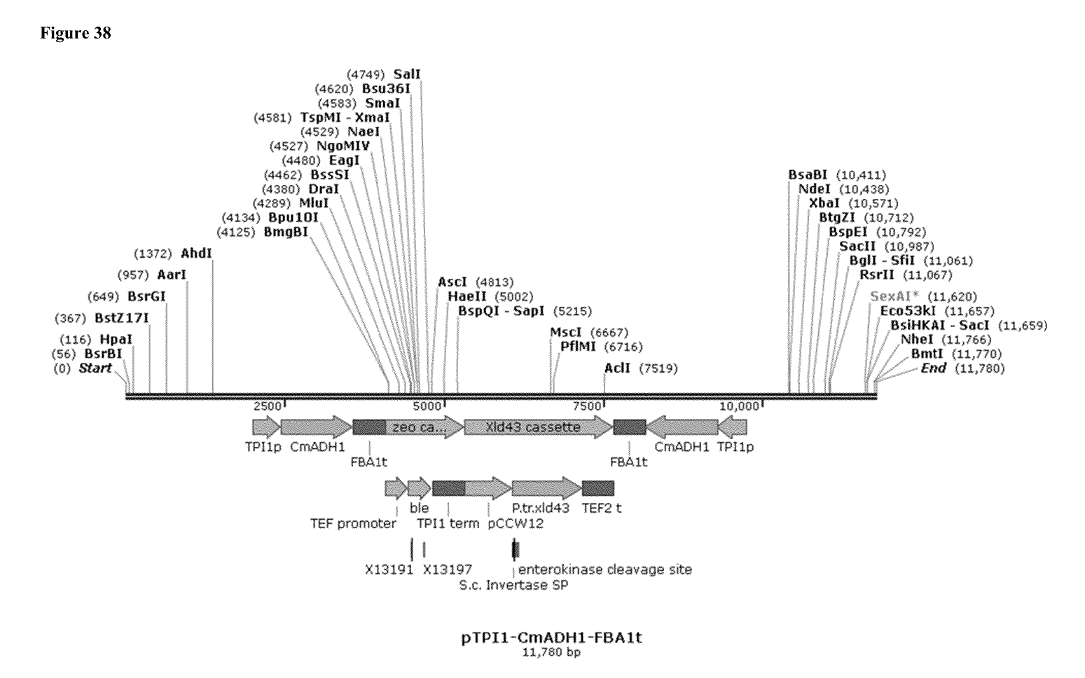

Additional genes that may have NADPH-specifc ADH activity include Entamoeba histolytica ADH1 and Cucumis melo ADH1. See Kumar, A., et al., “Cloning and expression of an NADP(+)-dependent alcohol dehydrogenase gene of Entamoeba histolytica” PNAS 89(21:10188-92 (1992) and Manriquez, D., et al., “Two highly divergent alcohol dehydrogenases of melon exhibit fruit ripening-specific expression and distinct biochemical characteristics,” Plant Molecular Biology 61(4):675-85 (2006). Constructs used to create strains expressing Entamoeba histolytica ADH1 or Cucumis melo ADH1 are depicted in FIGS. 35-38.

The nucleotide sequence for Entamoeba histolytica ADH1 is provided in SEQ ID NO:35. The nucleotide sequence for Cucumis melo ADH1 is provided in SEQ ID NO:36.

The activity of the above genes can be determined by using a gpd1/2 double knockout strain with an NADH-specific ACDH integrated into a host genome, e.g., M2594. The M2594 strain is derived from M2390 (described above) in which all chromosomal copies of GPD1 and GPD2 (M2390 is a diploid strain) have been replaced with an expression cassette with two copies of Bifidobacterium adolescentis adhE (the first AdhE reuses the original GFD promotor, while the second in reverse orientation is introduced with a new promotor, and both AdhE have a new terminator). See FIGS. 7-10.

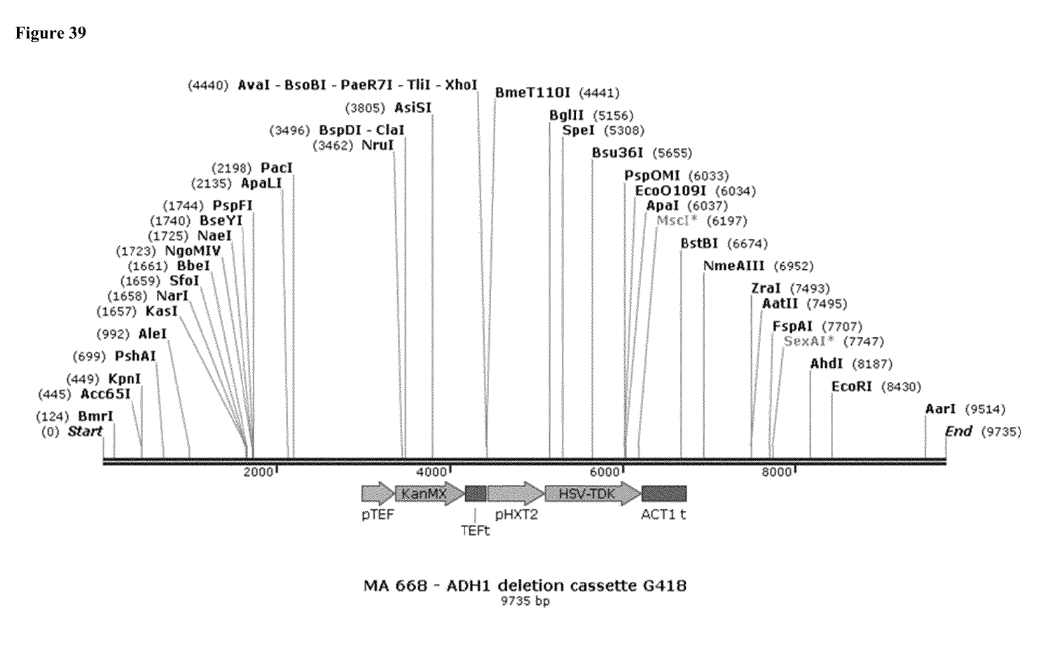

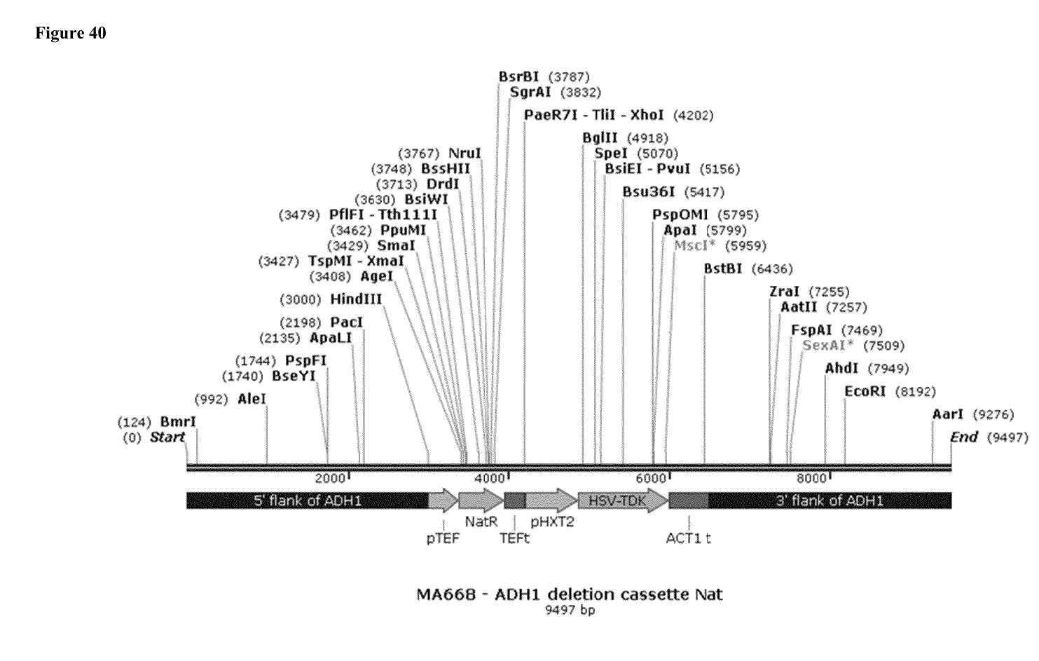

The candidate gene(s) can be expressed in high copy number and transformants screened for improved acetate uptake. This can be accomplished by integrating the gene candidates into chromosomal rDNA loci; a transformation method that allows integration of multiple copies of a gene cassette into the genome, given the multiple rDNA sequences in the genome that are highly homologous. The integration cassettes can include an antibiotic marker and xylosidase gene that can be used for selection of transformants. In addition, derivative strains of M2594 in which either one or both copies of the endogenous ADH 1 have been deleted can be employed. Constructs that can be used for the deletion of ADH 1 are depicted in FIGS. 39 and 40. Given that ADH1 is responsible for most of the yeast's NADH-specific alcohol dehydrogenase activity, reducing the expression of ADH1 may allow for the new genes to more readily compete with the high native levels of NADH-specific alcohol dehydrogenases. The screening of these strains can be performed with YPD or a Sigmacell medium, with HPLC to measure acetate levels.

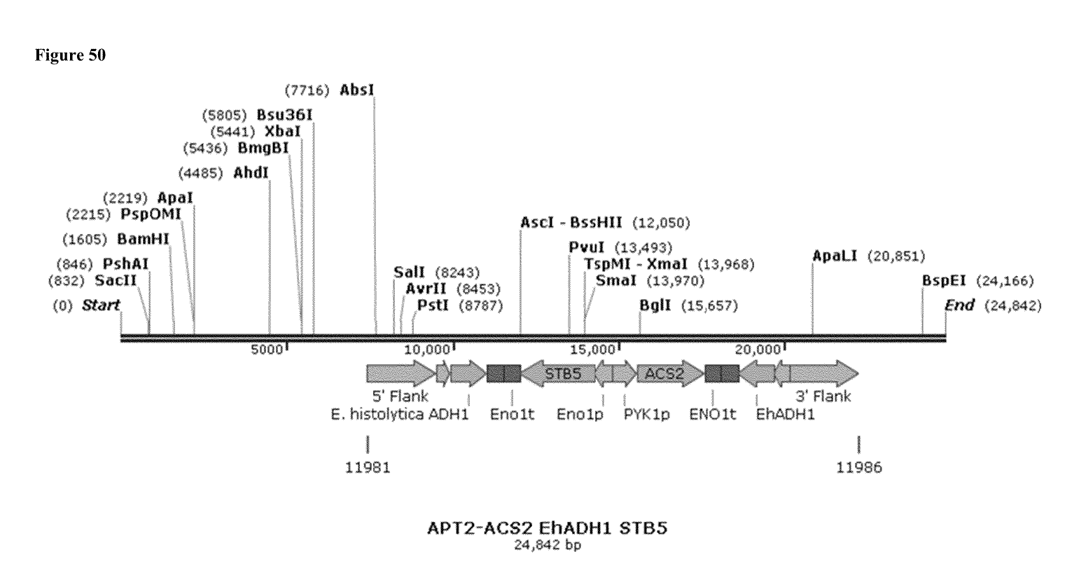

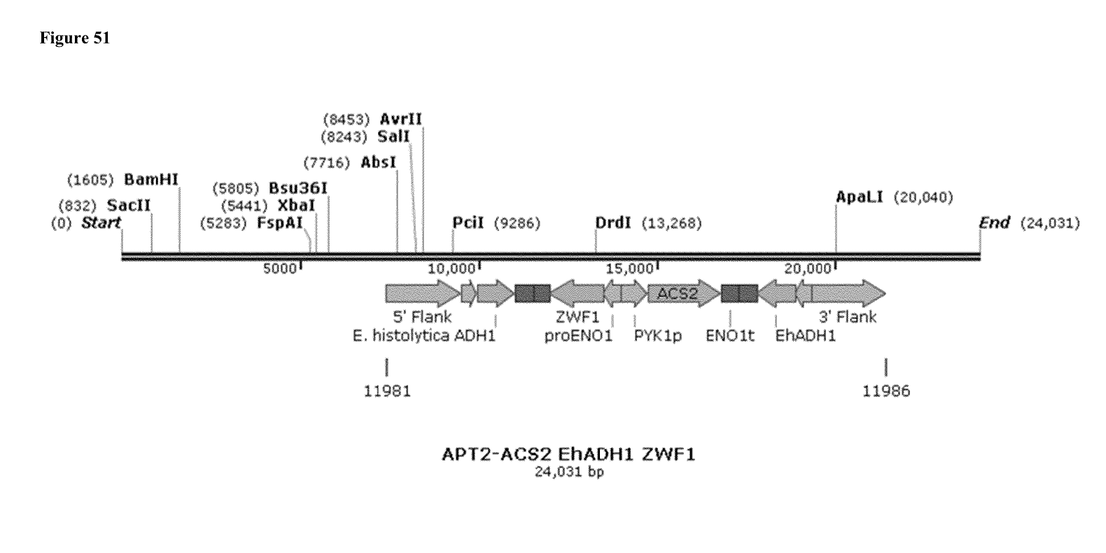

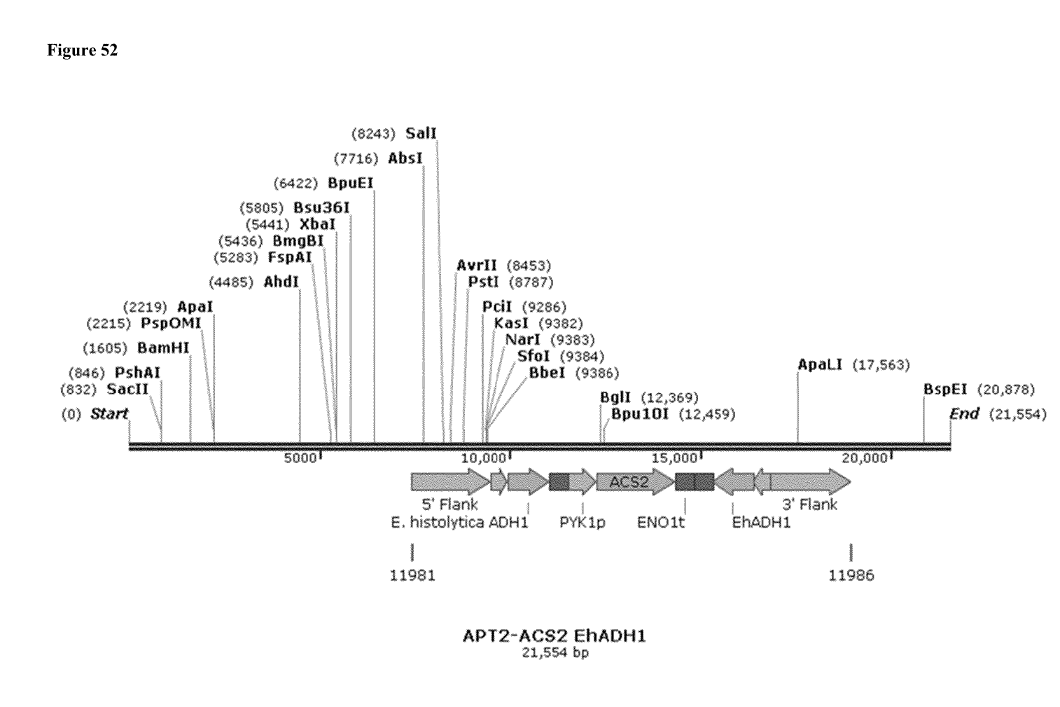

Overexpression of an acetyl-CoA synthetase, for example, a gene encoding ACS1 or ACS2, in the above strains with NADPH-specific ADH activity may lead to improved acetate-to-ethanol conversion. Examples of genes encoding ACS1 and ACS2 include those from yeast and other microorganisms, including but not limited to, Saccharomyces cerevisiae, Saccharomyces kluyveri, Zygosaccharomyces bailii, and Acetobacter aceti ACS1 and/or ACS2. See, e.g., Rodrigues, F., et al., “The Fate of Acetic Acid during Glucose Co-Metabolism by the Spoilage Yeast Zygosaccharomyces bailii,” PLOS One 7(12):e52402 (2012); Sousa, M. J., et al., “Mechanisms underlying the transport and intracellular metabolism of acetic acid in the presence of glucose in the yeast Zygosaccharomyces bailii,” Microbiology 144(3):665-70 (1998); Rodrigues, F., et al., “Isolation of an acetyl-CoA synthetase gene (ZbACS2) from Zygosaccharomyces bailii,” Yeast 21(4):325-31 (2004); Vilela-Moura, A., et al., “Reduction of volatile acidity of wines by selected yeast strains,” Appl. Microbiol. Biotechnol. 80(5):881-90 (2008); and O'Sullivan, J. and Ettlinger, L., “Characterization of the acetyl-CoA synthetase of Acetobacter aceti,” Biochimica et Biophysica Acta (BBA)—Lipid and Lipid Metabolism, 450(3):410-17 (1976). These genes, e.g., encoding the S. cerevisiae ACS2, are integrated in an expression vector to analyze its effect on acetate uptake and ethanol production. See FIGS. 50-52. ACS2 can be engineered with the E. histolytica ADH1 (SEQ ID NO:35) and/or the S. cerevisiae ZWF1 or STB5 (SEQ ID NOs:23 or 24, respectively) for effect on acetate uptake and ethanol.

The nucleotide sequence for Saccharomyces cerevisiae acs1 is provided in SEQ ID NO:37. The nucleotide sequence for Saccharomyces kluyveri acs1 is provided in SEQ ID NO:38. The nucleotide sequence for Saccharomyces cerevisiae acs2 is provided in SEQ ID NO:39. The nucleotide sequence for Saccharomyces kluyveri acs2 is provided in SEQ ID NO:40. The nucleotide sequence for Zygosaccharomyces bailii ACS is provided in SEQ ID NO:57. The nucleotide sequence for Acetobacter aceti ACS is provided in SEQ ID NO:58.

### Identifying Active NADPH-ADHs

As described above, due to high NADH-ADH activity in wild-type S. cerevisiae, and to achieve sufficiently high expression of NADPH-ADH, the NADH-ADH gene candidates were integrated in the rDNA sites, which allows for high-copy genomic integration. The integration cassettes included antibiotic markers and a xylosidase gene, as discussed above, and transformants were selected for Zeocin resistance. For each transformation, approximately two dozen transformants were screened for xylosidase activity, and the transformants with the highest activity were tested for acetate uptake. The background strain was M4868, based on M2594 (described above), in which endogenous ADH1 is marked with two antibiotic markers. Each candidate NADPH-ADH was tested with either a TPI1 promoter and FBA1 terminator, or an ENO1 promoter and ENO1 terminator. See FIGS. 32-38.

To test for acetate uptake, the transformants were grown overnight in an aerobic tube with 5 ml YPD media (40 g/l glucose, 10 g/l acetone, pH 5). The following day, cells were collected by centrifugation, washed with demineralized water, and resuspended in 2 ml demineralized water. 100 ul of the cell suspension was used to inoculate 150 ml medium bottles containing 20 ml of YPD media with 40 g/l glucose and 4 g/l acetate (added as potassium acetate), set to pH 5.5 with HCl. Bottles were capped and flushed with a gas mixture of 5% CO₂ and 95% N₂ to remove oxygen, and incubated at 35° C. in a shaker at 150 RPM for 48 hours. At 48 hours the bottles were sampled to determine glucose, acetate and ethanol concentrations, and pH using HPLC.

The results are shown below in Table 3. Each row represents a single bottle from a single transformant. All tested NADPH-ADHs, with the possible exception of the C. melo ADH1, improved acetate uptake. The highest acetate uptake was obtained with strain M4868 expressing ADH1 from E. histolytica using TPI1p and FBA1t.

TABLE 3
Acetate uptake for various NADPH-ADHs.

| | | Concentration | | Consumption | Consumption relative to |
|---|---|---|---|---|---|
| | | (g/l) | | (g/l) | M2594 (fold difference) |
| Background | ADH (in rDNA) | Acetate | Ethanol | Acetate | Acetate |
| M4868 | T. pseudethanolicus | 2.96 | 19.75 | 0.51 | 1.5 |
| | adhB (pENO1/ENO1t) | | | | |
| M4868 | T. pseudethanolicus | 3.00 | 19.89 | 0.47 | 1.4 |
| | adhB (pENO1/ENO1t) | | | | |
| M4868 | C. beijerinckii adhB | 2.77 | 20.11 | 0.70 | 2.1 |
| | (pTPI1/FBA1t) | | | | |
| M4868 | C. beijerinckii adhB | 2.56 | 20.16 | 0.91 | 2.7 |
| | (pTPI1/FBA1t) | | | | |
| M4868 | C. beijerinckii adhB | 2.82 | 20.03 | 0.65 | 2.0 |
| | (pTPI1/FBA1t) | | | | |
| M4868 | C. beijerinckii adhB | 2.81 | 20.13 | 0.66 | 2.0 |
| | (pTPI1/FBA1t) | | | | |
| M4868 | S. cerevisiae ARI1 | 3.07 | 20.00 | 0.40 | 1.2 |
| | (pENO1/ENO1t) | | | | |
| M4868 | S. cerevisiae ARI1 | 3.03 | 20.03 | 0.44 | 1.3 |
| | (pENO1/ENO1t) | | | | |
| M4868 | S. cerevisiae ARI1 | 3.00 | 19.90 | 0.47 | 1.4 |
| | (pTPI1/FBA1t) | | | | |
| M4868 | S. cerevisiae ARI1 | 2.94 | 19.97 | 0.53 | 1.6 |
| | (pTPI1/FBA1t) | | | | |
| M4868 | C. melo ADH1 | 3.09 | 19.90 | 0.38 | 1.1 |
| | (pENO1/ENO1t) | | | | |
| M4868 | C. melo ADH1 | 3.12 | 19.94 | 0.35 | 1.1 |
| | (pENO1/ENO1t) | | | | |
| M4868 | C. melo ADH1 | 3.15 | 19.83 | 0.32 | 1.0 |
| | (pTPI1/FBA1t) | | | | |
| M4868 | C. melo ADH1 | 3.11 | 19.83 | 0.36 | 1.1 |
| | (pTPI1/FBA1t) | | | | |
| M4868 | E. histolytica | 2.63 | 19.97 | 0.84 | 2.5 |
| | (pENO1/ENO1t) | | | | |
| M4868 | E. histolytica | 2.64 | 19.98 | 0.83 | 2.5 |
| | (pENO1/ENO1t) | | | | |
| M4868 | E. histolytica | 2.51 | 20.09 | 0.96 | 2.9 |
| | (pTPI1/FBA1t) | | | | |
| M4868 | E. histolytica | 2.45 | 20.23 | 1.02 | 3.1 |
| | (pTPI1/FBA1t) | | | | |
| M2594 | — | 3.14 | 20.01 | 0.33 | 1.0 |
| Medium | | 3.47 | | | |

Genotypes:
M2594: gpd1::adhE gpd2::adhE
M4868: gpd1::adhE gpd2::adhE

### Deletion of ADH1

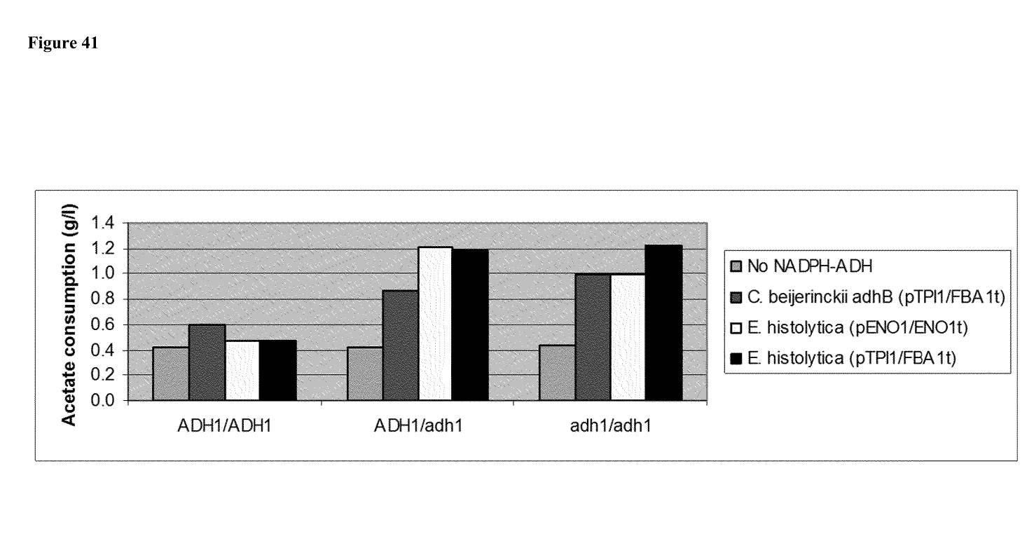

Using the NADPH-ADH results, mutants with one or both copies of the endogenous ADH1 deleted were tested. The screening process of above was repeated with two additional backgrounds: M2594 (with two functional copies of ADH1) and M4867 (with a single copy ADH1 deletion), with NADPH-ADHs, from E. histolytica and C. beijerinckii. These additional transformants demonstrated that expressing NADPH-ADH has little effect on acetate uptake in M2594, but increased acetate consumption in a single knockout of ADH1 (M4867) and in strain M4868 compared to M2594. The results are shown below in Table 4. The data for several isolates for each background/NADPH-ADH/promoter/terminator combination are shown in FIG. 41.

TABLE 4
Acetate uptake for ADH1 deletion mutants.

| | | | | Consumption |
|---|---|---|---|---|
| | | | | relative to |
| | Concentration | | Consumption | M2594 (fold |
| | (g/l) | | (g/l) | difference) |
| Modification | Acetate | Ethanol | Acetate | Acetate |
| C. beijerinckii adhB | 3.11 | 19.49 | 0.59 | 1.4 |
| (pTPI1/FBA1t) | | | | |
| E. histolytica | 3.23 | 19.34 | 0.48 | 1.1 |
| (pENO1/ENO1t) | | | | |
| E. histolytica | 3.26 | 19.52 | 0.45 | 1.0 |
| (pENO1/ENO1t) | | | | |
| E. histolytica | 3.23 | 19.33 | 0.47 | 1.1 |
| (pTPI1/FBA1t) | | | | |
| E. histolytica | 3.26 | 19.44 | 0.45 | 1.0 |
| (pTPI1/FBA1t) | | | | |
| C. beijerinckii adhB | 2.91 | 19.69 | 0.79 | 1.9 |
| (pTPI1/FBA1t) | | | | |
| C. beijerinckii adhB | 2.83 | 19.59 | 0.87 | 2.0 |
| (pTPI1/FBA1t) | | | | |
| E. histolytica | 3.08 | 19.50 | 0.63 | 1.5 |
| (pENO1/ENO1t) | | | | |
| E. histolytica | 3.10 | 19.59 | 0.61 | 1.4 |
| (pENO1/ENO1t) | | | | |
| E. histolytica | 3.01 | 19.59 | 0.70 | 1.6 |
| (pENO1/ENO1t) | | | | |
| E. histolytica | 2.50 | 19.81 | 1.20 | 2.8 |
| (pENO1/ENO1t) | | | | |
| E. histolytica | 2.52 | 19.76 | 1.18 | 2.8 |
| (pTPI1/FBA1t) | | | | |
| E. histolytica | 2.69 | 19.86 | 1.01 | 2.4 |
| (pTPI1/FBA1t) | | | | |
| wild-type | 3.56 | 18.76 | 0.14 | 0.3 |
| gpd1::adhE | 3.28 | 19.45 | 0.42 | 1.0 |
| gpd2::adhE | | | | |
| gpd1::adhE | 3.29 | 19.70 | 0.42 | 1.0 |
| gpd2::adhE | | | | |
| adh1/ADH1 | | | | |
| gpd1::adhE | 3.27 | 19.54 | 0.44 | 1.0 |
| gpd2::adhE | | | | |
| adh1/adh1 | | | | |
| M4868 + | 2.71 | 19.77 | 1.00 | 2.3 |
| C. beijerinckii adhB | | | | |
| (pTPI1/FBA1t) | | | | |
| M4868 + | 2.71 | 20.00 | 0.99 | 2.3 |
| E. histolytica | | | | |
| (pENO1/ENO1t) | | | | |
| M4868 + | 2.48 | 19.62 | 1.23 | 2.9 |
| E. histolytica | | | | |
| (pTPI1/FBA1t) | | | | |
| Medium | 3.70 | | | |

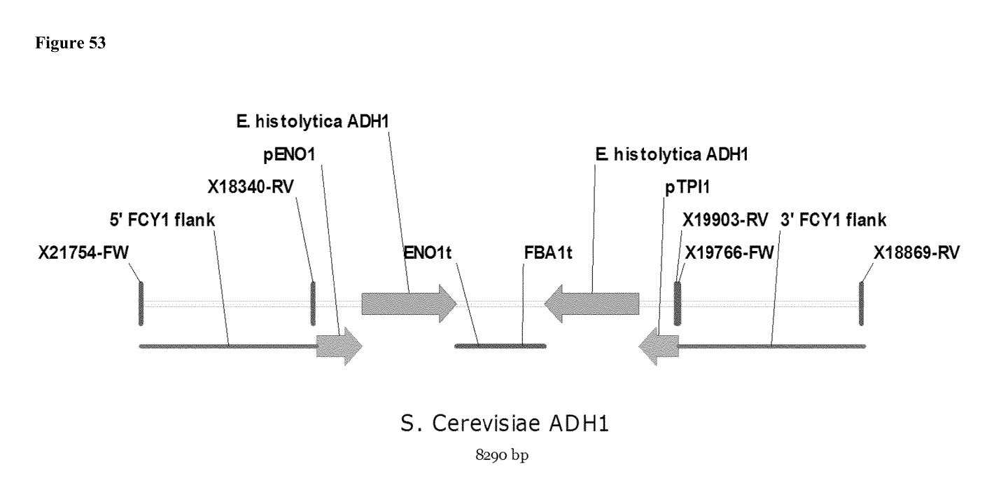

Additional strains that express the NADPH-ADH from E. histolytica without any changes to the endogenous NADH-ADH1 were created using the strategy depicted in FIG. 53. Strain M6571 is a restocked version of M2594 and is genotypically identical to M2594.

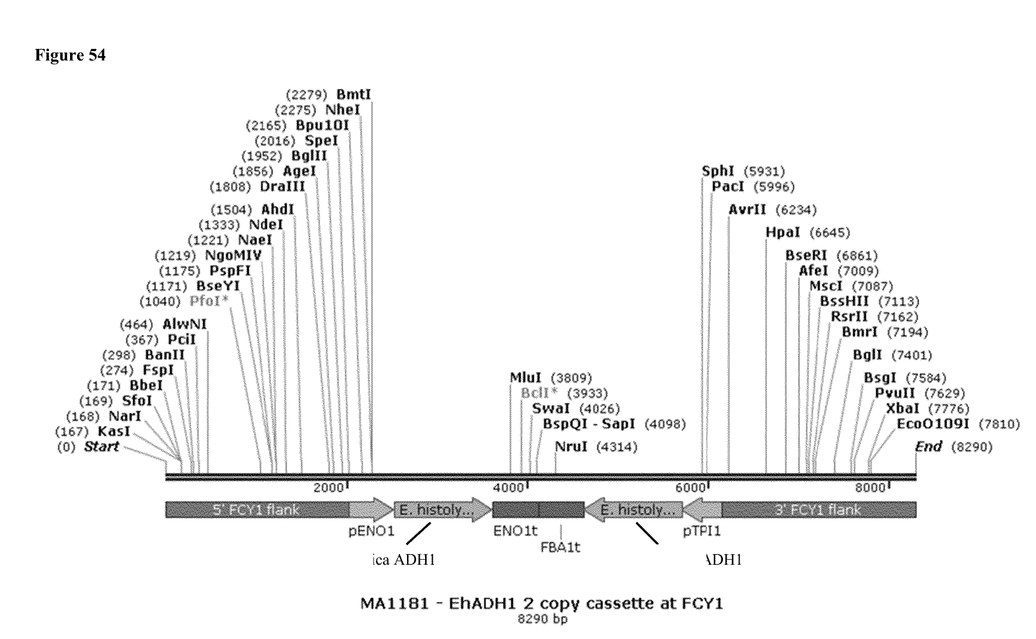

Strains M6950 and M6951 have the E. histolytica ADH1 expressed at the site of the endogenous FCY1 gene, using two promoter/terminator combinations in an opposed orientation. Strains M6950 and 6951 were constructed by integrating the assembly MA1181 into M2594, using methods described above, and replacing the original FCY1 ORF with a two-copy expression cassette of E. histolytica ADH1. See FIG. 54. Transformants were selected for 5FC resistance using FCY1 as a counterselectable marker. Experimental results for the various strains with 40 or 110 g/L glucose in bottles is provided in Tables 5 and 6. The 40 g/L glucose bottles were sparged with N₂/CO₂ prior to incubation, whereas the 110 g/L bottles were not.

TABLE 5
Acetate uptake for E. histolytica ADH1 expressing
strains grown in 40 g/L glucose.
YPD (40 g/l) Sampled after 48 hours

| | | | | | | Acetate |
|---|---|---|---|---|---|---|
| | | HPLC Concentrations (g/l) | | | | consumption |
| Bottle no. | Strain | Glucose | Glycerol | Acetate | Ethanol | (g/l) |
| 1 | M2390 | | 1.0 | 4.8 | 17.2 | 0.0 |
| 2 | M2594 | | 0.1 | 4.5 | 17.7 | −0.2 |
| 4 | M6950 | | 0.1 | 4.2 | 17.8 | −0.6 |
| 5 | M6951 | 0.1 | 0.1 | 4.2 | 17.7 | −0.6 |
| 10 | M5553 | | 0.1 | 4.6 | 17.6 | −0.2 |
| 11 | M5582 | | 0.1 | 4.1 | 17.9 | −0.7 |
| 12 | M5586 | | 0.2 | 3.7 | 18.0 | −1.0 |
| | Media | 35.9 | 0.1 | 4.7 | | |

TABLE 6
Acetate uptake for E. histolytica ADH1 expressing
strains grown in 100 g/L glucose.
YPD (110 g/l), not flushed; Sampled after 72 hours

| | | | | | | Acetate |
|---|---|---|---|---|---|---|
| | | Concentrations (g/l) | | | | consumption |
| Bottle no. | Strain | Glucose | Glycerol | Acetate | Ethanol | (g/l) |
| 13 | M2390 | | 2.5 | 4.6 | 51.0 | −0.3 |
| 14 | M2594 | | 0.2 | 3.9 | 52.8 | −1.0 |
| 16 | M6950 | | 0.2 | 2.5 | 53.5 | −2.4 |
| 17 | M6951 | | 0.2 | 2.4 | 53.7 | −2.5 |
| 22 | M5553 | | 0.1 | 4.1 | 53.2 | −0.8 |
| 23 | M5582 | | 0.3 | 2.1 | 53.9 | −2.8 |
| 24 | M5586 | 11.8 | 1.3 | 1.9 | 46.0 | −3.0 |
| | M6571 | | 0.1 | 4.1 | 52.8 | −0.8 |
| | Media | 110.1 | 0.1 | 4.9 | | |

As demonstrated in Table 6, acetate consumption in strains M6950 and M6951 is comparable to that of strain M5582, in which both copies of endogenous ADH1 are deleted and E. histolytica ADH1 is expressed (see Tables 7-9 below). Thus, while deleting one or both copies of endogenous ADH1 in microorganisms expressing exogenous NADPH-specific ADHs might be beneficial in the context of acetate consumption, it is not required to obtain a significant improvement in acetate uptake.

### Improving NADPH Availability

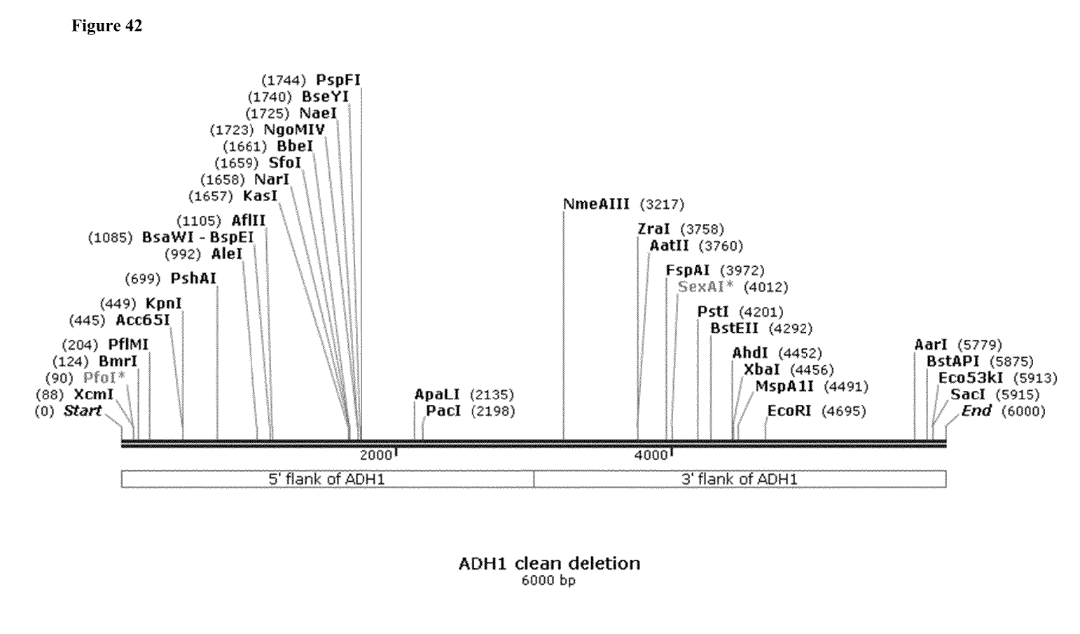

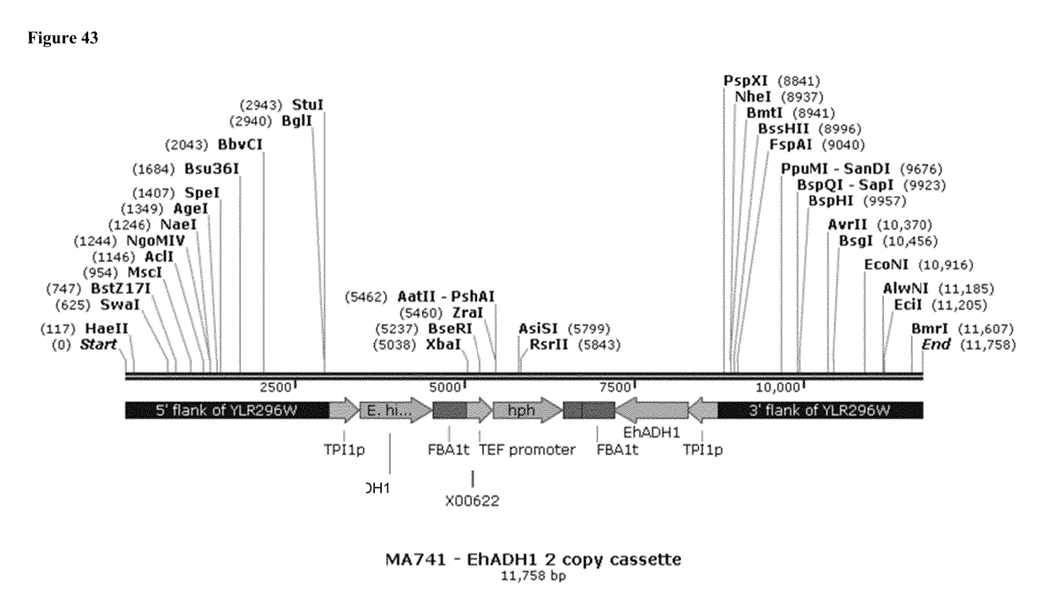

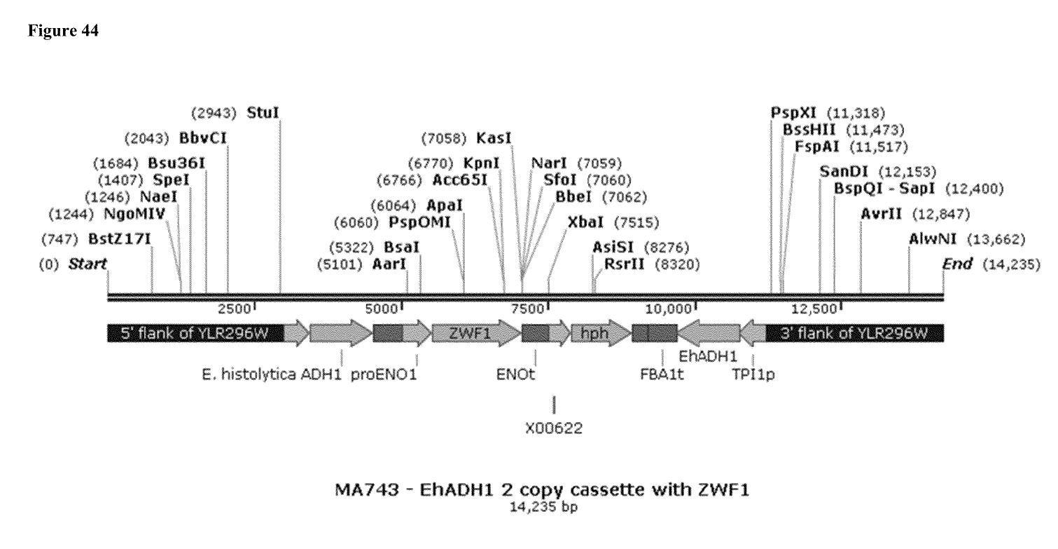

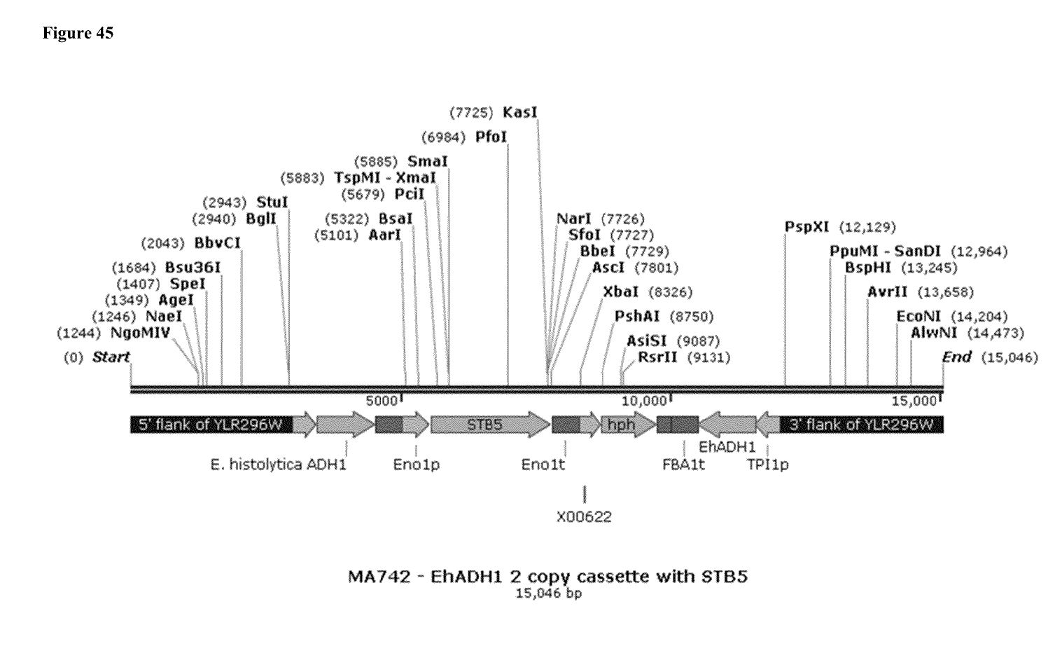

To determine if acetate uptake can be further increased above the NADPH-ADH results described above for the ADH1 double knockout strains, STB5 or ZWF1 were overexpressed. Strains were reconstructed, targeting the NADPH-ADH to the site of YLR296W, to eliminate uncertainty regarding the copy number of the rDNA integration cassettes (see FIGS. 43-45). To facilitate the strain construction, the ADH1 ORFs were cleanly deleted (not leaving any antibiotic markers; FIG. 42), resulting in strain M5553. Transformants expressed 4 copies of the E. histolytica ADH1 and two copies of ZWF1 or STB5.

Screening of several transformants indicated that STB5 overexpression slightly reduced acetate uptake, whereas ZWF1 overexpression increased acetate uptake, compared to overexpression of E. histolylica ADH1 alone. The results are shown below in Tables 7 and 8.

TABLE 7
Acetate uptake for strains overexpressing
E. histolytica ADH1 and either STB5 or ZWF1.

| | | Concentration | | Consumption |
|---|---|---|---|---|
| | | (g/l) | | (g/l) |
| Strain | Modification | Acetate | Ethanol | Acetate |
| M2390 | wild-type | 4.52 | 18.09 | 0.1 |
| M2594 | gpd1::adhE gpd2::adhE | 4.31 | 18.88 | 0.3 |
| M4868 | M2594 adh1 marked by | 4.30 | 18.85 | 0.3 |
| | antibiotic markers | | | |
| M5279 | M4868 + E. histolytica | 3.91 | 18.83 | 0.7 |
| | ADH1 (pENO1/ENO1t) | | | |
| | (rDNA) | | | |
| M5280 | M4868 + E. histolytica | 3.70 | 19.15 | 0.9 |
| | ADH1 (pTPI1/FBA1t) | | | |
| | (rDNA) | | | |
| M5553 | M2594 adh1/adh1 | 4.31 | 18.93 | 0.3 |
| M5582 | M5553 + E. histolytica | 3.72 | 19.22 | 0.9 |
| | ADH1 (4x) | | | |
| M5583 | M5553 + E. histolytica | 3.67 | 19.16 | 0.9 |
| | ADH1 (4x) | | | |
| M5584 | M5553 + E. histolytica | 3.88 | 19.20 | 0.7 |
| | ADH1 (4x) + STB5 (2x) | | | |
| M5585 | M5553 + E. histolytica | 3.85 | 19.21 | 0.8 |
| | ADH1 (4x) + STB5 (2x) | | | |
| M5586 | M5553 + E. histolytica | 3.44 | 19.12 | 1.2 |
| | ADH1 (4x) + ZWF1 (2x) | | | |

TABLE 8
Acetate uptake for strains overexpressing
E. histolytica ADH1 and either STB5 or ZWF1.

| | | Concentration | | Consumption |
|---|---|---|---|---|
| | | (g/l) | | (g/l) |
| Strain | Modification | Acetate | Ethanol | Acetate |
| M2390 | wild-type | 3.73 | 17.70 | 0.0 |
| M2594 | gpd1::adhE gpd2::adhE | 3.33 | 18.55 | 0.4 |
| M5280 | M4868 + E. histolytica | 2.76 | 18.75 | 1.0 |
| | ADH1 (pTPI1/FBA1t) | | | |
| | (rDNA) | | | |
| M5582 | M5553 + E. histolytica | 2.80 | 18.73 | 1.0 |
| | ADH1 (4x) | | | |
| M5583 | M5553 + E. histolytica | 2.80 | 18.73 | 1.0 |
| | ADH1 (4x) | | | |
| M5584 | M5553 + E. histolytica | 2.93 | 18.75 | 0.8 |
| | ADH1 (4x) + STB5 (2x) | | | |
| M5585 | M5553 + E. histolytica | 2.89 | 18.70 | 0.9 |
| | ADH1 (4x) + STB5 (2x) | | | |
| M5586 | M5553 + E. histolytica | 2.48 | 18.88 | 1.3 |
| | ADH1 (4x) + ZWP1 (2x) | | | |

### Higher Sugar Concentrations

To determine if acetate uptake can be increased above the NADPH-ADH results described above in the presence of an increased sugar concentration, strains were screened in YPD with 120 g/l glucose and 5.5 g/l acetate, pH 5.5. The bottles in these high-sugar concentration experiments were not flushed with a nitrogen/carbon dioxide mixture because flushing the bottles does not always result in finishing the fermentation, which can leave residual sugar behind. Acetate consumption increased up to 3.3 g/l under an increased sugar concentration. See Table 9.

TABLE 9
Acetate uptake at an increased sugar concentration.

| | | Concentration | | Consumption |
|---|---|---|---|---|
| | | (g/l) | | (g/l) |
| Strain | Modification | Acetate | Ethanol | Acetate |
| M2390 | wild-type | 5.3 | 52.4 | 0.0 |
| M2390 | wild-type | 5.4 | 52.1 | −0.1 |
| M2594 | gpd1::adhE | 4.3 | 54.6 | 1.0 |
| | gpd2::adhE | | | |
| M2594 | gpd1::adhE | 4.5 | 54.9 | 0.8 |
| | gpd2::adhE | | | |
| M5553 | M2594 adh1 | 4.3 | 54.7 | 1.0 |
| M5553 | M2594 adh1 | 4.5 | 54.7 | 0.8 |
| M5582 | M5553 + EhADH1 | 2.0 | 55.5 | 3.3 |
| | (4x) | | | |
| M5582 | M5553 + EhADH1 | 2.1 | 55.6 | 3.2 |
| | (4x) | | | |
| | Medium | 5.3 | | |

### Strain Construction

Construction of M2390 and M2594 are described above. Strain M4867 was constructed by deleting a single copy of ADH1 using the cassette depicted in FIG. 2. M4868 was constructed by deleting both copies of ADH1 using the cassettes depicted in FIGS. 39 and 40. Strain M5553 is similarly based on M2594, but has clean deletions of two copies of ADH1 (i.e., the promoter and terminator were left intact, but the open reading frame (ORF) was removed). See FIG. 2. The S. cerevisiae ADH1 nucleotide sequence for reference strain S288C is provided in SEQ ID NO:41.

Strains M5582, M5584 and M5586 are based on M5553, and overexpress ADH1 from E. histolytica as well as endogenous STB5 (M5584 only) or ZWF1 (M5586 only). See FIGS. 43-45. The sequence of these genes is provided above. Each of these integrations replaces the ORF of YLR296W. Integration cassettes containing either hygromycin or zeocin resistance markers allowed targeting of both YLR296W sites in the diploid strain. See FIGS. 43-45.

### Summary

As demonstrated above, deleting endogenous NADH-ADH and introducing heterologous NADPH-ADH improved conversion of acetate to ethanol. Without wishing to be bound by any theory, the improvement may be due to the introduction of a redox imbalance in sugar fermentation, leading to a net conversion of NADPH to NADH. A smaller but additional beneficial effect is that the acetate-to-ethanol pathway itself, for which a heterologous NADH-dependent acetaldehyde dehydrogenase is expressed, also relies on alcohol dehydrogenase. With NADPH-ADH, the conversion of acetate to ethanol consumes less NADH and more NADPH. Because the yeast strains were tested anaerobically, and because these strains are glycerol-3-phosphate dehydrogenase negative, the only way the cells can reoxidize NADH is by taking up acetate and converting it to ethanol. In addition, further improvements in acetate uptake were obtained by overexpressing ZWF1, whereas overexpressing STB5 had less of an effect.

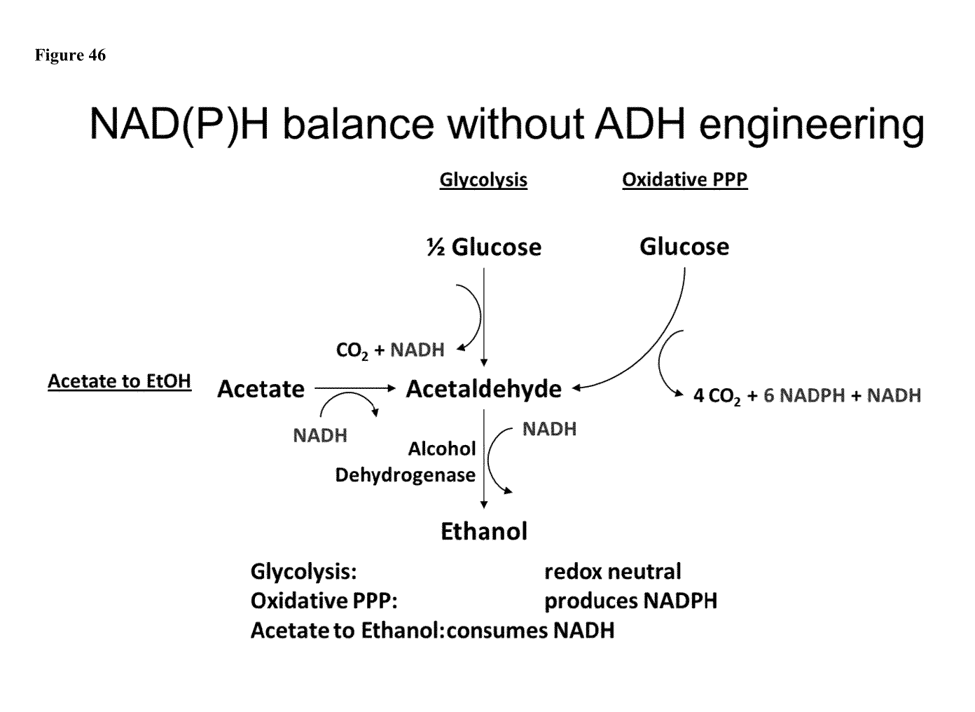

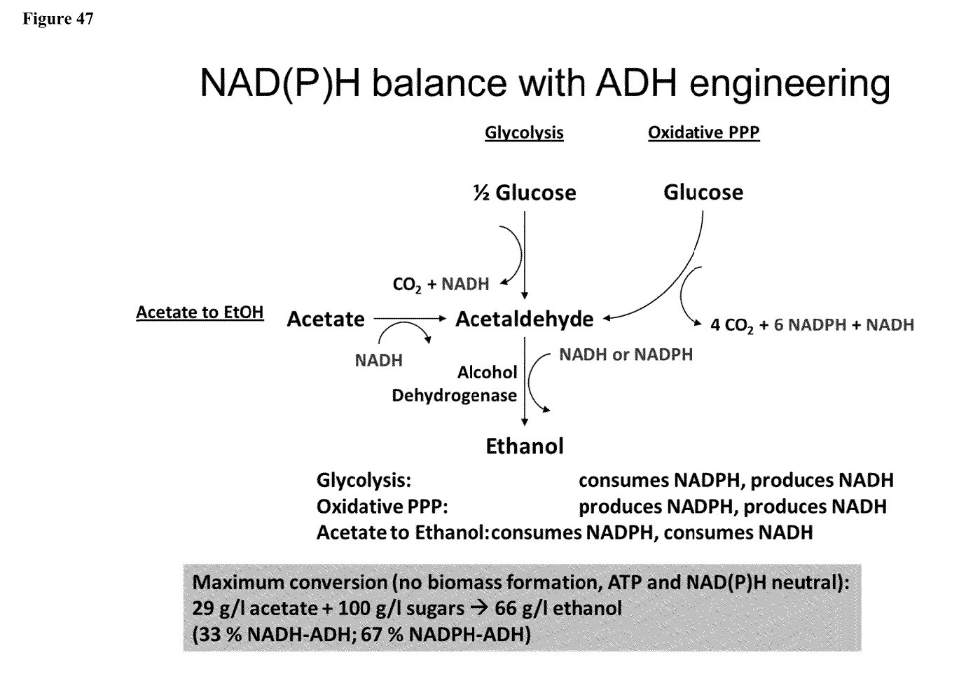

FIGS. 46 and 47 show how the use of redox cofactors is affected by expressing NADPH-ADH. In the extreme case where yeast balance the use of NADH and NADPH (i.e., as much NADH is consumed as is produced; same for NADPH), and where yeast directs all of the ATP it generates from sugar fermentation to the conversion of acetate to ethanol, 29 g/l acetate can be consumed per 100 g/l glucose (or xylose). In this case, two-thirds of the ADH activity is NADPH-dependent, and one-third is NADH-dependent. The above strains might be unable to grow when completely lacking in NADH-ADH activity, because this would produce more NADH than can be consumed with the limited amount of ATP available from sugar metabolism. The strains containing deletions in both copies of ADH1 (which results in partial replacement of cytosolic NADH-ADHs with NADPH-ADH) grew, however, so modifying the cofactor preference for ADH demonstrated cell viability and increased acetate consumption and ethanol production with an NADPH-preferring ADH.

### Example 3

The present prophetic example describes engineering of a recombinant microorganism to use the ribulose-monophosphate pathway (RuMP) for production of electron donors to be used in the conversion of acetate to ethanol or isopropanol.

Instead of relying on the endogenous oxidative branch of the PPP as described in Example 2, the heterologous RuMP pathway found in various bacteria and archaea, including Bacillus subtilis, Methylococcus capsulatus, and Thermococcus kodakaraensis, which also produces CO₂ while conferring electrons to redox carriers, can be introduced. See Yurimoto, H., et al., “Genomic organization and biochemistry of the ribulose monophosphate pathway and its application in biotechnology,” Appl. Microbiol. Biotechnol. 84:407-416 (2009).

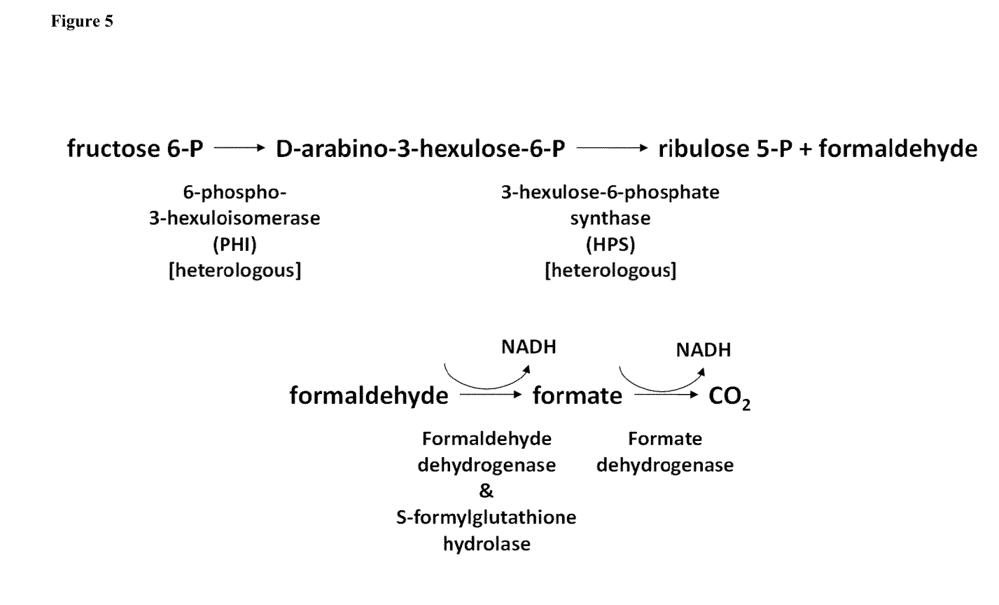

This pathway relies on the expression of two heterologous genes, 6-phospho-3-hexuloisomerase (PHI) and 3-hexulose-6-phosphate synthase (HPS). Examples of PHI and HPS enzymes include Mycobacterium gastri rmpB and Mycobacterium gastri rmpA, respectively. PHI converts fructose-6-P to D-arabino-3-hexulose-6-P, and HPS converts the latter to ribulose-5-P and formaldehyde. See FIG. 5. While this conversion is redox neutral, the produced formaldehyde can then be converted to CO₂ by the action of the endogenous enzymes formaldehyde dehydrogenase (SPA1) and S-formylglutathione hydrolase (YJL068C), which produce formate and NADH, and formate dehydrogenase (FDH1), which converts the formate to CO₂, producing a second NADH. These enzymes can be overexpressed or upregulated.

A beneficial effect of FDH1 overexpression on formate consumption has been demonstrated. See Geertman, J-M. A., et al., “Engineering NADH metabolism in Saccharomyces cerevisiae: formate as an electron donor for glycerol production by anaerobic, glucose-limited chemostat cultures,” FEMS Yeast Research 6(8):1193-1203 (2006). It is also possible to overexpress heterologous genes, like the formaldehyde and formate dehydrogenases from O. polymorpha, which improve formaldehyde consumption in S. cerevisiae. See Baerends, R. J. S., et al., “Engineering and Analysis of a Saccharomyces cerevisiae Strain That Uses Formaldehyde as an Auxiliary Substrate,” Appl. Environ. Microbiol. 74(1):3182-88 (2008). Overexpression of an NADH-dependent acetaldehyde dehydrogenase may also be employed to enable conversion of acetate to ethanol. Competition with glycerol formation (another NADH-consuming reaction) can be prevented by deleting gpd1 and gpd2.

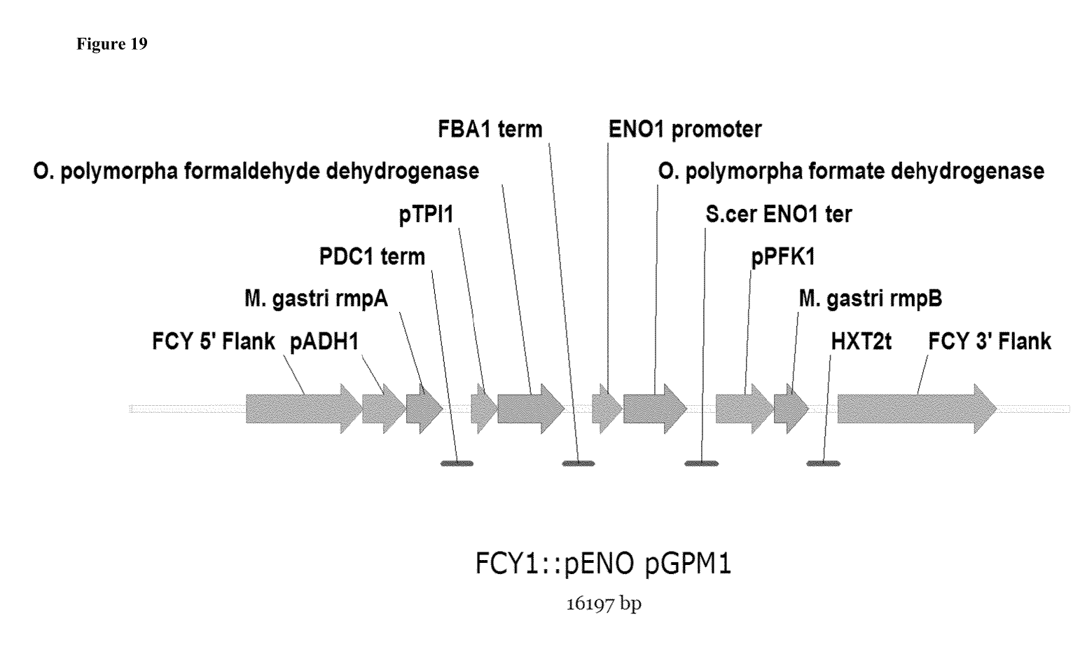

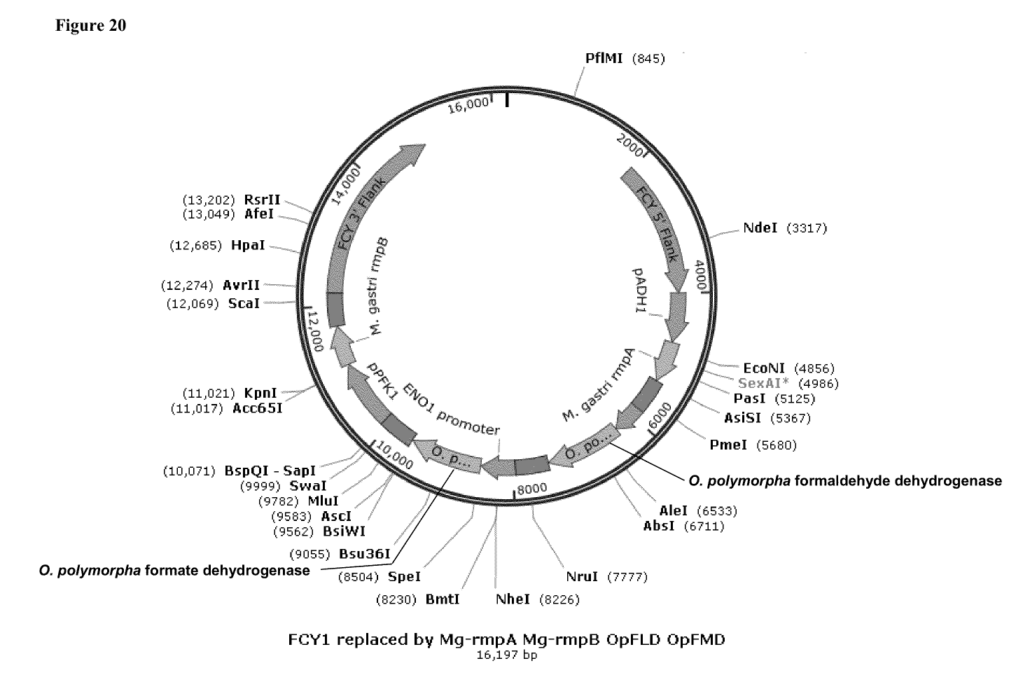

This strain is grown under anaerobic conditions in media containing C6 and/or C5 sugars, as well as acetate. See FIGS. 19 and 20. The RuMP pathway, combined with formaldehyde degradation to CO₂, can generate NADH, which will improve the conversion of acetate to ethanol via an NADH-dependent acetaldehyde dehydrogenase.

The sequence for Mycobacterium gastri rmpB (PHI) is provided in SEQ ID NO:42. The sequence for Mycobacterium gastri rmpA (HPS) is provided in SEQ ID NO:43. The sequence for Saccharomyces cerevisiae SFA1 is provided in SEQ ID NO:44. The sequence for Saccharomyces cerevisiae YJL068C is provided in SEQ ID NO:45. The sequence for Saccharomyces cerevisiae FDH1 is provided in SEQ ID NO:46. The sequence for Candida boidinii FDH3 is provided in SEQ ID NO:47.

To bring this strategy into practice, first the formaldehyde or formate degrading enzymes can be overexpressed or upregulated in a yeast such as S. cerevisiae, and then assayed to verify that the increased NADH production allows for increased acetate consumption in cultures supplemented with formaldehyde and/or formate. This assay involves the addition of formaldehyde or formate to the medium and determining whether these compounds are taken up by the yeast and if it produces more ethanol, using techniques described herein and in WO 2012/138942 (PCT/US2012/032443), incorporated by reference herein in its entirety. Once this has been demonstrated, functional expression of PHI and HPS that confer this benefit without the need for formaldehyde/formate supplementation can be screened. Functional expression of PHI and HPS in the pathway can be screened by measuring for improved acetate uptake and ethanol titers as described herein and in U.S. patent application Ser. No. 13/696,207, incorporated by reference herein in its entirety. FIG. 20 depicts a construct used to create a microorganism containing this engineered RuMP pathway.

FDH Expression

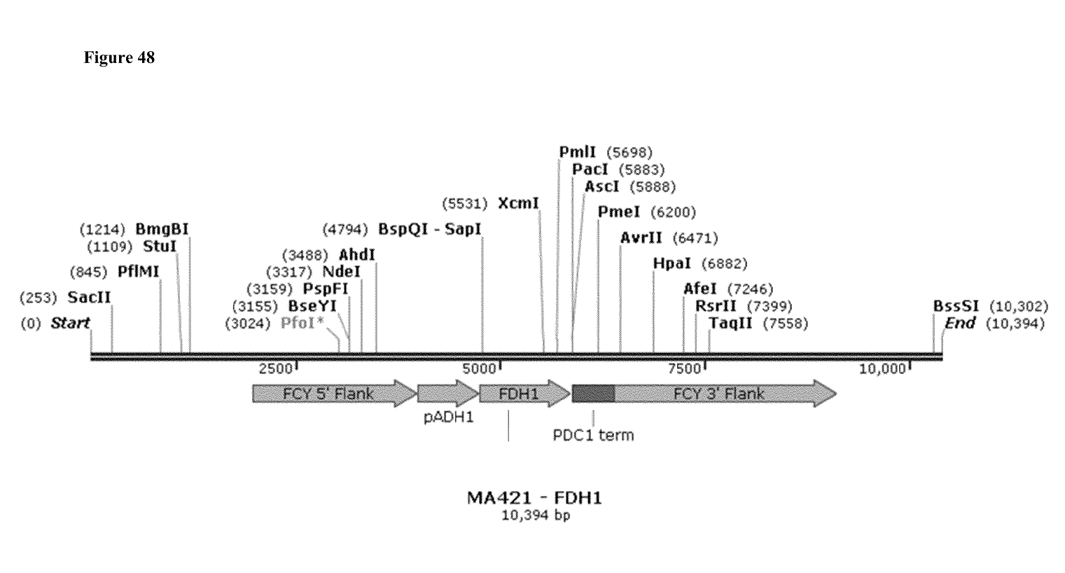

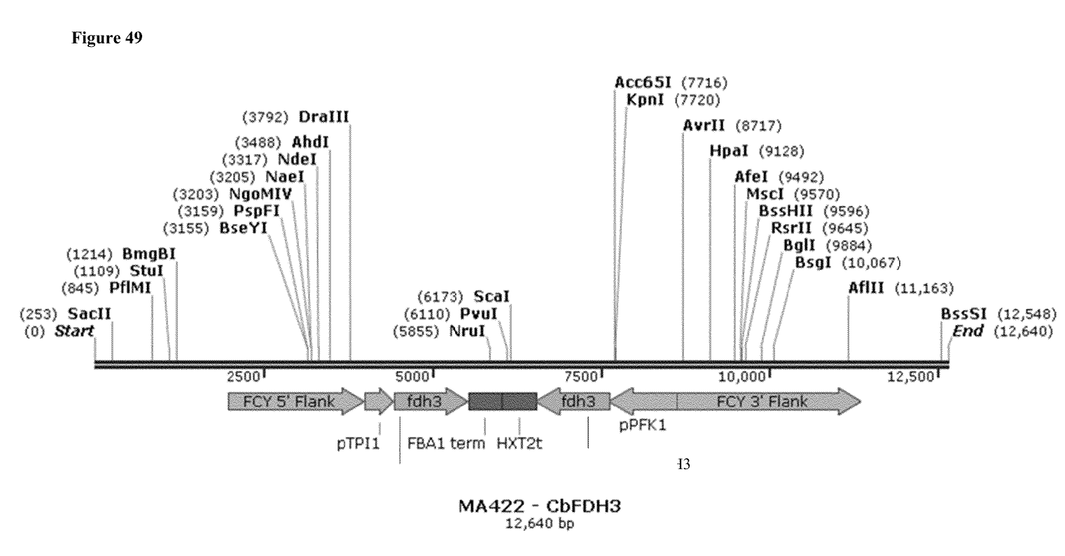

Acetate consumption and availability of NADH was measured by expression of a formate dehydrogenase from S. cerevisiae (FDH1; SEQ ID NO: 46) or from Candida boidinii (FDH3; SEQ ID NO: 47). Two cassettes, one with a single copy of the S. cerevisiae FDH1 (ADH1 promoter and PDC1 terminator) (FIG. 48), and one with two copies of the Candida boidinii FDH3 (TPI1 promoter, FBA1 terminator, and PFK1 promoter, HXT2 terminator) (FIG. 49), were expressed in M2594. Two verified transformants per cassette were tested in anaerobic bottles on YPD (40 g/l glucose, 3 g/l acetate, and 2 g/l formate, pH 4.8 (set with HCl)), which were sparged with 5% CO₂/95% N₂ after inoculation to remove oxygen, and incubated for 48 hours at 35° C. and 150 RPM.

Acetate and formate consumption were measured for the FDH transformants, as well as for the M2390 and M2594 background strains, according to the assay described above. The results are shown in Table 10. Both the S. cerevisiae FDH1 and the C. boidinii FDH3 transformants demonstrated improved acetate consumption compared to the M2390 strain. The C. boidinii FDH3 transformants showed the highest acetate consumption, which may be in part due to expression of two copies of the gene or promoter/terminator selection. Thus, expression of a formate degrading enzyme such as FDH increases acetate consumption and ethanol production.

TABLE 10
Acetate uptake for strains over expressing S. cerevisiae
FDH1 or C. boidinii FDH3.

| | | Concentration (g/l) | | | Consumption (g/l) | |
|---|---|---|---|---|---|---|
| Background | Modification | Acetate | Ethanol | Formate | Acetate | Formate |
| M2390 | Wild-type | 2.57 | 18.9 | 1.62 | 0.10 | 0.17 |
| M2594 | gpd1::adhE gpd2::adhE | 2.35 | 19.7 | 1.61 | 0.32 | 0.18 |
| M4109 | gpd1::adhE gpd2::adhE | 2.35 | 19.8 | 1.57 | 0.32 | 0.21 |
| | fcy1::FDH1 | | | | | |
| M4110 | gpd1::adhE gpd2::adhE | 2.30 | 19.5 | 1.57 | 0.37 | 0.22 |
| | fcy1::FDH1 | | | | | |
| M4111 | gpd1::adhE gpd2::adhE | 2.21 | 20.0 | 1.35 | 0.46 | 0.44 |
| | fcy1::C.boidinii FDH3 | | | | | |
| M4112 | gpd1::adhE gpd2::adhE | 2.19 | 19.7 | 1.32 | 0.48 | 0.47 |
| | fcy1::C.boidinii FDH3 | | | | | |
| | Medium | 2.67 | | 1.79 | | |

### Example 4

The present prophetic example describes engineering of a recombinant microorganism to use the dihydroxyacetone pathway (DHA) for production of electron donors to be used in the conversion of acetate to ethanol or isopropanol.

The DHA pathway is conceptually similar to the RuMP pathway of Example 3, as both rely on the formation of formaldehyde and the subsequent oxidation of the formaldehyde to CO₂, producing NADH. With the DHA pathway, formaldehyde is produced by the action of formaldehyde transketolase (EC 2.2.1.3), which interconverts dihydroxyacetone and glyceraldehyde-3-P into xylulose-5-P and formaldehyde. See FIG. 6. The required dihydroxyacetone can be produced by either glycerol dehydrogenase or dihydroxyacetone phosphatase:

glycerol+NAD(P)→dihydroxyacetone+NAD(P)H (catalyzed by glycerol dehydrogenase) or

dihydroxyacetone-P→dihydroxyacetone (catalyzed by dihydroxyacetone phosphatase)

dihydroxyacetone+glyceraldehyde-3-P→xylulose-5-P+formaldehyde (catalyzed by formaldehyde transketolase)

formaldehyde→CO₂+2 NADH (catalyzed by formaldehyde dehydrogenase, S-formylglutathione hydrolase, and formate dehydrogenase)

DHA degradation via formaldehyde transketolase has been described for S. cerevisiae, and baker's yeast has an endogenous glycerol dehydrogenase, encoded by GCY1. See Molin, M., and A. Blomberg, “Dihydroxyacetone detoxification in Saccharomyces cerevisiae involves formaldehyde dissimilation,” Mol. Microbiol. 60:925-938 (2006) and Yu, K. O., et al., “Engineering of glycerol utilization pathway for ethanol production by Saccharomyces cerevisiae,” Bioresource Technol. 101:4157-4161 (2010). Glycerol dehydrogenases from several organisms, including Hansenula polymorpha (gdh), E. coli (gldA) and Pichia angusta (gdh), have also been functionally expressed in S. cerevisiae. See Jung, J-Y., et al., “Production of 1,2-propanediol from glycerol in Saccharomyces cerevisiae,” J. Microbiol. Biotechnol. 21:846-853 (2011) and Nguyen, H. T. T. and Nevoigt, E., “Engineering of Saccharomyces cerevisiae for the production of dihydroxyacetone (DHA) from sugars: A proof of concept,” Metabolic Engineering 11:335-346 (2009). Dihydroxyacetone-P-specific phosphatase-activity has been found in the bacterium Zymomonas mobilis. See Horbach, S., et al., “Enzymes involved in the formation of glycerol 3-phosphate and the by-products dihydroxyacetone and glycerol in Zymomonas mobilis,” FEMS Microbiology Letters 120:37-44 (1994).

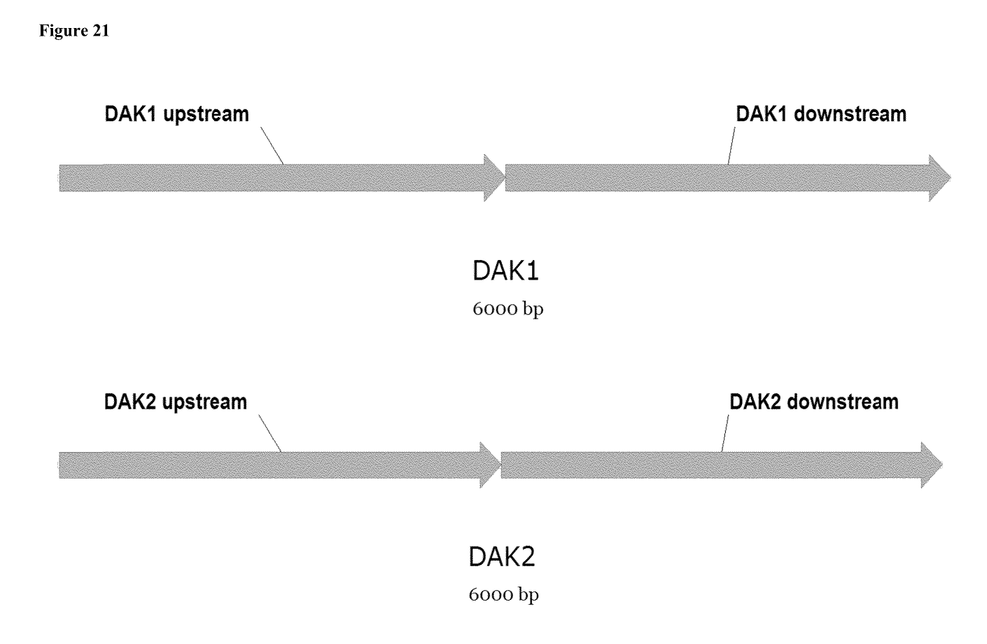

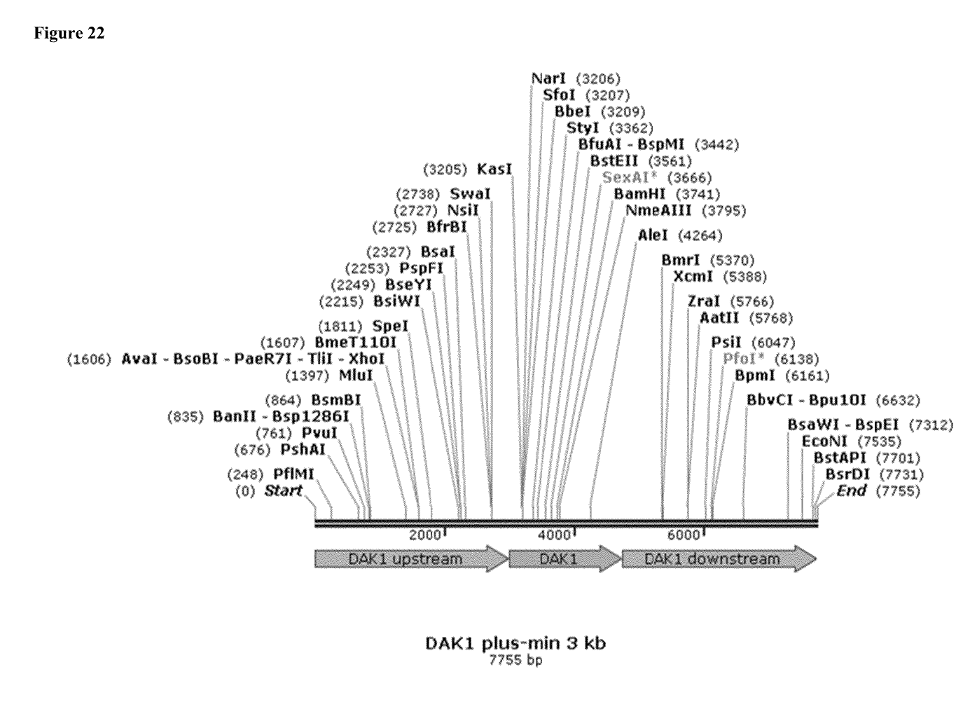

To prevent conversion of dihydroxyacetone to dihydroxyacetone phosphate, expression of the DAK1/DAK2 genes, which encode dihydroxyacetone kinases, can be downregulated For example, the DAK1/DAK2 genes can be deleted. See FIGS. 20-22. Dihydroxyacetone kinases convert DHA to DHAP. In this pathway, NADH is generated via the conversion of glycerol, produced from DHAP, to CO₂ and xylulose-5-P. Rephosphorylating DHA would result in a futile cycle. If a glycerol dehydrogenase is used and the medium contains glycerol (either introduced by the feedstock or released by the cells), the STL1-encoded glycerol/proton-symporter can be overexpressed or upregulated to take up glycerol from the medium. A source of DHA is required for this pathway to function. Extracellular glycerol is an attractive source, although it might not be present in all media, and it may not be economical to add it. In the case where glycerol is present, expressing a transporter is likely to improve the capacity of the cell to take up glycerol, especially at lower glycerol concentrations. See International Patent Application Publication No WO2011/149353, which is incorporated by reference herein in its entirety.

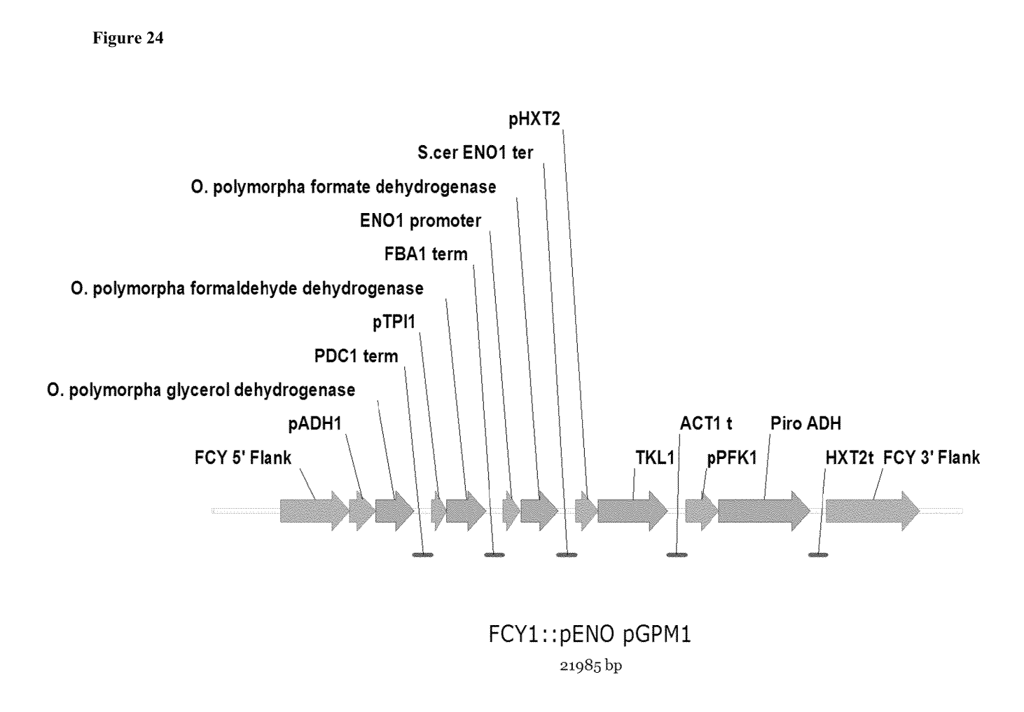

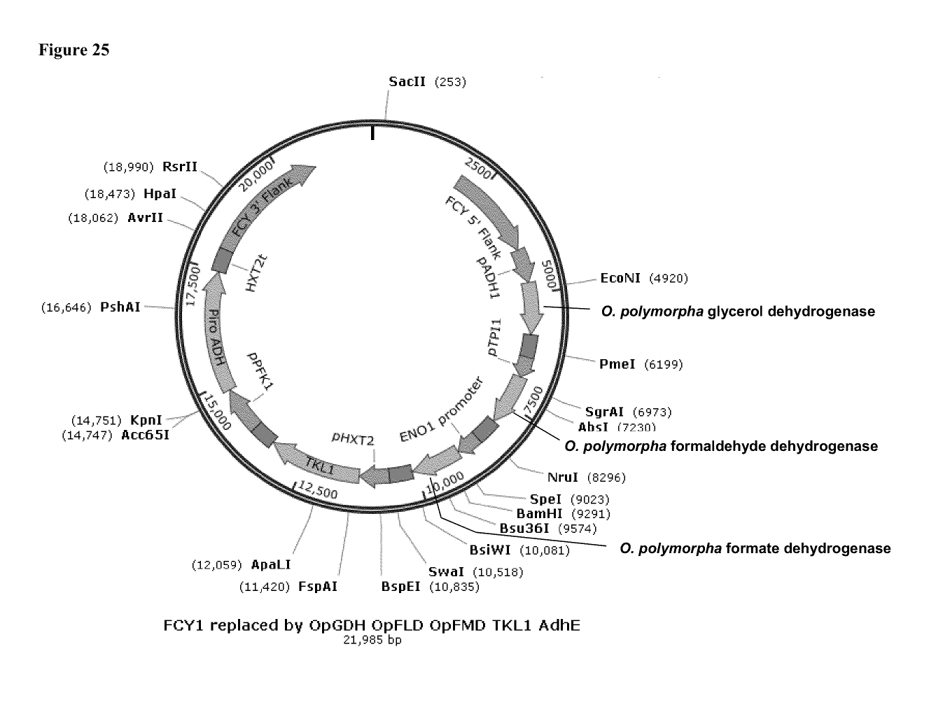

The desired strain comprises overexpression of glycerol dehydrogenase and transketolase to convert glycerol to xylulose-5-P and formaldehyde, and overexpression of formaldehyde dehydrogenase and formate dehydrogenase to convert formaldehyde to CO₂. In addition, deletion of both dihydroxyacetone kinases (DAK1 and DAK2) is desired to prevent (re)phosphorylation of dihydroxyacetone. Further, the strain overexpresses an NADH-dependent acetaldehyde dehydrogenase, e.g., Piromyces sp. E2 adhE, to enable conversion of acetate to ethanol. See FIGS. 24 and 25.

This strain can be grown under anaerobic conditions in media containing C6 and/or C5 sugars, as well as acetate. The dihydroxyacetone (DHA) pathway, combined with formaldehyde degradation to CO₂, can generate NADH and improve the conversion of acetate to ethanol via an NADH-dependent acetaldehyde dehydrogenase.

The sequence for O. polymorpha Glycerol dehydrogenase is provided in SEQ ID NO:48. The sequence for S. cerevisiae Transketolase TKL1 is provided in SEQ ID NO:18. The sequence for O. polymorpha Formaldehyde dehydrogenase FLD1 is provided in SEQ ID NO:49. The sequence for O. polymorpha Formate dehydrogenase is provided in SEQ ID NO:50. The sequence for the S. cerevisiae dihydroxyacetone kinase DAK1 is provided in SEQ ID NO:51. The sequence for the S. cerevisiae dihydroxyacetone kinase DAK2 is provided in SEQ ID NO:52. The nucleotide sequence upstream of the DAK1 gene used to create a DAK1 clean deletion is provided in SEQ ID NO:53. The nucleotide sequence downstream of the DAK1 gene used to create a DAK1 clean deletion is provided in SEQ ID NO:54. The nucleotide sequence upstream of the DAK2 gene used to create a DAK2 clean deletion is provided in SEQ ID NO:55. The nucleotide sequence downstream of the DAK2 gene used to create a DAK2 clean deletion is provided in SEQ ID NO:56.

### Example 5

The present example describes engineering of a recombinant microorganism to use a transhydrogenase for the production of electron donors to be used in the conversion of acetate to ethanol or isopropanol.

Transhydrogenases catalyze the interconversion of:

NADPH+NAD⇄NADP+NADH (equation 3)

As the (cytosolic) NADPH/NADP ratio in S. cerevisiae is typically assumed to be higher than the NADH/NAD ratio, introduction of a transhydrogenase should create a flux towards NADH formation. Transhydrogenases from Escherichia coli (udhA) and Azotobacter vinelandii (sthA) have been successfully expressed in S. cerevisiae, and observed changes in the metabolic profiles (increased glycerol, acetate and 2-oxoglutarate production, decreased xylitol production) indeed pointed to a net conversion of NADPH into NADH. See Anderlund, M., et al., “Expression of the Escherichia coli pntA and pntB Genes, Encoding Nicotinamide Nucleotide Transhydrogenase, in Saccharomyces cerevisiae and Its Effect on Product Formation during Anaerobic Glucose Fermentation,” Appl. Envirol Microbiol. 65:2333-2340 (1999); Heux, S., et al., “Glucose utilization of strains lacking PGI1 and expressing a transhydrogenase suggests differences in the pentose phosphate capacity among Saccharomyces cerevisiae strains,” FEMS Yeast Research 8:217-224 (2008); Jeppsson, M., et al., “The level of glucose-6-phosphate dehydrogenase activity strongly influences xylose fermentation and inhibitor sensitivity in recombinant Saccharomyces cerevisiae strains,” Yeast 20:1263-1272 (2003); Jeun, Y.-S., et al., “Expression of Azotobacter vinelandii soluble transhydrogenase perturbs xylose reductase-mediated conversion of xylose to xylitol by recombinant Saccharomyces cerevisiae,” Journal of Molecular Catalysis B: Enzymatic 26:251-256 (2003); and Nissen, T. L., et al.,” Expression of a cytoplasmic transhydrogenase in Saccharomyces cerevisiae results in formation of 2-oxoglutarate due to depletion of the NADPH pool,” Yeast 18:19-32 (2001).

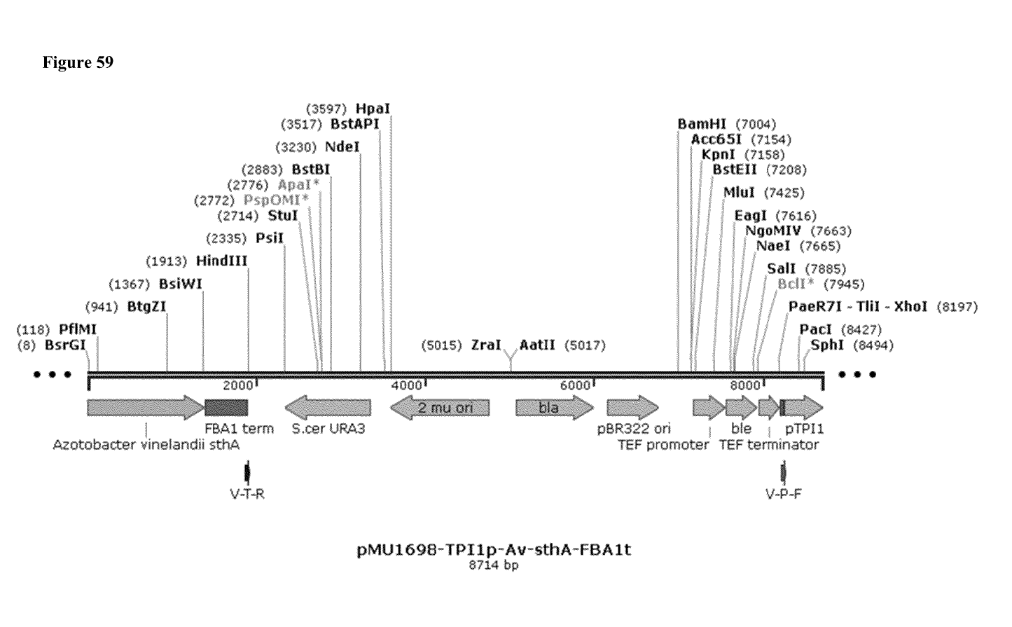

With this approach, additional NADH becomes available for acetate-to-ethanol conversion, and the consumed NADPH could be replenished by increasing the flux through the pentose phosphate pathway. The nucleotide sequence for E. coli udhA is provided as SEQ ID NO:59, and the amino acid sequence for E. coli udhA is provided as SEQ ID NO:60. The nucleotide sequence for codon-optimized Azotobacter vinelandii sthA is provided as SEQ ID NO:61, and the amino acid sequence for codon-optimized Azotobacter vinelandii sthA is provided as SEQ ID NO:62. A contstruct that can used to express Azotobacter vinelandii sthA is depicted in FIG. 59.

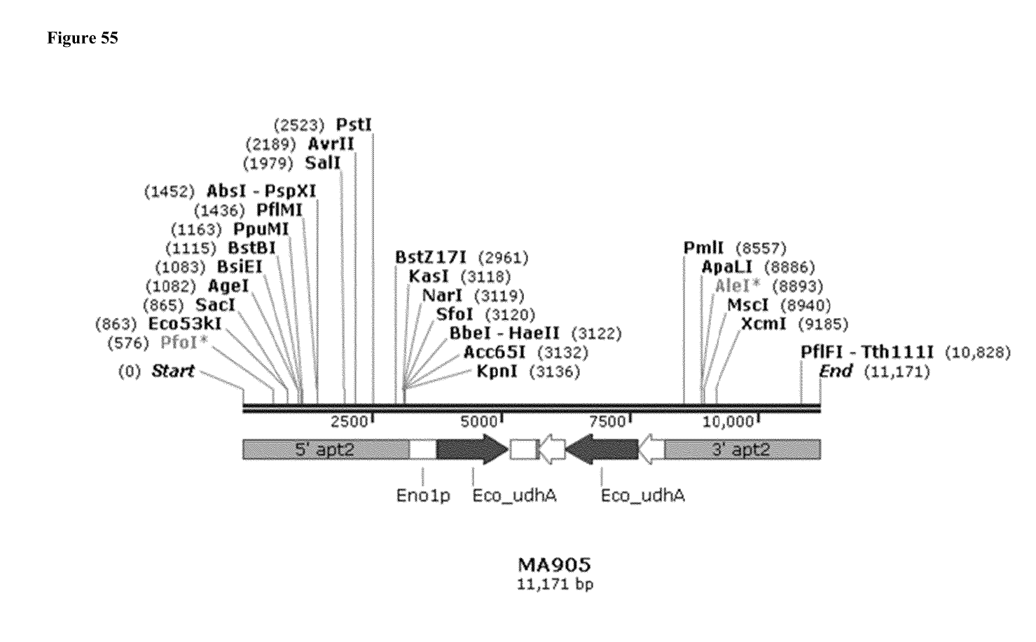

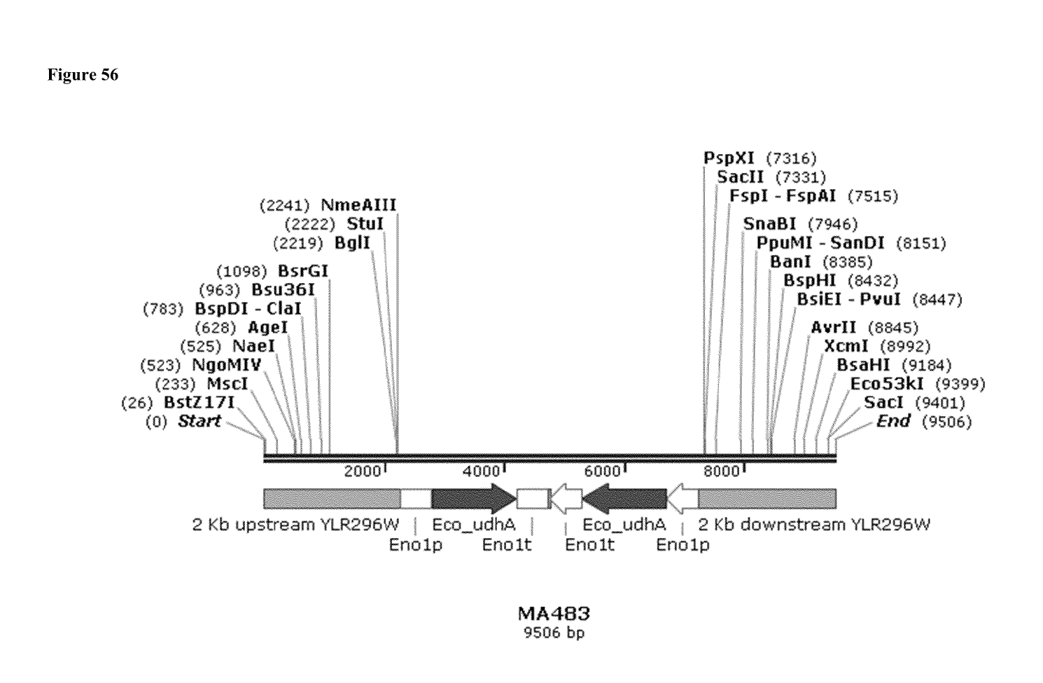

The following example describes the engineering of a recombinant microorganism to increase acetate conversion to ethanol by overexpressing the transhydrogenase, E. coli udhA, in xylose utilizing strains. E. coli udhA was overexpressed in the engineered xylose utilizing strains M3799 and M4044. M4044 is a glycerol-reduction strain derived from M3799 and contains a gpd2 gene deletion with the integration of two copies of B. adolescentis adhE. Strains M4044 and M3799 are described in commonly owned International Appl. No. PCT/US2013/000090, which is hereby incorporated by reference in its entirety. Strains M3799 and M4044 were pre-marked with dominant (kanMX and Nat) and negative (fcy1) selection markers at the apt2 and YLR296W sites, respectively. Two copies of the udhA were introduced into the pre-marked strains using the 5FC counterselection previously described. See FIGS. 55 and 56. The udhA+ strains M7215 and M7216 were generated by insertion of MA905 (FIG. 55) into the pre-marked M3799 strain. The udhA+ strains M4610 and M4611 were generated by insertion of MA483 (FIG. 56) into the pre-marked glycerol-reduction background strain M4044.

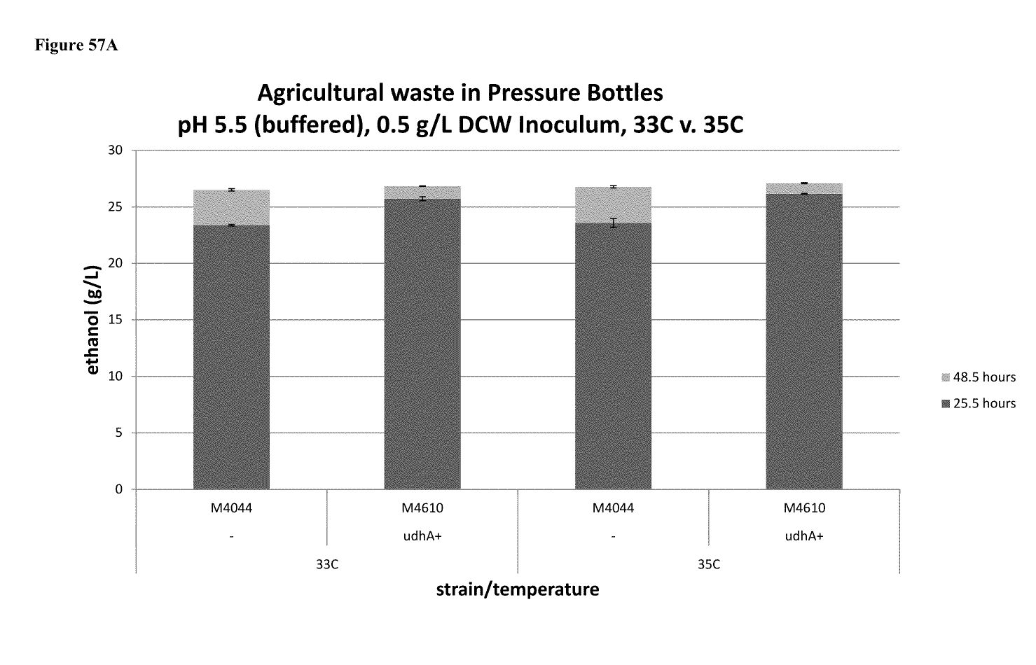

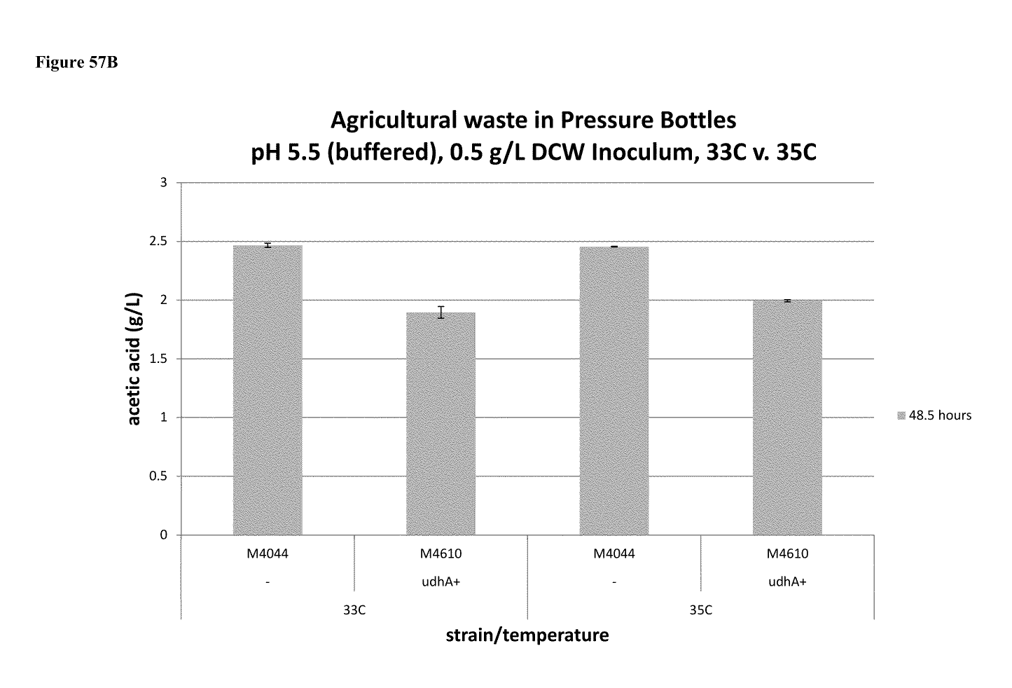

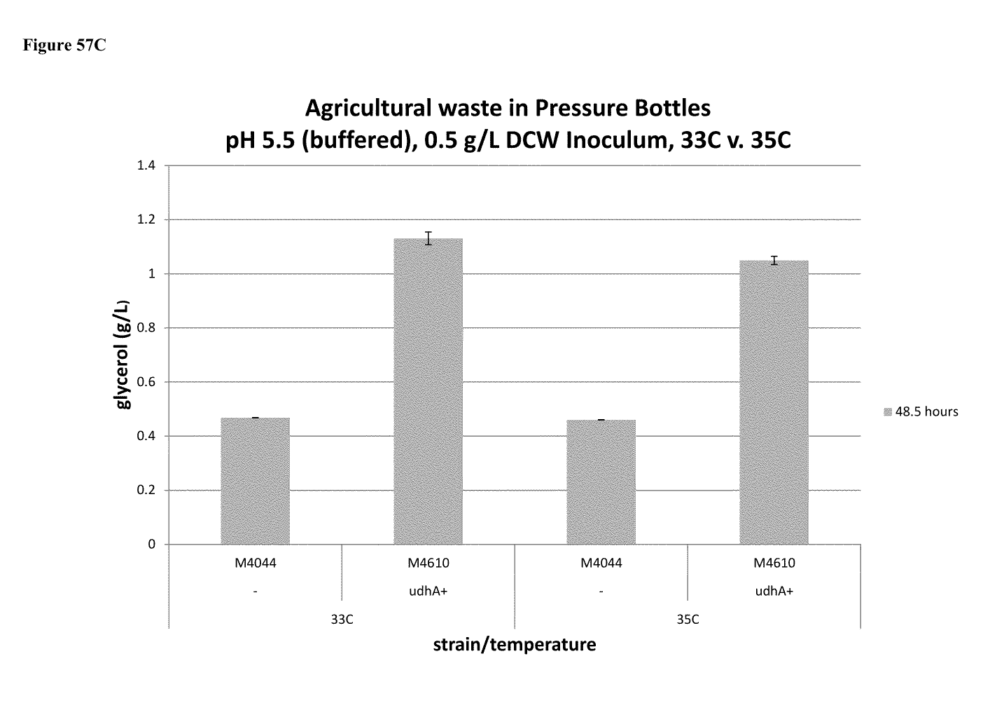

To determine if the udhA transhydrogenase was capable of influencing the acetate-to-ethanol conversion in a glycerol reduction strain expressing the B. adolescentis adhE (Δgpd2::adhE-adhE), strain M4610 (Δgpd2::adhE ΔYLR296W::udhA) was compared to the parental strain M4044 (Δgpd2::adhE) in fermentation on a pre-treated agricultural waste (FIGS. 57A-C). Fermentations were performed at 33° C. and 35° C. and were buffered with CaCO₃. Cells were inoculated at 0.5 g/L. M4610 (Δgpd2::adhE ΔYLR296W::udhA) fermentation had ˜0.5 g/L less acetic acid compared to the parental strain M4044 (Δgpd2::adhE), at both 33° C. and 35° C., indicating that the udhA strain M4610 was consuming more acetic acid than the parental strain M4044 (FIG. 57B). In addition, the udhA+ strain M4610 had a faster fermentation rate compared to M4044. At 25.5 hours of fermentation the udhA+ strain M4610 had 10% higher ethanol titer than the parental strain M4044. At the end of this fermentation (48.5 hours) the background strain had reached similar ethanol titers as the udhA strain (FIG. 57A). The glycerol production was also affected by the introduction of udhA. A non-glycerol reduction strain background run in this same fermentation was making ˜2 g/L of glycerol at 33° C. and ˜1.6 g/L at 35° C. (data not shown). The glycerol reduction strain M4044 made 30% of the total glycerol made by the non-glycerol reduction strains (0.47 g/L). The udhA+ strain M4610 produced 2-fold more glycerol (˜1 g/L) compared to M4044 (FIG. 57C). Without wishing to be bound by any one theory, this data suggests that udhA drives acetate consumption, leads to increased rate of ethanol production, and an overall increase in glycerol production. This is consistent with the role of udhA in converting NADPH to NADH because NADH is required for glycerol production (these strains still have gpd1) and acetate-to-ethanol conversion.

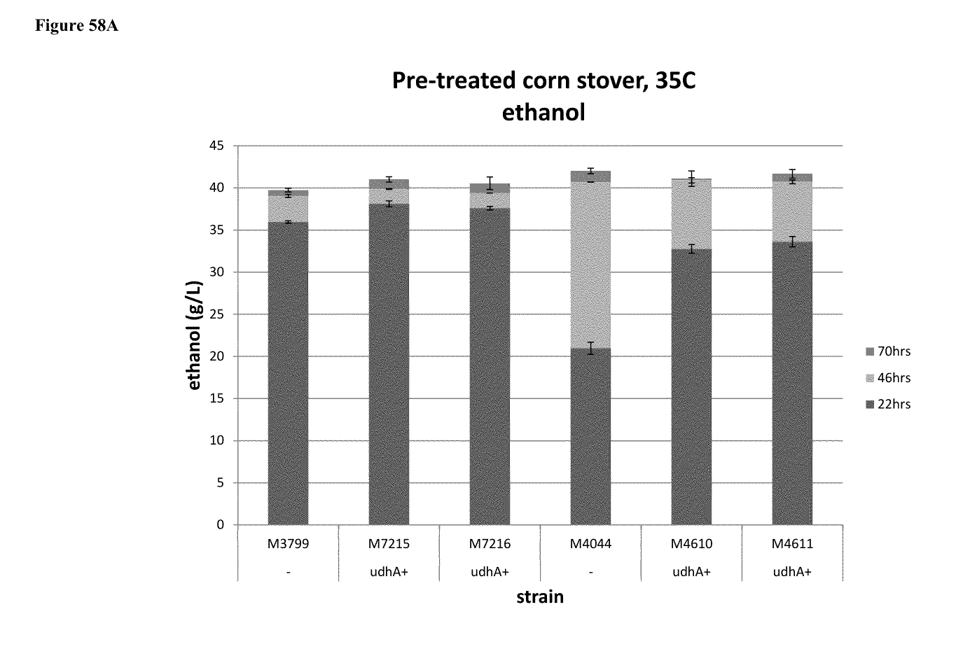

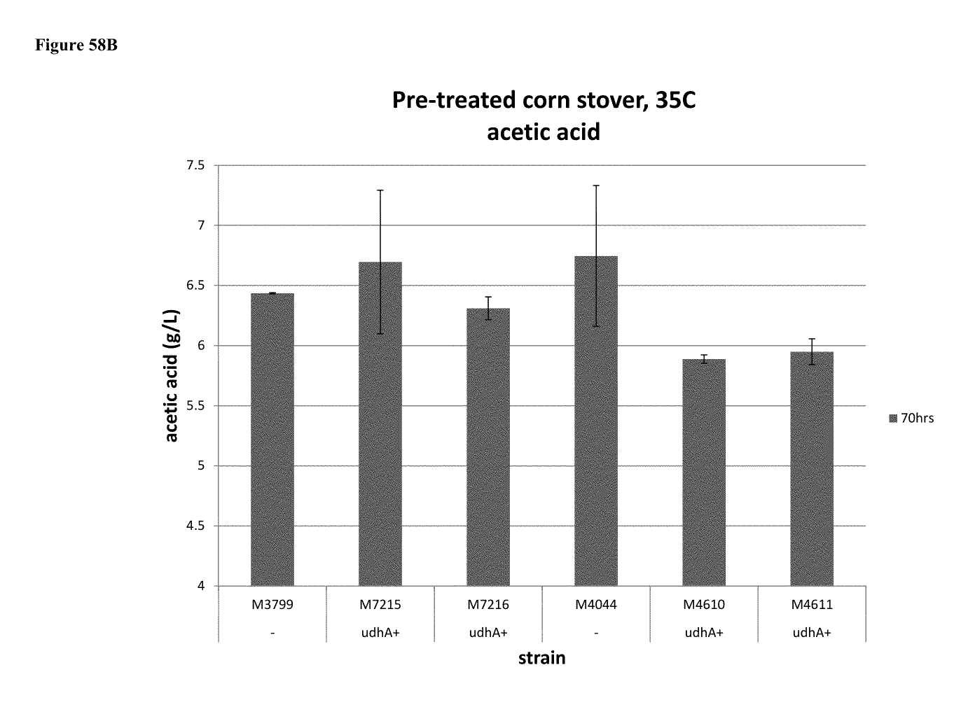

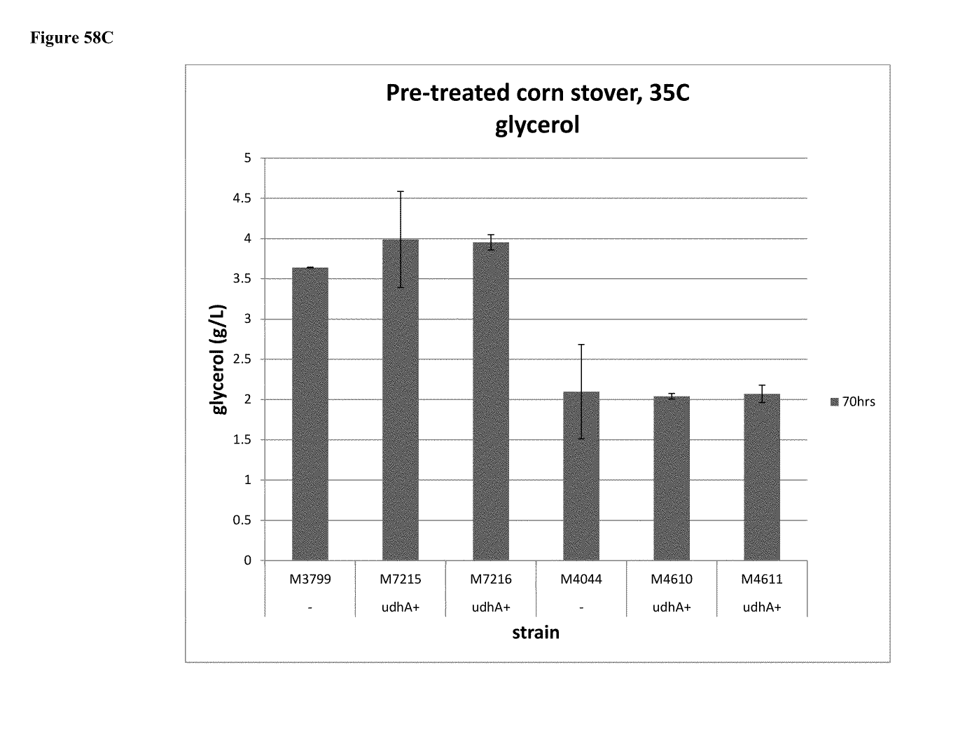

The glycerol reduction udhA strains as well as the udhA+ strains in the non-glycerol-reduction M3799 background were tested for their fermentation performance on pre-treated corn stover, another commercially relevant substrate. The data from these experiments are depicted in FIGS. 58A-C. Fermentations were performed at 35° C. for 70 hours in pressure bottles and were buffered with CaCO₃. Cells were inoculated at 0.5 g/L, and ethanol, acetic acid and glycerol levels were determined. The rate of ethanol production was increased for both the M3799 udhA+ strains, M7215 and M7216, as well as the udhA+ glycerol-reduction (Δgpd2::adhE ΔYLR296W::udhA) strains M4610 and M4611. At 22 hours the udhA+ strains M7215 and M7216 produced 4.5-6% more ethanol compared to the parental strain M3799 while the udhA+ glycerol reduction strains M4610 and M4611 had produced 56-60% more ethanol than the parental strain M4044 (FIG. 58A). M4044, did not show any acetic acid consumption on this material, but addition of udhA led to consumption of 0.8-0.85 g/L of acetate for strains M4610 and M4611 (FIG. 58B). While M7215 and M7216 did not show any acetate consumption as expected, they did show a slight increase (˜0.4 g/L) in glycerol production compared to their parental strain M3799 (FIG. 58C). The increase in glycerol production for the M3799 udhA strains and the increase in acetate consumption by the M4044 udhA strains on this material further suggest that udhA is functioning in these strains to convert NADPH to NADH.

These results suggest that the udhA is functioning in these strains to convert NADPH to NADH in both non-glycerol-reduction strains and in acetate-to-ethanol strains. The beneficial effect of a higher rate of ethanol production is likely attributable to an increased NADH availability for acetate-to-ethanol conversion (reducing the toxicity of acetate) and glycerol production (improving cell robustness). In addition, without being bound by an theory, consumption of NADPH by the transhydrogenase may benefit activity of xylose isomerase by reducing xylitol formation by any NADPH-dependent xylose reductases (because xylitol is a potent inhibitor of xylose isomerase).

### Example 6

Conceptually similar to the introduction of a transhydrogenase is the creation of a NADPH/NADH-cycling reaction. The reaction catalyzed by the overexpression of NADP- and NAD-dependent glutamate dehydrogenases is close to equilibrium, resulting in some conversion back and forth between NADPH and NADH. As the cytosolic NADPH/NADP ratio is expected to be higher than the NADH/NAD ratio, the reverse glutamate-forming reaction will preferentially use NADPH, and the forward glutamate-consuming reaction will preferentially use NAD, resulting in a net conversion of NADPH and NAD to NADP and NADH, the same reaction catalyzed by transhydrogenase. One such cycle consists of the combination of cytosolic NAD-specific and NADP-specific glutamate dehydrogenases (GDH), which catalyze the reversible reaction:

L-glutamate+H₂O+NAD(P)⁺ [symbol] 2-oxoglutarate+NH₃+NAD(P)H+H⁺

Overexpressing the native NAD-GDH encoded by GDH2 (SEQ ID NO: 1) has been shown to rescue growth in a phosphoglucose isomerase pgi1 S. cerevisiae deletion mutant, but only as long as glucose-6-phosphate dehydrogenase and the NADP-GDH encoded by GDH1 were left intact. See Boles, E., et al., “The role of the NAD-dependent glutamate dehydrogenase in restoring growth on glucose of a Saccharomyces cerevisiae phosphoglucose isomerase mutant,” European Journal of Biochemistry 217:469-477 (1993). This strongly suggests that the increased NADPH production, the result of redirection of glucose into the pentose phosphate pathway, which normally proves fatal, could be balanced by conversion of NADPH to NADH by this GDH-cycle, with the produced NADH being reoxidized via respiration.

As with transhydrogenase, when the cytosolic NADPH/NADP ratio is higher than the NADH/NAD ratio, introducing a GDH-cycling reaction would generate additional NADH at the expense of NADPH. The latter can then again be replenished by an increased flux through the pentose phosphate pathway.

GDH2 is overexpressed in a strain overexpressing an NADH-dependent acetaldehyde dehydrogenase. Competition with glycerol formation (another NADH-consuming reaction) is prevented by deleting gpd1 and gpd2. In one embodiment of the invention, adhE from Bifidobacterium adolescentis is integrated into the gpd1 and gpd2 loci, resulting in deletion of gpd1 and gpd2. See FIGS. 7-10.

This strain is grown under anaerobic conditions in media containing C6 and/or C5 sugars, as well as acetate. The strain may generate more NADH under these conditions than a strain which does not overexpress GDH2 (due to a net transfer of electrons from NADPH to NADH), allowing for improved conversion of acetate to ethanol via the NADH-dependent acetaldehyde dehydrogenase.

Following are particular embodiments of the disclosed invention.

E1. A recombinant microorganism comprising: a) one or more native and/or heterologous enzymes that function in one or more first engineered metabolic pathways to convert acetate to an alcohol, wherein said one or more native and/or heterologous enzymes is activated, upregulated or downregulated; and b) one or more native and/or heterologous enzymes that function in one or more second engineered metabolic pathways to produce an electron donor used in the conversion of acetate to an alcohol, wherein said one or more native and/or heterologous enzymes is activated, upregulated or down-regulated.

E2. The recombinant microorganism of E1, wherein said acetate is produced as a by-product of biomass processing.

E3. The recombinant microorganism of E1 or E2, wherein said alcohol is selected from the group consisting of ethanol, isopropanol, or a combination thereof.

E4. The recombinant microorganism of any of E1-E3, wherein said electron donor is selected from the group consisting of NADH, NADPH, or a combination thereof.

E5. The recombinant microorganism of any one of E1-E4, wherein said one or more second engineered metabolic pathways to produce an electron donor is a xylose fermentation pathway.

E6. The recombinant microorganism of E5, wherein said engineered xylose fermentation pathway comprises upregulation of the native and/or heterologous enzymes xylose reductase (XR) and xylitol dehydrogenase (XDH).

E7. The recombinant microorganism of E6, wherein said native and/or heterologous XDH enzyme is from Scheffersomyces stipitis.

E8. The recombinant microorganism of E7, wherein said XDH enzyme is encoded by a xyl2 polynucleotide.

E9. The recombinant microorganism of E6, wherein said native and/or heterologous XR enzyme is from Scheffersomyces stipitis, Neurospora crassa, or Candida boidinii.

E10. The recombinant microorganism of E9, wherein said XR enzyme is encoded by a xyl1 polynucleotide or an aldolase reductase.

E11. The recombinant microorganism of any one of E1-E10, wherein said first and second engineered metabolic pathways result in ATP production.

E12. The recombinant microorganism of any one of E1-E10, wherein said one or more first engineered metabolic pathways comprises activating or upregulating one or more heterologous enzymes selected from the group consisting of acetyl-CoA acetyltransferase (thiolase), acetoacetyl-CoA transferase, acetoacetate decarboxylase, a secondary alcohol dehydrogenase, and combinations thereof.

E13. The recombinant microorganism of any one of E1-E10, wherein one or more first engineered metabolic pathways comprises activating or upregulating a heterologous ADP-producing acetyl-CoA synthase enzyme.

E14. The recombinant microorganism of any one of E1-E10, wherein one or more first engineered metabolic pathways comprises activating or upregulating the acetate kinase/phosphotransacetylase (AK/PTA) couple.

E15. The recombinant microorganism of any one of E13 and E14, wherein said first and second engineered metabolic pathways result in ATP production.

E16. The recombinant microorganism of any one of E1-E4, wherein said one or more second engineered metabolic pathways to produce an electron donor is the oxidative branch of the pentose phosphate pathway (PPP).

E17. The recombinant microorganism of E16, wherein said engineered PPP comprises activation or upregulation of the native enzyme glucose-6-P dehydrogenase.

E18. The recombinant microorganism of E17, wherein said native glucose-6-P dehydrogenase enzyme is from Saccharomyces cerevisiae.

E19. The recombinant microorganism of E18, wherein said glucose-6-P dehydrogenase is encoded by a zwf1 polynucleotide.

E20. The recombinant microorganism of E1-E4, further comprising altering the expression of transcription factors that regulate expression of enzymes of the PPP pathway.

E21. The recombinant microorganism of E20, wherein the transcription factor is Stb5p.

E22. The recombinant microorganisms of E21, wherein the Stb5p is from Saccharomyces cerevisiae.

E23. The recombinant microorganism of any one of E1-E4, wherein said one or more second engineered metabolic pathways to produce an electron donor is a pathway that competes with the oxidative branch of the PPP.

E24. The recombinant microorganism of E23, wherein said engineered pathway that competes with the oxidative branch of the PPP comprises downregulation of the native enzyme glucose-6-P isomerase.

E25. The recombinant microorganism of E24, wherein said native glucose-6-P isomerase enzyme is from Saccharomyces cerevisiae.

E26. The recombinant microorganism of E25, wherein said glucose-6-P isomerase is encoded by a pgi1 polynucleotide.

E27. The recombinant microorganism of any one of E1-E4, wherein said one or more second engineered metabolic pathways to produce an electron donor comprises the ribulose-monophosphate pathway (RuMP).

E28. The recombinant microorganism of E27, wherein said engineered RuMP pathway converts fructose-6-P to ribulose-5-P and formaldehyde

E29. The recombinant microorganism of E28, wherein said engineered RuMP pathway comprises upregulating a heterologous enzyme selected from the group consisting of 6-phospho-3-hexuloisomerase, 3-hexulose-6-phosphate synthase, and the combination thereof.

E30. The recombinant microorganism of any one of E27-E29, wherein said one or more second engineered metabolic pathways to produce an electron donor comprises upregulating native enzymes that degrade formaldehyde or formate.

E31. The recombinant microorganism of E30, wherein the formaldehyde degrading enzymes convert formaldehyde to formate.

E32. The recombinant microorganism of E31, wherein the formaldehyde degrading enzymes are formaldehyde dehydrogenase and S-formylglutathione hydrolase.

E33. The recombinant microorganism of any of E30-E32, wherein the formate degrading enzyme converts formate to CO₂.

E34. The recombinant microorganism of E33, wherein the formate degrading enzyme is formate dehydrogenase.

E35. The recombinant microorganism of any one of E27-E34, wherein said one or more native and/or heterologous enzymes is from Mycobacterium gastri.

E36. The recombinant microorganism of any one of E1-E4, wherein said one or more second engineered metabolic pathways to produce an electron donor comprises the dihydroxyacetone (DHA) pathway.

E37. The recombinant microorganism of E36, wherein said engineered DHA pathway interconverts dihydroxyacetone and glyceraldehyde-3-P into xylose-5-P and formaldehyde.

E38. The recombinant microorganism of E37, wherein said engineered DHA pathway comprises upregulating the heterologous enzyme formaldehyde transketolase (EC 2.2.1.3).

E39. The recombinant microorganism of any one of E36-E38, wherein said one or more second engineered metabolic pathways to produce an electron donor comprises upregulating native and/or heterologous enzymes that produce dihydroxyacetone.

E40. The recombinant microorganism of E39, wherein said native and/or heterologous enzymes that produce dihydroxyacetone are selected from the group consisting of glycerol dehydrogenase, dihydroxyacetone phosphatase, and a combination thereof.

E41. The recombinant microorganism of E40, wherein said native and/or heterologous glycerol dehydrogenase is from a microorganism selected from the group consisting of Hansenula polymorpha, E coli, Pichia angusta, and Saccharomyces cerevisiae.

E42. The recombinant microorganism of E41, wherein said glycerol dehydrogenase is encoded by a polynucleotide selected from the group consisting of gdh, gldA, and gcy1.

E43. The recombinant microorganism of any one of E37-E42, wherein said formaldehyde is oxidized to form CO₂.

E44. The recombinant microorganism of any one of E39-E43, wherein said one or more second engineered metabolic pathways to produce an electron donor comprises downregulating a native dihydroxyacetone kinase enzyme.

E45. The recombinant microorganism of E44, wherein the dihydroxyacetone kinase is encoded by a polynucleotide selected from the group consisting of dak1, dak2, and a combination thereof.

E46. The recombinant microorganism of any one of E39-E45, wherein said microorganism further comprises overexpression of a glycerol/proton-symporter.

E47. The recombinant microorganism of E46, wherein said glycerol/proton-symporter is encoded by a stl1 polynucleotide.

E48. The recombinant microorganism of any one of E1-E47, wherein said microorganism further comprises overexpression of a native and/or heterologous transhydrogenase enzyme.

E49. The recombinant microorganism of E48, wherein said transhydrogenase catalyzes the interconversion of NADPH and NAD to NADP and NADH.

E50. The recombinant microorganism of any one of E48 and E49, wherein said transhydrogenase is from a microorganism selected from the group consisting of Escherichia coli and Azotobacter vinelandii.

E51. The recombinant microorganism of any one of E1-E47, wherein said microorganism further comprises overexpression of a native and/or heterologous glutamate dehydrogenase enzyme.

E52. The recombinant microorganism of E51, wherein said glutamate dehydrogenase is encoded by a gdh2 polynucleotide.

E53. The recombinant microorganism of any one of E1-E52, wherein one of said engineered metabolic pathways comprises the following steps: (a) conversion of acetate to acetyl-CoA and (b) conversion of acetyl-CoA to ethanol.

E54. The recombinant microorganism of any one of E1-E52, wherein said one or more downregulated native enzymes is encoded by a gpd1 polynucleotide, a gpd2 polynucleotide, or both a gpd1 polynucleotide and a gpd2 polynucleotide.

E55. The recombinant microorganism of any one of E1-E54, wherein said microorganism produces ethanol.

E56. The recombinant microorganism of any one of E1-E55, wherein said microorganism is selected from the group consisting of Saccharomyces cerevisiae, Kluyveromyces lactis, Kluyveromyces marxianus, Pichia pastoris, Yarrowia lipolytica, Hansenula polymorpha, Phaffia rhodozyma, Candida utilis, Arxula adeninivorans, Pichia stipitis, Debaryomyces hansenii, Debaryomyces polymorphus, Schizosaccharomyces pombe, Candida albicans, and Schwanniomycesoccidentalis.

E57. The recombinant microorganism of E56, wherein said microorganism is Saccharomyces cerevisiae.

E58. The recombinant microorganism of any one of E1-E57, wherein said acetate is converted to acetyl-CoA by an acetyl-CoA transferase (ACS).

E59. The recombinant microorganism of any one of E1-E57, wherein said acetate is converted to acetyl-P by an acetate kinase; and wherein said acetyl-P is converted to acetyl-CoA by a phosphotransacetylase.

E60. The recombinant microorganism of E59, wherein said acetate kinase and said phosphotransacetylase are from one or more of an Escherichia, a Thermoanaerobacter, a Clostridia, or a Bacillus species.

E61. The recombinant microorganism of any one of E1-E60, wherein said acetyl-CoA is converted to acetaldehyde by an acetaldehyde dehydrogenase; and wherein said acetaldehyde is converted to ethanol by an alcohol dehydrogenase.

E62. The recombinant microorganism of E61, wherein said acetaldehyde dehydrogenase is an NADPH-specific acetaldehyde dehydrogenase.

E63. The recombinant microorganism of E62, wherein said NADPH-specific acetaldehyde dehydrogenase is from T. pseudethanolicus.

E64. The recombinant microorganism of E63, wherein said NADPH-specific acetaldehyde dehydrogenase is T. pseudethanolicus adhB.

E65. The recombinant microorganism of E61, wherein said alcohol dehydrogenase is an NADPH-specific alcohol dehydrogenase.

E66. The recombinant microorganism of E65, wherein said NADPH-specific alcohol dehydrogenase is from a microorganism selected from the group consisting of T. pseudethanolicus, C. beijerinckii, Entamoeba histolytica, Cucumis melo, and S. cerevisiae.

E67. The recombinant microorganism of E66, wherein said NADPH-specific alcohol dehydrogenase is T. pseudethanolicus adhB.

E68. The recombinant microorganism of E66, wherein said NADPH-specific alcohol dehydrogenase is C. beijerinckii 2° Adh.

E69. The recombinant microorganism of E66, wherein said NADPH-specific alcohol dehydrogenase is S. cerevisiae ARI1.

E70. The recombinant microorganism of E66, wherein said NADPH-specific alcohol dehydrogenase is Entamoeba histolytica ADH1.

E71. The recombinant microorganism of E66, wherein said NADPH-specific alcohol dehydrogenase is Cucumis melo ADH1.

E72. The recombinant microorganism of any one of E1-E71, wherein said acetyl-CoA is converted to ethanol by a bifunctional acetaldehyde/alcohol dehydrogenase.

E73. The recombinant microorganism of any one of E58 or E61-E72, wherein said acetyl-CoA transferase (ACS) is encoded by an ACS1 polynucleotide.

E74. The recombinant microorganism of E61, wherein said acetaldehyde dehydrogenase is from C. phytofermentans.

E75. The recombinant microorganism of E72, wherein said bifunctional acetaldehyde/alcohol dehydrogenase is from E. coli, C. acetobutylicum, T. saccharolyticum, C. thermocellum, or C. phytofermentans.

E76. A recombinant microorganism comprising a) one or more native and/or heterologous enzymes that function in one or more engineered metabolic pathways to convert acetate to acetone, wherein said one or more native and/or heterologous enzymes is activated, upregulated or downregulated; and b) one or more native and/or heterologous enzymes that function in one or more second engineered metabolic pathways to produce an electron donor used in the conversion of acetate to isopropanol, wherein said one or more native and/or heterologous enzymes is activated, upregulated or down-regulated.

E77. The recombinant organism of E76, wherein said acetate is produced as a by-product of biomass processing.

E78 The recombinant microorganism of E76 or E77, wherein one of said engineered metabolic pathways comprises the following steps: (a) conversion of acetate to acetyl-CoA; (b) conversion of acetyl-CoA to acetoacetyl-CoA; (c) conversion of acetoacetyl-CoA to acetoacetate; (d) conversion of acetoacetate to acetone; and (e) conversion of acetone to isopropanol.

E79. The recombinant microorganism of any one of E76-E78, wherein said microorganism produces isopropanol.

E80. The recombinant microorganism of any one of E76-E79, wherein said microorganism is Escherichia coli.

E81. The recombinant microorganism of any one of E76-E79, wherein said microorganism is a thermophilic or mesophilic bacterium.

E82. The recombinant microorganism of E81, wherein said thermophilic or mesophilic bacterium is a species of the genera Thermoanaerobacterium, Thermoanaerobacter, Clostridium, Geobacillus, Saccharococcus, Paenibacillus, Bacillus, Caldicellulosiruptor, Anaerocellum, or Anoxybacillus.

E83. The recombinant microorganism of E82, wherein said microorganism is a bacterium selected from the group consisting of: Thermoanaerobacteriumthermosulfurigenes, Thermoanaerobacteriumaotearoense, Thermoanaerobacteriumpolysaccharolyticum, Thermoanaerobacteriumzeae, Thermoanaerobacteriumxylanolyticum, Thermoanaerobacterium saccharolyticum, Thermoanaerobiumbrockii, Thermoanaerobacteriumthermosaccharolyticum, Thermoanaerobacter thermohydrosulfuricus, Thermoanaerobacterethanolicus, Thermoanaerobacterbrocki, Clostridium thermocellum, Clostridium cellulolyticum, Clostridium phytofermentans, Clostridium straminosolvens, Geobacillus thermoglucosidasius, Geobacillus stearothermophilus, Saccharococcus caldoxylosilyticus, Saccharoccus thermophilus, Paenibacillus campinasensis, Bacillus jlavothermus, Anoxybacillus kamchatkensis, Anoxybacillus gonensis, Caldicellulosiruptor acetigenus, Caldicellulosiruptor saccharolyticus, Caldicellulosiruptor kristjanssonii, Caldicellulosiruptor owensensis, Caldicellulosiruptor lactoaceticus, and Anaerocellumthermophilum.

E84. The recombinant microorganism of E83, wherein said microorganism is selected from the group consisting of Clostridium thermocellum and Thermoanaerobacterium saccharolyticum.

E85. The recombinant microorganism of any one of E76-E79, wherein said microorganism is selected from the group consisting of Saccharomyces cerevisiae, Kluyveromyces lactis, Kluyveromyces marxianus, Pichia pastoris, Yarrowia lipolytica, Hansenula polymorpha, Phaffia rhodozyma, Candida utilis, Arxula adeninivorans, Pichia stipitis, Debaryomyces hansenii, Debaryomyces polymorphus, Schizosaccharomyces pombe, Candida albicans, and Schwanniomycesoccidentalis.

E86. The recombinant microorganism of E85, wherein said microorganism is Saccharomyces cerevisiae.

E87. The recombinant microorganism of any one of E76-E86, wherein said acetate is converted to acetyl-CoA by an acetyl-CoA synthetase.

E88. The recombinant microorganism of any one of E76-E86, wherein said acetate is converted to acetyl-P by an acetate kinase; and wherein said acetyl-P is converted to acetyl-CoA by a phosphotransacetylase.

E89. The recombinant microorganism of any one of E76-E88, wherein said acetyl-CoA is converted to acetoacetyl-CoA by a thiolase.

E90. The recombinant microorganism of any one of E76-E89, wherein said acetoacetyl-CoA is converted to acetoacetate by a CoA transferase.

E91. The recombinant microorganism of any one of E76-E90, wherein said acetoacetate is converted to acetone by an acetoacetate decarboxylase.

E92. The recombinant microorganism of E87, wherein said acetyl-CoA synthetase is encoded by a polynucleotide selected from the group consisting of a yeast ACS1 polynucleotide and a yeast ACS2 polynucleotide.

E93. The recombinant microorganism of E92, wherein said yeast ACS1 polynucleotide is from Saccharomyces cerevisiae or Saccharomyces kluyveri.

E94. The recombinant microorganism of E92, wherein said yeast ACS2 polynucleotide is from Saccharomyces cerevisiae or Saccharomyces kluyveri.

E95. The recombinant microorganism of E88, wherein said acetate kinase and said phosphotransacetylase are from T. saccharolyticum.

E96. The recombinant microorganism of any one of E89-E91, wherein said thiolase, said CoA transferase, and said acetoacetate decarboxylase are from C. acetobutylicum.

E97. The recombinant microorganism of E89, wherein said thiolase is from C. acetobutylicum or T. thermosaccharolyticum.

E98. The recombinant microorganism of E90, wherein said CoA transferase is from a bacterial source.

E99. The recombinant microorganism of E98, wherein said bacterial source is selected from the group consisting of Thermoanaerobacter tengcongensis, Thermoanaerbacterium thermosaccharolyticum, Thermosipho africanus, and Paenibacillus macerans.

E100. The recombinant microorganism of E91, wherein said acetoacetate decarboxylase is from a bacterial source.

E101. The recombinant microorganism of E100, wherein said bacterial source is selected from the group consisting of C. acetobutylicum, Paenibacillus macerans, Acidothermus cellulolyticus, Bacillus amyloliquefaciens, and Rubrobacter xylanophilus.

E102. The recombinant microorganism of any one of E1-E54 and E76-E101, wherein one of said engineered metabolic pathways comprises the following steps: (a) conversion of acetate to acetyl-CoA; (b) conversion of acetyl-CoA to acetoacetyl-CoA; (c) conversion of acetoacetyl-CoA to acetoacetate; (d) conversion of acetoacetate to acetone; and (e) conversion of acetone to isopropanol.

E103. The recombinant microorganism of E102, wherein said microorganism is selected from the group consisting of Saccharomyces cerevisiae, Kluyveromyces lactis, Kluyveromyces marxianus, Pichia pastoris, Yarrowia lipolytica, Hansenula polymorphs, Phaffia rhodozyma, Candida utilis, Arxuia adeninivorans, Pichia slipitis, Debaryomyces hansenii, Debaryomyces polymorphus, Schizosaceharomyces pombe, Candida albicans, and Schwanniornycesoceidentalis.

E104. The recombinant microorganism of E103, wherein said microorganism is Saccharomyces cerevisiae.

E105. The recombinant microorganism of any one of E102-E104, wherein said acetate is converted to acetyl-CoA by an acetyl-CoA synthetase.

E106. The recombinant microorganism of any one of E102-E105, wherein said acetyl-CoA is converted to acetoacetyl-CoA by a thiolase.

E107. The recombinant microorganism of any one of E102-E106, wherein said acetoacetyl-CoA is converted to acetoacetate by a CoA transferase.

E108. The recombinant microorganism of any one of E102-E107, wherein said acetoacetate is converted to acetone by an acetoacetate decarboxylase.

E109. The recombinant microorganism of any one of E102-E108, wherein said acetone is converted to isopropanol by an alcohol dehydrogenase.

E110. The recombinant microorganism of E105, wherein said acetyl-CoA synthetase is encoded by a polynucleotide selected from the group consisting of a yeast ACS1 polynucleotide and a yeast ACS2 polynucleotide.

E111. The recombinant microorganism of E107, wherein said CoA transferase is from a bacterial source.

E112. The recombinant microorganism of E108, wherein said acetoacetate decarboxylase is from a bacterial source.

E113. A process for converting biomass to ethanol, acetone, or isopropanol comprising contacting biomass with a recombinant microorganism according to any one of E1-E112.

E114. The process of E113, wherein said biomass comprises lignocellulosic biomass.

E115. The process of E114, wherein said lignocellulosic biomass is selected from the group consisting of grass, switch grass, cord grass, rye grass, reed canary grass, mixed prairie grass, miscanthus, sugar-processing residues, sugarcane bagasse, sugarcane straw, agricultural wastes, rice straw, rice hulls, barley straw, corn cobs, cereal straw, wheat straw, canola straw, oat straw, oat hulls, corn fiber, stover, soybean stover, corn stover, forestry wastes, recycled wood pulp fiber, paper sludge, sawdust, hardwood, softwood, agave, and combinations thereof.

E116. The process of E115, wherein said process reduces or removes acetate from the consolidated bioprocessing (CBP) media.

E117. The process of any one of E114-E116, wherein said reduction or removal of acetate occurs during fermentation.

E118. An engineered metabolic pathway for reducing or removing acetate from consolidated bioprocessing (CBP) media according to any one of E1-E112.

E119. The recombinant microorganism of any one of E27-E29, wherein said one or more second engineered metabolic pathways to produce an electron donor comprises upregulating an enzyme that degrades formate.

E120. The recombinant microorganism of E119, wherein the formate degrading enzyme converts formate to CO₂.

E121. The recombinant microorganism of E120, wherein the formate degrading enzyme is formate dehydrogenase.

E122. The recombinant microorganism of E121, wherein the formate dehydrogenase is from a yeast microorganism.

E123. The recombinant microorganism of E122, wherein the yeast microorganism is S. cerevisiae or Candida boidinii.

E124. The recombinant microorganism of E123, wherein the formate dehydrogenase from S. cerevisiae is FDH1.

E125. The recombinant microorganism of E123, wherein the formate dehydrogenase from Candida boidinii is FDH3.

E126. The recombinant microorganism of any one of E119-E125, wherein said microorganism consumes or uses more acetate than a microorganism not comprising said enzyme that degrades formate.

E127. The recombinant microorganism of E126, wherein said recombinant microorganism has an acetate uptake (g/L) under anaerobic conditions selected from: (a) at least about 1.0 fold more acetate uptake than that taken up by a recombinant microorganism not comprising said enzyme that degrades formate; (b) at least about 1.1 fold more acetate uptake than that taken up by a recombinant microorganism not comprising said enzyme that degrades formate; (c) at least about 1.2 fold more acetate uptake than that taken up by a recombinant microorganism not comprising said enzyme that degrades formate; (d) at least about 1.3 fold more acetate uptake than that taken up by a recombinant microorganism not comprising said enzyme that degrades formate; (e) at least about 1.4 fold more acetate uptake than that taken up by a recombinant microorganism not comprising said enzyme that degrades formate; (f) at least about 1.5 fold more acetate uptake than that taken up by a recombinant microorganism not comprising said enzyme that degrades formate; (g) at least about 2.0 fold more acetate uptake than that taken up by a recombinant microorganism not comprising said enzyme that degrades formate; (h) at least about 2.5 fold more acetate uptake than that taken up by a recombinant microorganism not comprising said enzyme that degrades formate; (i) at least about 3.0 fold more acetate uptake than that taken up by a recombinant microorganism not comprising said enzyme that degrades formate; (j) at least about 4.0 fold more acetate uptake than that taken up by a recombinant microorganism not comprising said enzyme that degrades formate; (k) at least about 5.0 fold more acetate uptake than that taken up by a recombinant microorganism not comprising said enzyme that degrades formate; or (l) at least about 10 fold more acetate uptake than that taken up by a recombinant microorganism not comprising said enzyme that degrades formate.

E128. The recombinant microorganism of E126, wherein said recombinant microorganism has an acetate uptake under anaerobic conditions selected from at least about 0.32 g/L, at least about 0.37 g/L, at least about 0.46 g/L, or at least about 0.48 g/L.

E129. The recombinant microorganism of any one of E65-E71, wherein said microorganism consumes or uses more acetate than a microorganism not comprising said NADPH-specific alcohol dehydrogenase.

E130. The recombinant microorganism of E129, wherein said recombinant microorganism has an acetate uptake (g/L) under anaerobic conditions selected from: (a) at least about 1.1 fold more acetate uptake than that taken up by a recombinant microorganism not comprising said NADPH-specific alcohol dehydrogenase; (b) at least about 1.2 fold more acetate uptake than that taken up by a recombinant microorganism not comprising said NADPH-specific alcohol dehydrogenase; (c) at least about 1.3 fold more acetate uptake than that taken up by a recombinant microorganism not comprising said NADPH-specific alcohol dehydrogenase; (d) at least about 1.4 fold more acetate uptake than that taken up by a recombinant microorganism not comprising said NADPH-specific alcohol dehydrogenase; (e) at least about 1.5 fold more acetate uptake than that taken up by a recombinant microorganism not comprising said NADPH-specific alcohol dehydrogenase; (f) at least about 1.6 fold more acetate uptake than that taken up by a recombinant microorganism not comprising said NADPH-specific alcohol dehydrogenase; (g) at least about 1.9 fold more acetate uptake than that taken up by a recombinant microorganism not comprising said NADPH-specific alcohol dehydrogenase; (h) at least about 2.0 fold more acetate uptake than that taken up by a recombinant microorganism not comprising said NADPH-specific alcohol dehydrogenase; (i) at least about 2.1 fold more acetate uptake than that taken up by a recombinant microorganism not comprising said NADPH-specific alcohol dehydrogenase; (j) at least about 2.3 fold more acetate uptake than that taken up by a recombinant microorganism not comprising said NADPH-specific alcohol dehydrogenase; (k) at least about 2.4 fold more acetate uptake than that taken up by a recombinant microorganism not comprising said NADPH-specific alcohol dehydrogenase; (l) at least about 2.5 fold more acetate uptake than that taken up by a recombinant microorganism not comprising said NADPH-specific alcohol dehydrogenase; (m) at least about 2.7 fold more acetate uptake than that taken up by a recombinant microorganism not comprising said NADPH-specific alcohol dehydrogenase; (n) at least about 2.8 fold more acetate uptake than that taken up by a recombinant microorganism not comprising said NADPH-specific alcohol dehydrogenase; (o) at least about 2.9 fold more acetate uptake than that taken up by a recombinant microorganism not comprising said NADPH-specific alcohol dehydrogenase; or (p) at least about 3.1 fold more acetate uptake than that taken up by a recombinant microorganism not comprising said NADPH-specific alcohol dehydrogenase.

E131. The recombinant microorganism of E129, wherein said recombinant microorganism has an acetate uptake under anaerobic conditions selected from at least about 0.35 g/L, at least about 0.36 g/L, at least about 0.38 g/L, at least about 0.40 g/L, at least about 0.44 g/L, at least about 0.45 g/L, at least about 0.47 g/L, at least about 0.48 g/L, at least about 0.51 g/L, at least about 0.53 g/L, at least about 0.59 g/L, at least about 0.61 g/L, at least about 0.63 g/L, at least about 0.65 g/L, at least about 0.66 g/L, at least about 0.70 g/L, at least about 0.79 g/L, at least about 0.8 g/L, at least about 0.83 g/l, at least about 0.84 g/L, at least about 0.87 g/L, at least about 0.9 g/L, at least about 0.91 g/L, at least about 0.96 g/L, at least about 0.99 g/L, at least about 1.00 g/L, at least about 1.01 g/L at least about 1.02 g/L, at least about 1.18 g/L, at least about 1.20 g/L, at least about 1.23 g/L, at least about 3.2 g/L, or at least about 3.3 g/L.

E132. The recombinant microorganism of E129, wherein said recombinant microorganism has an acetate uptake under anaerobic conditions from about 0.35 g/L to about 3.3 g/L.

E133. A recombinant microorganism comprising: a) one or more native and/or heterologous enzymes that function in one or more first engineered metabolic pathways to convert acetate to an alcohol, wherein said one or more native and/or heterologous enzymes is activated, upregulated or downregulated; and b) one or more native and/or heterologous zwf1 polynucleotides; wherein one or more native and/or heterologous enzymes is an NADPH-specific alcohol dehydrogenase.

E134. The recombinant microorganism of E133, wherein said NADPH-specific alcohol dehydrogenase is from a microorganism selected from the group consisting of T. pseudethanolicus, C. beijerinckii, Entamoeba histolytica, Cucumis melo, and S. cerevisiae.

E135. The recombinant microorganism of E133, wherein said NADPH-specific alcohol dehydrogenase is T. pseudethanolicus adhB.

E136. The recombinant microorganism of E133, wherein said NADPH-specific alcohol dehydrogenase is C. beijerinckii 2° Adh.

E137. The recombinant microorganism of E133, wherein said NADPH-specific alcohol dehydrogenase is S. cerevisiae ARI1.

E138. The recombinant microorganism of E133, wherein said NADPH-specific alcohol dehydrogenase is Entamoeba histolytica ADH1.

E139. The recombinant microorganism of E133, wherein said NADPH-specific alcohol dehydrogenase is Cucumis melo ADH1.

E140. The recombinant microorganism of any one of E133-E139, wherein said one or more native enzymes that function in one or more first engineered metabolic pathways to convert acetate to an alcohol is an NADH-specific alcohol dehydrogenase.

E141. The recombinant microorganism of any one of E133-E140, wherein said NADH-specific alcohol dehydrogenase is down-regulated.

E142. The recombinant microorganism of any one of E133-E141, wherein said NADH-specific alcohol dehydrogenase is selected from ADH1, ADH2, ADH3, ADH4, ADH5, or SFA1 from Saccharomyces.

E143. The recombinant microorganism of any one of E133-E142, wherein said microorganism consumes or uses more acetate than a microorganism not comprising said NADPH-specific alcohol dehydrogenase.

E144. The recombinant microorganism of E143, wherein said recombinant microorganism has an acetate uptake (g/L) under anaerobic conditions selected from: (a) at least about 1.1 fold more acetate uptake than that taken up by a recombinant microorganism not comprising said NADPH-specific alcohol dehydrogenase; (b) at least about 1.2 fold more acetate uptake than that taken up by a recombinant microorganism not comprising said NADPH-specific alcohol dehydrogenase; (c) at least about 1.3 fold more acetate uptake than that taken up by a recombinant microorganism not comprising said NADPH-specific alcohol dehydrogenase; (d) at least about 1.4 fold more acetate uptake than that taken up by a recombinant microorganism not comprising said NADPH-specific alcohol dehydrogenase; (e) at least about 1.5 fold more acetate uptake than that taken up by a recombinant microorganism not comprising said NADPH-specific alcohol dehydrogenase; (f) at least about 1.6 fold more acetate uptake than that taken up by a recombinant microorganism not comprising said NADPH-specific alcohol dehydrogenase; (g) at least about 1.9 fold more acetate uptake than that taken up by a recombinant microorganism not comprising said NADPH-specific alcohol dehydrogenase; (h) at least about 2.0 fold more acetate uptake than that taken up by a recombinant microorganism not comprising said NADPH-specific alcohol dehydrogenase; (i) at least about 2.1 fold more acetate uptake than that taken up by a recombinant microorganism not comprising said NADPH-specific alcohol dehydrogenase; (j) at least about 2.3 fold more acetate uptake than that taken up by a recombinant microorganism not comprising said NADPH-specific alcohol dehydrogenase; (k) at least about 2.4 fold more acetate uptake than that taken up by a recombinant microorganism not comprising said NADPH-specific alcohol dehydrogenase; (l) at least about 2.5 fold more acetate uptake than that taken up by a recombinant microorganism not comprising said NADPH-specific alcohol dehydrogenase; (m) at least about 2.7 fold more acetate uptake than that taken up by a recombinant microorganism not comprising said NADPH-specific alcohol dehydrogenase; (n) at least about 2.8 fold more acetate uptake than that taken up by a recombinant microorganism not comprising said NADPH-specific alcohol dehydrogenase; (o) at least about 2.9 fold more acetate uptake than that taken up by a recombinant microorganism not comprising said NADPH-specific alcohol dehydrogenase; or (p) at least about 3.1 fold more acetate uptake than that taken up by a recombinant microorganism not comprising said NADPH-specific alcohol dehydrogenase.

E145. The recombinant microorganism of any one of E133-E143, wherein said recombinant microorganism has an acetate uptake under anaerobic conditions selected from at least about 0.35 g/L, at least about 0.36 g/L, at least about 0.38 g/L, at least about 0.40 g/L, at least about 0.44 g/L, at least about 0.45 g/L, at least about 0.47 g/L, at least about 0.48 g/L, at least about 0.51 g/L, at least about 0.53 g/L, at least about 0.59 g/L, at least about 0.61 g/L, at least about 0.63 g/L, at least about 0.65 g/L, at least about 0.66 g/L, at least about 0.70 g/L, at least about 0.79 g/L, at least about 0.8 g/L, at least about 0.83 g/l, at least about 0.84 g/L, at least about 0.87 g/L, at least about 0.9 g/L, at least about 0.91 g/L, at least about 0.96 g/L, at least about 0.99 g/L, at least about 1.00 g/L, at least about 1.01 g/L at least about 1.02 g/L, at least about 1.18 g/L, at least about 1.20 g/L, at least about 1.23 g/L, at least about 3.2 g/L, or at least about 3.3 g/L.

E146. The recombinant microorganism of any one of E133-E143, wherein said recombinant microorganism has an acetate uptake under anaerobic conditions from about 0.35 g/L to about 3.3 g/L.

E147. The recombinant microorganism of any one of E133-E146, wherein the recombinant microorganism further comprises one or more native and/or heterologous acetyl-CoA synthetases, and wherein said one or more native and/or heterologous acetyl-CoA synthetases is activated or upregulated.

E148. The recombinant microorganism of E147, wherein said acetyl-CoA synthetase is encoded by a polynucleotide selected from the group consisting of an ACS1 polynucleotide and an ACS2 polynucleotide.

E149. The recombinant microorganism of E148, wherein said ACS1 polynucleotide or said ACS2 polynucleotide is from a yeast microorganism.

E150. The recombinant microorganism of E149, wherein said ACS1 polynucleotide is from Saccharomyces cerevisiae or Saccharomyces kluyveri.

E151. The recombinant microorganism of E149, wherein said ACS2 polynucleotide is from Saccharomyces cerevisiae or Saccharomyces kluyveri.

E152. A method for increasing acetate uptake from a biomass comprising contacting said biomass with a recombinant microorganism according to any one of E65 to E71 or E119 to E151.

E153. The method of E152 further comprising increasing the amount of sugars of the biomass.

E154. The method of E153, wherein said sugars are increased by the addition of an exogenous sugar source to the biomass.

E155. The method of E153 or E154, wherein said sugars are increased by the addition of one or more enzymes that use or break-down cellulose, hemicellulose and/or other biomass components.

E156. The method of any one of E153-E155, wherein said sugars are increased by the addition of a CBP microorganism that uses or breaks-down cellulose, hemicellulose and/or other biomass components.

E157. The recombinant microorganism of E5, wherein said xylose reductase (XR) has a preference for NADPH or is NADPH-specific.

E158. The recombinant microorganism of E5, wherein said xylitol dehydrogenase (XDH) has a preference for NADH or is NADH-specific.

E159. A recombinant microorganism comprising: one or more native and/or heterologous enzymes that function in one or more engineered metabolic pathways to convert acetate to an alcohol, wherein one of said native and/or heterologous enzymes is an NADPH-specific alcohol dehydrogenase.

E160. The recombinant microorganism of E159, wherein said NADPH-specific alcohol dehydrogenase is from a microorganism selected from the group consisting of T. pseudethanolicus, C. beijerinckii, Entamoeba histolytica, Cucumis melo, and S. cerevisiae.

E161. The recombinant microorganism of E159, wherein said NADPH-specific alcohol dehydrogenase is encoded by any one of SEQ ID NOs:30, 32, 33, 35, or 36 or a fragment, variant, or derivative thereof that retains the function of an alcohol dehydrogenase.

E162. A recombinant microorganism comprising: one or more native and/or heterologous enzymes that function in one or more engineered metabolic pathways to convert acetate to an alcohol, wherein a first native and/or heterologous enzyme is an NADPH-specific alcohol dehydrogenase and wherein a second native and/or heterologous enzyme is an acetyl-CoA synthetase.

E163. The recombinant microorganism of E162, wherein said NADPH-specific alcohol dehydrogenase is from Entamoeba histolytica.

E164. The recombinant microorganism of E162, wherein said NADPH-specific alcohol dehydrogenase is encoded by SEQ ID NO:35 or a fragment, variant, or derivative thereof that retains the function of an alcohol dehydrogenase.

E165. The recombinant microorganism of any one of E162-E164, wherein said acetyl-CoA synthetase is from a yeast microorganism or from a bacterial microorganism.

E166. The recombinant microorganism of any one of E162-E164, wherein said acetyl-CoA synthetase is from Saccharomyces cerevisiae, Saccharomyces kluyveri, Zygosaccharomyces bailii, or Acetobacter aceti.

E167. The recombinant microorganism of any one of E162-E164, wherein said acetyl-CoA synthetase is encoded by any one of SEQ ID NOs:37-40, 57, 58 or a fragment, variant, or derivative thereof that retains the function of an acetyl-CoA synthetase.

E168. A recombinant microorganism comprising: one or more native and/or heterologous enzymes that function in one or more engineered metabolic pathways to convert acetate to an alcohol, wherein a first native and/or heterologous enzyme is an NADPH-specific alcohol dehydrogenase and wherein a second native and/or heterologous enzyme is an NADH-specific alcohol dehydrogenase.

E169. The recombinant microorganism of E168, wherein said NADPH-specific alcohol dehydrogenase is from Entamoeba histolytica.

E170. The recombinant microorganism of E168, wherein said NADPH-specific alcohol dehydrogenase is encoded by SEQ ID NO:35 or a fragment, variant, or derivative thereof that retains the function of an alcohol dehydrogenase.

E171. The recombinant microorganism of E168, wherein said NADH-specific alcohol dehydrogenase is down-regulated.

E172. The recombinant microorganism of E171, wherein said down-regulated NADH-specific alcohol dehydrogenase is selected from ADH1, ADH2, ADH3, ADH4, ADH5, or SFA1 from Saccharomyces.

E173. A recombinant microorganism comprising: a one or more native and/or heterologous enzymes that function in one or more first engineered metabolic pathways to convert acetate to an alcohol, wherein said one or more native and/or heterologous enzymes is activated, upregulated or downregulated; and b) one or more native and/or heterologous enzymes that function in one or more second engineered metabolic pathways to produce an electron donor used in the conversion of acetate to an alcohol, wherein one of said native and/or heterologous enzymes is a formate dehydrogenase.

E174. The recombinant microorganism of E173, wherein the formate dehydrogenase is from a yeast microorganism.

E175. The recombinant microorganism of E174, wherein the yeast microorganism is S. cerevisiae or Candida boidinii.

E176. The recombinant microorganism of E175, wherein the formate dehydrogenase from S. cerevisiae is FDH1.

E177. The recombinant microorganism of E175, wherein the formate dehydrogenase from Candida boidinii is FDH3.

E178. The recombinant microorganism of E173, wherein the formate dehydrogenase from is encoded by SEQ ID NO:46, 47, or a fragment, variant, or derivative thereof that retains the function of a formate dehydrogenase.

E179. The recombinant microorganism of any one of E48-E50, wherein said microorganism consumes or uses more acetate than a microorganism not comprising overexpression of said native and/or heterologous transhydrogenase enzyme.

E180. The recombinant microorganism of any one of E48-E50, wherein said microorganism produces more ethanol than a microorganism not comprising overexpression of said native and/or heterologous transhydrogenase enzyme.

E181. The recombinant microorganism of any one of E48-E50, wherein said microorganism produces more glycerol than a microorganism not comprising overexpression of said native and/or heterologous transhydrogenase enzyme.

E182. The recombinant microorganism of E179, wherein the microorganism has an acetate uptake (g/L) selected from at least about 0.35 g/L, at least about 0.36 g/L, at least about 0.38 g/L, at least about 0.40 g/L, at least about 0.44 g/L, at least about 0.45 g/L, at least about 0.47 g/L, at least about 0.48 g/L, at least about 0.51 g/L, at least about 0.53 g/L, at least about 0.59 g/L, at least about 0.61 g/L, at least about 0.63 g/L, at least about 0.65 g/L, at least about 0.66 g/L, at least about 0.70 g/L, at least about 0.79 g/L, at least about 0.8 g/L, at least about 0.83 g/l, at least about 0.84 g/L, or at least about 085 g/L.

E183. The recombinant microorganism of E180, wherein the microorganism produces ethanol at a level selected from: (a) at least about 2% more ethanol produced by a recombinant microorganism not comprising said transhydrogenase; (b) at least about 3% more ethanol produced by a recombinant microorganism not comprising said transhydrogenase; (c) at least about 4% more ethanol produced by a recombinant microorganism not comprising said transhydrogenase; (d) at least about 4.5% more ethanol produced by a recombinant microorganism not comprising said transhydrogenase; (e) at least about 5% more ethanol produced by a recombinant microorganism not comprising said transhydrogenase; (f) at least about 6% more ethanol produced by a recombinant microorganism not comprising said transhydrogenase; (g) at least about 10% more ethanol produced by a recombinant microorganism not comprising said transhydrogenase; (h) at least about 15% more ethanol produced by a recombinant microorganism not comprising said transhydrogenase; (i) at least about 20% more ethanol produced by a recombinant microorganism not comprising said transhydrogenase; (j) at least about 25% more ethanol produced by a recombinant microorganism not comprising said transhydrogenase; (k) at least about 30% more ethanol produced by a recombinant microorganism not comprising said transhydrogenase; (l) at least about 35% more ethanol produced by a recombinant microorganism not comprising said transhydrogenase; (m) at least about 40% more ethanol produced by a recombinant microorganism not comprising said transhydrogenase; (n) at least about 45% more ethanol produced by a recombinant microorganism not comprising said transhydrogenase; (o) at least about 50% more ethanol produced by a recombinant microorganism not comprising said transhydrogenase; (p) at least about 55% more ethanol produced by a recombinant microorganism not comprising said transhydrogenase; (q) at least about 56% more ethanol produced by a recombinant microorganism not comprising said transhydrogenase; and (r) at least about 60% more ethanol produced by a recombinant microorganism not comprising said transhydrogenase.

E184. The recombinant microorganism of E181, wherein the microorganism produces glycerol (g/L) selected from at least about 0.10 g/L, at least about 0.15 g/L, at least about 0.20 g/L, at least about 0.25 g/L, at least about 0.30 g/L, at least about 0.35 g/L, at least about 0.36 g/L, at least about 0.38 g/L, or at least about 0.40 g/L.

E185. The recombinant microorganism of E181, wherein the microorganism produces glycerol (g/L) selected from: (a) at least about 1.1 fold more glycerol than that produced by a recombinant microorganism not comprising said transhydrogenase; (b) at least about 1.2 fold more glycerol than that produced by a recombinant microorganism not comprising said transhydrogenase; (c) at least about 1.3 fold more glycerol than that produced by a recombinant microorganism not comprising said transhydrogenase; (d) at least about 1.4 fold more glycerol than that produced by a recombinant microorganism not comprising said transhydrogenase; (e) at least about 1.5 fold more glycerol than that produced by a recombinant microorganism not comprising said transhydrogenase; (f) at least about 1.6 fold more glycerol than that produced by a recombinant microorganism not comprising said transhydrogenase; (g) at least about 1.9 fold more glycerol than that produced by a recombinant microorganism not comprising said transhydrogenase; and (h) at least about 2.0 fold more glycerol than that produced by a recombinant microorganism not comprising said transhydrogenase.

E186. A method for increasing acetate uptake from a biomass comprising contacting said biomass with a recombinant microorganism according to any one of E179-E185, wherein said biomass is pre-treated agricultural waste or pre-treated corn stover.

E187. A method for increasing ethanol production from a biomass comprising contacting, said biomass with a recombinant microorganism according to any one of E179-E185, wherein said biomass is pre-treated agricultural waste or pre-treated corn stover.

E188. A method for increasing glycerol production from a biomass comprising contacting said biomass with a recombinant microorganism according to any one of E179-E185, wherein said biomass is pre-treated agricultural waste or pre-treated corn stover.

### INCORPORATION BY REFERENCE

All of the references cited herein are hereby incorporated by reference in their entirety.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A recombinant yeast microorganism comprising an engineered metabolic pathway to convert acetate to ethanol comprising
(i) the conversion of acetate to acetyl-CoA and
(ii) the conversion of acetyl-CoA to ethanol,
wherein said microorganism expresses a heterologous enzyme that is an NADPH-specific alcohol dehydrogenase and wherein said microorganism expresses a native and/or heterologous enzyme that is encoded by a zwf1 polynucleotide recombinantly introduced into said microorganism.

2. The recombinant microorganism of claim 1, wherein said NADPH-specific alcohol dehydrogenase is from a microorganism selected from the group consisting of T. pseudethanolicus, C. beijerinckii, Entamoeba histolytica, Cucumis melo, and S. cerevisiae.

3. The recombinant microorganism of claim 1, wherein said acetate is converted to acetyl-CoA by an acetyl-CoA transferase (ACS).

4. The recombinant microorganism of claim 1, wherein said acetate is converted to acetyl-P by an acetate kinase; and wherein said acetyl-P is converted to acetyl-CoA by a phosphotransacetylase.

5. The recombinant microorganism of claim 1, wherein said acetyl-CoA is converted to acetaldehyde by an acetaldehyde dehydrogenase; and wherein said acetaldehyde is converted to ethanol by an alcohol dehydrogenase.

6. The recombinant microorganism of claim 1, wherein said acetyl-CoA is converted to ethanol by a bifunctional acetaldehyde/alcohol dehydrogenase.

7. A process for converting biomass to ethanol comprising contacting biomass with a recombinant microorganism according to claim 1.

8. An engineered metabolic pathway for reducing or removing acetate from consolidated bioprocessing (CBP) media according to claim 1.

9. A recombinant yeast microorganism comprising an engineered metabolic pathway to convert acetate to ethanol comprising
(i) the conversion of acetate to acetyl-CoA and
(ii) the conversion of acetyl-CoA to ethanol,
wherein said microorganism expresses a heterologous enzyme that is an NADPH-specific alcohol dehydrogenase and wherein said microorganism expresses a native and/or heterologous enzyme that is an acetyl-CoA synthetase recombinantly introduced into said microorganism.

10. The recombinant microorganism of claim 9, wherein said NADPH-specific alcohol dehydrogenase is from a microorganism selected from the group consisting of T. pseudethanolicus, C. beijerinckii, Entamoeba histolytica, Cucumis melo, and S. cerevisiae.

11. The recombinant microorganism of claim 9, wherein said acetyl-CoA synthetase is from Saccharomyces cerevisiae, Saccharomyces kluyveri, Zygosaccharomyces bailii, or Acetobacter aceti.

12. A recombinant yeast microorganism comprising an engineered metabolic pathway to convert acetate to ethanol comprising
(i) the conversion of acetate to acetyl-CoA and
(ii) the conversion of acetyl-CoA to ethanol,
wherein said microorganism expresses a heterologous enzyme that is an NADPH-specific alcohol dehydrogenase and wherein said microorganism expresses a native and/or heterologous enzyme that is an NADH-specific alcohol dehydrogenase recombinantly introduced into said microorganism.

13. The recombinant microorganism of claim 12, wherein said NADPH-specific alcohol dehydrogenase is from a microorganism selected from the group consisting of T. pseudethanolicus, C. beijerinckii, Entamoeba histolytica, Cucumis melo, and S. cerevisiae.

14. A recombinant yeast microorganism comprising an engineered metabolic pathway to convert acetate to ethanol comprising
(i) the conversion of acetate to acetyl-CoA and
(ii) the conversion of acetyl-CoA to ethanol,
wherein said microorganism expresses a heterologous enzyme that is an NADPH-specific alcohol dehydrogenase and wherein a native NADH-specific alcohol dehydrogenase in said microorganism is down-regulated.

15. The recombinant microorganism of claim 14, wherein said NADPH-specific alcohol dehydrogenase is from a microorganism selected from the group consisting of T. pseudethanolicus, C. beijerinckii, Entamoeba histolytica, Cucumis melo, and S. cerevisiae.

16. A recombinant yeast microorganism comprising an engineered metabolic pathway to convert acetate to ethanol comprising
(i) the conversion of acetate to acetyl-CoA and
(ii) the conversion of acetyl-CoA to ethanol,
wherein said microorganism
(a) expresses a native and/or heterologous enzyme that is an NADH-dependent acetaldehyde dehydrogenase recombinantly introduced into said microorganism,
(b) expresses a native and/or heterologous enzyme that is an NADPH-specific xylose reductase recombinantly introduced into said microorganism, and
(c) expresses a native and/or heterologous enzyme that is an NADH-specific xylitol dehydrogenase recombinantly introduced into said microorganism.


SEQUENCE LISTING (62 sequences)

SEQ ID NO: 1 - DNA, 3279 bp, Saccharomyces cerevisiae
  misc_feature (1)..(3279): GDH2
atgctttttgataacaaaaatcgcggtgctttaaactcactgaacacaccagatattgcttctttatcaatatcatccatgtcggactatcacgtgtttgattttcccggtaaggacctgcagagagaggaagtgatagatttgctagatcagcaagggtttattcccgacgatttgatcgaacaagaagtagattggttttataactcattgggtattgacgatttgttcttctcgagagaatctccccaattaatctcgaatatcatacattctttgtatgcttcaaagctagatttctttgcgaagtccaaattcaacggaattcagccaaggctattcagcattaaaaacaaaattataactaatgataatcatgccatctttatggaatctaatactggtgtcagcataagcgattctcagcaaaaaaactttaaatttgctagtgacgccgtcggaaacgatactttggagcatggtaaggataccatcaaaaaaaataggattgaaatggatgattcttgtccaccttatgaattagattccgaaattgatgaccttttcctggataacaagtctcaaaaaaactgcagattagtttctttttgggctccagaaagcgaattaaagctaacttttgtttatgagagtgtttaccctaatgatgatccagccggcgtagatatttcctctcaggatttgctgaaaggtgatattgaatcgattagtgataagaccatgtacaaagtttcgtcgaacgaaaataaaaaactatacggtctcttacttaagttggttaaagaaagagaaggtcctgtcattaagactactcgctccgtagaaaataaggatgaaattaggttattagtcgcttacaagcgattcaccactaagcgttattactctgctttgaactctttgttccactattacaagttgaaaccttctaagttctatttagagtcgtttaatgttaaggatgatgacatcattatcttttccgtttatttgaacgagaaccagcaattggaagatgttctacttcacgatgtggaggcagcattgaaacaggttgaaagagaagcttcattgctatacgctatcccaaacaattctttccatgaggtttaccagagacgtcaattctcgcccaaagaagctatatatgctcatattggtgctatattcattaaccattttgttaatcgtttaggctctgattatcaaaaccttttatctcaaatcaccattaagcgtaatgatactactcttttggagattgtagaaaacctaaaaagaaagttaagaaatgaaaccttaactcagcaaactattatcaacatcatgtcgaagcattacactataatttccaagttgtataaaaattttgctcaaattcactattatcataatagtactaaagatatggagaagacattatcttttcaaagactggaaaaagtggagccttttaagaatgaccaagagttcgaagcttacttgaataaattcattccaaatgattcacctgatttgttgatcctgaaaacactgaacatcttcaacaagtctattttgaagacaaatttctttattacaagaaaagtagcaatatcattcagattagatccttccctggtgatgacaaaattcgaatatccagagacaccctatggtatattttttgtcgttggtaatactttcaaagggttccatatcaggttcagagatatcgcaaggggcggtattcgtatagtctgttccaggaatcaggatatttatgatttgaattccaagaacgttattgatgagaactatcaattggcctctactcagcaacgtaaaaataaggatattccagagggtggctctaaaggtgtcatcttattgaacccaggattggtagaacatgaccagacatttgtcgccttttcccaatatgtggatgcaatgattgacattctaatcaacgatccattaaaggaaaactatgtcaaccttttaccaaaggaggaaatattattttttggcccagatgaaggaactgctggtttcgtggattgggcaactaaccatgctcgtgtgaggaactgcccatggtggaaatcatttttgactggaaaatccccatctttgggtggtattccccatgacgaatatggtatgacttctctgggtgttcgtgcttatgttaataaaatttacgaaactttaaacttgacaaattctactgtttacaaattccaaactggtggtccggatggtgatttgggatccaatgaaattcttttatcttcgccaaacgaatgttatttggcaattctggacggttcaggtgtcctgtgtgatcctaaaggtttagataaagatgaattatgccgcttggcacatgaaaggaaaatgatttccgatttcgacacttccaaattatcaaacaacggattttttgtttctgtggatgcaatggatatcatgctaccaaatggtacaattgtagctaacggcacaaccttcagaaacacctttcatactcaaattttcaaatttgtggatcatgtcgacatttttgttccatgcggtggtagaccaaactcaattactctaaataatctacattattttgttgacgaaaagactgggaaatgtaaaattccatatattgtggagggtgccaatctatttataacgcaacctgctaaaaatgctttggaggaacatggctgtattctgttcaaagatgcttctgcaaacaaaggtggtgtcacatcttcatcaatggaagtgttggcctcactagcgcttaacgataacgacttcgtgcacaaatttattggagatgttagtggtgagaggtctgcgttgtacaagtcgtacgttgtagaagtgcagtcaagaattcagaaaaatgctgaattagagtttggtcagttatggaatttgaatcaactaaatggaacccacatttcagaaatttcaaaccaattgtccttcactataaacaaattgaacgacgatctagttgcttctcaagagttgtggctcaatgatctaaaattaagaaactacctattgttggataaaataattccaaaaattctgattgatgttgctgggcctcagtccgtattggaaaacattccagagagctatttgaaagttcttctgtcgagttacttatcaagcacttttgtttaccagaacggtatcgatgttaacattggaaaattcttggaatttattggtgggttaaaaagagaagcggaggcaagtgcttga

SEQ ID NO: 2 - protein, 1092 aa, Saccharomyces cerevisiae
  misc_feature (1)..(1092): GDH2
MLFDNKNRGALNSLNTPDIASLSISSMSDYHVFDFPGKDLQREEVIDLLDQQGFIPDDLIEQEVDWFYNSLGIDDLFFSRESPQLISNIIHSLYASKLDFFAKSKFNGIQPRLFSIKNKIITNDNHAIFMESNTGVSISDSQQKNFKFASDAVGNDTLEHGKDTIKKNRIEMDDSCPPYELDSEIDDLFLDNKSQKNCRLVSFWAPESELKLTFVYESVYPNDDPAGVDISSQDLLKGDIESISDKTMYKVSSNENKKLYGLLLKLVKEREGPVIKTTRSVENKDEIRLLVAYKRFTTKRYYSALNSLFHYYKLKPSKFYLESFNVKDDDIIIFSVYLNENQQLEDVLLHDVEAALKQVEREASLLYAIPNNSFHEVYQRRQFSPKEAIYAHIGAIFINHFVNRLGSDYQNLLSQITIKRNDTTLLEIVENLKRKLRNETLTQQTIINIMSKHYTIISKLYKNFAQIHYYHNSTKDMEKTLSFQRLEKVEPFKNDQEFEAYLNKFIPNDSPDLLILKTLNIFNKSILKTNFFITRKVAISFRLDPSLVMTKFEYPETPYGIFFVVGNTFKGFHIRFRDIARGGIRIVCSRNQDIYDLNSKNVIDENYQLASTQQRKNKDIPEGGSKGVILLNPGLVEHDQTFVAFSQYVDAMIDILINDPLKENYVNLLPKEEILFFGPDEGTAGFVDWATNHARVRNCPWWKSFLTGKSPSLGGIPHDEYGMTSLGVRAYVNKIYETLNLTNSTVYKFQTGGPDGDLGSNEILLSSPNECYLAILDGSGVLCDPKGLDKDELCRLAHERKMISDFDTSKLSNNGFFVSVDAMDIMLPNGTIVANGTTFRNTFHTQIFKFVDHVDIFVPCGGRPNSITLNNLHYFVDEKTGKCKIPYIVEGANLFITQPAKNALEEHGCILFKDASANKGGVTSSSMEVLASLALNDNDFVHKFIGDVSGERSALYKSYVVEVQSRIQKNAELEFGQLWNLNQLNGTHISEISNQLSFTINKLNDDLVASQELWLNDLKLRNYLLLDKIIPKILIDVAGPQSVLENIPESYLKVLLSSYLSSTFVYQNGIDVNIGKFLEFIGGLKREAEASA

SEQ ID NO: 3 - DNA, 450 bp, Saccharomyces cerevisiae
  misc_feature (1)..(450): promoter pTPI1
ctacttattcccttcgagattatatctaggaacccatcaggttggtggaagattacccgttctaagacttttcagcttcctctattgatgttacacctggacaccccttttctggcatccagtttttaatcttcagtggcatgtgagattctccgaaattaattaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaa

SEQ ID NO: 4 - protein, 376 aa, Artificial sequence
  misc_feature (1)..(376): thymidine kinase gene from Herpes Simplex Virus Type 1
MASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHVGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQGGGSWWEDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEAN

SEQ ID NO: 5 - DNA, 957 bp, Scheffersomyces stipitis
  misc_feature (1)..(957): XYL1
atgccttctattaagttgaactctggttacgacatgccagccgtcggtttcggctgttggaaagtcgacgtcgacacctgttctgaacagatctaccgtgctatcaagaccggttacagattgttcgacggtgccgaagattacgccaacgaaaagttagttggtgccggtgtcaagaaggccattgacgaaggtatcgtcaagcgtgaagacttgttccttacctccaagttgtggaacaactaccaccacccagacaacgtcgaaaaggccttgaacagaaccctttctgacttgcaagttgactacgttgacttgttcttgatccacttcccagtcaccttcaagttcgttccattagaagaaaagtacccaccaggattctactgtggtaagggtgacaacttcgactacgaagatgttccaattttagagacctggaaggctcttgaaaagttggtcaaggccggtaagatcagatctatcggtgtttctaacttcccaggtgctttgctcttggacttgttgagaggtgctaccatcaagccatctgtcttgcaagttgaacaccacccatacttgcaacaaccaagattgatcgaattcgctcaatcccgtggtattgctgtcaccgcttactcttcgttcggtcctcaatctttcgttgaattgaaccaaggtagagctttgaacacttctccattgttcgagaacgaaactatcaaggctatcgctgctaagcacggtaagtctccagctcaagtcttgttgagatggtcttcccaaagaggcattgccatcattccaaagtccaacactgtcccaagattgttggaaaacaaggacgtcaacagcttcgacttggacgaacaagatttcgctgacattgccaagttggacatcaacttgagattcaacgacccatgggactgggacaagattcctatcttcgtctaa

SEQ ID NO: 6 - DNA, 966 bp, Candida boidinii
  misc_feature (1)..(966): Aldolase Reductase
atgtcaagcccacttttaactttaaacaatggcttaaagatgccacaaatcggttttggttgttggaaagtcgacaatgccacttgtgccgaaactatttatgaagccattaaagtcggttacagattattcgatggtgctatggattacggtaatgaaaaagaagttggtgaaggtgttaacaaagcgatcaaagatggtttagttaaaagagaagaattattcattgtttcaaaattatggaacaatttccatcatccagattcagttaaactagcaatcaaaaaagttctatctgatttaaatttagaatacattgatttattctatatgcatttcccaattgctcaaaaatttgttccaattgaaaagaaatatccaccaaatttttattgtggtgatggtgataaatggagttttgaagatgtcccacttttaacaacttggagagctatggaagaattggttgaagaaggtttagttaaatcaattggtatctctaactttgtcggtgctttgattcaagatttattaagaggttgtaaaattagaccagcagttttagaaattgaacatcacccatatttagttcaaccaagattaattgaatacgctaaaactgaaggtattcacgttaccgcatactcttcatttggtccacaatcatttgttgaattagaccatcctaaagttaaagactgtaccactctattcaaacatgaaacaattacttcaattgcttcagctcatgacgtccctccagctaaagtcttattgagatgggctactcaaagaggtttagcagttatcccaaaatctaataaaaaggaaagattattaggtaatttgaaaattaatgattttgatttaactgaagctgaacttgaaaaaattgaagcattagatattggtttaagatttaatgatccatggacttggggttacaatattccaacatttatttaa

SEQ ID NO: 7 - DNA, 969 bp, Neurospora crassa
  misc_feature (1)..(969): Xylose Reductase
atggttcctgccatcaaactgaactctggcttcgatatgcctcaagttggttttggtttgtggaaagtggatggatcaatcgcctcagatgtggtctataatgcaatcaaagccggctatagactgtttgacggtgcttgtgactatggaaacgaagtagaatgcggccaaggagtagccagggcaattaaggaaggaatagtgaaaagagaggaattgttcattgtctcaaagctatggaatacatttcacgatggggacagagtagagcctatcgttaggaagcaattagctgattggggtttggaatactttgacttatacttaattcatttcccagtagcgttagaatacgttgacccttctgttagatacccacctggctggcatttcgatggtaaaagtgaaattagaccatcaaaagccacaatccaggaaacatggaccgcaatggaatcccttgttgaaaagggactatccaaatcaataggtgtctctaatttccaagctcaattgctttacgatcttctaagatacgctaaagtcagaccagcaactttacagattgaacatcacccatacttggtgcaacaaaacctactgaatttggccaaagcggagggtatcgctgttactgcttactcttcatttggcccagcttcctttagagagtttaacatggaacatgcacagaagttacaaccactgctcgaagatccaactataaaggcaatcggtgataagtacaataaggaccctgctcaagttttgttgcgttgggcaacgcaacgagggcttgcgataattccaaaatctagtagagaagctaccatgaaatctaatttgaactctttagactttgatctaagcgaggaggatatcaaaacaatcagtgggtttgatagaggtattagattcaatcaaccaactaactatttttctgctgaaaatctctggattttcggttaa

SEQ ID NO: 8 - DNA, 1092 bp, Scheffersomyces stipitis
  misc_feature (1)..(1092): XYL2
atgactgctaacccttccttggtgttgaacaagatcgacgacatttcgttcgaaacttacgatgccccagaaatctctgaacctaccgatgtcctcgtccaggtcaagaaaaccggtatctgtggttccgacatccacttctacgcccatggtagaatcggtaacttcgttttgaccaagccaatggtcttgggtcacgaatccgccggtactgttgtccaggttggtaagggtgtcacctctcttaaggttggtgacaacgtcgctatcgaaccaggtattccatccagattctccgacgaatacaagagcggtcactacaacttgtgtcctcacatggccttcgccgctactcctaactccaaggaaggcgaaccaaacccaccaggtaccttatgtaagtacttcaagtcgccagaagacttcttggtcaagttgccagaccacgtcagcttggaactcggtgctcttgttgagccattgtctgttggtgtccacgcctctaagttgggttccgttgctttcggcgactacgttgccgtctttggtgctggtcctgttggtcttttggctgctgctgtcgccaagaccttcggtgctaagggtgtcatcgtcgttgacattttcgacaacaagttgaagatggccaaggacattggtgctgctactcacaccttcaactccaagaccggtggttctgaagaattgatcaaggctttcggtggtaacgtgccaaacgtcgttttggaatgtactggtgctgaaccttgtatcaagttgggtgttgacgccattgccccaggtggtcgtttcgttcaagtcggtaacgctgctggtccagtcagcttcccaatcaccgttttcgccatgaaggaattgactttgttcggttctttcagatacggattcaacgactacaagactgctgttggaatctttgacactaactaccaaaacggtagagaaaatgctccaattgactttgaacaattgatcacccacagatacaagttcaaggacgctattgaagcctacgacttggtcagagccggtaagggtgctgtcaagtgtctcattgacggccctgagtaa

SEQ ID NO: 9 - DNA, 3024 bp, Piromyces sp.
  misc_feature (1)..(3024): adhE
acgctacttatttttataacatcgttgtctaaaaaaaaattataatttattaatttttttttattaagtaaaatatattttttgagaatatacattttatttaataaaaaacttaataaaacaaaaaagctataatactataatatcattgaatattataaaatttttttatatttttaatatctatttcacccaattttattaattttttaataaaataaaataatataatcaaaatgtccggattacaaatgttccaaaacctttctctttacggtagtctcgccgaaatcgatactagcgaaaagcttaacgaagctatggacaaattaactgctgcccaagaacaattcagagaatacaaccaagaacaagttgacaaaatcttcaaggctgttgctttagctgcttctcaaaaccgtgttgctttcgctaagtacgcacacgaagaaacccaaaagggtgttttcgaagataaggttatcaagaacgaattcgctgctgattacatttaccacaagtactgcaatgacaagaccgccggtatcattgaatatgatgaagccaatggtcttatggaaattgctgaaccagttggtccagttgttggtattgctccagttactaacccaacttctactatcatctacaagtctttaattgccttaaagacccgtaactgtattatcttctcaccacatccaggagctcacaaggcctctgttttcgttgttaaggtcttacaccaagctgctgttaaggctggtgccccagaaaactgtattcaaatcatcttcccaaagatggatttaactactgaattattacaccaccaaaagactcgtttcatttgggctactggtggtccaggtttagttcacgcctcttacacttctggtaagccagctcttggtggtggtccaggtaatgctccagctcttattgatgaaacttgtgatatgaacgaagctgttggttctatcgttgtttctaagactttcgattgtggtatgatctgtgccactgaaaacgctgttgtcgttgtcgaatctgtctacgaaaacttcgttgctaccatgaagaagcgtggtgcctacttcatgactccagaagaaaccaagaaggcttctaaccttcttttcggagaaggtatgagattaaatgctaaggctgttggtcaaactgccaagactttagctgaaatggccggtttcgaagtcccagaaaacaccgttgttctctgtggtgaagcttctgaagttaaattcgaagaaccaatggctcacgaaaagttaactactatcctcggtatctacaaggctaaggactttgacgatggtgtcagattatgtaaggaattagttactttcggtggtaagggtcacactgctgttctctacaccaaccaaaacaacaaggaccgtattgaaaagtaccaaaacgaagttccagccttccacatcttagttgacatgccatcttccctcggttgtattggtgatatgtacaacttccgtcttgctccagctcttaccattacttgtggtactatgggtggtggttcctcctctgataacattggtccaaagcacttacttaacatcaagcgtgttggtatgagacgcgaaaacatgctttggttcaagattccaaagtctgtctacttcaagcgtgctatcctttctgaagctttatctgacttacgtgacacccacaagcgtgctatcattattaccgatagaactatgactatgttaggtcaaactgacaagatcattaaggcttgtgaaggtcatggtatggtctgcactgtctacgataaggttgtcccagatccaactatcaagtgtattatggaaggtgttaatgaaatgaacgtcttcaagccagatttagctattgctcttggtggtggttctgctatggatgccgctaagatgatgcgtttattctacgaatacccagaccaagacttacaagatattgctactcgtttcgtcgatatccgtaagcgtgttgttggttgtccaaagcttggtagacttattaagactcttgtctgtatcccaactacctctggtactggtgccgaagttactccattcgctgtcgttacctctgaagaaggtcgtaagtacccattagtcgactacgaacttactccagatatggctattgttgatccagaattcgctgttggtatgccaaagcgtttaacttcttggactggtattgatgctcttacccacgccattgaatcttacgtttctattatggctactgacttcactagaccatactctctccgtgctgttggtcttatcttcgaatccctttcccttgcttacaacaacggtaaggatattgaagctcgtgaaaagatgcacaatgcttctgctattgctggtatggcctttgccaacgctttccttggttgttgtcactctgttgctcaccaacttggttccgtctaccacattccacacggtcttgccaacgctttaatgctttctcacatcattaagtacaacgctactgactctccagttaagatgggtaccttcccacaatacaagtacccacaagctatgcgtcactacgctgaaattgctgaactcttattaccaccaactcaagttgttaagatgactgatgttgataaggttcaatacttaattgaccgtgttgaacaattaaaggctgacgttggtattccaaagtctattaaggaaactggaatggttactgaagaagacttcttcaacaaggttgaccaagttgctatcatggccttcgatgaccaatgtactggtgctaacccacgttacccattagtttctgaattaaaacaattaatgattgatgcctggaacggtgttgtcccaaagctctaaattaatcgtttaaatgaaagaaacaagaaaaattaaatcattgaattttaaaaaagaagtgatacccagaagcaaaagttcaaaaggttcttgccttcctttcgtgaaggttgtttaataatgaaaaaaa

SEQ ID NO: 10 - protein, 713 aa, Entamoeba histolytica
  misc_feature (1)..(713): Acetyl-CoA synthetase
MQFEPLFNPKSVPVIGASDRKESVGYAVMNNMIKGGYKGNLYPVGRKPELFGKKCYAKIGKIEEKVDLAVIAIPAKFVPGVCIECGEAGVKGLIIITAGFAEAGEEGKKMCIEIQATCQKYNMRMIGPNCLGIINPRDGVNASFASVMPEAGGVAFISQSGALCTAILDWAANQHVGFSYFVSIGSSIDTDYADLFEFFAKDPKVTSILMYIESIKDAKKFVLRAREFAADKPIILLKAGKSSEGAAAAMSHTGSLAGNDAVYDAVFDRCGCIRVDSICDLWDCAHVLATQNIPQNNRLCIITNAGGPGVISTDRLVSVHGHLAKLSESTMNELNAFLSPFWSHSNPVDVLGDATAGVYQKTLDIVIKDPQIDGVVVVLTPQAMTDPVAVAKSLVEHGPYQNQSLPHGWVNQKSEAGVKILEEGKIPNFETPERAVTAFGYIMRHPDIAAKLKEIPKYLDVQVDYEGAKKLIADVVADGRTTFTEYEGKMMFSKYGIPIKGMAKASTEDEAVAEAMKIGTPVVMKILSPDIMHKTDVGGVKVKLTTEEEIRKAYRDIMTSVKEKKPEARIHGVLLEKMVGFKYECIIGCKKDPLFGPVIVFGMGGVTVELYKDTNIALPPIGLQEADRLIDGTKISKLLRGYRGMPACDVEGLKKILVQFSKMIMDFPEISEVDINPLAVSYEEFLVLDAKIVLDKNMIGKEVPKYSHLVIQP

SEQ ID NO: 11 - protein, 726 aa, Giardia intestinalis
  misc_feature (1)..(726): Acetyl-CoA synthetase
MRQNYSTKYKKMGKLSFLTNPASVAVIGASPNAGKVGNTVVTNIKESGYTGKVYPINPTATEILGYKTYKSVLDVPDSIDVVIVVIPSKAVLAAAKECAQKKVKSLVVITAGFKEIGGEGVQMEQDLTKICKDAGIRLVGPNCLGIVTPNLNCTFASAKPSKGSIAFLSQSGAMLTSILDWALTNGIGFSNFISLGNKADVDEVDLIMEVAEDPNTDIILLYLESIVDGRKFLEQIPTCVHKKPVIILKSGTSAAGAAAASSHTGALAGNDIAFDLAFEKAGVLRAATMSDLFDLGRLFVSHRLPKGDNFVIVTNAGGPGIVTTDAFETYHVGMAALSDKTKEALAKVLPGEASVKNPVDIVGDAPPKRYEDALEICFKEPPETVAGAVILVTPQGQTKPCEVAELCTRMYAKYPDRLVVSAFMGGLTMQEPSKILNNAKMPVFPFPEPAIHATGAVLKYRKIKNRKTLAEKKVEVFKVDNERIKKIIAGARADGRTVLLSHETSEIFTLYGVNAPKTKLATNEAEAATFAKEVTFPVVMKIVSPQIIHKSDCGGVKLNIKTEAEATAAFKEIMANAAKNGPKGAVLKGVEIQQMVDFSKYQKTTEMIVGVNRDPTWGPMIMVGQGGIYANYIKDVAFDLAYKYDREDAEAQLKKTKIYEILNGVRGQPRSDIKGLLDTMVKLAQLVNDFSEITELDMNPLLVFEEQKEGKNPGIAAVDVKITLSH

SEQ ID NO: 12 - protein, 462 aa, Pyrococcus furiosus
  misc_feature (1)..(462): Acetyl-CoA synthetase
MSLEALFNPKSVAVIGASAKPGKIGYAIMKNLIEYGYEGKIYPVNIKGGEIEINGRKFKVYKSVLEIPDEVDMAVIVVPAKFVPQVLEECGQKGVKVVPIISSGFGELGEEGKKVEQQLVETARKYGMRILGPNIFGVVYTPAKLNATFGPTDVLPGPLALISQSGALGIALMGWTILEKIGLSAVVSVGNKADIDDADLLEFFKDDENTRAILIYMEGVKDGRRFMEVAKEVSKKKPIIVIKAGRSERGAKAAASHTGSLAGSDKVYSAAFKQSGVLRAYTIGEAFDWARALSNLPEPQGDNVVIITNGGGIGVMATDAAEEEGLHLYDNLEELKIFANHMPPFGSYKNPVDLTGMADGKSYEGAIRDALAHPEMHSIAVLYCQTAVLDPRELAEIVIREYNESGRKKPLVVAIVGGIEAKEAIDMLNENGIPAYPEPERAIKALSALYKWSKWKAKHKEK

SEQ ID NO: 13 - protein, 232 aa, Pyrococcus furiosus
  misc_feature (1)..(232): Acetyl-CoA synthetase
MDRVAKAREIIEKAKAENRPLVEPEAKEILKLYGIPVPEFKVARNEEEAVKFSGEIGYPVVMKIVSPQIIHKSDAGGVKINIKNDEEAREAFRTIMQNARNYKPDADLWGVIIYRMLPLGREVIVGMIRDPQFGPAVMFGLGGIFVEILKDVSFRVAPITKEDALEMIREIKAYPILAGARGEKPVNIEALADIIVKVGELALELPEIKEIDINPIFAYEDSAIAVDARMIL

SEQ ID NO: 14 - protein, 462 aa, Pyrococcus furiosus; identical to SEQ ID NO: 12
  misc_feature (1)..(462): Acetyl-CoA synthetase

SEQ ID NO: 15 - protein, 232 aa, Pyrococcus furiosus; identical to SEQ ID NO: 13
  misc_feature (1)..(232): Acetyl-CoA synthetase

SEQ ID NO: 16 - protein, 335 aa, Saccharomyces cerevisiae
  misc_feature (1)..(335): TAL1
MSEPAQKKQKVANNSLEQLKASGTVVVADTGDFGSIAKFQPQDSTTNPSLILAAAKQPTYAKLIDVAVEYGKKHGKTTEEQVENAVDRLLVEFGKEILKIVPGRVSTEVDARLSFDTQATIEKARHIIKLFEQEGVSKERVLIKIASTWEGIQAAKELEEKDGIHCNLTLLFSFVQAVACAEAQVTLISPFVGRILDWYKSSTGKDYKGEADPGVISVKKIYNYYKKYGYKTIVMGASFRSTDEIKNLAGVDYLTISPALLDKLMNSTEPFPRVLDPVSAKKEAGDKISYISDESKFRFDLNEDAMATEKLSEGIRKFSADIVTLFDLIEKKVTA

SEQ ID NO: 17 - protein, 600 aa, Saccharomyces cerevisiae
  misc_feature (1)..(600): XKS1
MLCSVIQRQTREVSNTMSLDSYYLGFDLSTQQLKCLAINQDLKIVHSETVEFEKDLPHYHTKKGVYIHGDTIECPVAMWLEALDLVLSKYREAKFPLNKVMAVSGSCQQHGSVYWSSQAESLLEQLNKKPEKDLLHYVSSVAFARQTAPNWQDHSTAKQCQEFEECIGGPEKMAQLTGSRAHFRFTGPQILKIAQLEPEAYEKTKTISLVSNFLTSILVGHLVELEEADACGMNLYDIRERKFSDELLHLIDSSSKDKTIRQKLMRAPMKNLIAGTICKYFIEKYGFNTNCKVSPMTGDNLATICSLPLRKNDVLVSLGTSTTVLLVTDKYHPSPNYHLFIHPTLPNHYMGMICYCNGSLARERIRDELNKERENNYEKTNDWTLFNQAVLDDSESSENELGVYFPLGEIVPSVKAINKRVIFNPKTGMIEREVAKFKDKRHDAKNIVESQALSCRVRISPLLSDSNASSQQRLNEDTIVKFDYDESPLRDYLNKRPERTFFVGGASKNDAIVKKFAQVIGATKGNFRLETPNSCALGGCYKAMWSLLYDSNKIAVPFDKFLNDNFPWHVMESISDVDNENWDRYNSKIVPLSELEKTLI

SEQ ID NO: 18 - protein, 680 aa, Saccharomyces cerevisiae
  misc_feature (1)..(680): TKL1
MTQFTDIDKLAVSTIRILAVDTVSKANSGHPGAPLGMAPAAHVLWSQMRMNPTNPDWINRDRFVLSNGHAVALLYSMLHLTGYDLSIEDLKQFRQLGSRTPGHPEFELPGVEVTTGPLGQGISNAVGMAMAQANLAATYNKPGFTLSDNYTYVFLGDGCLQEGISSEASSLAGHLKLGNLIAIYDDNKITIDGATSISFDEDVAKRYEAYGWEVLYVENGNEDLAGIAKAIAQAKLSKDKPTLIKMTTTIGYGSLHAGSHSVHGAPLKADDVKQLKSKFGFNPDKSFVVPQEVYDHYQKTILKPGVEANNKWNKLFSEYQKKFPELGAELARRLSGQLPANWESKLPTYTAKDSAVATRKLSETVLEDVYNQLPELIGGSADLTPSNLTRWKEALDFQPPSSGSGNYSGRYIRYGIREHAMGAIMNGISAFGANYKPYGGTFLNFVSYAAGAVRLSALSGHPVIWVATHDSIGVGEDGPTHQPIETLAHFRSLPNIQVWRPADGNEVSAAYKNSLESKHTPSIIALSRQNLPQLEGSSIESASKGGYVLQDVANPDIILVATGSEVSLSVEAAKTLAAKNIKARVVSLPDFFTFDKQPLEYRLSVLPDNVPIMSVEVLATTCWGKYAHQSFGIDRFGASGKAPEVFKFFGFTPEGVAERAQKTIAFYKGDKLISPLKKAF

SEQ ID NO: 19 - protein, 238 aa, Saccharomyces cerevisiae
  misc_feature (1)..(238): RPE1
MVKPIIAPSILASDFANLGCECHKVINAGADWLHIDVMDGHFVPNITLGQPIVTSLRRSVPRPGDASNTEKKPTAFFDCHMMVENPEKWVDDFAKCGADQFTFHYEATQDPLHLVKLIKSKGIKAACAIKPGTSVDVLFELAPHLDMALVMTVEPGFGGQKFMEDMMPKVETLRAKFPHLNIQVDGGLGKETIPKAAKAGANVIVAGTSVFTAADPHDVISFMKEEVSKELRSRDLLD

SEQ ID NO: 20 - protein, 258 aa, Saccharomyces cerevisiae
  misc_feature (1)..(258): RKI1
MAAGVPKIDALESLGNPLEDAKRAAAYRAVDENLKFDDHKIIGIGSGSTVVYVAERIGQYLHDPKFYEVASKFICIPTGFQSRNLILDNKLQLGSIEQYPRIDIAFDGADEVDENLQLIKGGGACLFQEKLVSTSAKTFIVVADSRKKSPKHLGKNWRQGVPIEIVPSSYVRVKNDLLEQLHAEKVDIRQGGSAKAGPVVTDNNNFIIDADFGEISDPRKLHREIKLLVGVVETGLFIDNASKAYFGNSDGSVEVTEK

SEQ ID NO: 21 - DNA, 2122 bp, Saccharomyces cerevisiae
  misc_feature (1)..(2122): GRE3
acagacttcgtaaagagcaatcggagatgaatttaagaaatacaatcaaaagaaaagagaaattttatgatagtcaagaacaaattcttgagttacaagagggagacgttgatgattcgttgatttggaacgttcctatggcatcattatctactaattcatttctagcgtctgctaagcccgatgatatgaataacttggctggcaagaatgacttatcagaatataccggaggtttggtaaatgataactctgaaatttcttatacaaaacaaaatcataggtactcgaacatctcttttgcgagtacaacatcaaacgcctcattattggactttaatgagatgcctacgtctccgattccaggtttgaacaaagtaactgattttcagttcattcaagacacaaccaagagtctagcctctgtttatttgcattcttccaataggctttcaagatctaagctgtccgaaagaacaaagtcttccgatttcttgccaattgaactaaaagaagctcaaaatcaaggcatggaagatttgatacttgtctcggagaacaaactagatgtggtcagccattcaagaccgagttggttaccacccaaggatcgccaggaaaaaaagcttcatgaaaggcaaattaacaaaagcatgagtgttgcttcccttgaccaactaggaaaaaataaagacagagaagaaaagttgattagagatgaaacaaataggcaaaaatatgtgttattattggacagagatataactagaaactcctccttacaaagcctaagtaaaatggtttgggacactccatttagtgacgaaactaggtcaacaatttacagcgaaattttacagagcaagactaggtttattaccaaaaactatattcaaccatttcatgagctacaggagcttttaacaaaaatgggagactttcctaaaaacaaggaaattgaaatatcgcagctaatcgaaacaagtttgaggcgaaaagtgagcggtttacatgatatatgtcctgatttgatgcttttattgaagataaaatctatctcatcacagggtatagtcaccggtgatgaactccttttccatcatttcttggtgagtgaatcatttcagaacctggggctaaacgagatttggaatattgttaatttagtacaaatgacgtgttttaatgatctttgtaaagaaaagttcgatgcaaaggttttagaacgtaagggtgtcgtagccggttatttatcgcaaaacgaggagttcaaggatgaatttaatacggagtgtataaactctaccacctggtggaacatcctagaacgtattgatcataagctttttatgtggatcatggatattatagtagtcaacaattcccagagctacaaaaatagcccaatcaacgaagatgagtttgttaacaaggattgggaatattaccgctcgaagaaagtggtaataaactacaagatcttgatttcatttgcattaaatgtattgttaaattaccactttggattcactgatttaagaagtctttgtaacgtgaatgaccagagattttgcattccagtattcatcaatgatgaattcgtagacgcagatactgtaaatgccgtgttcatcaagaaatgggcgcattactacaagaagttttgatattttttgtaactgtaatttcactcatgcacaagaaaaaaaaaactggattaaaagggagcccaaggaaaactcctcagcatatatttagaagtctcctcagcatatagttgtttgttttctttacacattcactgtttaataaaacttttataatatttcattatcggaactctagattctatacttgtttcccaattgttgctggtagtaaacgtatacgtcataaaagggaaaagccacatgcggaagaattttatggaaaaaaaaaaaacctcgaagttactacttctagggggcctatcaagtaaattactcctggtacactgaagtatataagggatatagaagcaaatagttgtcagtgcaatccttcaagacgattgggaaaatactgtaatataaatcgtaaaggaaaattggaaattttttaaagatgtcttcactggttactc

SEQ ID NO: 22 - DNA, 2160 bp, Saccharomyces cerevisiae
  misc_feature (1)..(2160): downstream sequence to delete GRE3
gcctgatccagccagtaaaatccatactcaacgacgatatgaacaaatttccctcattccgatgctgtatatgtgtataaatttttacatgctcttctgtttagacacagaacagctttaaataaaatgttggatatactttttctgcctgtggtgtcatccacgcttttaattcatctcttgtatggttgacaatttggctattttttaacagaacccaacggtaattgaaattaaaagggaaacgagtgggggcgatgagtgagtgatactaaaatagacaccaagagagcaaagcggtcccaaaatcatttgagtaaccggatatctatcgggatattaatagcagcttccatttcaactaaaacaacagcaagatatgagcgacaagatatcctttctacctcccgaacccatccaactacttgacgaagactccacggagcctgaactcgacattgactcacaacaagaaaatgagggacccatcagtgcgtcaaacagcaatgatagcactagccatagtaatgattgcggtgccacaattaccagaacaagacctagacgaagcagttctatcaatgcaaactttagttttcaaaaggctcatgtcagcgattgcaccatagtcaatggcgaccatggaacaaagtttgctgtctggagaattaccgtatttcttgaacccaacttgaaggcttttgcggccaagagggaaagctataaaatccaaacctataaacgatactccgatttcgtcagattacgagagaatttgctcacaagaatcaagacagcgaaacctgagaaacttaactgtttgcagattccacaccttcccccttcagtgcagtggtacagttcttggaaatatcaagaagtgaatctgaacaaggactggctggcaaaaagacagagagggctcgagtacttcctcaatcacatcatccttaacagcagcctcgtagaaatgaccaaagatatactcatacagtttctagagccttcaaaacgagttgcatagctcaccatccctatccaaccgactattcttctcatcgactactactatcccatttaactcgggcgcgttgttaattaatcactcgatggggaatgccttgagctgaccgcaatgaaaacttttaggggatcgtccaacattaaaggaagaacgaaacggactccacagtttctaatataaataaacaatgataaaacatatagtttcgccattcaggacgaattttgttggcatcagcaagtccgtgctgtcaaggatgattcatcacaaggttacaatcataggttctggccccgctgcccacaccgctgctatatacttggcaagagcagagatgaagcccacattatatgagggaatgatggccaacggaattgctgctggtggccaattgacaacaaccaccgatatcgaaaatttcccagggtttcctgaatcgttgagtggcagtgaactgatggagaggatgaggaaacaatctgccaagtttggcactaacataattaccgagactgtctctaaagtcgatttatcttcaaaaccattcagattatggaccgaatttaatgaggatgcagagcctgtgaccactgatgctataatcttggccacgggtgcttccgctaagagaatgcatttaccaggggaggaaacctactggcagcagggaatatctgcctgtgctgtatgtgatggtgcagtccctatctttagaaacaagccattggccgttattggtggtggtgactctgcgtgtgaggaagcggaatttcttacgaagtatgcgtcgaaagtatatatattagtaagaaaggatcattttcgtgcatctgtaataatgcagagacgaattgagaaaaatccaaacatcattgttttgttcaacacagttgcattagaagctaagggtgatggtaagttattgaatatgttgagaattaagaatactaaaagtaatgtggagaacgatttagaagtaaatggactattttacgcaataggtcacagccctgccacagatatagttaaaggacaagtagatgaagaagagacggggtatataaaaactgtgcctggatcgtctctgacttctgtgccaggtttttttgctgcaggtgacgttcaggactctaggt

SEQ ID NO: 23 - DNA, 1518 bp, Saccharomyces cerevisiae
  misc_feature (1)..(1518): zwf1
atgagtgaaggccccgtcaaattcgaaaaaaataccgtcatatctgtctttggtgcgtcaggtgatctggcaaagaagaagacttttcccgccttatttgggcttttcagagaaggttaccttgatccatctaccaagatcttcggttatgcccggtccaaattgtccatggaggaggacctgaagtcccgtgtcctaccccacttgaaaaaacctcacggtgaagccgatgactctaaggtcgaacagttcttcaagatggtcagctacatttcgggaaattacgacacagatgaaggcttcgacgaattaagaacgcagatcgagaaattcgagaaaagtgccaacgtcgatgtcccacaccgtctcttctatctggccttgccgccaagcgtttttttgacggtggccaagcagatcaagagtcgtgtgtacgcagagaatggcatcacccgtgtaatcgtagagaaacctttcggccacgacctggcctctgccagggagctgcaaaaaaacctggggcccctctttaaagaagaagagttgtacagaattgaccattacttgggtaaagagttggtcaagaatcttttagtcttgaggttcggtaaccagtttttgaatgcctcgtggaatagagacaacattcaaagcgttcagatttcgtttaaagagaggttcggcaccgaaggccgtggcggctatttcgactctataggcataatcagagacgtgatgcagaaccatctgttacaaatcatgactctcttgactatggaaagaccggtgtcttttgacccggaatctattcgtgacgaaaaggttaaggttctaaaggccgtggcccccatcgacacggacgacgtcctcttgggccagtacggtaaatctgaggacgggtctaagcccgcctacgtggatgatgacactgtagacaaggactctaaatgtgtcacttttgcagcaatgactttcaacatcgaaaacgagcgttgggagggcgtccccatcatgatgcgtgccggtaaggctttgaatgagtccaaggtggagatcagactgcagtacaaagcggtcgcatcgggtgtcttcaaagacattccaaataacgaactggtcatcagagtgcagcccgatgccgctgtgtacctaaagtttaatgctaagacccctggtctgtcaaatgctacccaagtcacagatctgaatctaacttacgcaagcaggtaccaagacttttggattccagaggcttacgaggtgttgataagagacgccctactgggtgaccattccaactttgtcagagatgacgaattggatatcagttggggcatattcaccccattactgaagcacatagagcgtccggacggtccaacaccggaaatttacccctacggatcaagaggtccaaagggattgaaggaatatatgcaaaaacacaagtatgttatgcccgaaaagcacccttacgcttggcccgtgactaagccagaagatacgaaggataattag

SEQ ID NO: 24 - DNA, 2232 bp, Saccharomyces cerevisiae
  misc_feature (1)..(2232): stb5
atggatggtcccaattttgcacatcaaggcgggagatcacaacgtactactgaattgtattcgtgcgcacgatgcagaaaattaaagaagaagtgtggtaaacaaataccgacatgtgcaaactgcgataaaaatggggcacactgttcatatccaggtagagccccaagacgtaccaagaaggagttagcggatgctatgctacgaggggaatatgttccagtgaaaaggaacaagaaggtaggaaaaagcccattgagcactaagagcatgccaaactcttctagtccgctatccgcaaatggcgctataactcccgggttttcgccttacgaaaacgatgatgcacataagatgaaacagctaaaaccgtcagatccaataaatcttgtcatgggggcaagtccaaattctagcgaaggtgtctcatcgctaatttcggtgctaacatcgctgaatgataattctaatccttcttcgcacttatcctctaatgaaaattccatgattccttcccgatcattgccagcttccgtgcagcaaagttcgacaacttcatcattcggaggatataacacgccttcaccactaattagcagtcatgtgcctgcgaacgcccaagccgtaccgctacaaaacaacaatcgcaatactagcaacggggataacggcagtaatgttaaccacgataataacaatggcagtaccaacacaccgcaattgagtcttaccccatatgcaaacaattcagcccccaatgggaaattcgattctgtgccggttgatgcatcctcgatcgaatttgaaactatgtcctgttgctttaaaggtggtagaacaacatcgtgggtcagagaggatggctcgttcaagtcaattgatagatccttactggacaggttcattgccgcatacttcaaacacaatcaccgtctatttcccatgattgataaaatagcattcctaaatgacgccgcgacaattactgatttcgaaaggttatatgacaacaaaaactaccctgacagctttgttttcaaagtatacatgatcatggctattggttgtacaactttacagcgtgctggtatggtttctcaggacgaagagtgtctgagtgaacatttggcttttttggccatgaaaaaatttcgtagtgttataattttacaagatatcgaaactgtacgatgcctattgttgttgggtatttattcgttttttgagccaaagggctcctcgtcatggacaattagtggtatcatcatgcgattgaccataggattaggtttaaatagagagttaactgccaaaaaactcaagagcatgtctgctttagaagcagaggcaagatatagagtgttttggagtgcttactgctttgaaaggctagtatgcacctcgttgggccgtatatccgggatcgacgacgaagacatcactgtgccactaccgagggcgttgtatgtggatgaaagagacgatttagagatgaccaagttgatgatatcattaaggaaaatgggcggtcgcatttataaacaagtccactctgtaagtgcagggcgacaaaagttaaccatcgaacaaaagcaggaaatcataagtggattacgcaaggagctagacgaaatttattctcgagaatcagaaagaaggaaactgaaaaaatctcaaatggatcaggtggagagggagaacaattctactacaaatgtaatatccttccatagttctgagatttggctagcaatgagatactcccagttgcaaatcttactatacagaccatctgcattgatgccaaaaccgcccattgactcactatccactctaggagagttttgcttgcaagcctggaaacatacttatacactgtacaagaagcggttattacccttgaactggataacccttttcagaacattaaccatttgtaacactatcttatactgtctttgccagtggagcatcgacctcattgaaagtaaaatcgaaattcaacagtgtgtggaaatactaagacatttcggtgaaagatggatttttgctatgagatgcgcggatgttttccaaaacattagcaacacaattctcgatattagtttaagccatggtaaagttcctaatatggaccaattaacaagagagttatttggcgccagcgattcatatcaagacatattagacgaaaacaatgttgacgtctcttgggttgataaacttgtatga

SEQ ID NO: 25 - protein, 910 aa, Bifidobacterium adolescentis
  misc_feature (1)..(910): adhE
MADAKKKEEPTKPTPEEKLAAAEAEVDALVKKGLKALDEFEKLDQKQVDHIVAKASVAALNKHLVLAKMAVEETHRGLVEDKATKNIFACEHVTNYLAGQKTVGIIREDDVLGIDEIAEPVGVVAGVTPVTNPTSTAIFKSLIALKTRCPIIFGFHPGAQNCSVAAAKIVRDAAIAAGAPENCIQWIEHPSIEATGALMKHDGVATILATGGPGMVKAAYSSGKPALGVGAGNAPAYVDKNVDVVRAANDLILSKHFDYGMICATEQAIIADKDIYAPLVKELKRRKAYFVNADEKAKLEQYMFGCTAYSGQTPKLNSVVPGKSPQYIAKAAGFEIPEDATILAAECKEVGENEPLTMEKLAPVQAVLKSDNKEQAFEMCEAMLKHGAGHTAAIHTNDRDLVREYGQRMHACRIIWNSPSSLGGVGDIYNAIAPSLTLGCGSYGGNSVSGNVQAVNLINIKRIARRNNNMQWFKIPAKTYFEPNAIKYLRDMYGIEKAVIVCDKVMEQLGIVDKIIDQLRARSNRVTFRIIDYVEPEPSVETVERGAAMMREEFEPDTIIAVGGGSPMDASKIMWLLYEHPEISFSDVREKFFDIRKRAFKIPPLGKKAKLVCIPTSSGTGSEVTPFAVITDHKTGYKYPITDYALTPSVAIVDPVLARTQPRKLASDAGFDALTHAFEAYVSVYANDFTDGMALHAAKLVWDNLAESVNGEPGEEKTRAQEKMHNAATMAGMAFGSAFLGMCHGMAHTIGALCHVAHGRTNSILLPYVIRYNGSVPEEPTSWPKYNKYIAPERYQEIAKNLGVNPGKTPEEGVENLAKAVEDYRDNKLGMNKSFQECGVDEDYYWSIIDQIGMRAYEDQCAPANPRIPQIEDMKDIAIAAYYGVSQAEGHKLRVQRQGEAATEEASERA

SEQ ID NO: 26 - DNA, 1882 bp, Saccharomyces cerevisiae
  misc_feature (1)..(1882): upstream sequence to delete Gpd1
aagcctacaggcgcaagataacacatcaccgctctcccccctctcatgaaaagtcatcgctaaagaggaacactgaaggttcccgtaggttgtctttggcacaaggtagtacatggtaaaaactcaggatggaataattcaaattcaccaatttcaacgtcccttgtttaaaaagaaaagaatttttctctttaaggtagcactaatgcattatcgatgatgtaaccattcacacaggttatttagcttttgatccttgaaccattaattaacccagaaatagaaattacccaagtggggctctccaacacaatgagaggaaaggtgactttttaagggggccagaccctgttaaaaacctttgatggctatgtaataatagtaaattaagtgcaaacatgtaagaaagattctcggtaacgaccatacaaatattgggcgtgtggcgtagtcggtagcgcgctcccttagcatgggagaggtctccggttcgattccggactcgtccaaattattttttactttccgcggtgccgagatgcagacgtggccaactgtgtctgccgtcgcaaaatgatttgaattttgcgtcgcgcacgtttctcacgtacataataagtattttcatacagttctagcaagacgaggtggtcaaaatagaagcgtcctatgttttacagtacaagacagtccatactgaaatgacaacgtacttgacttttcagtattttctttttctcacagtctggttatttttgaaagcgcacgaaatatatgtaggcaagcattttctgagtctgctgacctctaaaattaatgctattgtgcaccttagtaacccaaggcaggacagttaccttgcgtggtgttactatggccggaagcccgaaagagttatcgttactccgattattttgtacagctgatgggaccttgccgtcttcattttttttttttttcacctatagagccgggcagagctgcccggcttaactaagggccggaaaaaaaacggaaaaaagaaagccaagcgtgtagacgtagtataacagtatatctgacacgcacgtgatgaccacgtaatcgcatcgcccctcacctctcacctctcaccgctgactcagcttcactaaaaaggaaaatatatactctttcccaggcaaggtgacagcggtccccgtctcctccacaaaggcctctcctggggtttgagcaagtctaagtttacgtagcataaaaattctcggattgcgtcaaataataaaaaaagtaaccccacttctacttctacatcggaaaaacattccattcacatatcgtctttggcctatcttgttttgtcctcggtagatcaggtcagtacaaacgcaacacgaaagaacaaaaaaagaagaaaacagaaggccaagacagggtcaatgagactgttgtcctcctactgtccctatgtctctggccgatcacgcgccattgtccctcagaaacaaatcaaacacccacaccccgggcacccaaagtccccacccacaccaccaatacgtaaacggggcgccccctgcaggccctcctgcgcgcggcctcccgccttgcttctctccccttccttttctttttccagttttccctattttgtccctttttccgcacaacaagtatcagaatgggttcatcaaatctatccaacctaattcgcacgtagactggcttggtattggcagtttcgtagttatatatatactaccatgagtgaaactgttacgttaccttaaattctttctccctttaattttcttttatcttactctcctacataagacatcaagaaacaattgtatattgtacaccccccccctccacaaacacaaatattgataatataaag

SEQ ID NO: 27 - DNA, 1852 bp, Saccharomyces cerevisiae
  misc_feature (1)..(1852): downstream sequence to delete Gpd1
atttattggagaaagataacatatcatactttcccccacttttttcgaggctcttctatatcatattcataaattagcattatgtcatttctcataactactttatcacgttagaaattacttattattattaaattaatacaaaatttagtaaccaaataaatataaataaatatgtatatttaaattttaaaaaaaaaatcctatagagcaaaaggattttccattataatattagctgtacacctcttccgcattttttgagggtggttacaacaccactcattcagaggctgtcggcacagttgcttctagcatctggcgtccgtatgtatgggtgtattttaaataataaacaaagtgccacaccttcaccaattatgtctttaagaaatggacaagttccaaagagcttgcccaaggctcgacaaggatgtactttggaatatctatattcaagtacgtggcgcgcatatgtttgagtgtgcacacaataaaggtttttagatattttgcggcgtcctaagaaaataaggggtttcttaaaaaataacaatagcaaacaaagttccttacgatgatttcagatgtgaatagcatggtcatgatgagtatatacgtttttataaataattaaaagttttcctcttgtctgtttttttgttggctcgtggttgttctcgaaaaaggagagttttcattttcgaaataggtgattatcatcatgttgttatcaccccacgacgaagataatacggagctcaccgttttctttttttttccctttggctgaaatttcccaccagaacaaacgtgacaaaattatctttgaatccaaagtagcttatatatatacgtagaagtgtttcgagacacacatccaaatacgaggttgttcaatttaaacccaagaatacataaaaaaaatatagatatattaacttagtaaacaatgactgcaagcacaccatccaatgtcatgacattgttcttgttaaggcatggacaaagtgaattgaatcacgagaatatattctgtggttggattgacgctaagctaaccgaaaaaggtaaagaacaagctcgtcattctgccgagctaatcgaacaatattgtaaagctaataatttgagattaccccagattggttacacctcacgtttaattaggacccaacagaccatagaaacgatgtgtgaagaatttaagttaaagccacaactgcaggttgtttacgactttaataaaatcaaacttggagacgaatttggcagtgatgacaaggataatatgaaaatcccgattcttcaaacttggaggctaaatgaacgtcattacggttcctggcagggccagaggaaaccgaatgttttaaaagaatatggtaaggataaatatatgttcattaggagagattacgagggtaagccaccacctgtagatcttgaccgtgagatgattcaacaagaaaatgagaagggctcttctactgggtacgaattcaaggagccaaacagacaaataaaatatgaattggaatgcagcaatcatgacattgtattaccggattccgaatctcttcgtgaagtggtttatagattgaatccttttctacaaaatgtcatattaaaattagccaatcaatatgatgaatcttcatgcctgattgtgggccatggaagttcagtgagatcgctactgaaaattctggagggtatatcagatgatgacatcaagaatgttgatattccaaatggtatccccttagtcgttgaattagataagaataatggtcttaagtttatcagaaaattctacctagatcctgaatctgctaagatcaatgctgag

SEQ ID NO: 28 - DNA, 540 bp, Saccharomyces cerevisiae
  misc_feature (1)..(540): Gpd2 promtor region
caaggaattaccatcaccgtcaccatcaccatcatatcgccttagcctctagccatagccatcatgcaagcgtgtatcttctaagattcagtcatcatcattaccgagtttgttttccttcacatgatgaagaaggtttgagtatgctcgaaacaataagacgacgatggctctgccattgttatattacgcttttgcggcgaggtgccgatgggttgctgaggggaagagtgtttagcttacggacctattgccattgttattccgattaatctattgttcagcagctcttctctaccctgtcattctagtattttttttttttttttttggttttacttttttttcttcttgcctttttttcttgttactttttttctagttttttttccttccactaagctttttccttgatttatccttgggttcttctttctactcctttagattttttttttatatattaatttttaagtttatgtattttggtagattcaattctctttccctttccttttccttcgctccccttccttatca

SEQ ID NO: 29 - DNA, 2074 bp, Saccharomyces cerevisiae
  misc_feature (1)..(2074): downstream sequence to delete Gpd2
tctgatctttcctgttgcctctttttcccccaaccaatttatcattatacacaagttctacaactactactagtaacattactacagttattataattttctattctctttttctttaagaatctatcattaacgttaatttctatatatacataactaccattatacacgctattatcgtttacatatcacatcaccgttaatgaaagatacgacaccctgtacactaacacaattaaataatcgccataaccttttctgttatctatagcccttaaagctgtttcttcgagctttttcactgcagtaattctccacatgggcccagccactgagataagagcgctatgttagtcactactgacggctctccagtcatttatgtgattttttagtgactcatgtcgcatttggcccgtttttttccgctgtcgcaacctatttccattaacggtgccgtatggaagagtcatttaaaggcaggagagagagattactcatcttcattggatcagattgatgactgcgtacggcagatagtgtaatctgagcagttgcgagacccagactggcactgtctcaatagtatattaatgggcatacattcgtactcccttgttcttgcccacagttctctctctctttacttcttgtatcttgtctccccattgtgcagcgataaggaacattgttctaatatacacggatacaaaagaaatacacataattgcataaaataatgtctaagggaaaagtttgtttggcttattctggtggtttagatacctccgtcattttggcttggctactagaccaaggctacgaagttgtagctttcatggctaatgtagggcaagaagaagatttcgatgccgccaaggaaaaggccttgaagatcggtgcctgcaagttcgtttgtgtggattgtcgtgaagattttgtcaaggatattctattcccagctgtacaggtcaacgctgtgtacgaagacgtttatctgttgggtacctctttggcaagacctgttattgccaaagcccaaattgacgtcgctaaacaggagggctgtttcgcggtctctcatggttgtaccggtaaaggtaatgatcaaatcagattcgaattgtcattttacgctctgaagccagacgttaagtgtattacaccatggagaatgcctgaatttttcgaaagatttgctggcagaaaggatttgttagactatgctgcacaaaagggtattcccgtcgcccaaaccaaggccaagccatggtctactgacgaaaaccaagcccacatttcttacgaggcaggtatcttggaagacccagataccaccccaccaaaggacatgtggaaattgatcgtcgatccaatggatgctccggaccaaccacaagatttgaccattgactttgaacgtggtcttccagtcaagttgacctacaccgacaacaagacttccaaggaagtttccgttaccaagcctttggatgttttcttggccgcatccaacttagcaagggccaacggtgttggtagaatcgatattgtagaagatcgttacattaacttgaaatccagaggttgttacgaacaggctccattgactgttttgagaaaagctcatgttgatttggaaggtttgactttagacaaagaagtccgtcaattgagagactcattcgtcacaccaaactactccagattgatatataacggtttcctacttcacccagagtgtgagtacatcagatctatgatccaaccatcccaaaatagcgttaacggtactgtcagggttagactgtataagggtaacgtcatcattctgggcagatctacaaagactgaaaagttgtacgatccgacagaatcctctatggatgagttgaccggtttcttacctaccgataccaccggtttcattgccatccaggccattagaattaaaaaatacggtgaatccaaaaaaaccaaaggtgaagagttgactttgtaagtccgctagttcatcgcctcaagatagataacgatctcttcctccacctcctatttctgcacactcttgtga

SEQ ID NO: 30 - DNA, 1630 bp, Thermoanaerobacter pseudethanolicus
  misc_feature (1)..(1630): adhB
tgaacaatagacaacccctttctgtgatcttgttttttgcaaatgctattttatcacaagagatttctctagttcttttttacttaaaaaaaccctacgaaattttaaactatgtcgaataaattattgataatttttaactatgtgctattatattattgcaaaaaatttaacaatcatcgcgtaagctagttttcacattaatgacttacccagtattttaggaggtgtttaatgatgaaaggttttgcaatgctcagtatcggtaaagttggctggattgagaaggaaaagcctgctcctggcccatttgatgctattgtaagacctctagctgtggccccttgcacttcggacattcataccgtttttgaaggagccattggcgaaagacataacatgatactcggtcacgaagctgtaggtgaagtagttgaagtaggtagtgaggtaaaagattttaaacctggtgatcgcgttgttgtgccagctattacccctgattggtggacctctgaagtacaaagaggatatcaccagcactccggtggaatgctggcaggctggaaattttcgaatgtaaaagatggtgtttttggtgaattttttcatgtgaatgatgctgatatgaatttagcacatctgcctaaagaaattccattggaagctgcagttatgattcccgatatgatgaccactggttttcacggagctgaactggcagatatagaattaggtgcgacggtagcagttttgggtattggcccagtaggtcttatggcagtcgctggtgccaaattgcgtggagccggaagaattattgccgtaggcagtagaccagtttgtgtagatgctgcaaaatactatggagctactgatattgtaaactataaagatggtcctatcgaaagtcagattatgaatctaactgaaggcaaaggtgtcgatgctgccatcatcgctggaggaaatgctgacattatggctacagcagttaagattgttaaacctggtggcaccatcgctaatgtaaattattttggcgaaggagaggttttgcctgttcctcgtcttgaatggggttgcggcatggctcataaaactataaaaggcgggctatgccccggtggacgtctaagaatggaaagactgattgaccttgttttttataagcctgtcgatccttctaagctcgtcactcacgttttccagggatttgacaatattgaaaaagcctttatgttgatgaaagacaaaccaaaagacctaatcaaacctgttgtaatattagcataaaaatggggacttagtccatttttatgctaataaggctaaatacactggtttttttatatgacacatcggccagtaaactcttggtaaaaaaataacaaaaaatagttattttcttaacatttttacgccattaacacttgataacatcatcgaagaagtaaataaacaactattaaataaaagaagaaggaggattatcatgttcaaaattttagaaaaaagagaattggcaccttccatcaagttgtttgtaatagaggcaccactagtagccaaaaaagcaaggccaggccaattcgttatgctaaggataaaagaaggaggagaaagaatt

SEQ ID NO: 31 - protein, 351 aa, Clostridium beijerinckii
  misc_feature (1)..(351): secondary alcohol dehydrogenases
MKGFAMLGINKLGWIEKERPVAGSYDAIVRPLAVSPCTSDIHTVFEGALGDRKNMILGHEAVGEVVEVGSEVKDFKPGDRVIVPCTTPDWRSLEVQAGFQQHSNGMLAGWKFSNFKDGVFGEYFHVNDADMNLAILPKDMPLENAVMITDMMTTGFHGAELADIQMGSSVVVIGIGAVGLMGIAGAKLRGAGRIIGVGSRPICVEAAKFYGATDILNYKNGHIVDQVMKLTNGKGVDRVIMAGGGSETLSQAVSMVKPGGIISNINYHGSGDALLIPRVEWGCGMAHKTIKGGLCPGGRLRAEMLRDMVVYNRVDLSKLVTHVYHGFDHIEEALLLMKDKPKDLIKAVVIL

SEQ ID NO: 32 - DNA, 1056 bp, Clostridium beijerinckii
  misc_feature (1)..(1056): codon-optimized secondary alcohol dehydrogenase
atgaaagggtttgctatgttgggtattaacaaattaggttggatcgaaaaggaaagacctgttgccgggtcctacgatgccatagttcgtccattagcagtgtctccatgcactagtgatattcataccgtttttgaaggtgctttaggcgacagaaagaatatgattttgggtcatgaggcggttggcgaagtagttgaagtcggctcagaagttaaagatttcaaaccaggtgatagggtaattgtaccatgtacaacgcctgattggagatctcttgaagtccaagcaggattccaacaacactccaatgggatgctggcagggtggaaattttctaacttcaaggatggagttttcggcgaatacttccacgtgaatgatgctgatatgaatttggctatcttaccaaaggacatgcctctcgaaaacgcagtgatgatcacagacatgatgacaacaggttttcatggcgctgaattagccgacattcaaatgggatcttcagtagtcgttatcggtataggcgctgtgggattaatgggaatcgcaggggctaaactaagaggtgccggtagaattattggagttggtagtaggccaatctgcgtagaagcagcgaagttttacggtgccaccgatattctgaattacaaaaatggacacatagtagaccaagtaatgaaactaacaaacggaaaaggtgttgatagagtgatcatggcaggtggtggttctgagactttgtcacaggctgtgtcaatggtcaaacctggtggtatcatttcaaatatcaattaccatggcagcggtgatgctctacttatacctagagtcgaatggggatgtgggatggctcataaaactatcaagggcggattgtgtccaggcggtagactgcgagcggagatgctcagagatatggtcgtttataacagagtcgatctttctaagcttgttactcatgtttatcacggctttgatcatatcgaggaggccttattgttgatgaaagacaagccaaaggacttgattaaagcagttgtgatactataa

SEQ ID NO: 33 - DNA, 1824 bp, Saccharomyces cerevisiae
  misc_feature (1)..(1824): ARI1
gtgaaagctcggaatacatatttatgacggaagaagacagaaatctacggggcagttggatcggtgagccaaaagagtgttttaccttagaccttgcaacatcttctatataccgaagaaggaagaacatgatattcttctggtaaactcttatccagtggaaaacgcacccagcatattcgaaaatatatcaactatctccccttttcatactaataattaatagcattcatattgaaataataaaaaagatacgtttaatacttacgccagctctctagttacagtttcctaacgcatacgtcatcaatttgttaagatcggcttcgctctataaaaatgtcggccgaatttctataaattcggccgaaattagcacaggattttccgcggttccgacccctatcctagaaacacggaaaaacttgctaataattccggaatttattctatgcaactttatgaagacaaattactataaatgaaccgctcattcagaaaaactatgtctcgagctcaatggatcttactacatagtttataaaaacagtaattgtgcattgtacaactgtgctaaacaaacttaaaaaagtaataattatgaccactgataccactgttttcgtttctggcgcaaccggtttcattgctctacacattgtgaacgatctgttgaaagctggctatacagtcatcggctcaggtagatctcaagaaaaaaatgatggcttgctcaaaaaatttaataacaatcccaaactatcgatggaaattgtggaagatattgctgctccaaacgcctttgatgaagttttcaaaaaacatggtaaggaaattaagattgtgctacacactgcctccccattccattttgaaactaccaattttgaaaaggatttactaacccctgcagtgaacggtacaaaatctatcttggaagcgattaaaaaatatgctgcagacactgttgaaaaagttattgttacttcgtctactgctgctctggtgacacctacagacatgaacaaagaagatttggtgatcacggaggagagttggaataaggatacatgggacagttgtcaagccaacgccgttgccgcatattgtggctcgaaaaagtttgctgaaaaaactgcttgggaatttcttaaagaaaacaagtctagtgtcaaattcacactatccactatcaatccgggattcgtttttggtcctcaaatgtttgcagattcgctaaaacatggcataaatacctcctcaggtatcgtatctgagttaattcattccaaggtaggtggagaattttataattactgtggcccatttattgacgtgcgtgacgtttctaaagcccacctagttgcaattgaaaaaccagaatgtaccggccaaagattagtattgagtgaaggtttattctgctgtcaagaaatcgttgacatcttgaacgaggaattccctcaattaaagggcaagatagctacaggtgaacctgcgaccggtccaagctttttagaaaaaaactcttgcaagtttgacaattctaagacaaaaaaactactgggattccagttttacaatttaaaggattgcatagttgacaccgcggcgcaaatgtcagaagttcaaaatgaagcctaagtatcacgctaattgaagttttttttgatcactccaataggcaaatctatagatatataaaaaatatagacaagactttttttttacattgccagttttcttttttcctttttagtatctattcaaatgggcgaccctattgtctgatttcattagcttcatcacacaaaagtgccacga

SEQ ID NO: 34 - protein, 347 aa, Saccharomyces cerevisiae
  misc_feature (1)..(347): ARI1
MTTDTTVFVSGATGFIALHIVNDLLKAGYTVIGSGRSQEKNDGLLKKFNNNPKLSMEIVEDIAAPNAFDEVFKKHGKEIKIVLHTASPFHFETTNFEKDLLTPAVNGTKSILEAIKKYAADTVEKVIVTSSTAALVTPTDMNKEDLVITEESWNKDTWDSCQANAVAAYCGSKKFAEKTAWEFLKENKSSVKFTLSTINPGFVFGPQMFADSLKHGINTSSGIVSELIHSKVGGEFYNYCGPFIDVRDVSKAHLVAIEKPECTGQRLVLSEGLFCCQEIVDILNEEFPQLKGKIATGEPATGPSFLEKNSCKFDNSKTKKLLGFQFYNLKDCIVDTAAQMSEVQNEA

SEQ ID NO: 35 - DNA, 1125 bp, Entamoeba histolytica
  misc_feature (1)..(1125): ADH1
gcacgaggaaaaaccacaatgaaaggacttgctatgcttggaattggaagaattggatggattgaaaagaaaatcccagaatgtggaccacttgatgcattagttagaccattagcacttgcaccatgtacatcagatacacataccgtttgggcaggagctattggagatagacatgatatgattcttggacatgaagcggttggacaaattgttaaagttggatcattagttaagagattaaaagttggagataaagttattgtaccagctattacaccagattggggagaagaagaatcgcaaagaggatatccaatgcattcaggaggaatgcttggaggatggaaattctcaaatttcaaggatggagttttttcagaagttttccatgttaatgaagcagatgccaatcttgcacttcttccaagagatattaaaccagaagatgcagttatgttatcagatatggtaactactggattccatggagcagaattagctaatattaaacttggagatactgtttgtgttattggtattggaccagttggattaatgtcagttgcaggagcaaaccatcttggagcaggaagaatctttgcagtaggatcaagaaaacattgttgtgatattgcattggaatatggagcaacagatattattaattataaaaatggagatattgtagaacaaattcttaaagctacagacggcaaaggagttgataaagtcgttattgcaggaggtgatgttcatacatttgcacaagcagtcaaaatgattaaaccaggatcagatattggaaatgttaattatcttggagaaggagataatattgatattccaagaagtgaatggggagttggaatgggtcataaacacattcatggaggtttaaccccaggtggaagagtcagaatggaaaaattagcatcacttatttcaactggtaaattagatacttctaaacttattacacatagatttgaaggattagaaaaagttgaagatgcattaatgttaatgaagaataaaccagcagaccttatcaaaccagttgtcagaattcattatgatgatgaagatactcttcattaaattcattaattcaaagtattaaac

SEQ ID NO: 36 - DNA, 1502 bp, Cucumis melo
  misc_feature (1)..(1502): ADH1
tcccaaaattcaaatccttttacctataaatactctcactcacttcttccttcatcatcatcatcgtcatttctctttctaacccaaatcaaattttgtttctctctctctctctctctcttcaaatccctttcaccataacccacaactatgtccactgccggtcaggtcatcaaatgcaaagctgctgtggctcgggaggccggaaagccacttgtcattgaaaaagttgaagtggcaccaccgcaagctaatgaagtccgattgaagatccttttcacttctctctgtcataccgatgtttatttctgggaagccaagggacaaaccccattgtttcctcgtatttttggacataaggctggaggaattgttgagagtgttggagaaggagtgaaagatcttcaaccaggagatcatgttcttcctattttcactggtgaatgtggggattgtagtcattgtcaatctgaagaaagcaatatgtgtgatcttcttcgaatcaataccgatcgtggagttatgatcaatgatggcaaaactagattctccaaaaatggacaacccattcatcattttgttggaacctccacttttagtgaatacactgttgttcatgttggttgcttggctaagatcaaccctgctgcccctcttgacaaagtttgtgttcttagctgcggcatttccacaggccttggtgccactttgaatgttgcaaagcctaaaaagggtcaatctgttgcgatctttggacttggagttgttggacttgctgctgctgaaggagcaagaattgctggtgcatctaggatcattggtgttgacctgaacccggctcgattcgaagaagcaaagaaatttggttgcaacgaatttgtgaatccaaaggatcacaacaagccagttcaagaggtgattgctgagatgacgaacggaggagttgaccgaagcgtcgagtgtacgggaagcatccaagcaatgatcgcagcatttgaatgcgttcacgatgggtggggtgttgctgttcttgtgggagtcccaaacaaagacgatgcattcaaaactcatcctatgaatttccttaacgaaagaactctaaagggtacattcttcggcaactacaaaccccgaaccgacattccgggggtggtcgagaagtacttgagcaaggagctggaattggagaagttcattacacatacagtgtcattttctgagatcaacaaggcgtttgattacatgctgaaaggggagtcgattcgatgcattattagaatggataattgaaataataactgtgggatgagatgaaaataagggaataagattatgtggtgattgaaagaggctggagagttctggttttccttatttctttctaagtttgtgtttaatgttttctgagagtggaatgttcgcgatagtgttatcgcctttttgtcaatttcatccaactcttgaaatattgtagtcatattataatcgaaaaaaaaaaaaaa

SEQ ID NO: 37 - DNA, 2142 bp, Saccharomyces cerevisiae
  misc_feature (1)..(2142): acs1
atgtcgccctctgccgtacaatcatcaaaactagaagaacagtcaagtgaaattgacaagttgaaagcaaaaatgtcccagtctgccgccactgcgcagcagaagaaggaacatgagtatgaacatttgacttcggtcaagatcgtgccacaacggcccatctcagatagactgcagcccgcaattgctacccactattctccacacttggacgggttgcaggactatcagcgcttgcacaaggagtctattgaagaccctgctaagttcttcggttctaaagctacccaatttttaaactggtctaagccattcgataaggtgttcatcccagaccctaaaacgggcaggccctccttccagaacaatgcatggttcctcaacggccaattaaacgcctgttacaactgtgttgacagacatgccttgaagactcctaacaagaaagccattattttcgaaggtgacgagcctggccaaggctattccattacctacaaggaactacttgaagaagtttgtcaagtggcacaagtgctgacttactctatgggcgttcgcaagggcgatactgttgccgtgtacatgcctatggtcccagaagcaatcataaccttgttggccatttcccgtatcggtgccattcactccgtagtctttgccgggttttcttccaactccttgagagatcgtatcaacgatggggactctaaagttgtcatcactacagatgaatccaacagaggtggtaaagtcattgagactaaaagaattgttgatgacgcgctaagagagaccccaggcgtgagacacgtcttggtttatagaaagaccaacaatccatctgttgctttccatgcccccagagatttggattgggcaacagaaaagaagaaatacaagacctactatccatgcacacccgttgattctgaggatccattattcttgttgtatacgtctggttctactggtgcccccaagggtgttcaacattctaccgcaggttacttgctgggagctttgttgaccatgcgctacacttttgacactcaccaagaagacgttttcttcacagctggagacattggctggattacaggccacacttatgtggtttatggtcccttactatatggttgtgccactttggtctttgaagggactcctgcgtacccaaattactcccgttattgggatattattgatgaacacaaagtcacccaattttatgttgcgccaactgctttgcgtttgttgaaaagagctggtgattcctacatcgaaaatcattccttaaaatctttgcgttgcttgggttcggtcggtgagccaattgctgctgaagtttgggagtggtactctgaaaaaataggtaaaaatgaaatccccattgtagacacctactggcaaacagaatctggttcgcatctggtcaccccgctggctggtggtgttacaccaatgaaaccgggttctgcctcattccccttcttcggtattgatgcagttgttcttgaccctaacactggtgaagaacttaacaccagccacgcagagggtgtccttgccgtcaaagctgcatggccatcatttgcaagaactatttggaaaaatcatgataggtatctagacacttatttgaacccttaccctggctactatttcactggtgatggtgctgcaaaggataaggatggttatatctggattttgggtcgtgtagacgatgtggtgaacgtctctggtcaccgtctgtctaccgctgaaattgaggctgctattatcgaagatccaattgtggccgagtgtgctgttgtcggattcaacgatgacttgactggtcaagcagttgctgcatttgtggtgttgaaaaacaaatctagttggtccaccgcaacagatgatgaattacaagatatcaagaagcatttggtctttactgttagaaaagacatcgggccatttgccgcaccaaaattgatcattttagtggatgacttgcccaagacaagatccggcaaaattatgagacgtattttaagaaaaatcctagcaggagaaagtgaccaactaggcgacgtttctacattgtcaaaccctggcattgttagacatctaattgattcggtcaagttgtaa

SEQ ID NO: 38 - DNA, 2127 bp, Saccharomyces kluyveri
  misc_feature (1)..(2127): acs1
atgtcacccgctgtcgtcaaagtaggacaggcagaagattcgcaatcggatgttatccagaagctgaaggctcagaacaagagtggcgaagctgcacacttggagtacgagcatttgactagtgttcctgtgatcgagcagaagccggttaccgatcggttggctccagagttacaacagcactacaagcctcatttgtctggtcttgatgagtacaagcaactgtataaggaatcgttggagaatccagggaaattttttggtgagcgtgccagcacgttgttggactgggtcaaaccgtttgaccaggtttttatggctgatgatgagggcaaaccggcgtttgacaacaacgcgtggtttaccaacggtcagttgaacgcctgttacaacatggttgatagacatgctattaaaactccaaacaaagccgctattatttatgaagccgacgaaccgggcgaaggttacattttgacttatagagagttgttggaacaggtctgcagagttgcacaggtattgacacattccatgggggttcgcaagggggacaccgttgccgtttacatgcccatgattccccaggccttggtcaccttgttggctatctcccgtatcggtgccatccactctgtcgtgtttgccgggttcagttccaattccctacgtgaccgtatcaacgacgcctactcgaaagtcgtgattaccactgacgaatccaagagaggcggaaaagtgattgaaacgaaaaggattgtggacgatgctctaaaggaaacacctcaggtggaacacgttcttgtctacaaacgtacgcacagtccaaaggtcaacttccatgccccaagagatttggactgggacgttgaagtcaagaagtacaaggcttactctcctatcgaaccggttgattcggaacatcccttgtttttgttgtacacctccggttctacaggtgctccaaagggtgttcaacactcaaccgctggatatctattgcaggcaatgctatccatgcgctatacctttgatacccacaaggaggatatcttcttcaccgcgggtgacattggttggatcactggacacacctatgtcgtttatggtccgttgttgaccggttgtaccactatggtttttgaaggcactcctgcataccctaactactcgaggtattgggaaattgttgacaagtacaaggttacccagttctacgttgctccaaccgccttgcgtttgttgaagagggctggtgattctttcacagagggctactctttgaaatccttgcgttgtctaggtaccgttggtgaacccattgctgcagaagtttgggagtggtattccgaaaagattggtcgcaatgaaatacccatcattgacacttactggcagacggaatctggttctcatctagtcaccccaatggctggcggtgttacaccaatgaagccaggttctgcttctttcccattctttggtatcgagttggccgtgttggacccggccagtggcgaagagttgaagggtgaacccgttgaaggtgtcttggctatcaaaaaaccatggccatcttttgctaggaccatctggaaaaaccatgacagatatctggatacttacttgaacccttacccaggctactacttcactggtgacggtgctgcccgtgacaaggatgggtttatttggattttgggacgtgtcgatgacgttgtaaacgtttcgggccaccgtctatccactgctgaaatcgaagctgcaatcatcgaagatgacatggttgccgaatgtgccgttgtcggctatgcagatgacttgactggtcaagcggttgccgcctttgttgtgttgaagaataagaacagctgggccactgcgagcgaagatgagttacaaagcatcaagaagcacttgattctaactgtcagaaaggatattggcccattcgcggcaccaaaattaattgtgttggttgacgacttgccaaagactagatccggtaaaatcatgagacgtattctaagaaagattctatccggtgaagccgatcagctcggtgatgtttccactttgtcgaacccaggcatcgtcaagcatttgatcgattctgtgaaattttga

SEQ ID NO: 39 - DNA, 2052 bp, Saccharomyces cerevisiae
  misc_feature (1)..(2052): acs2
atgacaatcaaggaacataaagtagtttatgaagctcacaacgtaaaggctcttaaggctcctcaacatttttacaacagccaacccggcaagggttacgttactgatatgcaacattatcaagaaatgtatcaacaatctatcaatgagccagaaaaattctttgataagatggctaaggaatacttgcattgggatgctccatacaccaaagttcaatctggttcattgaacaatggtgatgttgcatggtttttgaacggtaaattgaatgcatcatacaattgtgttgacagacatgcctttgctaatcccgacaagccagctttgatctatgaagctgatgacgaatccgacaacaaaatcatcacatttggtgaattactcagaaaagtttcccaaatcgctggtgtcttaaaaagctggggcgttaagaaaggtgacacagtggctatctatttgccaatgattccagaagcggtcattgctatgttggctgtggctcgtattggtgctattcactctgttgtctttgctgggttctccgctggttcgttgaaagatcgtgtcgttgacgctaattctaaagtggtcatcacttgtgatgaaggtaaaagaggtggtaagaccatcaacactaaaaaaattgttgacgaaggtttgaacggagtcgatttggtttcccgtatcttggttttccaaagaactggtactgaaggtattccaatgaaggccggtagagattactggtggcatgaggaggccgctaagcagagaacttacctacctcctgtttcatgtgacgctgaagatcctctatttttattatacacttccggttccactggttctccaaagggtgtcgttcacactacaggtggttatttattaggtgccgctttaacaactagatacgtttttgatattcacccagaagatgttctcttcactgccggtgacgtcggctggatcacgggtcacacctatgctctatatggtccattaaccttgggtaccgcctcaataattttcgaatccactcctgcctacccagattatggtagatattggagaattatccaacgtcacaaggctacccatttctatgtggctccaactgctttaagattaatcaaacgtgtaggtgaagccgaaattgccaaatatgacacttcctcattacgtgtcttgggttccgtcggtgaaccaatctctccagacttatgggaatggtatcatgaaaaagtgggtaacaaaaactgtgtcatttgtgacactatgtggcaaacagagtctggttctcatttaattgctcctttggcaggtgctgtcccaacaaaacctggttctgctaccgtgccattctttggtattaacgcttgtatcattgaccctgttacaggtgtggaattagaaggtaatgatgtcgaaggtgtccttgccgttaaatcaccatggccatcaatggctagatctgtttggaaccaccacgaccgttacatggatacttacttgaaaccttatcctggtcactatttcacaggtgatggtgctggtagagatcatgatggttactactggatcaggggtagagttgacgacgttgtaaatgtttccggtcatagattatccacatcagaaattgaagcatctatctcaaatcacgaaaacgtctcggaagctgctgttgtcggtattccagatgaattgaccggtcaaaccgtcgttgcatatgtttccctaaaagatggttatctacaaaacaacgctactgaaggtgatgcagaacacatcacaccagataatttacgtagagaattgatcttacaagttaggggtgagattggtcctttcgcctcaccaaaaaccattattctagttagagatctaccaagaacaaggtcaggaaagattatgagaagagttctaagaaaggttgcttctaacgaagccgaacagctaggtgacctaactactttggccaacccagaagttgtacctgccatcatttctgctgtagagaaccaatttttctctcaaaaaaagaaataa

SEQ ID NO: 40 - DNA, 2052 bp, Saccharomyces kluyveri
  misc_feature (1)..(2052): acs2
atgtctgctaaagaacacaaagttgtccatgaggctcacaacgtcgagcctcgttatgctccagaacatttctacaagagtcaaccaggaaagggatatgtcaatgacttgacccattaccgtcagatgtacgagcaatccattaacgacccagaaggtttttttggcccattggcccaagaatatttgcattgggacagaccgtttactaaggttcaatcgggttccctagaaaacggcgatgttgcctggtttttaaacggtgaattaaatgcttcttacaactgtgttgatagacatgcttttgccaacccatctaagcctgctatcatttacgaagccgacgatgaaaaggaaaatagagttatcacctttggtgaattgttgagacaagtctccgaagttgccggtgtgttgaagagctggggtgttaaaaaaggtgacacagttgccgtttacatgccaatgattccagaagctgttgttgctatgttagcagttgctcgtttgggtgctatccactctgttatctttgctggtttctcatccggttctctaaaagagcgtgttgttgatgctggttgtaaagttgtcattacctgtgatgaaggccgtagaggtggtaagactgttcacgccaagaagatcgtcgacgaaggtttgtctggtgttgactctgtgtcccacattttggttttccaaagaactggttctcaaggtatcccaatgaaaccaggcagagatttctggtggcacgaagaatccgaaaagcacaggggctatttgccacctgtcccagtcaactctgaagatccattattcctattgtatacctcaggttctaccggatctccaaaaggtgtcgtccacacaactggtggttacttgttgggtgctgccttgaccactagatacgttttcgacattcatccagaagatgttttgttcactgcgggtgatgtgggttggattactgggcacacctatgccttgtacggtccactagctttgggtactgctaccattatctttgaatctactccagcttatccagactacggtagatactggagaatcattgaacgtcataaggccactcacttctacgttgctccaactgctttgagattgatcaagcgtgtgggtgaagctgaaattggtaaatatgatatctcgtccctaagagttctaggttctgtcggtgaaccgatctctccagatttatgggaatggtatcacgaaaagattggtaacaagaactgtgttatctgtgacactatgtggcaaacagaatctggttctcatctgattgccccactggcaggtgccgttccaaccaagccaggttctgctactgttccattttttggcgttaacacctgtatcattgatccagtttccggtgaggaattaaagggcaatgatgttgaaggtgtcttggctgttaaagctccatggccatccatggctagatctgtctggaacaaccactcccgttacttcgaaacctatatgaagccatatccaggctactactttactggtgatggtgctggtagggatcacgatggttactactggattaggggtagagttgacgatgttgttaacgtttctggtcacagattatccaccgctgaaatcgaagctgctttggtggaacacgaaggcgtctctgaatctgccgttgtcggtatcaccgatgaattaactggtcaagctgttgttgcttttgtctctttgaaggacggttatttgcaagaaaacgctgccgaaggggatgctgctcacattactccagataacttgcgtcgtgaactaattttgcaagttagaggtgagattggtccattcgctgcccccaagaccgttatcgttgttaaggacttgccaaagactagatctggtaagatcatgaggagaatcttgagaaagattgcctccaacgaagctgagcaattaggcgatttgtctactttggccaaccaagatgttgttccatcaattatctatgctgtcgaaaaccaattttttgctcaaaagaagaaataa

SEQ ID NO: 41 - DNA, 1047 bp, Saccharomyces cerevisiae
  misc_feature (1)..(1047): ADH1
atgtctatcccagaaactcaaaaaggtgttatcttctacgaatcccacggtaagttggaatacaaagatattccagttccaaagccaaaggccaacgaattgttgatcaacgttaaatactctggtgtctgtcacactgacttgcacgcttggcacggtgactggccattgccagttaagctaccattagtcggtggtcacgaaggtgccggtgtcgttgtcggcatgggtgaaaacgttaagggctggaagatcggtgactacgccggtatcaaatggttgaacggttcttgtatggcctgtgaatactgtgaattgggtaacgaatccaactgtcctcacgctgacttgtctggttacacccacgacggttctttccaacaatacgctaccgctgacgctgttcaagccgctcacattcctcaaggtaccgacttggcccaagtcgcccccatcttgtgtgctggtatcaccgtctacaaggctttgaagtctgctaacttgatggccggtcactgggttgctatctccggtgctgctggtggtctaggttctttggctgttcaatacgccaaggctatgggttacagagtcttgggtattgacggtggtgaaggtaaggaagaattattcagatccatcggtggtgaagtcttcattgacttcactaaggaaaaggacattgtcggtgctgttctaaaggccactgacggtggtgctcacggtgtcatcaacgtttccgtttccgaagccgctattgaagcttctaccagatacgttagagctaacggtaccaccgttttggtcggtatgccagctggtgccaagtgttgttctgatgtcttcaaccaagtcgtcaagtccatctctattgttggttcttacgtcggtaacagagctgacaccagagaagctttggacttcttcgccagaggtttggtcaagtctccaatcaaggttgtcggcttgtctaccttgccagaaatttacgaaaagatggaaaagggtcaaatcgttggtagatacgttgttgacacttctaaataa

SEQ ID NO: 42 - protein, 199 aa, Mycobacterium gastri
  misc_feature (1)..(199): rmpB (PHI)
MTQAAEADGAVKVVGDDITNNLSLVRDEVADTAAKVDPEQVAVLARQIVQPGRVFVAGAGRSGLVLRMAAMRLMHFGLTVHVAGDTTTPAISAGDLLLVASGSGTTSGVVKSAETAKKAGARIAAFTTNPDSPLAGLADAVVIIPAAQKTDHGSHISRQYAGSLFEQVLFVVTEAVFQSLWDHTEVEAEELWTRHANLE

SEQ ID NO: 43 - protein, 207 aa, Mycobacterium gastri
  misc_feature (1)..(207): rmpA (HPS)
MKLQVAIDLLSTEAALELAGKVAEYVDIIELGTPLIEAEGLSVITAVKKAHPDKIVFADMKTMDAGELEADIAFKAGADLVTVLGSADDSTIAGAVKAAQAHNKGVVVDLIGIEDKATRAQEVRALGAKFVEMHAGLDEQAKPGFDLNGLLAAGEKARVPFSVAGGVKVATIPAVQKAGAEVAVAGGAIYGAADPAAAAKELRAAIA

SEQ ID NO: 44 - DNA, 1161 bp, Saccharomyces cerevisiae
  misc_feature (1)..(1161): SFA1
atgtccgccgctactgttggtaaacctattaagtgcattgctgctgttgcgtatgatgcgaagaaaccattaagtgttgaagaaatcacggtagacgccccaaaagcgcacgaagtacgtatcaaaattgaatatactgctgtatgccacactgatgcgtacactttatcaggctctgatccagaaggacttttcccttgcgttctgggccacgaaggagccggtatcgtagaatctgtaggcgatgatgtcataacagttaagcctggtgatcatgttattgctttgtacactgctgagtgtggcaaatgtaagttctgtacttccggtaaaaccaacttatgtggtgctgttagagctactcaagggaaaggtgtaatgcctgatgggaccacaagatttcataatgcgaaaggtgaagatatataccatttcatgggttgctctactttttccgaatatactgtggtggcagatgtctctgtggttgccatcgatccaaaagctcccttggatgctgcctgtttactgggttgtggtgttactactggttttggggcggctcttaagacagctaatgtgcaaaaaggcgataccgttgcagtatttggctgcgggactgtaggactctccgttatccaaggtgcaaagttaaggggcgcttccaagatcattgccattgacattaacaataagaaaaaacaatattgttctcaatttggtgccacggattttgttaatcccaaggaagatttggccaaagatcaaactatcgttgaaaagttaattgaaatgactgatgggggtctggattttacttttgactgtactggtaataccaaaattatgagagatgctttggaagcctgtcataaaggttggggtcaatctattatcattggtgtggctgccgctggtgaagaaatttctacaaggccgttccagctggtcactggtagagtgtggaaaggctctgcttttggtggcatcaaaggtagatctgaaatgggcggtttaattaaagactatcaaaaaggtgccttaaaagtcgaagaatttatcactcacaggagaccattcaaagaaatcaatcaagcctttgaagatttgcataacggtgattgcttaagaaccgtcttgaagtctgatgaaataaaatag

SEQ ID NO: 45 - DNA, 900 bp, Saccharomyces cerevisiae
  misc_feature (1)..(900): YJL068C
atgaaggttgttaaggaatttagtgtctgtggtggcagattgatcaagttgtcacataactcgaactctaccaagaccagcatgaacgtcaatatctatttgcctaagcactattacgcccaagattttccaagaaataagcgtatcccaactgtgttttacctttctggcttgacgtgcacgccagacaacgcctctgagaaggctttttggcagtttcaagctgacaagtacggatttgcaatagtctttccggatacgtccccacgtggtgacgaagtagccaatgatcctgagggctcctgggattttggacagggcgccggattctatctaaatgccacccaagaaccatacgcccaacattaccagatgtacgactacattcacaaagaactcccacaaacattagattctcattttaacaagaacggtgacgtaaagctggacttcttggacaatgttgccatcacaggccattcgatggggggatatggtgcaatttgtgggtatttgaagggctattccggaaagagatacaaatcttgttctgccttcgcccctatcgtgaacccttccaacgttccctggggtcaaaaagcgtttaaaggttatctgggcgaagaaaaagcccagtgggaagcgtacgacccatgtttattaatcaagaatattagacatgtgggcgacgacagaattttgatccatgtaggagactccgatccctttttggaagaacacttgaaaccggaattactacttgaggcggtgaaagccacttcatggcaggactacgtggaaataaaaaaagttcacggctttgatcactcctattactttgtcagcactttcgttccagaacatgctgaatttcatgcgcgaaacttgggtttgatttga

SEQ ID NO: 46 - DNA, 1131 bp, Saccharomyces cerevisiae
  misc_feature (1)..(1131): FDH1
atgtcgaagggaaaggttttgctggttctttacgaaggtggtaagcatgctgaagagcaggaaaagttattggggtgtattgaaaatgaacttggtatcagaaatttcattgaagaacagggatacgagttggttactaccattgacaaggaccctgagccaacctcaacggtagacagggagttgaaagacgctgaaattgtcattactacgccctttttccccgcctacatctcgagaaacaggattgcagaagctcctaacctgaagctctgtgtaaccgctggcgtcggttcagaccatgtcgatttagaagctgcaaatgaacggaaaatcacggtcaccgaagttactggttctaacgtcgtttctgtcgcagagcacgttatggccacaattttggttttgataagaaactataatggtggtcatcaacaagcaattaatggtgagtgggatattgccggcgtggctaaaaatgagtatgatctggaagacaaaataatttcaacggtaggtgccggtagaattggatatagggttctggaaagattggtcgcatttaatccgaagaagttactgtactacgactaccaggaactacctgcggaagcaatcaatagattgaacgaggccagcaagcttttcaatggcagaggtgatattgttcagagagtagagaaattggaggatatggttgctcagtcagatgttgttaccatcaactgtccattgcacaaggactcaaggggtttattcaataaaaagcttatttcccacatgaaagatggtgcatacttggtgaataccgctagaggtgctatttgtgtcgcagaagatgttgccgaggcagtcaagtctggtaaattggctggctatggtggtgatgtctgggataagcaaccagcaccaaaagaccatccctggaggactatggacaataaggaccacgtgggaaacgcaatgactgttcatatcagtggcacatctctggatgctcaaaagaggtacgctcagggagtaaagaacatcctaaatagttacttttccaaaaagtttgattaccgtccacaggatattattgtgcagaatggttcttatgccaccagagcttatggacagaagaaataa

SEQ ID NO: 47 - DNA, 1095 bp, Candida boidinii
  misc_feature (1)..(1095): FDH3
atgaagattgtcttagttctttatgatgctggtaagcacgctgctgatgaagaaaaattatatggttgtactgaaaataaattaggtattgccaattggttaaaagatcaaggtcatgaactaattactacttctgataaagaaggtgaaacaagcgaattggataaacatatcccagatgctgatattatcatcaccactcctttccatcctgcttatatcactaaggaaagacttgacaaggctaagaacttaaaattagtcgttgtcgctggtgttggttctgatcacattgatttagattatattaatcaaacaggtaagaaaatctcagtcctggaagttacaggttctaatgttgtctctgttgctgaacacgttgtcatgaccatgcttgtcttggttagaaatttcgttccagcacatgaacaaattattaaccacgattgggaggttgctgctatcgctaaggatgcttacgatatcgaaggtaaaactatcgctaccattggtgctggtagaattggttacagagtcttggaaagattactcccatttaatccaaaagaattattatactacgattatcaagctttaccaaaagaagctgaagaaaaagttggtgctagaagagttgaaaatattgaagaattagttgctcaagctgatatcgttacagttaatgctccattacacgcaggtacaaaaggtttaattaataaggaattattatctaaatttaaaaaaggtgcttggttagtcaataccgcaagaggtgctatttgtgttgctgaagatgttgcagcagctttagaatctggtcaattaagaggttacggtggtgatgtttggttcccacaaccagctccaaaggatcacccatggagagatatgagaaataaatatggtgctggtaatgccatgactcctcactactctggtactactttagacgctcaaacaagatacgctgaaggtactaaaaatattttggaatcattctttaccggtaaatttgattacagaccacaagatattatcttattaaatggggaatacgttactaaagcttacggtaaacacgataagaaatag

SEQ ID NO: 48 - protein, 380 aa, Ogataea polymorpha
  misc_feature (1)..(380): Glycerol dehydrogenase
MKGLLYYGTNDIRYSETVPEPEIKNPNDVKIKVSYCGICGTDLKEFTYSGGPVFFPKQGTKDKISGYELPLCPGHEFSGTVVEVGSGVTSVKPGDRVAVEATSHCSDRSRYKDTVAQDLGLCMACQSGSPNCCASLSFCGLGGASGGFAEYVVYGEDHMVKLPDSIPDDIGALVEPISVAWHAVERARFQPGQTALVLGGGPIGLATILALQGHHAGKIVCSEPALIRRQFAKELGAEVFDPSTCDDANAVLKAMVPENEGFHAAFDCSGVPQTFTTSIVATGPSGIAVNVAVWGDHPIGFMPMSLTYQEKYATGSMCYTVKDFQEVVKALEDGLISLDKARKMITGKVHLKDGVEKGFKQLIEHKENNVKILVTPNEVS

SEQ ID NO: 49 - protein, 380 aa, Ogataea polymorpha
  misc_feature (1)..(380): Formaldehyde dehydrogenase FLD1
MSTVGKTITCKAAVAWEAGKDLTIETIEVAPPKAHEVRVKIAYTGVCHTDGYTLSGNDPEGQFPVIFGHEGAGVVESVGEGVTSVKVGDHVVCLYTPECRECKFCKSGKTNLCGKIRATQGKGVMPDGTSRFTCKGKTLLHYMGCSTFSQYTVLADISVVAVDPKAPMDRTCLLGCGITTGYGAAINTAKISEGDNIGVFGAGCIGLSVIQGAVKKKAGKIIVIDVNDAKKDWAFKFGATDFVNPTKLPEGQSIVDKLIEMTDGGCDFTFDCTGNVQVMRNALEACHKGWGESIIIGVAPAGKEISTRPFQLVTGRVWRGCAFGGIKGRTQMPDLVQDYMDGEIKVDEFITHRHPLNDINQAFHDMHKGDCIRAVVTMDE

SEQ ID NO: 50 - protein, 362 aa, Ogataea polymorpha
  misc_feature (1)..(362): Formate dehydrogenase
MKVVLVLYDAGKHAQDEERLYGCTENALGIRDWLEKQGHELVVTSDKEGQNSVLEKNISDADVIISTPFHPAYITKERIDKAKKLKLLVVAGVGSDHIDLDYINQSGRDISVLEVTGSNVVSVAEHVVMTMLVLVRNFVPAHEQIISGGWNVAEIAKDSFDIEGKVIATIGAGRIGYRVLERLVAFNPKELLYYDYQSLSKEAEEKVGARRVHDIKELVAQADIVTINCPLHAGSKGLVNAELLKHFKKGAWLVNTARGAICVAEDVAAAVKSGQLRGYGGDVWFPQPAPKDHPWRSMANKYGAGNAMTPHYSGSVIDAQVRYAQGTKNILESFFTQKFDYRPQDIILLNGKYKTKSYGADK

SEQ ID NO: 51 - DNA, 1755 bp, Saccharomyces cerevisiae
  misc_feature (1)..(1755): dihydroxyacetone kinase DAK1
atgtccgctaaatcgtttgaagtcacagatccagtcaattcaagtctcaaagggtttgcccttgctaacccctccattacgctggtccctgaagaaaaaattctcttcagaaagaccgattccgacaagatcgcattaatttctggtggtggtagtggacatgaacctacacacgccggtttcattggtaagggtatgttgagtggcgccgtggttggcgaaatttttgcatccccttcaacaaaacagattttaaatgcaatccgtttagtcaatgaaaatgcgtctggcgttttattgattgtgaagaactacacaggtgatgttttgcattttggtctgtccgctgagagagcaagagccttgggtattaactgccgcgttgctgtcataggtgatgatgttgcagttggcagagaaaagggtggtatggttggtagaagagcattggcaggtaccgttttggttcataagattgtaggtgccttcgcagaagaatattctagtaagtatggcttagacggtacagctaaagtggctaaaattatcaacgacaatttggtgaccattggatcttctttagaccattgtaaagttcctggcaggaaattcgaaagtgaattaaacgaaaaacaaatggaattgggtatgggtattcataacgaacctggtgtgaaagttttagaccctattccttctaccgaagacttgatctccaagtatatgctaccaaaactattggatccaaacgataaggatagagcttttgtaaagtttgatgaagatgatgaagttgtcttgttagttaacaatctcggcggtgtttctaattttgttattagttctatcacttccaaaactacggatttcttaaaggaaaattacaacataaccccggttcaaacaattgctggcacattgatgacctccttcaatggtaatgggttcagtatcacattactaaacgccactaaggctacaaaggctttgcaatctgattttgaggagatcaaatcagtactagacttgttgaacgcatttacgaacgcaccgggctggccaattgcagattttgaaaagacttctgccccatctgttaacgatgacttgttacataatgaagtaacagcaaaggccgtcggtacctatgactttgacaagtttgctgagtggatgaagagtggtgctgaacaagttatcaagagcgaaccgcacattacggaactagacaatcaagttggtgatggtgattgtggttacactttagtggcaggagttaaaggcatcaccgaaaaccttgacaagctgtcgaaggactcattatctcaggcggttgcccaaatttcagatttcattgaaggctcaatgggaggtacttctggtggtttatattctattcttttgtcgggtttttcacacggattaattcaggtttgtaaatcaaaggatgaacccgtcactaaggaaattgtggctaagtcactcggaattgcattggatactttatacaaatatacaaaggcaaggaagggatcatccaccatgattgatgctttagaaccattcgttaaagaatttactgcatctaaggatttcaataaggcggtaaaagctgcagaggaaggtgctaaatccactgctacattcgaggccaaatttggcagagcttcgtatgtcggcgattcatctcaagtagaagatcctggtgcagtaggcctatgtgagtttttgaagggggttcaaagcgccttgtaa

SEQ ID NO: 52 - DNA, 1776 bp, Saccharomyces cerevisiae
  misc_feature (1)..(1776): dihydroxyacetone kinase DAK2
atgtctcacaaacaattcaaatcagatggaaacatcgttactccctacctacttggccttgctcgaagcaatcccggccttacagtgattaagcacgacagagtggttttccggactgcgtcagctcctaattcagggaaccctcctaaagtttcattggtttctggaggtggcagtggtcatgagccaacgcatgccggttttgttggtgaaggtgccttagatgcgattgcagcaggtgccatttttgcttctccttcaactaaacagatctattctgctattaaagctgttgaatctcctaagggtaccttgatcattgtaaaaaattacaccggtgatattatacattttggtctcgctgctgaaagagctaaagctgctggaatgaaagtcgaactggttgctgtaggagatgatgtctctgtcggtaagaagaaaggttctttagtcgggcgtcgaggtctcggagccaccgtattggtgcataaaattgctggggcagccgcttctcatggactggagttggcagaagttgccgaagttgctcagtcagtagttgacaatagtgtcacaattgcggcatctcttgatcactgcacggttcctggccacaaacctgaagccattttgggcgagaatgagtatgaaatcggtatgggtattcataacgagtctggtacctataagtcttctccgctgccatcgatttctgagctcgtttcccagatgcttcctcttcttctcgatgaggatgaagaccgttcttatgtgaagtttgagcccaaagaggacgtagttcttatggttaacaacatgggtggtatgtctaatctagaattgggttatgctgcagaggtcatttctgaacaattgattgataagtatcaaattgtgcccaagagaacgattactggagcattcattactgcattgaatggtcctgggtttggtatcactcttatgaacgcttcaaaagctggtggcgatatccttaagtatttcgattatcctaccacagcgagtggatggaatcaaatgtaccattctgccaaagattgggaggtacttgccaaagggcaggttcccaccgccccctctttaaagacattgaggaatgaaaaaggttcgggtgtgaaagctgattatgacacttttgctaaaattttgcttgctgggattgcaaaaattaacgaggttgaaccaaaggttacttggtacgataccattgcaggagatggtgattgcggaactactcttgtgtccggtggtgaagcattggaagaagctattaaaaaccatacgttgcgcctcgaggatgctgctcttggtatcgaagatattgcgtatatggttgaggattctatgggtggtacgtccggtggtctgtactctatctatctttctgctctcgcacaaggagttagggattctggggacaaggaacttactgcggaaactttcaaaaaggcatcaaacgttgcactagatgctttgtataagtatacgagagcccgtcctggttacaggactctgatcgatgctctgcaaccttttgtcgaagcgctgaaagccgggaagggtcccagagccgccgcccaagctgcttatgatggtgccgaaaagacaaggaagatggatgcccttgttgggcgtgcttcttacgtggctaaggaggagctgagaaaactcgacagcgaaggtggattaccagatccaggagcagttggtcttgctgcactactcgatggatttgttacagctgctgggtactag

SEQ ID NO: 53 - DNA, 2999 bp, Saccharomyces cerevisiae
  misc_feature (1)..(2999): upstream sequence of DAK1
ctttctagttcctctgggaaaggaatctctaagtcatttgtatcatacaagaagcttttcagaaagacattcaaggacatgtagacatgttccctgaggacttcaattttcctcttaaccatcatttcaattaaagattcattagaagactcaacgtccaaaggatatgatgctatttcatcggtaatgaatttgtatctcatggctaaaaacacacttaataatgatttattagtctttccatcaaatggtggaattgaattcaagtactgtagtattttctttagtgagtttactgatacattagtagacagtaattttaccagcccggataacatggtagtactaatttcgtttaaaactttttcgcaaacttcatccacaatggttgaaccagggaatcttgacctcagggaagtagtgtgtgtgtatagcatgacggcctcctggtaatgacctgttctgatacatgtgcgggccaaaaatggtagttccatcaaatccgtaatgctatccaaattttctagtacagtgactaatgtgtcagatctaatgtcgtccttgtcatcttctttagttgaaattctgtttcttaacctacttaatgccttgtgaaactcatcctccttcttctttcttgccagattgttactctcagtcgtcatgatgcgacctgtgtcgttgtcttctttatcgtcttccaaaaagtcatcaatagaaacgctttcattgtttatatcgtcatttgtaacgtttcgatcggctgccttattaatattagtatccaattcccataattgctccaaagacttggctatatcatccaattgagccctatcatcgttttcaagaatatttccgataatctgagacgtattatctagtagtgttttccttatctttctatcaagggcagataattcagcatcaatttctgctatgtcttcagttatgctaccaggaacagccctagatgaaaaataactttcatagtctttcgtgttagattgtaggatatcctgtaggaaatcaagacttaaccgtttttgttcctctgttaaatcatcagaaatgaggctgtttagtatcagctccattttactcaatgattatgtttattgttgaaatatgttccctcaaatgtcctaacacttctatgattattttttctgtgcttctcttttcagtatgttactacgctatatttttagacattgaagccatgatcgcgagatcgatctaatgtacgtataaaaagaaaatggacttcaagagtacaactaactaaaggaaaatccaactcttcctataaatttagaattagcatattcaaaaaagaagaaacaaagcactcacgatgggggtcgaacccataatcttctgattagaagtcagacgcgttgccattacgccacgcgagctaaatttcttgaattgttgggtaaacaaatattactaatacaaatgttattagaaaccaaaaatgcacttttccggggttaatatatatgattgagtgatggatgaagaatgagataattgtttaaattctatagttgtcaagcgctgtgataccagatacacaaaatgtactagaggttctcctcgagaatatgaaaatccacaatagagaaccgatatttctgtgtaggaatattattatttcttctttcattttgtatgttctcgttcattttcctagcacattatcaaccctttcatttcaatttccattaaaattggtgactgtttctcaatatttatttgaccgtcttatacccaatatggtaatataccagtaatataaatactagtcgttagatgatagttgcttcttattccgaaaatgagtatggaagtgttgcatatgatagggcggctacagtgatggtaaacataagatactttagcgggaaattagcaactggaagttaaattatctagacataagtgtggcggtcacgctgaacgcaggagatcggatagattgataagctgatcaagaacattgatcggtttgttgtttaaagaatggtttttgaaaacgtttgaccagttgcttctcccagacgcttaccgatatgatgataaagataatatcttcaattgaataccccgtggatcagcacgaataacagaaaaaaagggtgaaattcaccgtaagcatgatacgcactacgttcttcttacctttgccaacgtgttgtctttgacgtacgtaattatgggagatcgttgatgattagccccagctcactttcttcttaatgactgacccgctactatcaaaattaaggtgtcaaatatcatgatgaatgaggtctctaggcgactcaattatacatcttttagagatttttttactacttgcagataatttctcaagggattagattcaaatctggcttgtcaattacgcccttttcaagctcatcaaattgcgtatgtcattcatgcttccattaggaaccatagaagcatggctgaaatggcaatatacggcttcccaatttcaactctaaagtaatggcggtcgaatttaatctatattttacagttttatacgtactttaaaagcaatcagtaaacacctctggtgctattcaagggttttttgcctttatttgttactgtcaattgtctggcgctgtgataaaaaacaaggcataaagctcccccgtcatgaacattaagactcgctagacgagagagtgaaatataatgcatttcctgatttaaatgcgctacaaacatggtgtaaatctggcccggagtgagtgcttgccaatttggcttctaagggagaaagatcaaaccactcccaattgcgtcattttgaaagagtggccacctcgcgagcgtctgtcgaactaactgatgaataaatatataaggagaaaatcacttcaacttcgctacaagtagtcactatttgtagcaactgtaaacgaacacatcaaagaataagattacattctatatctaagactaaattttaa

SEQ ID NO: 54 - DNA, 3000 bp, Saccharomyces cerevisiae
  misc_feature (1)..(3000): downstream sequence of DAK1
gtacttggctcacgaatacatatcaagatacttatgatatatatatatagaaaaagcttacttttcttggagttattgttattatcatcgcgaagaacgattgtataacccggttcaacgcgaaacgaatcgttaaactggtgaaatgttaacgcgagtgtcagagatatacatagtatgagagtagctagatgttgaatcggtggtaagaacaagaaggaaataccgttaacaagtgaaggaacaatctagtattgttgaacaagaattatgagtaccgactttgatagaatttacttgaaccaatctaaatttagcggtagattccgtattgctgattctgggttagggtggaaaattagtaccagtggtggctctgcagcaaatcaggcaagaaaaccatttttattaccagccacagaattatctaccgtccaatggagtaggggctgcaggggttacgacttgaagataaataccaaaaatcaaggtgttatccaactagatggattttctcaggatgactataacttaatcaagaatgatttccatcgccgttttaatattcaggtagagcaaagagaacattccttacgtggttggaactggggtaagacagaccttgccaggaatgaaatggtttttgctttaaatggtaaaccaacttttgaaattccttatgctagaataaataatacaaatttgacctctaaaaatgaagtaggaatagaatttaatattcaagatgaagagtaccaaccagccggtgacgaattggtagagatgaggttctatattcctggtgttattcaaacaaacgtcgatgaaaacatgaccaaaaaggaagagtcaagcaacgaggtcgtaccaaagaaagaagatggtgctgaaggagaagatgtacaaatggcagtagaggaaaagagtatggcagaagcattctatgaagaactaaaggaaaaggcagacatcggggaagtcgctggtgatgcaatagtttccttccaagacgtcttttttaccacgccaagaggtcgttatgatatcgatatttacaagaactccattagactcaggggtaagacctatgaatacaaattgcaacatcgtcaaatacaaagaattgtttcgttaccaaaggcagatgatatccatcacttattggttttggcaattgaacctcctttacgtcaaggacagaccacctacccctttcttgtcttacaatttcagaaagatgaggaaacagaagtgcaattgaatctagaagatgaagattatgaggaaaattataaggataaattgaaaaaacaatatgatgctaaaactcatatagttttaagtcatgtattaaaaggtctgactgaccgtagagtcattgttcctggagaatataaatccaaatatgatcagtgtgcagtttcatgttctttcaaagcaaacgaaggttatttgtatccattagataacgctttcttctttttaactaagccaactttgtacataccattcagtgatgttagcatggtaaacatttcaagagcaggacaaacttctacgtcatcgaggacgtttgatttggaagtggtactgcgttcaaatagaggttctaccacttttgccaacatcagtaaggaagagcagcaattattggaacaattcctaaagtctaaaaacctaagggtgaagaatgaagatagagaggtacaagaaaggttacaaaccgctttaggttcagacagtgacgaagaggatattaatatgggttccgctggtgaagatgatgaatcagtagatgaggattttcaggtcagctctgataatgacgcagacgaagttgcagaagagtttgattcagatgcggctttaagtgatgctgaggggggtagcgacgaagaaaggccttcgaagaagcctaaggtagaatagtaataattttagactgtataagttaaatttattgatattgtgtaaaaactaactaatatattttgccaattgatattatcatgacatggtgagtgtaagacaccacctcttaattactggtgttattctatacatttatttgaaattggttttgttttgcaaaatatttatgttttgttaatctcctctaccctttcaatgcttgaaaaatactttcaacttttcgattgggtgatgaaaaaaagacaaatagtgtaaagggttcaaaaataaataacaagcaagagaaagggactttgcttttctcatttagtcaccagtaagttatgtcatggtgtagaataacgaattacagaaaactaatataactgatgaaagaccagggagtaaaatggctttgactcagtttgaaaatgatttggaaatattaagagatatgtacccagaactggaaatgaaatcggtaaaagtagaggaggaaggtgaattccctcaaagaattaacggaaagttactgttcaagatatcactattggccgatgtaaatattgagttcggcgagcaacatatgttactttcaaacttatctaatgaatgcgtggagttcaccatatatagctgtcattatccggacattcgacggtgtgttgttatggatatcaaatccttatggatatcaacagatgaaaagaagatgttaattgacaaagcgctgagactcgttgaagaaactgtagatatgagtattgagttcgcggattcgtttacctccatccttatcctcatctttgggtttcttatagatgatacagctatattactattccctaatggaataagaaagtgcctgacacaggatcagtatgacttgtttaagcagataagtgaggaagccaccctccaaaaagtgagcagatctaactaccattgttgtatttgtatggaaatggaaaagggtgttagaatgatcaaattgccatgtgaaaatgcgaatgtagaacactatctttgcagaggatgcgccaaatcttatttcactgcaatgattcaggaaaaccgaatatccagtgt

SEQ ID NO: 55 - DNA, 3000 bp, Saccharomyces cerevisiae
  misc_feature (1)..(3000): upstream sequence of DAK2
gataatgacaataattttctttgagcacaattagtttatttggcaacttgctctttattattagtaaatatagcacatattcatataaaacacatcttcagtgggattacctagttgatggtgccaggaattttcctatcgtcaacgagctgaaaattttattatttttattaataacataatgtgagactcctggtttgacatttaattttacgtattagtcgaattttgttcttgcctacaataaaaagacaattaagccgcaggcagtctcattctttattacaaaaacaaaacgatagaatttagagcacaagtaagagatggtaacaaagtcacggctcccggatgtagtatgtcgtcaaataataagttcgtgaaattaataattaggttataaatcgtaaaaaattgaaaatattaattatgacgaagtaggcacagatttcttgctgccagtgttgctgttgctgttaacaccagattcatctgagacagtgccatcattctggtggaactccgcatgtaaaagtttacctaccctattctcaatataaccgttgtctgggtagttgactggggattcgcatccagtaaaaatgaaaatgtcatatatgagtgctccagcaataccgccggcaattggaccaccccaggctccccatgtccaccaccaatgtgtgagatgaaaagcatgtggaccatagccaatcatggaagcaaatatgcgaggaccgagatctcttgcaggattgattgtgaaacttgtttgatatccaagggccataccaattgcagcgactaagaatccaataattaatgcggtcataccattgccaggtggagcattactatcatccaatagcgccatcaaacaacccacaagtatagaggctcctatgaattcgtcaaagaaggcatttctccacgtgacgtaagactttggatcagtaaacaaacacgcaccggtcgccgttgttcttatgtgcggacctccctcaaattctgtgatagagctccaaaaataaccataagccatagctcctccaaaatatgcaccgataatctgagcaacaatatatacgggcacctttttccaggggaattttcgaaaaattgccattgaaatcgtaacagcagggttaatatgaccaccactaataccgcctgcgacgtaaacaccaagcatacaaccgaacccccatgcaaatgatagggattcataggaaccaccactaccttttgttacagttgcttgaagattaccaccaacaccaaaaatgacaagaactagtgtcccgagaaactccgcaaacggttctcgcatatgatagcgaatttttgcccaaaagttaggaaatgtcattatatccgcgtcttcatcttctgaggcaccaatttcattaccatctaatgcactcgtacttttattttcctcttcaataagttcttcaggaagcttcgtataaaccggagtagaaccatcgagggtttgcatgttctttaattttctggattctgcatcggctatggaggatgcgtagctttcatcgcctaatgaaaaattgacattatgcgaagtacgctttttcttacgtgaaacattctcaatatgcgtgttagagggcttttgtgggttttcaggctctaacttagtaggtttcacatctgcaccaacagtattatcgcgttgcgtttgagcttcgatgtcaccaagctttttacccggtgctgcacctctaaggagccttttccagtctttcactgacgtagtagaacccccccttgaaatatcggtcgaccctcgtctcgatgatgcgcgggaccttgcatccatattcttttgtatcatctttgctgccaagcctcgatccaatacatgcggtaacggctgatttaaactccaaacaggcctgttcctgctactacccatagttggatttaattgccgatatagtgggtctacataattattactgttgccttgatttacccccctggatagataatcttgcattatacgtgcttgctcagcatctgaaaaactgtttaaagtcttcaaattaggcaaagaataagtaaaacccatggaagggacctgtggttgtaccgaatccttcctctcgtcaaccacttttttcaagggtgtcaaagcagaaagtttatttctcgatgatcctccagtttcaattgcttttcgagtctcacctagctttcgcctcaagagagtgctgtcattttcgttattttccctagattttttgacactttcttcccaagctattttaccattaggttcttcttttagcgttggtggccgtgtactctcggaagaggaggatgacctcccagattcgtaactcattacttgggtctagatcatatatcagaggagcgttatactgtgcgattatacgcttctttttatatgaataagggggagacatggtgaaaaggtaccagaacttttgatcgaccaagactaggtaaagctcaaacaacgtttataactcaaatttccggggtaagtggggtaccggaaaattatgatattccggagcggagttatcaacggagaaaactaggccttctgatggaacttaatttaaaaaattaatcacaacctatgcatattattcccgcagagggtgattgtgagtaaatccctgcacagaaacaattcccgccaggccataactagattctaaattatttaactcataatttcatgaaatcgtatcgtagtaccaaatagggagatattgagccaagtaaattcttacgtcaccatagttggataattaagtacttgatattgtataaggatctcaacaatacgagaaggggaaaataccgcaatgtgtgattgaattttcaaactttggatcattaaatatatataaatgaacccagatcagccctttttttttctagtattgtctgtaaagtgtattttacctcaaaatctgacaaaacccaactacaattgactaaataatc

SEQ ID NO: 56 - DNA, 3000 bp, Saccharomyces cerevisiae
  misc_feature (1)..(3000): downstream sequence of DAK2
aattgctcgtacacactagaagccaaacataacagctttaaaggctttcatttttgaactttttaaaaaattgaatactccaactgaaggtgaactagttgtgtctctgaatatatttttatagatatacgaattgatgaagtaccgcaaattaagctaaaaagtaatgcttcttgcagcttttaattgttctttctgcaatctacaattacttttcttgattccttctccgttcccctgtgttgtctggaagtataatttgtccaggaagattttttgaatagccattgttttctttaaattaaatcggagtgtttaaatccattccaatctcttttttctcgcaagtcaacaaacaggtgttaactttcttttccccgctgttttcttacctatgaatagtctcaattcctttttagaagatctgcacattctctgatactatgaacaagttctaggatagcaatctaagttttatgattctcttatttcggattcgatttcaataaagatcgtagtattagaagtatagaatgtattgtaattttttttcctaatcttattaattcatggaaggcattgaactcaacagcatattttaaatgtttgtatcttgttttctctttcaaaaaaaaaatggtgtcattcattattttatggtcaaccctatacatcaatttttctctgaaaatattgacaaataaagtagttgattcttgttctaccaattagtgatattatgcatgactgttaacaactttttgactaatctctgaaatcatatgaagatcttgctgcatttcatgcatctaagaaatcaacctatatcaacagatttcaataattactctaaacttatgctgtaacttagaaagtaaccagcctgtgttgactgattgagttgcgtattaactgcgcctagtcatttcaacacttataatttgcttcagcttaagtgtggttcatctttttttttctggaaactttgcatgccctcaaagcatgagtagttagttatctttttgacaatgatctcttttgaaaatatctactgtagatttgcatggacgcacgtcgcccatacgccaaactttggcaatgatactcgttattcgtaatatcagtccgtcaaggtgctgtgatttctctattttatattgcctattattttttcaaatgatttgagccgttttaaattgagtatgcaatgagtcttttgaatcaaccgtaaggcagttccataaccactgccacgaatacgtttcactaccttgaagaatctctaatgtaggccgtattcttcgcacttagttctgacgatgtagacatctcattatataagagcataagcgcctgtttctagaatcatttcttcgtgacccagctttttgagttatttcgcggtattttgaaacatttctcgagcttgacgtgaacatccttatatttcatgacaaactcgatcattggaacatccctgcctcgattttagagctagtatcaaatttcaatctctttgtgatggagccccgctcctatttcaaaagagaagtttcttgtatgcatatgttattgaagtctgattatagcaagtgcaatgtcgtctcaattattttaactatttttagccatacatgttagttatcctcaaagagagcctccagactgggaagcagtgtttgtcatttcaaataagtagatttcacagtttgtatgattttcgaagccaggattcattgggctttgagtaaagagaagccgcgtattacgaacagcttacgatattgtaaaatattcccttattgtggtgccccaatggatacatgccagagaaatgtctgtgaaattgaacaattacaatgacgagagcaagtaatccggcggccttgtctctctttcactagtaccgtctatatctcttgagcgccaatatgcgaaagctttcacaaggttgatgttcatggtattcggcgtcgatagcgaattgcttactaagaaacattagggtgcagtacagccttgtttttccagttcgactaacctttttcttggcagtatggagactgactaggtctcccaaacattcattgtaactgctgtttaaagattttgttctaacctaaattcaagtgagaagctgaacatgtgtctctacttatgatatcacgacagcaaatactaatcttgccataaatagtctagcgttttgcaacttacctctagatatattttatttcttgaggaaccgttttcgtcggtaataacaaaatactactgaaacgccacagcattgagagaatacgttatcgattacggctttcttctcgctccagatgtcgcgggtaagatattcacctcaaacttttcttgttgagtgtcgtcacaaatctagaacctacatgccatctcaacgatttttctggagaaaggcctcactccgttccgtacgtaatgcatagataaagtatcaggatcttcacgatgctcgagagttacttagtagtctgagtttatgcgaaaaaaactccgccgttgtaataatcgggaatacacagaagtagtactgcactatcactgggatactcaaaaaccttctttttaacttttctatcccacaaatagaacataggaaagaacattgactcctccacttgaagttaaattacaggaacaaacgcctaactataatttcgacattgttgcatcaacgaatcgaccgaaagaaaaatctggagttgcagttatcacttgtatgtgcactaagatttatatttttactcctgagatctgccaaatcggtagcttattgaactgcgttcctttttcccctgagttctcgaggtacctgcggctttgtctgtgccatctcccccactttaaagtaccccacgttactaccgcgtttttccccacccccggcttaataaattagctatatcttgttgacttaaatacggagaaaagaagaaaaccttcaagaaatgcttcattgtcttgtca

SEQ ID NO: 57 - DNA, 2028 bp, Zygosaccharomyces bailii
  misc_feature (1)..(2028): ACS
atgacagtcaaagaacacaaggtagtgcacgaggcacaaaacgtagaagcgctgcatgcgccagagcatttttacaagtcacaaccaggccccagctacatcaaggacatgaagcagtacaaggagatgtacaaacaatctgtggaagacccagaaaacttctttggtgaaaaggctagggagctgctggattgggacagaccttttacgagaagcaagtacggttcgttggaaaatggcgatgtcacgtggtttttaaatggtgaattgaacgcagcctacaactgtgttgacaggcacgcttttgcaaacccagacaagcccgcattgatctacgaggctgacgaggaggctgacaacaggatgataaccttcagcgagctgctgagacaggtttcgcgggtcgctggggttctacaaagctggggagtgaaaaagggcgacactgtggcagtgtacttgcccatgattcctgaagcggtggtggccatgttggccattgcaagacttggtgccatccactctgtggtgttcgctggcttctctgctggctctttgaaagaccgtgtggtagatgctggttgtaaagtggtaatcacgtgcgacgaaggtaagagaggcggtaagacagttcacactaaaaagatcgtggacgaaggtttgaacggtatcagccttgtctctcacattcttgtcttccagagaaccgggagcgaaggtatccccatgaccgccggtagggattactggtggcatgaggagaccgccaagcagagaagttacttgcctcctgtgccttgcaattccgaagatccattgttcttgctatacacttctgggtctacgggctcccctaaaggtgttgtccattctaccgccggttaccttttgggtgccgctatgaccaccagatatgtcttcgacatccatccagaagacgttctctttaccgccggtgacgttggctggatcactggccacacctatgctctatatggcccattggttctcggtacggccagtatcatctttgaatctacccctgcctacccagattatggtaggtattggagaattatccagcgtcacaaggcaacacatttctatgtggctcctacagctttgagactcatcaaacgtgttggtgaagctgaaatccccaaatacgacatctcgtcgcttcgtgtgcttgggtctgttggtgagcccatctccccagagctttgggagtggtactatgaaaaagttggtaacaaaaactgtgtcatttgcgatacgatgtggcagacagaatctggctctcatttgatcgcccctcaagctggtgcagttccaacgaaaccaggttccgccactgtacctttctttggtgtggacgcttgcatcatcgatcctgttactggtattgagttgcaaggcaacgatgtggaaggtgtcctagcggtcaaatcttcctggccatcaatggctcgttctgtctggcaaaatcatcaccgttacgtcgacacatatttgaagccatacccaggttattactttacaggtgatggtgccgggagggaccacgatggctactactggattagaggcagagtggacgacgtggtaaatgtctcaggtcacagactttctacagctgagatcgaagcctctttgaccaatcatgataatgtctctgagtctgctgtagtcggcattgctgatgaattgacaggtcagtcagttattgcctttgtctctttgaaggacggttcttccagggaatcttctgccgtcgtagctatgcgtcgcgaattggttctccaggttagaggtgaaattggtcccttcgcagcccctaagtgtgtcattttggtcaaggacttgcccaaaaccagatcaggcaaaattatgagaagagttctaaggaaagtggcctctaacgaagcggaccagttgggtgatctatctaccatggcgaactccgaggttgttccatctatcattgccgctgtagatgaacaattctttgctgagaaaaagaaataa

SEQ ID NO: 58 - DNA, 1773 bp, Acetobacter aceti
  misc_feature (1)..(1773): ACS
atgcttccatggacgacatacgaggcgatgtatgacgcagccctgaaccagccagagcagttctggctggctgcggcacagcgcgtcacatggaagcaggcccctgtgaccgcatgcaggacacggtcggatggctggcatgactggtttcccgacgccacgctcaatacctgccataacgccgtggaccggcatgtggagaatgggcgcggagggcaggcggcattgatctggcattcctgcgccaccagggaacgtcaggttataacctacagggagttgcagagcagggttgccggatttgccggtggtctgcgctcgttgggggtggagaaaggcgagcgtgtcctgatcgccatgccgaccatgatcgagacggtcatcgccatgctggcctgtgcacggatcggcgctgtacatgtcgtggtctttgccggttacgctgggcctgaactggcgcgacggatcgatgatgcggcaccgaaagtcatcatcatcgccagttgcagctttcaggggcagacgcccgttccgtccgtgcccgccctgaacgaggcgctggctgcggcgacgcactgcccacaggcctgcgtgatcgtgcagcgcgaagcgtgcccggtttcgcttctaccggtgcgggatcatgattttcacacgctggaacagtccgcaccagcagagccgctcatgctgcgctccgaagatcccctgtatattcttcacacgtccggcacgacgggcaatgcgaagggcattgtgcgtgacaatggtggccatgctgtcgctctcgccttgtccatggatctgatctacggctgcaaacccggtgataccttcttcacgacatcggatctgggttgggtggtcggccattcctatggcgtctatgcgccgctgatcagcggctgcaccagcgtgattgtggaaggcggtgcttcagcttctgcgatccgcatgctctgtcacgaacacgcagtgaaatgcctgttcaccacaccaacacagatgcggctgatgcgacaggagagtcgccatctgtcaggggcgatactgcccgcgctggcccgaatcttcgtggccggggagtatgctgacccaacattgctggagtggacgcggtcctatttccgcaaacccgtagtcaatcactggtggcagactgaaaccggatggagcatcaccgcgcatttttttggtctgcccgagcgtgagccggtctcgctcatgaatgacatcgggcggcctgcaccgggattctgtccggccattgtgccgtccatagccgatgagcagtatggggagatcgtcctttctttgccgttgccacctggttgtctcgctgggatgtggaaggatggtgctatccgccttccgtccacttatcttgatgaaataggtagatattaccgcacctttgatgaaggtatgatcgaggccaaccgcgccgtgcatatgctcgggcgttctgacgatgttatcaaggtcgcaggccggaggatttccggcgtacagatcgaaaagatcattgccacccatccagccgttcatacctgcgccgtggtcgcgatccccgatgaactgcgaggccagcgacctgtcgcctatgtggtcgttgaccctgaggcctcctgcgaaccatcttctgaggaaatcgtcgtgctggtcaacgaagtcctcgggcgttgggttggtctgaaggaagtccgtttcatcaggcatctaccgaccacggtatctggcaagatcacaaggaaacgtctgctggtgtcctga

SEQ ID NO: 59 - DNA, 1401 bp, Escherichia coli
  misc_feature (1)..(1401): udhA
atgcctcacagttatgactacgacgctattgttataggttcaggaccaggtggagaaggtgcagccatgggattagttaaacaaggggccagagttgcggtgatcgaaagataccagaacgttggaggtggctgtacccattgggggaccatcccttctaaggctctgagacatgctgtcagtagaataatcgagtttaatcaaaatcctctttactcagatcattctcgactactaagatcttcatttgcagacatcctgaatcacgcagataacgtaatcaatcaacaaactaggatgagacaaggtttttacgaacgtaatcattgcgaaattctacaagggaatgctagatttgtggatgaacacactctggcgttagattgtccagacggtagtgtcgaaactcttacagcagaaaaattcgtcatagcctgtggttcaagaccttaccatccaacagatgttgatttcacacatcctagaatctacgactccgattctattctgtcaatgcaccatgaaccaaggcacgtattgatatatggtgctggagtcattggttgtgaatacgcaagcatctttagaggcatggatgttaaagtagacttgattaatacaagagacagactccttgcgtttttagatcaggagatgtctgattccctctcataccacttctggaactctggtgtagtgataagacataacgaggaatacgaaaagattgagggttgcgacgatggtgtaatcatgcatcttaagtctggcaaaaagttgaaagcagattgcttattgtacgctaatggcagaactggcaacacagactctttagcattacaaaatatcggcttggagactgattctcgtgggcaactaaaggttaattcaatgtaccaaacagcccagccacatgtttacgcagttggtgatgttattggctatccaagcttagcatccgcagcttacgatcagggtagaatagctgcccaagccctagttaagggcgaagctacagcacacttaattgaagatatcccaaccggaatctacacaattccagaaatttcctctgtaggaaaaactgaacaacagcttacggctatgaaagtcccttatgaagtgggtagggcccaattcaaacatttggcaagagcccaaatagtcgggatgaacgtgggaacattgaaaatcttgtttcacagagaaactaaagagattttgggcattcattgttttggagaaagagctgctgaaatcatccatattggacaagccatcatggagcaaaagggcggtggtaatactatcgaatacttcgttaacaccacattcaattatccaacgatggctgaggcttatagagtggctgctctaaacggtttgaaccgactgttttaa

SEQ ID NO: 60 - protein, 466 aa, Escherichia coli
  misc_feature (1)..(466): udhA
MPHSYDYDAIVIGSGPGGEGAAMGLVKQGARVAVIERYQNVGGGCTHWGTIPSKALRHAVSRIIEFNQNPLYSDHSRLLRSSFADILNHADNVINQQTRMRQGFYERNHCEILQGNARFVDEHTLALDCPDGSVETLTAEKFVIACGSRPYHPTDVDFTHPRIYDSDSILSMHHEPRHVLIYGAGVIGCEYASIFRGMDVKVDLINTRDRLLAFLDQEMSDSLSYHFWNSGVVIRHNEEYEKIEGCDDGVIMHLKSGKKLKADCLLYANGRTGNTDSLALQNIGLETDSRGQLKVNSMYQTAQPHVYAVGDVIGYPSLASAAYDQGRIAAQALVKGEATAHLIEDIPTGIYTIPEISSVGKTEQQLTAMKVPYEVGRAQFKHLARAQIVGMNVGTLKILFHRETKEILGIHCFGERAAEIIHIGQAIMEQKGGGNTIEYFVNTTFNYPTMAEAYRVAALNGLNRLF

SEQ ID NO: 61 - DNA, 1395 bp, Azotobacter vinelandii
  misc_feature (1)..(1395): codon-optimized sthA
atggcagtgtacaattatgatgtcgttgttataggaacaggtcctgctggagaaggagcagctatgaatgcagtaaaagcaggtagaaaagttgctgttgtggacgaccgaccacaggttggtggtaactgcactcatctagggactattccatctaaagcacttagacattctgtcagacagattatgcaatacaataacaatccattgtttagacaaataggtgaaccaagatggttctctttcgctgatgtgttgaagtcagcggaacaagtgatcgccaagcaagtctcatccagaactgggtattacgcaaggaatagaattgatacctttttcggtacagcttcattttgtgacgagcatacaattgaagtggttcatttgaatgggatggttgaaactcttgttgccaaacagtttgtaatagccactggctccagaccttatagaccagctgatgttgattttacacacccaaggatatacgattcagataccattttatccttagggcatacacctagaaggctcattatctacggagccggtgttataggttgtgaatacgccagcatcttttctggcttaggagtgctcgtcgatctaatcgacaatagagatcaattgttgtcttttctggatgatgaaatctctgactctctatcataccatttgagaaacaataatgtcctaattcgtcacaacgaggagtatgaaagagtcgaaggtctggataacggtgtaatccttcacctgaagagcggtaaaaagatcaaagctgatgcgttcctatggtcaaatggcagaactggtaatactgacaaactaggcttggagaacattggtttaaaggccaacggaagagggcagatccaagtagacgaacactatcgtacagaggtttctaacatatacgcagctggcgatgtaatcggctggccatctttagctagtgccgcctacgaccaagggcgatcagctgctggaagtattaccgaaaatgactcctggagattcgtagatgatgtccctacaggtatctacaccattcctgaaatttcatctgtcggcaagacggaaagagaattaacacaagctaaagttccatacgaagtgggtaaagcattctttaaaggtatggccagagcacaaatcgctgtagagaaggctggaatgctgaagatcttgttccatagagaaacgttagagatactcggcgttcattgttttggttatcaagcaagtgaaatcgttcacattggccaagctattatgaatcaaaaaggtgaagcaaacacattgaaatacttcatcaatactacttttaactacccaacaatggccgaggcttacagagtagcagcgtacgatggacttaatcgtttgttttaa

SEQ ID NO: 62 - protein, 464 aa, Azotobacter vinelandii
  misc_feature (1)..(464): codon-optimized sthA
MAVYNYDVVVIGTGPAGEGAAMNAVKAGRKVAVVDDRPQVGGNCTHLGTIPSKALRHSVRQIMQYNNNPLFRQIGEPRWFSFADVLKSAEQVIAKQVSSRTGYYARNRIDTFFGTASFCDEHTIEVVHLNGMVETLVAKQFVIATGSRPYRPADVDFTHPRIYDSDTILSLGHTPRRLIIYGAGVIGCEYASIFSGLGVLVDLIDNRDQLLSFLDDEISDSLSYHLRNNNVLIRHNEEYERVEGLDNGVILHLKSGKKIKADAFLWSNGRTGNTDKLGLENIGLKANGRGQIQVDEHYRTEVSNIYAAGDVIGWPSLASAAYDQGRSAAGSITENDSWRFVDDVPTGIYTIPEISSVGKTERELTQAKVPYEVGKAFFKGMARAQIAVEKAGMLKILFHRETLEILGVHCFGYQASEIVHIGQAIMNQKGEANTLKYFINTTFNYPTMAEAYRVAAYDGLNRLF